(19) United States
(12) Patent Application Publication
(10) Pub. No.: US 2012/0094343 A1
(43) Pub. Date: Apr. 19, 2012

(54) Gene Knockout Mesophilic and Thermophilic Organisms, and Methods of Use Thereof

(75) Inventors: David A. Hogsett, Grantham, NH (US); Vineet Rajgarhia, Lebanon, NH (US); Arthur J. Shaw, IV, West Lebanon, NH (US); Nicky C. Caiazza, Rancho Santa Fe, CA (US)

(73) Assignee: Mascoma Corporation, Lebanon, NH (US)

(21) Appl. No.: 13/129,085

(22) PCT Filed: Nov. 12, 2009
(86) PCT No.: PCT/US09/64128

§ 371 Date: Jan. 9, 2012

Related U.S. Application Data
(60) Provisional application No. 61/113,978, filed on Nov. 12, 2008.

(51) Int. Cl. C12P 7/56; C12P 7/54; C12N 1/21; C12N 15/11; C12N 15/74

(52) U.S. Cl. 435/139; 536/23.1; 435/320.1; 435/252.3; 435/252.31; 435/140

(57) ABSTRACT

One aspect of the invention relates to a genetically modified thermophilic or mesophilic microorganism, wherein a first native gene is partially, substantially, or completely deleted, silenced, inactivated, or down-regulated, which first native gene encodes a first native enzyme involved in the metabolic production of an organic acid or a salt thereof, thereby increasing the native ability of said thermophilic or mesophilic microorganism to produce lactate or acetate as a fermentation product. In certain embodiments, the aforementioned microorganism further comprises a first non-native gene, which first non-native gene encodes a first non-native enzyme involved in the metabolic production of lactate or acetate. Another aspect of the invention relates to a process for converting lignocellulosic biomass to lactate or acetate, comprising contacting lignocellulosic biomass with a genetically modified thermophilic or mesophilic microorganism.

### BACKGROUND OF THE INVENTION

### Field of the Invention

Energy conversion, utilization and access underlie many of the great challenges of our time, including those associated with sustainability, environmental quality, security, and poverty. New applications of emerging technologies are required to respond to these challenges. Biotechnology, one of the most powerful of the emerging technologies, can give rise to important new energy conversion processes. Plant biomass and derivatives thereof are a resource for the biological conversion of energy to forms useful to humanity.

Among forms of plant biomass, lignocellulosic biomass (“biomass”) is particularly well-suited for energy applications because of its large-scale availability, low cost, and environmentally benign production. In particular, many energy production and utilization cycles based on cellulosic biomass have near-zero greenhouse gas emissions on a life-cycle basis. The primary obstacle impeding the more widespread production of energy from biomass feedstocks is the general absence of low-cost technology for overcoming the recalcitrance of these materials to conversion into useful products. Lignocellulosic biomass contains carbohydrate fractions (e.g., cellulose and hemicellulose) that can be converted into ethanol or other products such as lactic acid and acetic acid. In order to convert these fractions, the cellulose and hemicellulose must ultimately be converted or hydrolyzed into monosaccharides; it is the hydrolysis that has historically proven to be problematic.

Biologically mediated processes are promising for energy conversion. Biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g., glucose, mannose, and galactose); and (4) the fermentation of pentose sugars (e.g., xylose and arabinose). These four transformations occur in a single step in a process configuration called consolidated bioprocessing (CBP), which is distinguished from other less highly integrated configurations in that it does not involve a dedicated process step for cellulase and/or hemicellulase production.

CBP offers the potential for lower cost and higher efficiency than processes featuring dedicated cellulase production. The benefits result in part from avoided capital costs, substrate and other raw materials, and utilities associated with cellulase production. In addition, several factors support the realization of higher rates of hydrolysis, and hence reduced reactor volume and capital investment using CBP, including enzyme-microbe synergy and the use of thermophilic organisms and/or complexed cellulase systems. Moreover, cellulose-adherent cellulolytic microorganisms are likely to compete successfully for products of cellulose hydrolysis with non-adhered microbes, e.g., contaminants, which could increase the stability of industrial processes based on microbial cellulose utilization. Progress in developing CBP-enabling microorganisms is being made through two strategies: engineering naturally occurring cellulolytic microorganisms to improve product-related properties, such as yield and titer; and engineering non-cellulolytic organisms that exhibit high product yields and titers to express a heterologous cellulase and hemicellulase system enabling cellulose and hemicellulose utilization.

Many bacteria have the ability to ferment simple hexose sugars into a mixture of acidic and pH-neutral products via the process of glycolysis. The glycolytic pathway is abundant and comprises a series of enzymatic steps whereby a six carbon glucose molecule is broken down, via multiple intermediates, into two molecules of the three carbon compound pyruvate. This process results in the net generation of ATP (biological energy supply) and the reduced cofactor NADH.

Pyruvate is an important intermediary compound of metabolism. For example, under aerobic conditions pyruvate may be oxidized to acetyl coenzyme A (acetyl CoA), which then enters the tricarboxylic acid cycle (TCA), which in turn generates synthetic precursors, CO₂ and reduced cofactors. The cofactors are then oxidized by donating hydrogen equivalents, via a series of enzymatic steps, to oxygen resulting in the formation of water and ATP. This process of energy formation is known as oxidative phosphorylation.

Under anaerobic conditions (no available oxygen), fermentation occurs in which the degradation products of organic compounds serve as hydrogen donors and acceptors. Excess NADH from glycolysis is oxidized in reactions involving the reduction of organic substrates to products, such as lactate and ethanol. In addition, ATP is regenerated from the production of organic acids, such as acetate, in a process known as substrate level phosphorylation. Therefore, the fermentation products of glycolysis and pyruvate metabolism include a variety of organic acids, alcohols and CO₂.

Most facultative anaerobes metabolize pyruvate aerobically via pyruvate dehydrogenase (PDH) and the tricarboxylic acid cycle (TCA). Under anaerobic conditions, the main energy pathway for the metabolism of pyruvate is via pyruvate-formate-lyase (PFL) pathway to give formate and acetyl-CoA. Acetyl-CoA is then converted to acetate, via phosphotransacetylase (PTA) and acetate kinase (ACK) with the co-production of ATP, or reduced to ethanol via acetalaldehyde dehydrogenase (AcDH) and alcohol dehydrogenase (ADH). In order to maintain a balance of reducing equivalents, excess NADH produced from glycolysis is re-oxidized to NAD⁺ by lactate dehydrogenase (LDH) during the reduction of pyruvate to lactate. NADH can also be re-oxidized by AcDH and ADH during the reduction of acetyl-CoA to ethanol, but this is a minor reaction in cells with a functional LDH. A simplified version of the central metabolic pathway leading to mixed fermentation end products in, for example, cellulolytic clostridia, is presented in FIG. 54.

Metabolic engineering of microorganisms could, for example, result in the creation of a targeted knockout of the genes encoding for the production of enzymes, such as lactate dehydrogenase. In this case, “knock out” of the genes means partial, substantial, or complete deletion, silencing, inactivation, or down-regulation. If the conversion of pyruvate to lactate (the salt form of lactic acid) by the action of LDH was not available in the early stages of the glycolytic pathway, then the pyruvate could be more efficiently converted to acetyl CoA by the action of pyruvate dehydrogenase or pyruvate-ferredoxin oxidoreductase. If the further conversion of acetyl CoA to ethanol by alcohol or aldeyhyde dehydrogenase was also not available, i.e., if the genes encoding for the production of ADH was knocked out, then the acetyl CoA could be more efficiently converted to acetate (the salt form of acetic acid). On the other hand, if the action of LDH was maintained, and the further conversion of acetylCoA to either acetate or ethanol was not available, i.e., if the genes encoding for the production of PTA and ACK were knocked out, and/or the gene encoding for the production of ADH was knocked out, then the pyruvate could be more efficiently converted to lactate by LDH. Accordingly, a genetically modified strain of microorganism with such targeted gene knockouts, which eliminates the production of certain organic acids, would have an increased ability to produce lactate or acetate as a fermentation product.

Ethanologenic organisms, such as Zymomonas mobilis, Zymobacter palmae, Acetobacter pasteurianus, or Sarcina ventriculi, and some yeasts (e.g., Saccharomyces cerevisiae), are capable of a second type of anaerobic fermentation, commonly referred to as alcoholic fermentation, in which pyruvate is metabolized to acetaldehyde and CO₂ by pyruvate decarboxylase (PDC). Acetaldehyde is then reduced to ethanol by ADH regenerating NAD⁺. Alcoholic fermentation results in the metabolism of one molecule of glucose to two molecules of ethanol and two molecules of CO₂. If the conversion of pyruvate to undesired organic acids could be avoided, as detailed above, then such a genetically modified microorganism would have an increased ability to produce lactate or acetate as a fermentation product.

The generation of higher yields of lactic and/or acetic acid has certain advantages. For example, lactic acid can be used as a preservative, acidulant, and flavor in food, textile, and pharmaceutical industries. It has also been increasing in importance as a feedstock for the manufacture of polylactic acid (PLA), which could be a good substitute for synthetic plastic derived from petroleum feedstock. While the chemical synthesis of lactic acid always leads to a racemic mixture, a major disadvantage, fermentative production of lactic acid offers great advantage in producing optically pure l- or d-lactic and also dl-lactic acid, depending on the strain selected for fermentation. Acetic acid is an important chemical reagent and industrial chemical that is used in the production of polyethylene terephthalate, cellulose acetate, and polyvinyl acetate. Acetic acid is produced both synthetically and by bacterial fermentation. Today, the biological route accounts for only about 10% of world production, but it remains important for vinegar production, as the world food purity laws stipulate that vinegar used in foods must be of biological origin.

### BRIEF SUMMARY OF THE INVENTION

One aspect of the invention relates to an isolated nucleic acid molecule comprising the nucleotide sequence of any one of SEQ ID NOS:1-5, 30-31, 47-61, 79-83, 85-86, 88-89, 96-97, 99, 101, or 103, or a complement thereof. Another aspect of the invention relates to an isolated nucleic acid molecule comprising a nucleotide sequence which shares at least 80% identity to a nucleotide sequence of any one of SEQ ID NOS:1-5, 30-31, and 47-61, 79-83, 85-86, 88-89, 96-97, 99, 101, or 103, or a complement thereof. In certain embodiments, the invention relates to the aforementioned nucleic acid molecule which shares at least about 95% sequence identity to the nucleotide sequence of any one of SEQ ID NOS:1-5, 30-31, 47-61, 79-83, 85-86, 88-89, 96-97, 99, 101, or 103, or a complement thereof.

Another aspect of the present invention relates to a genetic construct comprising any one of SEQ ID NOS:1-5, 30-31 47-61, 79-83, 85-86, 88-89, 96-97, 99, 101, or 103, operably linked to a promoter expressible in a thermophilic or mesophilic bacterium. The present invention also relates to a recombinant thermophilic or mesophilic bacterium comprising the aforementioned genetic construct.

The present invention also encompasses a vector comprising any one of the aforementioned nucleic acid molecules. The present invention also encompasses a host cell comprising any one of the aforementioned nucleic acid molecules. In certain embodiments, the invention relates to the aforementioned host cell, wherein said host cell is a thermophilic or mesophilic bacterial cell.

Another aspect of the invention relates to a genetically modified thermophilic or mesophilic microorganism, wherein a first native gene is partially, substantially, or completely deleted, silenced, inactivated, or down-regulated, which first native gene encodes a first native enzyme involved in the metabolic production of an organic acid or a salt thereof, thereby increasing the native ability of said thermophilic or mesophilic microorganism to produce lactate or acetate as a fermentation product. In certain embodiments, the present invention relates to the aforementioned genetically modified microorganism, wherein said microorganism is a Gram-negative bacterium or a Gram-positive bacterium. In certain embodiments, the present invention relates to the aforementioned genetically modified microorganism, wherein said microorganism is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus. In certain embodiments, the present invention relates to the aforementioned genetically modified microorganism, wherein said microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellum thermophilum. In certain embodiments, the present invention relates to the aforementioned genetically modified microorganism, wherein said microorganism is Clostridium thermocellum, Clostridium cellulolyticum, or Thermoanaerobacterium saccharolyticum.

In certain embodiments, the present invention relates to the aforementioned genetically modified microorganism, wherein a first non-native gene is inserted, which first non-native gene encodes a first non-native enzyme that confers the ability to metabolize a hexose sugar, thereby allowing said thermophilic or mesophilic microorganism to produce ethanol as a fermentation product from a hexose sugar. In certain embodiments, the present invention relates to the aforementioned genetically modified microorganism, wherein said organic acid is selected from the group consisting of lactic acid, acetic acid, or ethanol. In certain embodiments, the present invention relates to the aforementioned genetically modified microorganism, wherein said organic acid is lactic acid. In certain embodiments, the present invention relates to the aforementioned genetically modified microorganism, wherein said organic acid is acetic acid. In certain embodiments, the present invention relates to the aforementioned genetically modified microorganism, wherein said organic acid is ethanol. In certain embodiments, the present invention relates to the aforementioned genetically modified microorganism, wherein said first native enzyme is selected from the group consisting of lactate dehydrogenase, acetate kinase, phosphotransacetylase, pyruvate formate lyase, aldehyde dehydrogenase, and alcohol dehydrogenase. In certain embodiments, the present invention relates to the aforementioned genetically modified microorganism, wherein said first native enzyme is lactate dehydrogenase. In certain embodiments, the present invention relates to the aforementioned genetically modified microorganism, wherein said first native enzyme is acetate kinase. In certain embodiments, the present invention relates to the aforementioned genetically modified microorganism, wherein said first native enzyme is phosphotransacetylase. In certain embodiments, the present invention relates to the aforementioned genetically modified microorganism, wherein said first native enzyme is pyruvate formate lyase. In certain embodiments, the present invention relates to the aforementioned genetically modified microorganism, wherein said first native enzyme is aldehyde dehydrogenase or alcohol dehydrogenase

In certain embodiments, the present invention relates to the aforementioned genetically modified microorganism, wherein a second native gene is partially, substantially, or completely deleted, silenced, inactivated, or down-regulated, which second native gene encodes a second native enzyme involved in the metabolic production of an organic acid or a salt thereof. In certain embodiments, the present invention relates to the aforementioned genetically modified microorganism, wherein said second native enzyme is acetate kinase or phosphotransacetylase. In certain embodiments, the present invention relates to the aforementioned genetically modified microorganism, wherein said second native enzyme is lactate dehydrogenase. In certain embodiments, the present invention relates to the aforementioned genetically modified microorganism, wherein said second native enzyme is lactate pyruvate formate lyase. In certain embodiments, the present invention relates to the aforementioned genetically modified microorganism, wherein said second native enzyme is aldehyde deydrogenase or alcohol dehydrogenase.

Yet another aspect of the invention relates to a genetically modified thermophilic or mesophilic microorganism, wherein (a) a first native gene is partially, substantially, or completely deleted, silenced, inactivated, or down-regulated, which first native gene encodes a first native enzyme involved in the metabolic production of an organic acid or a salt thereof, and (b) a first non-native gene is inserted, which first non-native gene encodes a first non-native enzyme involved in the metabolic production of lactate or acetate, thereby allowing said thermophilic or mesophilic microorganism to produce lactate or acetate as a fermentation product.

In certain embodiments, the present invention relates to the aforementioned genetically modified microorganism, wherein said first non-native gene encodes a first non-native enzyme that confers the ability to metabolize a hexose sugar, thereby allowing said thermophilic or mesophilic microorganism to metabolize a hexose sugar. In certain embodiments, the present invention relates to the aforementioned genetically modified microorganism, wherein said first non-native gene encodes a first non-native enzyme that confers the ability to metabolize a pentose sugar, thereby allowing said thermophilic or mesophilic microorganism to metabolize a pentose sugar. In certain embodiments, the present invention relates to the aforementioned genetically modified microorganism, wherein said first non-native gene encodes a first non-native enzyme that confers the ability to metabolize a hexose sugar; and a second non-native gene is inserted, which second non-native gene encodes a second non-native enzyme that confers the ability to metabolize a pentose sugar, thereby allowing said thermophilic or mesophilic microorganism to metabolize a hexose sugar and a pentose sugar.

In certain embodiments, the present invention relates to the aforementioned genetically modified microorganism, wherein said organic acid is lactic acid. In certain embodiments, the present invention relates to the aforementioned genetically modified microorganism, wherein said organic acid is acetic acid. In certain embodiments, the present invention relates to the aforementioned genetically modified microorganism, wherein said organic acid is ethanol. In certain embodiments, the present invention relates to the aforementioned genetically modified microorganism, wherein said first non-native enzyme is pyruvate decarboxylase (PDC), lactate dehydrogenase, acetate kinase, phosphotransacetylase, pyruvate formate lyase, aldehyde dehydrogenase, and alcohol dehydrogenase.

In certain embodiments, the present invention relates to the aforementioned genetically modified microorganism, wherein said second non-native enzyme is xylose isomerase. In certain embodiments, the present invention relates to the aforementioned genetically modified microorganism, wherein said first non-native gene corresponds to SEQ ID NOS:6, 10, or 14. In certain embodiments, the present invention relates to the aforementioned genetically modified microorganism, wherein said non-native enzyme is xylulokinase. In certain embodiments, the present invention relates to the aforementioned genetically modified microorganism, wherein said non-native gene corresponds to SEQ ID NOS:7, 11, or 15. In certain embodiments, the present invention relates to the aforementioned genetically modified microorganism, wherein said non-native enzyme is L-arabinose isomerase. In certain embodiments, the present invention relates to the aforementioned genetically modified microorganism, wherein said non-native gene corresponds to SEQ ID NOS:8 or 12. In certain embodiments, the present invention relates to the aforementioned genetically modified microorganism, wherein said non-native enzyme is L-ribulose-5-phosphate 4-epimerase. In certain embodiments, the present invention relates to the aforementioned genetically modified microorganism, wherein said non-native gene corresponds to SEQ ID NO:9 or 13. In certain embodiments, the present invention relates to the aforementioned genetically modified microorganism, wherein said microorganism is able to convert at least 60% of carbon from metabolized biomass into ethanol.

In certain embodiments, the present invention relates to the aforementioned genetically modified microorganism, wherein said microorganism is selected from the group consisting of: (a) a thermophilic or mesophilic microorganism with a native ability to hydrolyze cellulose; (b) a thermophilic or mesophilic microorganism with a native ability to hydrolyze xylan; and (c) a thermophilic or mesophilic microorganism with a native ability to hydrolyze cellulose and xylan. In certain embodiments, the present invention relates to the aforementioned genetically modified microorganism, wherein said microorganism has a native ability to hydrolyze cellulose. In certain embodiments, the present invention relates to the aforementioned genetically modified microorganism, wherein said microorganism has a native ability to hydrolyze cellulose and xylan. In certain embodiments, the present invention relates to the aforementioned genetically modified microorganism, wherein a first non-native gene is inserted, which first non-native gene encodes a first non-native enzyme that confers the ability to hydrolyze xylan. In certain embodiments, the present invention relates to the aforementioned genetically modified microorganism, wherein said microorganism has a native ability to hydrolyze xylan. In certain embodiments, the present invention relates to the aforementioned genetically modified microorganism, wherein a first non-native gene is inserted, which first non-native gene encodes a first non-native enzyme that confers the ability to hydrolyze cellulose. In certain embodiments, the present invention relates to the aforementioned genetically modified microorganism, wherein said organic acid is selected from the group consisting of lactic acid, acetic acid, and ethanol. In certain embodiments, the present invention relates to the aforementioned genetically modified microorganism, wherein said organic acid is lactic acid. In certain embodiments, the present invention relates to the aforementioned genetically modified microorganism, wherein said organic acid is acetic acid. In certain embodiments, the present invention relates to the aforementioned genetically modified microorganism, wherein said organic acid is ethanol.

In certain embodiments, the present invention relates to the aforementioned genetically modified microorganism, wherein said first native enzyme is selected from the group consisting of lactate dehydrogenase, acetate kinase, phosphotransacetylase, pyruvate formate lyase, aldehyde dehydrogenase, and alcohol dehydrogenase. In certain embodiments, the present invention relates to the aforementioned genetically modified microorganism, wherein said first native enzyme is lactate dehydrogenase. In certain embodiments, the present invention relates to the aforementioned genetically modified microorganism, wherein said first native enzyme is acetate kinase. In certain embodiments, the present invention relates to the aforementioned genetically modified microorganism, wherein said first native enzyme is phosphotransacetylase. In certain embodiments, the present invention relates to the aforementioned genetically modified microorganism, wherein said first native enzyme is pyruvate formate lyase. In certain embodiments, the present invention relates to the aforementioned genetically modified microorganism, wherein said first native enzyme is aldehyde dehydrogenase or alcohol dehydrogenase.

In certain embodiments, the present invention relates to the aforementioned genetically modified microorganism, wherein a second native gene is partially, substantially, or completely deleted, silenced, inactivated, or down-regulated, which second native gene encodes a second native enzyme involved in the metabolic production of an organic acid or a salt thereof. In certain embodiments, the present invention relates to the aforementioned genetically modified microorganism, wherein said second native enzyme is acetate kinase or phosphotransacetylase. In certain embodiments, the present invention relates to the aforementioned genetically modified microorganism, wherein said second native enzyme is lactate dehydrogenase. In certain embodiments, the present invention relates to the aforementioned genetically modified microorganism, wherein said second native enzyme is lactate pyruvate formate lyase. In certain embodiments, the present invention relates to the aforementioned genetically modified microorganism, wherein said second native enzyme is aldehyde deydrogenase or alcohol dehydrogenase.

In certain embodiments, the present invention relates to the aforementioned genetically modified microorganism, wherein (a) a first native gene is partially, substantially, or completely deleted, silenced, inactivated, or down-regulated, which first native gene encodes a first native enzyme involved in the metabolic production of an organic acid or a salt thereof, and (b) a first non-native gene is inserted, which first non-native gene encodes a first non-native enzyme involved in the hydrolysis of a polysaccharide, thereby allowing said thermophilic or mesophilic microorganism to produce ethanol as a fermentation product. In certain embodiments, the present invention relates to the aforementioned genetically modified microorganism, wherein said first non-native gene encodes a first non-native enzyme that confers the ability to hydrolyze cellulose, thereby allowing said thermophilic or mesophilic microorganism to hydrolyze cellulose. In certain embodiments, the present invention relates to the aforementioned genetically modified microorganism, wherein said first non-native gene encodes a first non-native enzyme that confers the ability to hydrolyze xylan, thereby allowing said thermophilic or mesophilic microorganism to hydrolyze xylan. In certain embodiments, the present invention relates to the aforementioned genetically modified microorganism, wherein said first non-native gene encodes a first non-native enzyme that confers the ability to hydrolyze cellulose; and a second non-native gene is inserted, which second non-native gene encodes a second non-native enzyme that confers the ability to hydrolyze xylan, thereby allowing said thermophilic or mesophilic microorganism to hydrolyze cellulose and xylan. In certain embodiments, the present invention relates to the aforementioned genetically modified microorganism, wherein said organic acid is lactic acid. In certain embodiments, the present invention relates to the aforementioned genetically modified microorganism, wherein said organic acid is acetic acid. In certain embodiments, the present invention relates to the aforementioned genetically modified microorganism, wherein said organic acid is ethanol. In certain embodiments, the present invention relates to the aforementioned genetically modified microorganism, wherein said first non-native enzyme is pyruvate decarboxylase (PDC), lactate dehydrogenase, acetate kinase, phosphotransacetylase, pyruvate formate lyase, aldehyde dehydrogenase, and alcohol dehydrogenase. In certain embodiments, the present invention relates to the aforementioned genetically modified microorganism, wherein said microorganism is able to convert at least 60% of carbon from metabolized biomass into lactate or acetate.

In certain embodiments, the present invention relates to any of the aforementioned genetically modified microorganisms, wherein said microorganism is mesophilic. In certain embodiments, the present invention relates to any of the aforementioned genetically modified microorganisms, wherein said microorganism is thermophilic.

Another aspect of the invention relates to a process for converting lignocellulosic biomass to lactate or acetate, comprising contacting lignocellulosic biomass with any one of the aforementioned genetically modified thermophilic or mesophilic microorganisms. In certain embodiments, the present invention relates to the aforementioned process, wherein said lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof. In certain embodiments, the present invention relates to the aforementioned process, wherein said lignocellulosic biomass is selected from the group consisting of corn stover, sugarcane bagasse, switchgrass, and poplar wood. In certain embodiments, the present invention relates to the aforementioned process, wherein said lignocellulosic biomass is corn stover. In certain embodiments, the present invention relates to the aforementioned process, wherein said lignocellulosic biomass is sugarcane bagasse. In certain embodiments, the present invention relates to the aforementioned process, wherein said lignocellulosic biomass is switchgrass. In certain embodiments, the present invention relates to the aforementioned process, wherein said lignocellulosic biomass is poplar wood. In certain embodiments, the present invention relates to the aforementioned process, wherein said lignocellulosic biomass is willow. In certain embodiments, the present invention relates to the aforementioned process, wherein said lignocellulosic biomass is paper sludge.

### BRIEF DESCRIPTION OF THE DRAWINGS/FIGURES

FIG. 1 depicts the glycolysis pathway.

FIG. 2 depicts pentose and glucuronate interconversions and highlights the enzymes, xylose isomerase (XI or 5.3.1.5) and xylulokinase (XK or 2.7.1.17), in the D-xylose to ethanol pathway.

FIG. 3 depicts pentose and glucuronate interconversions and highlights the enzymes, L-arabinose isomerase (5.3.1.4) and L-ribulose-5-phosphate 4-epimerase (5.1.3.4), in the L-arabinose utilization pathway.

FIG. 4 depicts pentose and glucuronate interconversions and shows that the genes for xylose isomerase, xylulokinase, L-arabinose isomerase, and L-ribulose-5-phosphate 4-epimerase are present in C. cellulolyticum.

FIG. 5 depicts pentose and glucuronate interconversions and shows that xylose isomerase and xylulokinase are present, while L-arabinose isomerase and L-ribulose-5-phosphate 4-epimerase are absent in C. phytofermentans.

FIG. 6 shows an alignment of Clostridium thermocellum (SEQ ID NO: 114), Clostridium cellulolyticum (SEQ ID NO: 115), Thermoanaerobacterium saccharolyticum (SEQ ID NO: 116), C. stercorarium (SEQ ID NO: 117), C. stercorarium II (SEQ ID NO: 118), Caldiscellulosiruptor kristjanssonii (SEQ ID NO: 119), C. phytofermentans (SEQ ID NO: 120), indicating about 73-89% homology at the level of the 16S rDNA gene.

FIG. 7 shows the construction of a double crossover knockout vector for inactivation of the ack gene in Clostridium thermocellum based on the plasmid pIKM1.

FIG. 8 shows the construction of a double crossover knockout vector for inactivation of the ack gene in Clostridium thermocellum based on the replicative plasmid pNW33N.

FIG. 9 shows the construction of a double crossover knockout vector for inactivation of the ldh gene in Clostridium thermocellum based on the plasmid pIKM1.

FIG. 10 shows the construction of a double crossover knockout vector for inactivation of the ldh gene in Clostridium thermocellum based on the replicative plasmid vector pNW33N.

FIG. 11 shows the construction of a double crossover suicide vector for inactivation of the ldh gene in Clostridium thermocellum based on the plasmid pUC19.

FIGS. 12A and 12B show product formation and OD₆₀₀ for C. straminisolvens grown on cellobiose and Avicel®, respectively.

FIGS. 13A and 1313 show product formation and OD₆₀₀ for C. thermocellum grown on cellobiose and Avicel®, respectively.

FIGS. 14A and 14B show product formation and OD₆₀₀ for C. cellulolyticum grown on cellobiose and Avicel®, respectively.

FIGS. 15A and 15B show product formation and OD₆₀₀ for C. stercorarium subs. leptospartum grown on cellobiose and Avicel®, respectively.

FIGS. 16A and 16B show product formation and OD₆₀₀ for Caldicellulosiruptor kristjanssonii grown on cellobiose and Avicel®, respectively.

FIGS. 17A and 17B show product formation and OD₆₀₀ for Clostridium phytofermentans grown on cellobiose and Avicel®, respectively.

FIG. 18 shows total metabolic byproducts after 48 hours of fermentation of 2.5 g/L xylan and 2.5 g/L cellobiose.

FIG. 19 shows a map of the ack gene and the region amplified by PCR for gene disruption.

FIG. 20 shows a map of the ldh 2262 gene and the region amplified by PCR for gene disruption.

FIG. 21 shows an example of C. cellulolyticum (C. cell.) ldh (2262) double crossover knockout fragment.

FIG. 22 shows a map of the ack gene of Clostridium phytofermentans and the region amplified by PCR for gene disruption.

FIG. 23 shows an example of a putative double crossover knockout construct with the mLs gene as a selectable marker in Clostridium phytofermentans.

FIG. 24 shows a map of the ldh 1389 gene and the region amplified by PCR for gene disruption.

FIG. 25 shows an example of a putative double crossover knockout construct with the mLs gene as a selectable marker.

FIG. 26 is a diagram representing by 250-550 of pMOD™-2<MCS>.

FIG. 27 shows the product concentration profiles for 1% Avicel® using C. straminisolvens. The ethanol-to-acetate ratio is depicted as E/A and the ratio of ethanol-to-total products is depicted as E/T.

FIG. 28 shows an example of a vector for retargeting the L1.LtrB intron to insert in C. cell. ACK gene (SEQ ID NO:21).

FIG. 29 shows an example of vector for retargeting the L1.LtrB intron to insert in C. cell. LDH2744 gene (SEQ ID NO:23).

FIG. 30 shows an alignment of T. pseudoethanolicus 39E (SEQ ID NO: 122), T. sp strain 59 (SEQ ID NO: 123), T. saccharolyticum B6A-RI (SEQ ID NO: 124), T. saccharolyticum YS485 (SEQ ID NO: 125) and consensus (SEQ ID NO: 126) at the level of the 16S rDNA gene.

FIG. 31 shows an alignment of T. sp. strain 59 (SEQ ID NO: 36), T. pseudoethanolicus (SEQ ID NO: 35), T. saccharolyticum B6A-RI (SEQ ID NO: 38), T. saccharolyticum YS485 (SEQ ID NO: 32) and consensus (SEQ ID NO: 127) at the level of the pta gene.

FIG. 32 shows an alignment of T. sp. strain 59 (SEQ ID NO: 37), T. pseudoethanolicus (SEQ ID NO: 34), T. saccharolyticum B6A-RI (SEQ ID NO: 39), T. saccharolyticum YS485 (SEQ ID NO: 33) and consensus (SEQ ID NO: 128) at the level of the ack gene.

FIG. 33 shows an alignment of T. sp. strain 59 (SEQ ID NO: 41), T. pseudoethanolicus 39E (SEQ ID NO: 42), T. saccharolyticum B6A-RI (SEQ ID NO: 43), T. saccharolyticum YS485 (SEQ ID NO: 40) and consensus (SEQ ID NO: 129) at the level of the ldh gene.

FIG. 34 shows a schematic of the glycolysis/fermentation pathway.

FIG. 35 shows an example of a pMU340 plasmid.

FIG. 36 shows an example of a pMU102 Z. mobilis PDC-ADH plasmid.

FIG. 37 shows an example of a pMU102 Z. palmae PDC, Z. mobilis ADH plasmid.

FIG. 38 shows the plasmid map of pMU360. The DNA sequence of pMU360 is set forth as SEQ ID NO:61.

FIG. 39 shows the lactate levels in nine colonies of thiamphenicol-resistant transformants.

FIG. 40 shows an example of a T. sacch. pfl KO single crossover plasmid (SEQ ID NO:47).

FIG. 41 shows an example of a T. sacch. pfl KO double crossover plasmid (SEQ ID NO:48).

FIG. 42 shows an example of a C. therm. pfl KO single crossover plasmid (SEQ ID NO:49).

FIG. 43 shows an example of a C. therm. pfl KO double crossover plasmid (SEQ ID NO:50).

FIG. 44 shows an example of a C. phyto. pfl KO single crossover plasmid (SEQ ID NO:51).

FIG. 45 shows an example of a C. phyto. pfl KO double crossover plasmid (SEQ ID NO:52).

FIG. 46 shows an example of a T. sacch. #59 L-ldh KO single crossover plasmid (SEQ ID NO:53).

FIG. 47 shows an example of a T. sacch. #59 L-ldh KO double crossover plasmid (SEQ ID NO:54).

FIG. 48 shows an example of a T. sacch. #59 pta/ack KO single crossover plasmid (SEQ ID NO:55).

FIG. 49 shows an example of a T. sacch. #59 pta/ack KO double crossover plasmid (SEQ ID NO:56).

FIG. 50 shows an example of a T. pseudo. L-ldh KO single crossover plasmid (SEQ ID NO:57).

FIG. 51 shows an example of a T. pseudo. L-ldh KO double crossover plasmid (SEQ ID NO:58).

FIG. 52 shows an example of a T. pseudo. ack KO single crossover plasmid (SEQ ID NO:59).

FIG. 53 shows an example of a T. pseudo. pta/ack KO double crossover plasmid (SEQ ID NO:60).

FIG. 54 shows a schematic of a simplified version of central metabolic pathways leading to mixed acid fermentation end products of cellulolytic clostridia.

FIG. 55 shows an example of a single crossover knockout plasmid of pta in C. thermocellum.

FIG. 56 shows an example of a single crossover knockout plasmid of ack in C. thermocellum.

FIG. 57 shows an example of a double crossover knockout plasmid of pta in C. thermocellum.

FIG. 58 shows an example of a double crossover knockout plasmid of ack in C. thermocellum.

FIG. 59 shows an example of a double crossover knockout plasmid of pta-ack in C. thermocellum.

FIG. 60 shows an example of a single crossover knockout plasmid of ldh in C. thermocellum.

FIG. 61 shows an example of a double crossover knockout plasmid of ldh in C. thermocellum.

FIG. 62 shows an example of a single crossover knockout plasmid of adhE in C. thermocellum.

FIG. 63 shows an example of a double crossover knockout plasmid of adhE in C. thermocellum.

FIG. 64 shows an example of a TargeTron plasmid of pfl 2064 in C. cellulolyticum.

FIG. 65 shows an example of a TargeTron plasmid of pfl 2216 in C. cellulolyticum.

FIG. 66 shows an example of a TargeTron plasmid of pta in C. cellulolyticum.

FIG. 67 shows an example of a TargeTron plasmid of ldh 2262 in C. cellulolyticum.

FIG. 68 shows an example of a TargeTron plasmid of adhE 873 in C. cellulolyticum.

FIG. 69 shows an example of a double crossover knockout plasmid of AdhE in T. saccharolyticum.

FIG. 70 shows an example of a single crossover knockout plasmid of AdhE in T. saccharolyticum.

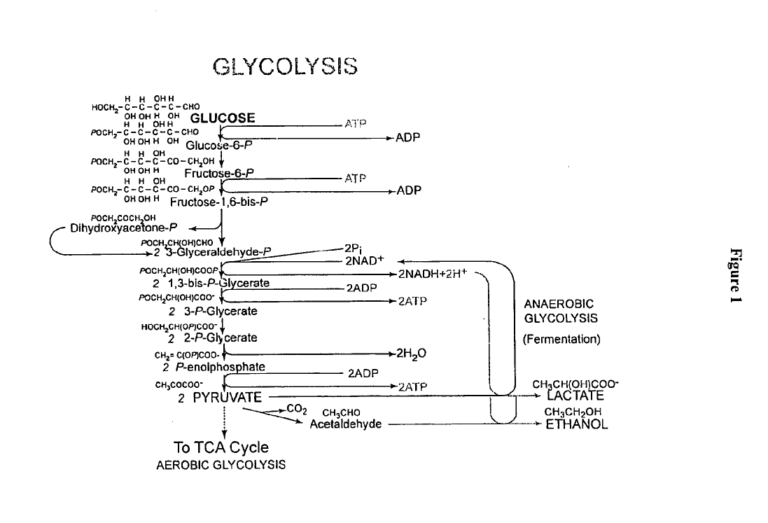

### BRIEF DESCRIPTION OF THE DRAWINGS/FIGURES

Table 1 summarizes representative highly cellulolytic organisms.

Table 2 summarizes representative native cellulolytic and xylanolytic organisms.

Table 3 shows a categorization of bacterial strains based on their substrate utilization.

Table 4 shows insertion location and primers to retarget Intron to C. cellulolyticum acetate kinase.

Table 5 shows insertion location and primers to retarget Intron to C. cellulolyticum lactate dehydrogenase.

Table 6 shows fermentation performance of engineered Thermoanaerobacter and Thermoanaerobacterium strains.

Table 7 shows representative genes involved in the fermentation pathway of C. thermocellum and C. celluloyticum.

### DETAILED DESCRIPTION OF THE INVENTION

Aspects of the present invention relate to the engineering of thermophilic or mesophilic microorganisms for use in the production of lactate or acetate from lignocellulosic biomass. The use of thermophilic bacteria for lactate or acetate production offers many advantages over traditional processes based upon mesophilic ethanol producers. For example, the use of thermophilic organisms provides significant economic savings over traditional process methods due to lower lactate or acetate separation costs, reduced requirements for external enzyme addition, and reduced processing times.

Aspects of the present invention relate to a process by which the cost of lactate or acetate production from cellulosic biomass-containing materials can be reduced by using a novel processing configuration. In particular, the present invention provides numerous methods for increasing lactate or acetate production in a genetically modified microorganism.

In certain other embodiments, the present invention relates to genetically modified thermophilic or mesophilic microorganisms, wherein a gene or a particular polynucleotide sequence is partially, substantially, or completely deleted, silenced, inactivated, or down-regulated, which gene or polynucleotide sequence encodes for an enzyme that confers upon the microorganism the ability to produce organic acids as fermentation products, thereby increasing the ability of the microorganism to produce lactate or acetate as the major fermentation product. Further, by virtue of a novel integration of processing steps, commonly known as consolidated bioprocessing, aspects of the present invention provide for more efficient production of lactate or acetate from cellulosic-biomass-containing raw materials. The incorporation of genetically modified thermophilic or mesophilic microorganisms in the processing of said materials allows for fermentation steps to be conducted at higher temperatures, improving process economics. For example, reaction kinetics are typically proportional to temperature, so higher temperatures are generally associated with increases in the overall rate of production. Additionally, higher temperature facilitates the removal of volatile products from the broth and reduces the need for cooling after pretreatment.

In certain embodiments, the present invention relates to genetically modified or recombinant thermophilic or mesophilic microorganisms with increased ability to produce enzymes that confer the ability to produce lactate or acetate as a fermentation product, the presence of which enzyme(s) modify the process of metabolizing lignocellulosic biomass materials to produce lactate or acetate as the major fermentation product. In one aspect of the invention, one or more non-native genes are inserted into a genetically modified thermophilic or mesophilic microorganism, wherein said non-native gene encodes an enzyme involved in the metabolic production of lactate or acetate, for example, such enzyme may confer the ability to metabolize a pentose sugar and/or a hexose sugar. For example, in one embodiment, the enzyme may be involved in the D-xylose or L-arabinose pathway, thereby allowing the microorganism to metabolize a pentose sugar, i.e., D-xylose or L-arabinose. By inserting (e.g., introducing or adding) a non-native gene that encodes an enzyme involved in the metabolism or utilization of D-xylose or L-arabinose, the microorganism has an increased ability to produce lactate or acetate relative to the native organism.

The present invention also provides novel compositions that may be integrated into the microorganisms of the invention. In one embodiment, an isolated nucleic acid molecule of the invention comprises a nucleic acid molecule which is a complement of a nucleotide sequence shown in any one of SEQ ID NOS:1-106. In another embodiment, an isolated nucleic acid molecule of the invention comprises a nucleic acid molecule which is a complement of a nucleotide sequence shown in any one of SEQ ID NOS:1-106, or a portion of any of these nucleotide sequences. A nucleic acid molecule which is complementary to a nucleotide sequence shown in any one of SEQ ID NOS:1-106, or the coding region thereof, is one which is sufficiently complementary to a nucleotide sequence shown in any one of SEQ ID NOS:1-106, or the coding region thereof, such that it can hybridize to a nucleotide sequence shown in any one of SEQ ID NOS:1-106, or the coding region thereof, thereby forming a stable duplex.

In still another preferred embodiment, an isolated nucleic acid molecule of the present invention comprises a nucleotide sequence which is at least about 50%, 54%, 55%, 60%, 62%, 65%, 70%, 75%, 78%, 80%, 85%, 86%, 87%, 88%, 89%, 90%, 91%, 92%, 93%, 94%, 95%, 96%, 97%, 98%, 99%, or more homologous to the nucleotide sequences (e.g., to the entire length of the nucleotide sequence) shown in any one of SEQ ID NOS:1-106, or a portion of any of these nucleotide sequences.

Moreover, the nucleic acid molecules of the invention may comprise only a portion of the nucleic acid sequence of any one of SEQ ID NOS:1-106, or the coding region thereof; for example, the nucleic acid molecule may be a fragment which can be used as a probe or primer or a fragment encoding a biologically active portion of a protein. In another embodiment, the nucleic acid molecules may comprise at least about 12 or 15, preferably about 20 or 25, more preferably about 30, 35, 40, 45, 50, 55, 60, 65, or 75 consecutive nucleotides of any one of SEQ ID NOS:1-106.

### DEFINITIONS

The term “heterologous polynucleotide segment” is intended to include a polynucleotide segment that encodes one or more polypeptides or portions or fragments of polypeptides. A heterologous polynucleotide segment may be derived from any source, e.g., eukaryotes, prokaryotes, viruses, or synthetic polynucleotide fragments.

The terms “promoter” or “surrogate promoter” is intended to include a polynucleotide segment that can transcriptionally control a gene-of-interest that it does not transcriptionally control in nature. In certain embodiments, the transcriptional control of a surrogate promoter results in an increase in expression of the gene-of-interest. In certain embodiments, a surrogate promoter is placed 5′ to the gene-of-interest. A surrogate promoter may be used to replace the natural promoter, or may be used in addition to the natural promoter. A surrogate promoter may be endogenous with regard to the host cell in which it is used, or it may be a heterologous polynucleotide sequence introduced into the host cell, e.g., exogenous with regard to the host cell in which it is used.

The terms “gene(s)” or “polynucleotide segment” or “polynucleotide sequence(s)” are intended to include nucleic acid molecules, e.g., polynucleotides which include an open reading frame encoding a polypeptide, and can further include non-coding regulatory sequences, and introns. In addition, the terms are intended to include one or more genes that map to a functional locus. In addition, the terms are intended to include a specific gene for a selected purpose. The gene may be endogenous to the host cell or may be recombinantly introduced into the host cell, e.g., as a plasmid maintained episomally or a plasmid (or fragment thereof) that is stably integrated into the genome. In addition to the plasmid form, a gene may, for example, be in the form of linear DNA. In certain embodiments, the gene of polynucleotide segment is involved in at least one step in the bioconversion of a carbohydrate to ethanol, acetate, or lactate. Accordingly, the term is intended to include any gene encoding a polypeptide, such as the enzymes acetate kinase (ACK), phosphotransacetylase (PTA), lactate dehydrogenase (LDH), pyruvate formate lyase (PFL), aldehyde dehydrogenase (ADH) and/or alcohol dehydrogenase (ADH), enzymes in the D-xylose pathway, such as xylose isomerase and xylulokinase, enzymes in the L-arabinose pathway, such as L-arabinose isomerase and L-ribulose-5-phosphate 4-epimerase. The term gene is also intended to cover all copies of a particular gene, e.g., all of the DNA sequences in a cell encoding a particular gene product.

The term “transcriptional control” is intended to include the ability to modulate gene expression at the level of transcription. In certain embodiments, transcription, and thus gene expression, is modulated by replacing or adding a surrogate promoter near the 5′ end of the coding region of a gene-of-interest, thereby resulting in altered gene expression. In certain embodiments, the transcriptional control of one or more gene is engineered to result in the optimal expression of such genes, e.g., in a desired ratio. The term also includes inducible transcriptional control as recognized in the art.

The term “expression” is intended to include the expression of a gene at least at the level of mRNA production.

The term “expression product” is intended to include the resultant product, e.g., a polypeptide, of an expressed gene.

The term “increased expression” is intended to include an alteration in gene expression at least at the level of increased mRNA production and, preferably, at the level of polypeptide expression. The term “increased production” is intended to include an increase in the amount of a polypeptide expressed, in the level of the enzymatic activity of the polypeptide, or a combination thereof.

The terms “activity,” “activities,” “enzymatic activity,” and “enzymatic activities” are used interchangeably and are intended to include any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity may be located in the intracellular space of the cell, cell-associated, secreted into the extracellular milieu, or a combination thereof. Techniques for determining total activity as compared to secreted activity are described herein and are known in the art.

The term “xylanolytic activity” is intended to include the ability to hydrolyze glycosidic linkages in oligopentoses and polypentoses.

The term “cellulolytic activity” is intended to include the ability to hydrolyze glycosidic linkages in oligohexoses and polyhexoses. Cellulolytic activity may also include the ability to depolymerize or debranch cellulose and hemicellulose.

As used herein, the term “lactate dehydrogenase” or “LDH” is intended to include the enzyme capable of converting pyruvate into lactate. It is understood that LDH can also catalyze the oxidation of hydroxybutyrate.

As used herein the term “alcohol dehydrogenase” or “ADH” is intended to include the enzyme capable of converting acetaldehyde into an alcohol, such as ethanol.

As used herein, the term “phosphotransacetylase” or “PTA” is intended to include the enzyme capable of converting Acetyl CoA into acetate.

As used herein, the term “acetate kinase” or “ACK” is intended to include the enzyme capable of converting Acetyl CoA into acetate.

As used herein, the term “pyruvate formate lyase” or “PFL” is intended to include the enzyme capable of converting pyruvate into Acetyl CoA.

The term “pyruvate decarboxylase activity” is intended to include the ability of a polypeptide to enzymatically convert pyruvate into acetaldehyde (e.g., “pyruvate decarboxylase” or “PDC”). Typically, the activity of a selected polypeptide encompasses the total enzymatic activity associated with the produced polypeptide, comprising, e.g., the superior substrate affinity of the enzyme, thermostability, stability at different pHs, or a combination of these attributes.

The term “ethanologenic” is intended to include the ability of a microorganism to produce ethanol from a carbohydrate as a fermentation product. The term is intended to include, but is not limited to, naturally occurring ethanologenic organisms, ethanologenic organisms with naturally occurring or induced mutations, and ethanologenic organisms which have been genetically modified.

The terms “fermenting” and “fermentation” are intended to include the enzymatic process (e.g., cellular or acellular, e.g., a lysate or purified polypeptide mixture) by which ethanol is produced from a carbohydrate, in particular, as a product of fermentation.

The term “secreted” is intended to include the movement of polypeptides to the periplasmic space or extracellular milieu. The term “increased secretion” is intended to include situations in which a given polypeptide is secreted at an increased level (i.e., in excess of the naturally-occurring amount of secretion). In certain embodiments, the term “increased secreted” refers to an increase in secretion of a given polypeptide that is at least about 10% or at least about 100%, 200%, 300%, 400%, 500%, 600%, 700%, 800%, 900%, 1000%, or more, as compared to the naturally-occurring level of secretion.

The term “secretory polypeptide” is intended to include any polypeptide(s), alone or in combination with other polypeptides, that facilitate the transport of another polypeptide from the intracellular space of a cell to the extracellular milieu. In certain embodiments, the secretory polypeptide(s) encompass all the necessary secretory polypeptides sufficient to impart secretory activity to a Gram-negative or Gram-positive host cell. Typically, secretory proteins are encoded in a single region or locus that may be isolated from one host cell and transferred to another host cell using genetic engineering. In certain embodiments, the secretory polypeptide(s) are derived from any bacterial cell having secretory activity. In certain embodiments, the secretory polypeptide(s) are derived from a host cell having Type II secretory activity. In certain embodiments, the host cell is a thermophilic bacterial cell.

The term “derived from” is intended to include the isolation (in whole or in part) of a polynucleotide segment from an indicated source or the purification of a polypeptide from an indicated source. The term is intended to include, for example, direct cloning, PCR amplification, or artificial synthesis from or based on a sequence associated with the indicated polynucleotide source.

By “thermophilic” is meant an organism that thrives at a temperature of about 45° C. or higher.

By “mesophilic” is meant an organism that thrives at a temperature of about 20-45° C.

The term “organic acid” is art-recognized. “Organic acid,” as used herein, also includes certain organic solvents such as ethanol. The term “lactic acid” refers to the organic acid 2-hydroxypropionic acid in either the free acid or salt form. The salt form of lactic acid is referred to as “lactate” regardless of the neutralizing agent, i.e., calcium carbonate or ammonium hydroxide. The term “acetic acid” refers to the organic acid methanecarboxylic acid, also known as ethanoic acid, in either free acid or salt form. The salt form of acetic acid is referred to as “acetate.”

Certain embodiments of the present invention provide for the “insertion,” (e.g., the addition, integration, incorporation, or introduction) of certain genes or particular polynucleotide sequences within thermophilic or mesophilic microorganisms, which insertion of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

Certain embodiments of the present invention provide for the “inactivation” or “deletion” of certain genes or particular polynucleotide sequences within thermophilic or mesophilic microorganisms, which “inactivation” or “deletion” of genes or particular polynucleotide sequences may be understood to encompass “genetic modification(s)” or “transformation(s)” such that the resulting strains of said thermophilic or mesophilic microorganisms may be understood to be “genetically modified” or “transformed.” In certain embodiments, strains may be of bacterial, fungal, or yeast origin.

The term “CBP organism” is intended to include microorganisms of the invention, e.g., microorganisms that have properties suitable for CBP.

In one aspect of the invention, the genes or particular polynucleotide sequences are inserted to activate the activity for which they encode, such as the expression of an enzyme. In certain embodiments, genes encoding enzymes in the metabolic production of ethanol, e.g., enzymes that metabolize pentose and/or hexose sugars, may be added to a mesophilic or thermophilic organism. In certain embodiments of the invention, the enzyme may confer the ability to metabolize a pentose sugar and be involved, for example, in the D-xylose pathway and/or L-arabinose pathway.

In one aspect of the invention, the genes or particular polynucleotide sequences are partially, substantially, or completely deleted, silenced, inactivated, or down-regulated in order to inactivate the activity for which they encode, such as the expression of an enzyme. Deletions provide maximum stability because there is no opportunity for a reverse mutation to restore function. Alternatively, genes can be partially, substantially, or completely deleted, silenced, inactivated, or down-regulated by insertion of nucleic acid sequences that disrupt the function and/or expression of the gene (e.g., P1 transduction or other methods known in the art). The terms “eliminate,” “elimination,” and “knockout” are used interchangeably with the terms “deletion,” “partial deletion,” “substantial deletion,” or “complete deletion.” In certain embodiments, strains of thermophilic or mesophilic microorganisms of interest may be engineered by site directed homologous recombination to knockout the production of organic acids. In still other embodiments, RNAi or antisense DNA (asDNA) may be used to partially, substantially, or completely silence, inactivate, or down-regulate a particular gene of interest.

In certain embodiments, the genes targeted for deletion or inactivation as described herein may be endogenous to the native strain of the microorganism, and may thus be understood to be referred to as “native gene(s)” or “endogenous gene(s).” An organism is in “a native state” if it has not been genetically engineered or otherwise manipulated by the hand of man in a manner that intentionally alters the genetic and/or phenotypic constitution of the organism. For example, wild-type organisms may be considered to be in a native state. In other embodiments, the gene(s) targeted for deletion or inactivation may be non-native to the organism.

### Biomass

The terms “lignocellulosic material,” “lignocellulosic substrate,” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants, and sugar-processing residues.

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; and forestry wastes, such as but not limited to recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Particularly advantageous lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Paper sludge is also a viable feedstock for lactate or acetate production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

### Pyruvate Formate Lyase (PFL)

Pyruvate formate lyase (PFL) is an important enzyme (found in Escherichia coli and other organisms) that helps regulate anaerobic glucose metabolism. Using radical chemistry, it catalyzes the reversible conversion of pyruvate and coenzyme-A into formate and acetyl-CoA, a precursor of ethanol. Pyruvate formate lyase is a homodimer made of 85 kDa, 759-residue subunits. It has a 10-stranded beta/alpha barrel motif into which is inserted a beta finger that contains major catalytic residues. The active site of the enzyme, elucidated by x-ray crystallography, holds three essential amino acids that perform catalysis (Gly734, Cys418, and Cys419), three major residues that hold the substrate pyruvate close by (Arg435, Arg176, and Ala272), and two flanking hydrophobic residues (Trp333 and Phe432).

Studies have found structural similarities between the active site of pyruvate formate lyase and that of Class I and Class III ribonucleotide reductase (RNR) enzymes. The roles of the 3 catalytic residues are as follows: Gly734 (glycyl radical)—transfers the radical on and off Cys418, via Cys419; Cys418 (thiyl radical)—performs acylation chemistry on the carbon atom of the pyruvate carbonyl; Cys419 (thiyl radical)—performs hydrogen-atom transfers.

The proposed mechanism for pyruvate formate lyase begins with radical transfer from Gly734 to Cys418, via Cys419. The Cys418 thiyl radical adds covalently to C2 (second carbon atom) of pyruvate, generating an acetyl-enzyme intermediate (which now contains the radical). The acetyl-enzyme intermediate releases a formyl radical that undergoes hydrogen-atom transfer with Cys419. This generates formate and a Cys419 radical. Coenzyme-A undergoes hydrogen-atom transfer with the Cys419 radical to generate a coenzyme-A radical. The coenzyme-A radical then picks up the acetyl group from Cys418 to generate acetyl-CoA, leaving behind a Cys418 radical. Pyruvate formate lyase can then undergo radical transfer to put the radical back onto Gly734. Each of the above mentioned steps are also reversible.

Two additional enzymes regulate the “on” and “off” states of pyruvate formate lyase to regulate anaerobic glucose metabolism: PFL activase (AE) and PFL deactivase (DA). Activated pyruvate formate lyase allows formation of acetyl-CoA, a small molecule important in the production of energy, when pyruvate is available. Deactivated pyruvate formate lyase, even with substrates present, does not catalyze the reaction. PFL activase is part of the radical SAM (S-adenosylmethionine) superfamily.

The enzyme turns pyruvate formate lyase “on” by converting Gly734 (G-H) into a Gly734 radical (G*) via a 5′-deoxyadenosyl radical (radical SAM). PFL deactivase (DA) turns pyruvate formate lyase “off” by quenching the Gly734 radical. Furthermore, pyruvate formate lyase is sensitive to molecular oxygen (O₂), the presence of which shuts the enzyme off.

### Lactate

Lactate is produced by NADH-dependent reduction of pyruvate in an enzymatic reaction catalyzed by lactate dehydrogenase (Ldh). Both C. thermocellum and C. cellulolyticum make lactate under standard fermentation conditions and have well annotated genes encoding Ldh (see Table 7). Lactate yield can be increased by partial, substantial, or complete deletion, silencing, inactivation, or down-regulation of single genes or combinations of genes in competing pathways leading to acetate, ethanol, and formate production. Key genes to be targeted in these pathways include pta and ack (individual and/or combined mutations) for acetate, adh's for ethanol, and pfl for formate. All of the above genes have been annotated in the published genomes of C. thermocellum and C. cellulolyticum (See Table below). In certain cases (pfl for C. cellulolyticum and adh for both organisms) multiple homologous genes are predicted for a given step.

TABLE 7

| | | Published | Published |
|---|---|---|---|
| | | C. thermocellum | C. cellulolyticum |
| Target | gene | 27405 genome | H10 genome |
| Lactate dehydrogenase | ldh | Cthe1053 | Gene 2262 |
| phosphotransacetylase | pta | Cthe1029 | Gene 132 |
| Acetate Kinase | ack | Cthe1028 | Gene 131 |
| Pyruvate Formate Lyase | pfl | Cthe505 | Gene2064 and |
| | | | Gene 2216 |
| Alcohol dehydrogenase(s) | adh | Cthe 423, 394, | Gene 873, 534, |
| | | 2579, 101, 2238 | 988, 2512 |

### Acetate

Acetate is produced from AcetylCoA in two reaction steps catalyzed by phosphotransacetylyase (Pta) and acetate kinase (Ack). The reactions mediated by these enzymes are shown below:

Pta reaction: acetyl-CoA+phosphate=CoA+acetyl phosphate (EC 2.3.1.8)

Ack reaction: ADP+acetyl phosphate=ATP+acetate (EC 2.7.2.1)

Both C. thermocellum and C. cellulolyticum make acetate under standard fermentation conditions and have well annotated genes encoding Pta and Ack (see Table 7 supra). Acetate yield can be increased by partial, substantial, or complete deletion, silencing, inactivation, or down-regulation of single genes or combinations of genes in competing pathways leading to lactate, and ethanol production. Key genes to be targeted in these pathways include ldh for lactate, adh's for ethanol. All of the above genes have been annotated in the published genomes of C. thermocellum and C. cellulolyticum (See Table 7 supra). In certain cases (adh for both organisms) multiple homologous genes are predicted for a given step. Furthermore, the production of acetate and hydrogen are linked as a result of redox balance. Thus, high the same mutations that produce high acetate yield will also increase hydrogen yield, which could be useful for bio-hydrogen production.

### Xylose Metabolism

Xylose is a five-carbon monosaccharide that can be metabolized into useful products by a variety of organisms. There are two main pathways of xylose metabolism, each unique in the characteristic enzymes they utilize. One pathway is called the “Xylose Reductase-Xylitol Dehydrogenase” or XR-XDH pathway. Xylose reductase (XR) and xylitol dehydrogenase (XDH) are the two main enzymes used in this method of xylose degradation. XR, encoded by the XYL1 gene, is responsible for the reduction of xylose to xylitol and is aided by cofactors NADH or NADPH. Xylitol is then oxidized to xylulose by XDH, which is expressed through the XYL2 gene, and accomplished exclusively with the cofactor NAD+. Because of the varying cofactors needed in this pathway and the degree to which they are available for usage, an imbalance can result in an overproduction of xylitol byproduct and an inefficient production of desirable ethanol. Varying expression of the XR and XDH enzyme levels have been tested in the laboratory in the attempt to optimize the efficiency of the xylose metabolism pathway.

The other pathway for xylose metabolism is called the “Xylose Isomerase” (XI) pathway. Enzyme XI is responsible for direct conversion of xylose into xylulose, and does not proceed via a xylitol intermediate. Both pathways create xylulose, although the enzymes utilized are different. After production of xylulose both the XR-XDH and XI pathways proceed through enzyme xylulokinase (XK), encoded on gene XKS1, to further modify xylulose into xylulose-5-P where it then enters the pentose phosphate pathway for further catabolism.

Studies on flux through the pentose phosphate pathway during xylose metabolism have revealed that limiting the speed of this step may be beneficial to the efficiency of fermentation to ethanol. Modifications to this flux that may improve ethanol production include a) lowering phosphoglucose isomerase activity, b) deleting the GND1 gene, and c) deleting the ZWF1 gene (Jeppsson et al., 2002). Since the pentose phosphate pathway produces additional NADPH during metabolism, limiting this step will help to correct the already evident imbalance between NAD(P)H and NAD+ cofactors and reduce xylitol byproduct. Another experiment comparing the two xylose metabolizing pathways revealed that the XI pathway was best able to metabolize xylose to produce the greatest ethanol yield, while the XR-XDH pathway reached a much faster rate of ethanol production (Karhumaa et al., 2007).

### Microorganisms

The present invention includes multiple strategies for the development of microorganisms with the combination of substrate-utilization and product-formation properties required for CBP. The “native cellulolytic strategy” involves engineering naturally occurring cellulolytic microorganisms to improve product-related properties, such as yield and titer. The “recombinant cellulolytic strategy” involves engineering natively non-cellulolytic organisms that exhibit high product yields and titers to express a heterologous cellulase system that enables cellulose utilization or hemicellulose utilization or both.

### Cellulolytic Microorganisms

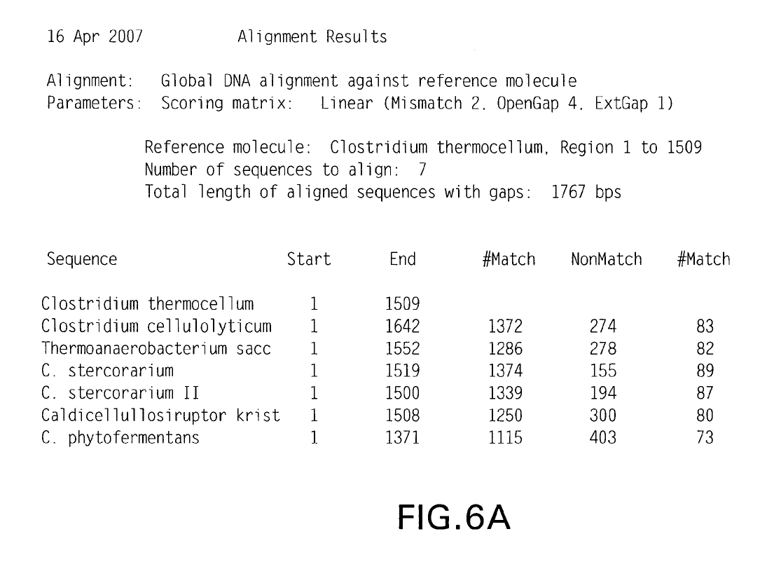

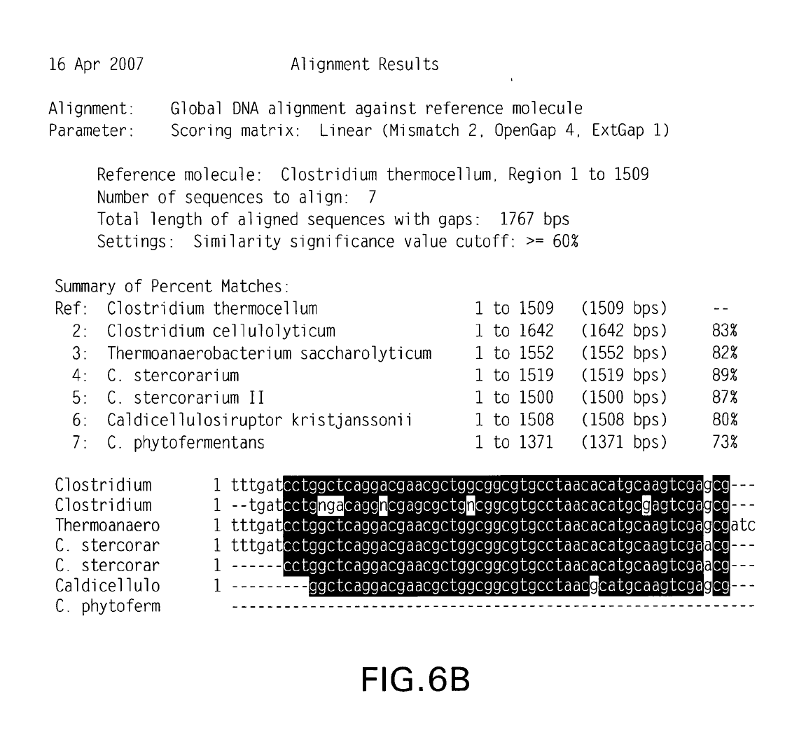

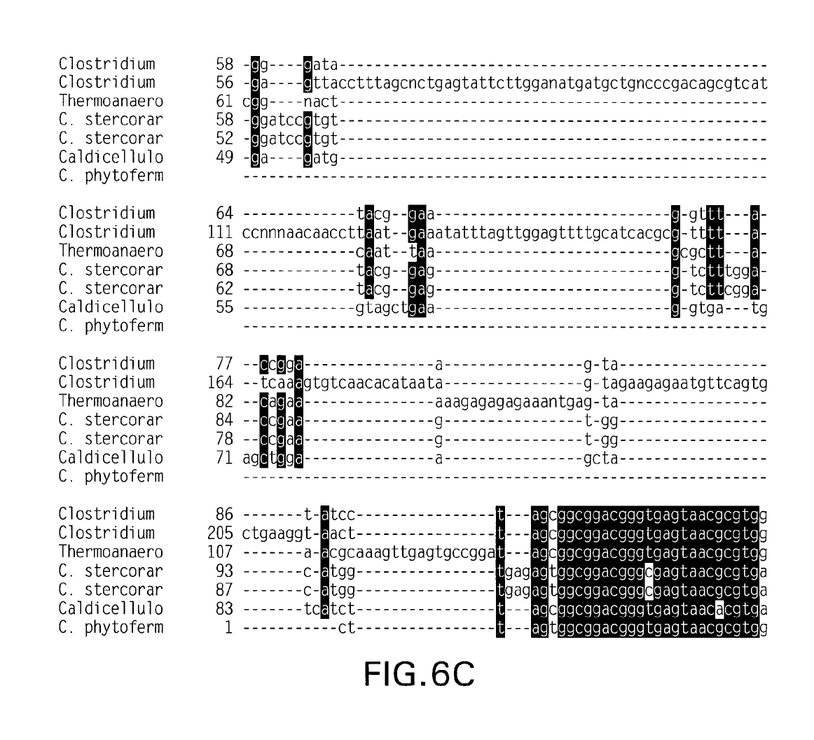

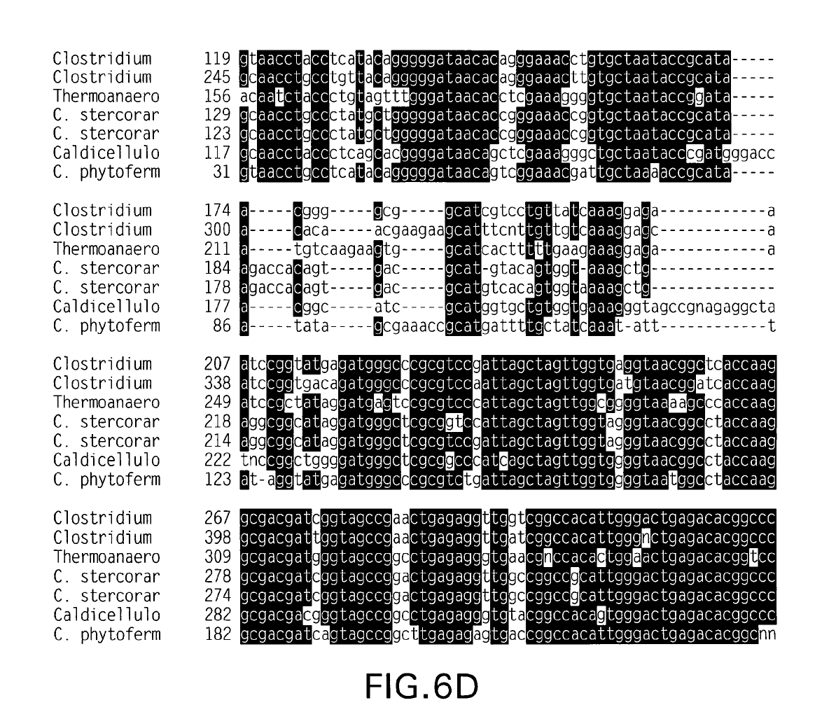

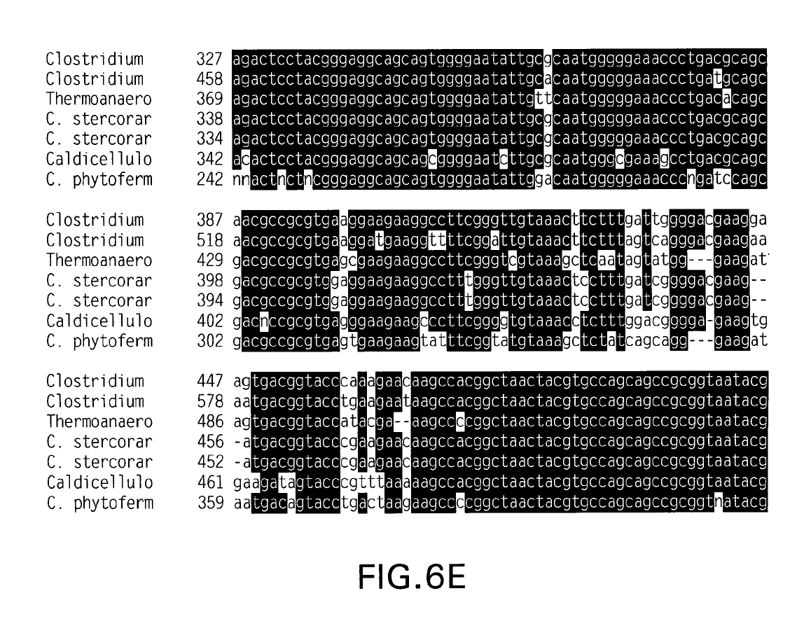

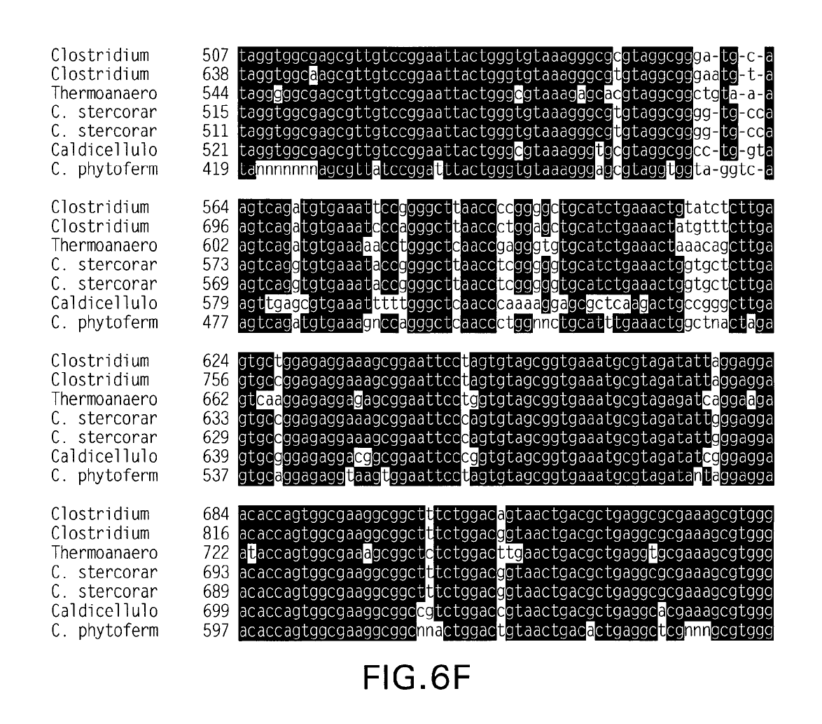

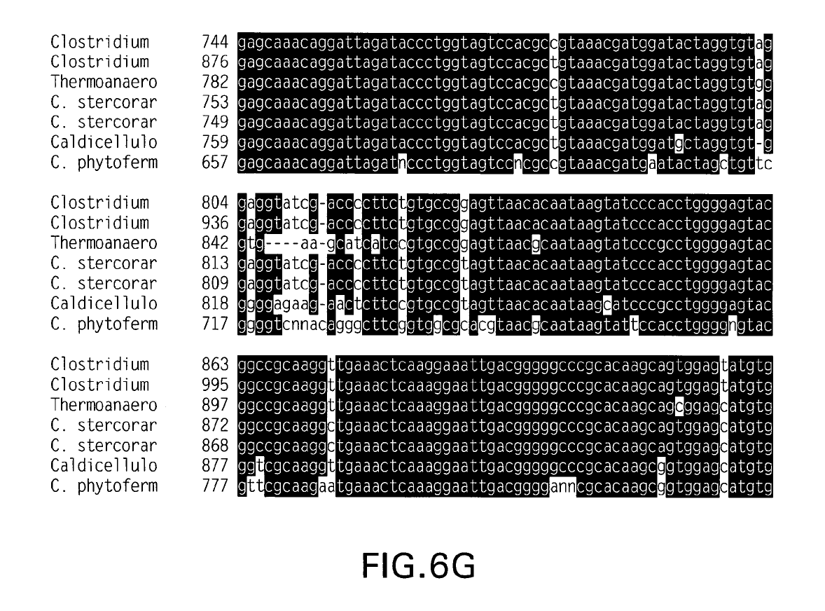

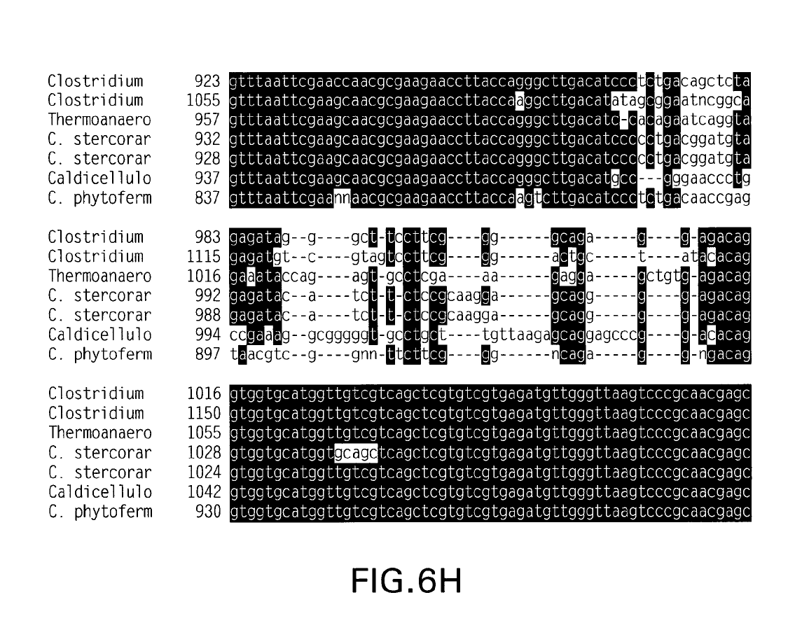

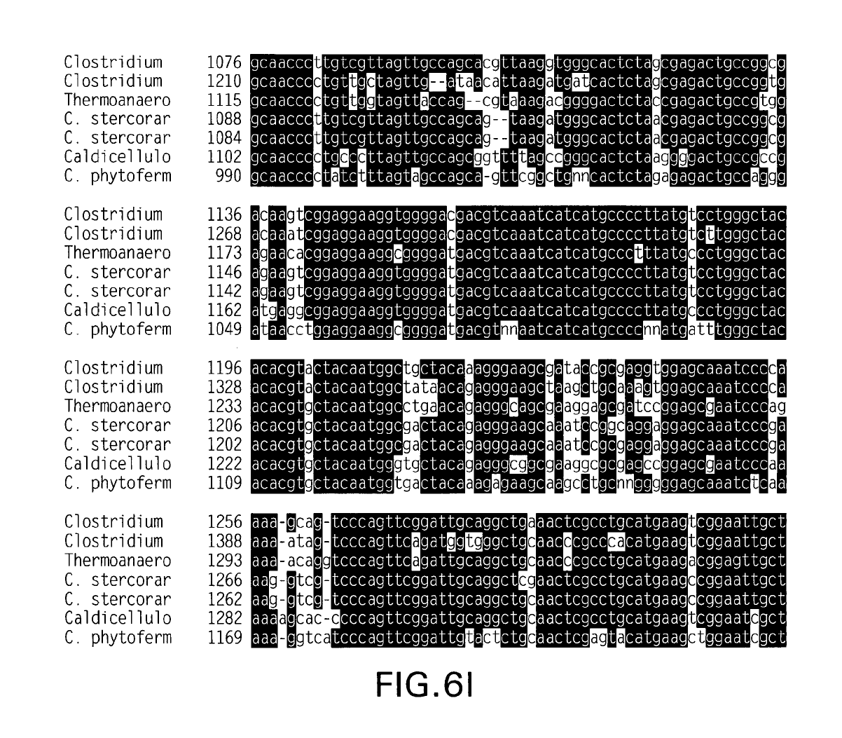

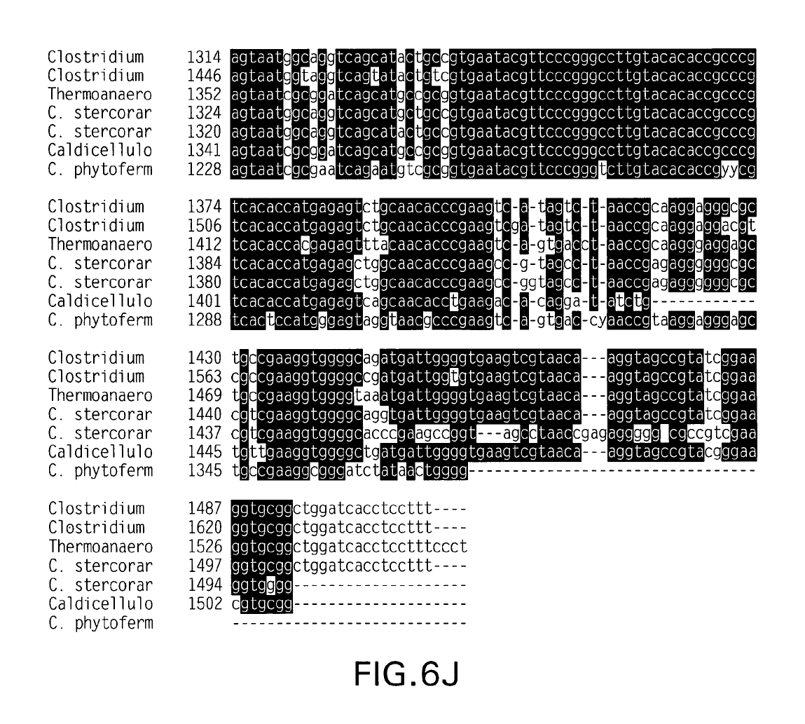

Several microorganisms reported in the literature to be cellulolytic or have cellulolytic activity have been characterized by a variety of means, including their ability to grow on microcrystalline cellulose as well as a variety of other sugars. Additionally, the organisms may be characterized by other means, including but not limited to, their ability to depolymerize and debranch cellulose and hemicellulose. Clostridium thermocellum (strain DSMZ 1237) was used to benchmark the organisms of interest. As used herein, C. thermocellum may include various strains, including, but not limited to, DSMZ 1237, DSMZ 1313, DSMZ 2360, DSMZ 4150, DSMZ 7072, and ATCC 31924. In certain embodiments of the invention, the strain of C. thermocellum may include, but is not limited to, DSMZ 1313 or DSMZ 1237. In another embodiment, particularly suitable organisms of interest for use in the present invention include cellulolytic microorganisms with a greater than 70% 16S rDNA homology to C. thermocellum. Alignment of Clostridium thermocellum, Clostridium cellulolyticum, Thermoanaerobacterium saccharolyticum, C. stercorarium, C. stercorarium II, Caldiscellulosiruptor kristjanssonii, C. phytofermentans indicate a 73-85% homology at the level of the 16S rDNA gene (FIG. 6).

Clostridium straminisolvens has been determined to grow nearly as well as C. thermocellum on Avicel®. Table 1 summarizes certain highly cellulolytic organisms.

TABLE 1

| | DSMZ | T optimum; | pH optimum; | | Aero- | | |
|---|---|---|---|---|---|---|---|
| Strain | No. | or range | or range | Gram Stain | tolerant | Utilizes | Products |
| Clostridium | 1313 | 55-60 | 7 | positive | No | cellobiose, | acetic acid, lactic acid, |
| thermocellum | | | | | | cellulose | ethanol, H₂, CO₂ |
| Clostridium | 16021 | 50-55; | 6.5-6.8; | positive | Yes | cellobiose, | acetic acid, lactic acid, |
| straminisolvens | | 45-60 | 6.0-8.5 | | | cellulose | ethanol, H₂, CO₂ |

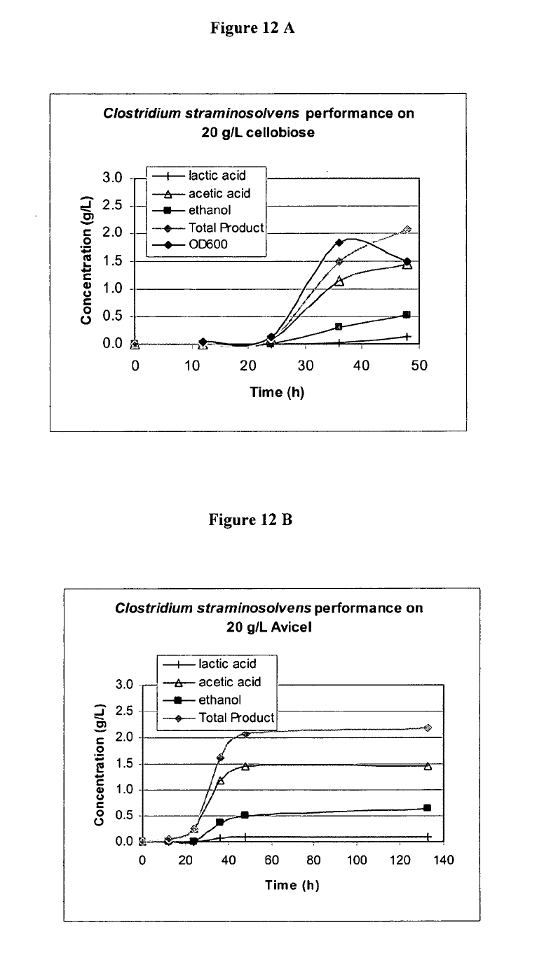

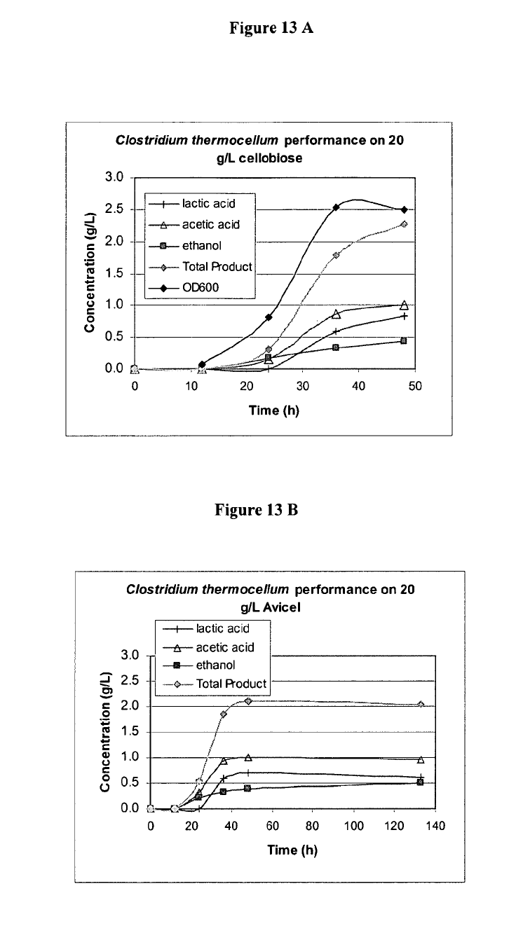

Organisms were grown on 20 g/L cellobiose or 20 g/L Avicel®. C. thermocellum was grown at 60° C. and C. straminisolvens was grown at 55° C. Both were pre-cultured from −80° C. freezer stock (origin DSMZ) on M122 with 50 mM MOPS. During mid to late log growth phase pre-cultures were used to inoculate the batch cultures in 100 mL serum bottles to a working volume of 50 mL. Liquid samples were removed periodically for HPLC analysis of metabolic byproducts and sugar consumption. OD₆₀₀ was taken at each of these time points. FIGS. 12A and 12B show product formation and OD₆₀₀ for C. straminisolvens on cellobiose and Avicel®, respectively. Substantial cellobiose (37%) was consumed with 48 hours before OD dropped and product formation leveled off. FIGS. 13A and 13B show product formation and OD₆₀₀ for C. thermocellum on cellobiose and Avicel®, respectively. C. thermocellum consumed ˜60% of cellobiose within 48 hours, at which point product formation leveled out. Inhibition due to formation of organic acids caused incomplete utilization of substrates.

Certain microorganisms, including, for example, C. thermocellum and C. straminisolvens, cannot metabolize pentose sugars, such as D-xylose or L-arabinose, but are able to metabolize hexose sugars. Both D-xylose and L-arabinose are abundant sugars in biomass with D-xylose accounting for approximately 16-20% in soft and hard woods and L-arabinose accounting for approximately 25% in corn fiber. Accordingly, one object of the invention is to provide genetically-modified cellulolytic microorganisms, with the ability to metabolize pentose sugars, such as D-xylose and L-arabinose, thereby to enhance their use as biocatalysts for fermentation in the biomass-to-acetic acid or lactic acid industries.

### Cellulolytic and Xylanolytic Microorganisms

Several microorganisms determined from literature to be both cellulolytic and xylanolytic have been characterized by their ability to grow on microcrystalline cellulose and birchwood xylan as well as a variety of other sugars. Clostridium thermocellum was used to benchmark the organisms of interest. Of the strains selected for characterization Clostridium cellulolyticum, Clostridium stercorarium subs. leptospartum, Caldicellulosiruptor kristjanssonii and Clostridium phytofermentans grew weakly on Avicel® and well on birchwood xylan. Table 2 summarizes some of the native cellulolytic and xylanolytic organisms.

TABLE 2

| | | T optimum; | pH optimum; | | Aero- | | |
|---|---|---|---|---|---|---|---|
| Strain | Source/No. | or range | or range | Gram Stai | tolerant | Utilizes | Products |
| Clostridium | DSM 5812 | 34 | 7.2 | negative | no | Cellulose, xylan, | acetic acid, lactic acid, |
| cellulolyticum | | | | | | arabinose, mannose, | ethanol, H₂, CO₂ |
| | | | | | | galactose, xylose, | |
| | | | | | | glucose, cellobiose | |
| Clostridium | DSM 9219 | 60-65 | 7.0-7.5 | negative | no | Cellulose, cellobiose, | acetic acid, lactic acid, |
| stercorarium | | | | | | lactose, xylose, | ethanol, H₂, CO₂ |
| subs. | | | | | | melibiose, raffinose, | |
| leptospartum | | | | | | ribose, fructose, | |
| | | | | | | sucrose | |
| Caldicellulosiruptor | DSM 12137 | 78; 45-82 | 7; 5.8-8.0 | negative | No | cellobiose, glucose, | acetic acid, H₂, CO₂, |
| kristjanssonii | | | | | | xylose, galactose, | lactic acid, ethanol formate |
| | | | | | | mannose, cellulose | |
| Clostridium | ATCC | 37; 5-45 | 8.5; 6-9 | Negative | no | Cellulose, xylan, | acetic acid, H₂, CO₂, |
| phytofermentans | 700394 | | | (gram type | | cellobiose, fructose, | lactic acid, ethanol formate |
| | | | | positive) | | galactose, glucose, | |
| | | | | | | lactose, maltose, | |
| | | | | | | mannose, ribose, | |
| | | | | | | xylose | |

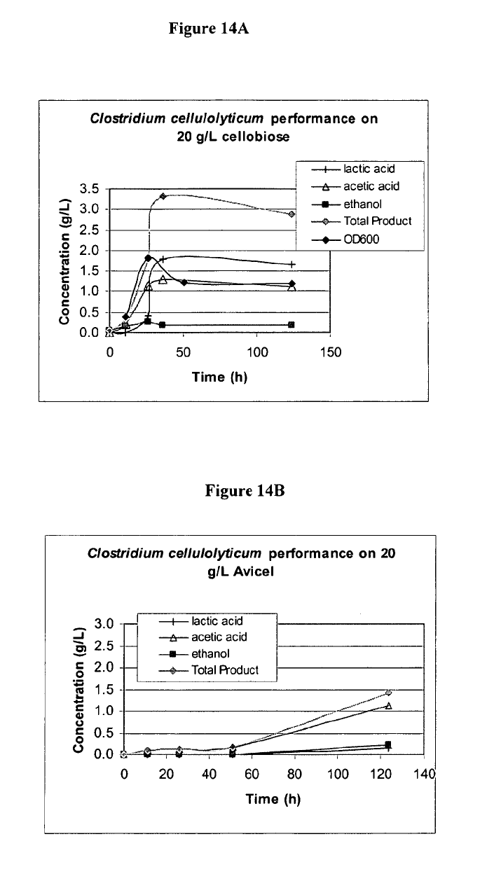

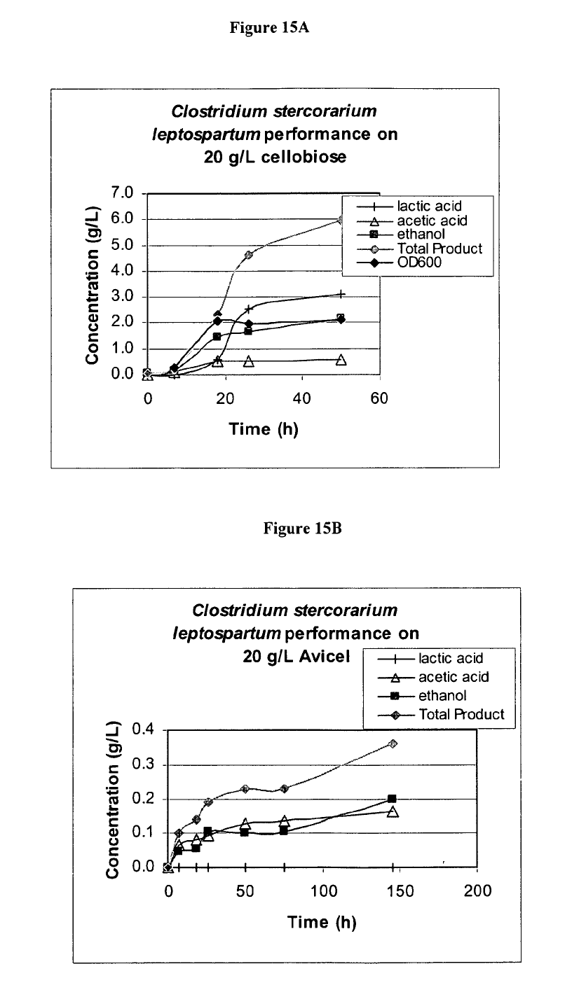

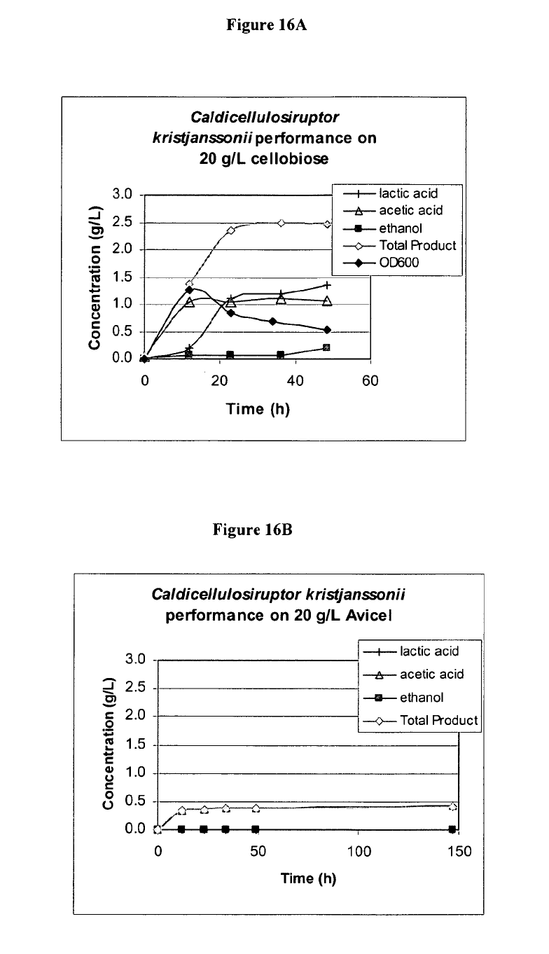

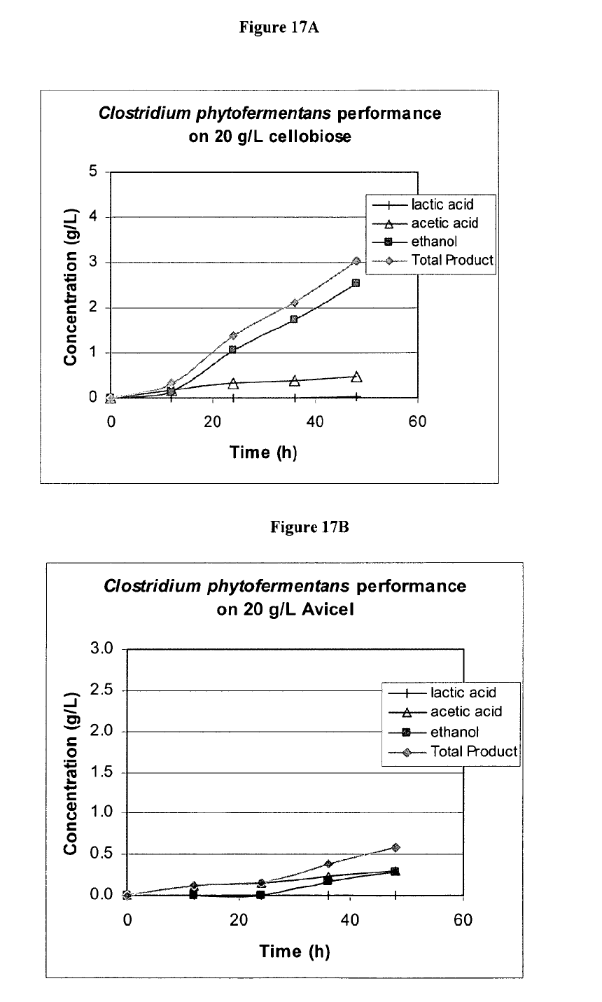

Organisms were grown on 20 g/L cellobiose, 20 g/L Avicel® or 5 g/L birchwood xylan. C. cellulolyticum was grown at 37° C., C. stercorarium subs. leptospartum was grown at 60° C., Caldicellulosiruptor kristjanssonii was grown at 75° C. and Clostridium phytofermentans was grown at 37° C. All were pre-cultured from −80° C. freezer stock in M122c supplemented with 50 mM MOPS. During mid to late log growth phase pre-cultures were used to inoculate the batch cultures in 100 mL serum bottles to a working volume of 50 mL. Liquid samples were removed periodically for HPLC analysis of metabolic byproducts and sugar consumption. OD₆₀₀ was taken at each of these time points. FIGS. 14A-17B show product formation and OD₆₀₀ for growth on cellobiose and Avicel®.

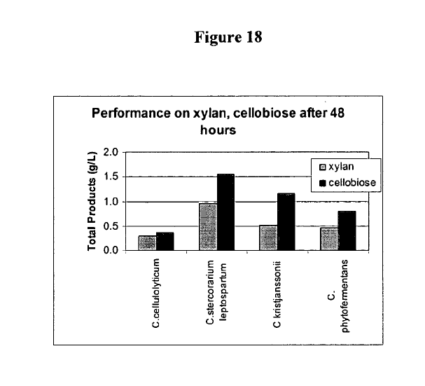

In a separate experiment organisms were grown on 2.5 g/L single sugars including cellobiose, glucose, xylose, galactose, arabinose, mannose and lactose as well as 5 g/L Avicel® and birchwood xylan. In FIG. 18 product formation is compared on cellobiose and birchwood xylan after two days. Table 3 summarizes how bacterial strains may be categorized based on their substrate utilization.

TABLE 3

| | cellobiose | glucose | xylose | galactose | arabinose | mannose | lactose |
|---|---|---|---|---|---|---|---|
| C. cellulolyticum | x | x | x | x | x | | |
| C. stercorarium subs. | x | x | x | x | x | x | x |
| leptospartum | | | | | | | |
| C. kristjanssonii | x | x | x | x | | x | x |
| C. phytofermentans | x | x | x | x | | x | |

### Transgenic Conversion of Microorganisms

The present invention provides compositions and methods for the transgenic conversion of certain microorganisms. When genes encoding enzymes involved in the metabolic pathway of lactate or acetate, including, for example, D-xylose and/or L-arabinose, are introduced into a bacterial strain that lacks one or more of these genes, for example, C. thermocellum or C. straminisolvens, one may select transformed strains for growth on D-xylose or growth on L-arabinose. It is expected that genes from other Clostridial species should be expressed in C. thermocellum and C. straminisolvens. Target gene donors may include microorganisms that confer the ability to metabolize hexose and pentose sugars, e.g., C. cellulolyticum, Caldicellulosiruptor kristjanssonii, C. phytofermentans, C. stercorarium, and Thermoanaerobacterium saccharolyticum.

The genomes of T. saccharolyticum, C. cellulolyticum, and C. phytofermentans are available. Accordingly, the present invention provides sequences which correspond to xylose isomerase and xylulokinase in each of the three hosts set forth above. In particular, the sequences corresponding to xylose isomerase (SEQ ID NO:6), xylulokinase (SEQ ID NO:7), L-arabinose isomerase (SEQ ID NO:8), and L-ribulose-5-phosphate 4-epimerase (SEQ ID NO:9) from T. saccharolyticum are set forth herein. Similarly, the sequences corresponding to xylose isomerase (SEQ ID NO:10), xylulokinase (SEQ ID NO:11), L-arabinose isomerase (SEQ ID NO:12), and L-ribulose-5-phosphate 4-epimerase (SEQ ID NO:13) from C. cellulolyticum are provided herein. C. phytofermentans utilizes the D-xylose pathway and does not utilize L-arabinose. Accordingly, the sequences corresponding to xylose isomerase (SEQ ID NO:14) and xylulokinase (SEQ ID NO:15) from C. phytofermentans are set forth herein.

C. kristjanssonii does metabolize xylose. To this end, the xylose isomerase (SEQ ID NO:71) and xylulokinase (SEQ ID NO:70) genes of C. kristjanssonii have been sequenced and are provided herein. C. straminisolvens has not been shown to grow on xylose, however it does contain xylose isomerase (SEQ ID NO:73) and xylulokinase (SEQ ID NO:72) genes, which may be functional after adaptation on xylose as a carbon source.

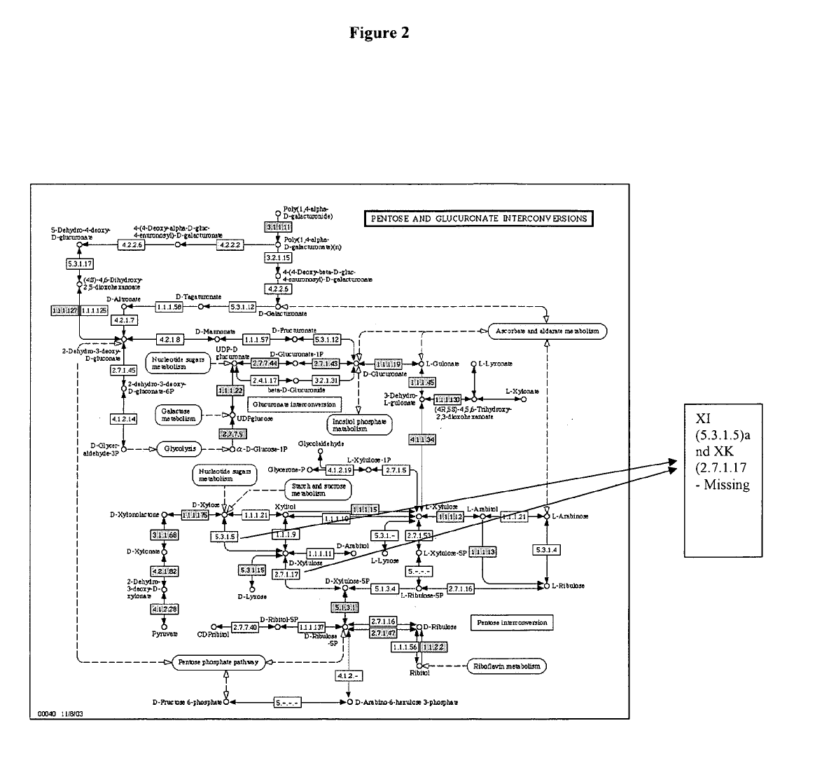

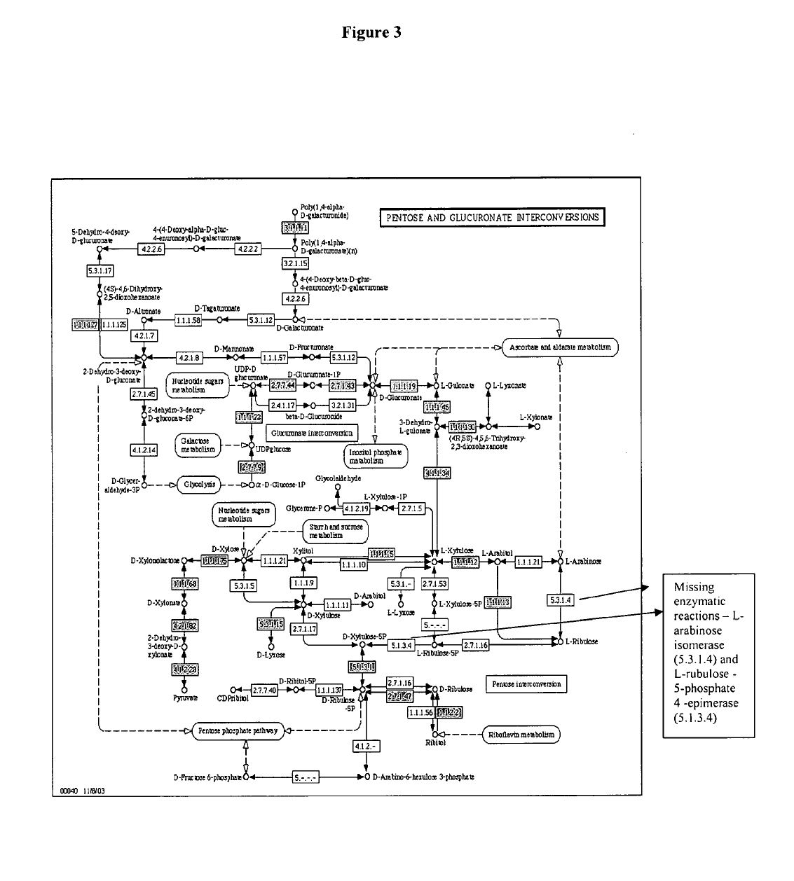

C. thermocellum and C. straminisolvens may lack one or more known genes or enzymes in the D-xylose to ethanol pathway and/or the L-arabinose utilization pathway. FIGS. 2 and 3 depict two key enzymes that are missing in each of these pathways in C. thermocellum. C. straminisolvens has xylose isomerase and xylulokinase, but the functionality of these enzymes is not known. Genomic sequencing has not revealed a copy of either L-arabinose isomerase or L-ribulose-5-phosphate 4-epimerase in C. straminosolvens.

C. thermocellum and C. straminisolvens are unable to metabolize xylulose which could reflect the absence (C. thermocellum) or lack of activity and/or expression (C. straminsolvens) of genes for xylose isomerase (referred to in FIG. 2 as “XI” or 5.3.1.5), which converts D-xylose to D-xylulose, and xylulokinase (also referred to in FIG. 2 as “XK” or 2.7.1.1), which converts D-xylulose to D-xylulose-5-phosphate. Furthermore, transport of xylose may be a limitation for C. straminsolvens. This potential limitation could be overcome by expression sugar transport genes from xylose utilizing organisms such as T. saccharolyticum and C. kristjanssonii.

C. thermocellum and C. straminisolvens are also unable to metabolize L-arabinose which could reflect the absence of genes for L-arabinose isomerase (also referred to in FIG. 3 as 5.3.1.4) and L-ribulose-5-phosphate 4-epimerase (also referred to in FIG. 3 as 5.1.3.4).

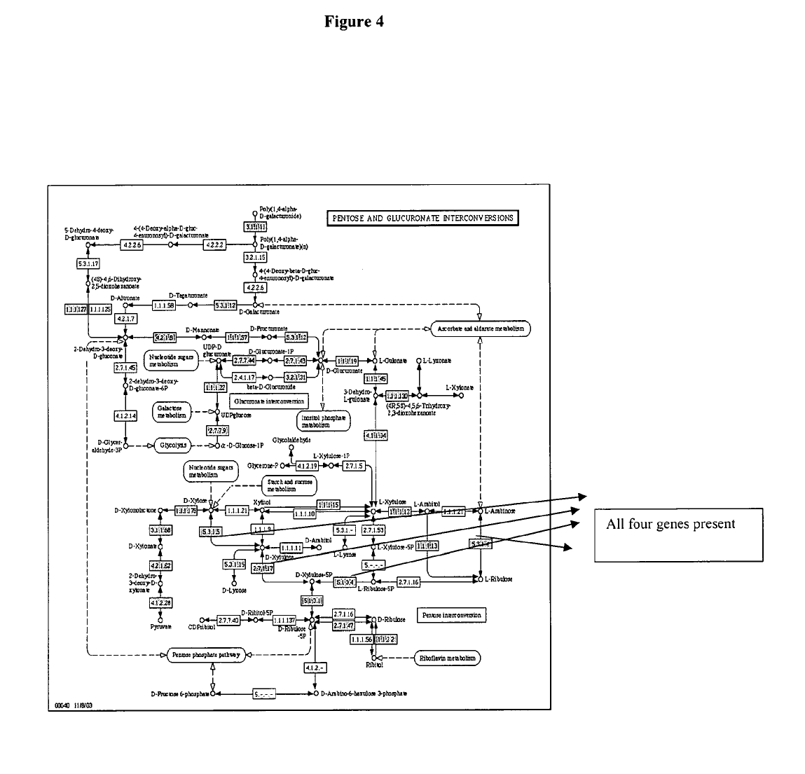

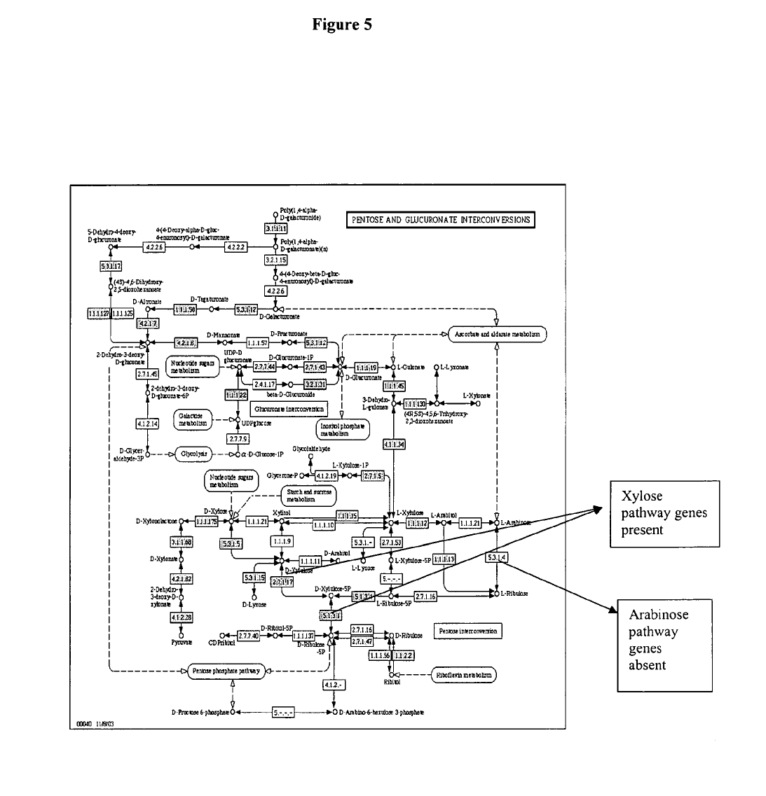

The four genes described above, e.g., xylose isomerase, xylulokinase, L-arabinose isomerase and L-ribulose-5-phosphate 4-epimerase, are present in several Clostridial species and Thermoanaerobacterium saccharolyticum species, including, but not limited to, Clostridium cellulolyticum (see FIG. 4), Thermoanaerobacterium saccharolyticum, C. stercorarium, Caldiscellulosiruptor kristjanssonii, and C. phytofermentans; these strains are good utilizers of these sugars. It will be appreciated that the foregoing bacterial strains may be used as donors of the genes described herein.

C. phytofermentans express the two xylose pathway genes described above (xylose isomerase and xylulokinase), but lack or do not express the arabinose pathway genes described above (L-arabinose isomerase and L-ribulose-5-phosphate 4-epimerase) (see FIG. 5).

Accordingly, it is an object of the invention to modify some of the above-described bacterial strains so as to optimize sugar utilization capability by, for example, introducing genes for one or more enzymes required for the production of ethanol from biomass-derived pentoses, e.g., D-xylose or L-arabinose metabolism. Promoters, including the native promoters of C. thermocellum or C. straminisolvens, such as triose phosphate isomerase (TPI), GAPDH, and LDH, may be used to express these genes. The sequences that correspond to native promoters of C. thermocellum include (TPI) (SEQ ID NO:16), GAPDH (SEQ ID NO:17), and LDH (SEQ ED NO:18). Once the gene has been cloned, codon optimization may be performed before expression. Cassettes containing, for example, the native promoter, a xylanolytic gene or arabinolytic gene, and a selectable marker may then be used to transform C. thermocellum or C. straminisolvens and select for D-xylose and L-arabinose growth on medium containing D-xylose or L-arabinose as the sole carbohydrate source.

### Transposons

To select for foreign DNA that has entered a host it is preferable that the DNA be stably maintained in the organism of interest. With regard to plasmids, there are two processes by which this can occur. One is through the use of replicative plasmids. These plasmids have origins of replication that are recognized by the host and allow the plasmids to replicate as stable, autonomous, extrachromosomal elements that are partitioned during cell division into daughter cells. The second process occurs through the integration of a plasmid onto the chromosome. This predominately happens by homologous recombination and results in the insertion of the entire plasmid, or parts of the plasmid, into the host chromosome. Thus, the plasmid and selectable marker(s) are replicated as an integral piece of the chromosome and segregated into daughter cells. Therefore, to ascertain if plasmid DNA is entering a cell during a transformation event through the use of selectable markers requires the use of a replicative plasmid or the ability to recombine the plasmid onto the chromosome. These qualifiers cannot always be met, especially when handling organisms that do not have a suite of genetic tools.

One way to avoid issues regarding plasmid-associated markers is through the use of transposons. A transposon is a mobile DNA element, defined by mosaic DNA sequences that are recognized by enzymatic machinery referred to as a transposase. The function of the transposase is to randomly insert the transposon DNA into host or target DNA. A selectable marker can be cloned onto a transposon by standard genetic engineering. The resulting DNA fragment can be coupled to the transposase machinery in an in vitro reaction and the complex can be introduced into target cells by electroporation. Stable insertion of the marker onto the chromosome requires only the function of the transposase machinery and alleviates the need for homologous recombination or replicative plasmids.

The random nature associated with the integration of transposons has the added advantage of acting as a form of mutagenesis. Libraries can be created that comprise amalgamations of transposon mutants. These libraries can be used in screens or selections to produce mutants with desired phenotypes. For instance, a transposon library of a CBP organism could be screened for the ability to produce less ethanol, or more lactic acid and/or more acetate.

### Native Cellulolytic Strategy

Naturally occurring cellulolytic microorganisms are starting points for CBP organism development via the native strategy. Anaerobes and facultative anaerobes are of particular interest. The primary objective is to engineer product yields and lactate or acetate titers to satisfy the requirements of an industrial process. Metabolic engineering of mixed-acid fermentations in relation to, for example, ethanol production, has been successful in the case of mesophilic, non-cellulolytic, enteric bacteria. Recent developments in suitable gene-transfer techniques allow for this type of work to be undertaken with cellulolytic bacteria.

### Recombinant Cellulolytic Strategy

Non-cellulolytic microorganisms with desired product-formation properties (e.g., high lactate or acetate yield and titer) are starting points for CBP organism development by the recombinant cellulolytic strategy. The primary objective of such developments is to engineer a heterologous cellulase system that enables growth and fermentation on pretreated lignocellulose. The heterologous production of cellulases has been pursued primarily with bacterial hosts producing ethanol at high yield (engineered strains of E. coli, Klebsiella oxytoca, and Zymomonas mobilis) and the yeast Saccharomyces cerevisiae. Cellulase expression in strains of K. oxytoca resulted in increased hydrolysis yields—but not growth without added cellulase—for microcrystalline cellulose, and anaerobic growth on amorphous cellulose. Although dozens of saccharolytic enzymes have been functionally expressed in S. cerevisiae, anaerobic growth on cellulose as the result of such expression has not been definitively demonstrated.

Aspects of the present invention relate to the use of thermophilic or mesophilic microorganisms as hosts for modification via the native cellulolytic strategy. Their potential in process applications in biotechnology stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic or mesophilic (including bacteria, procaryotic microorganism, and fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Clostridium phytofermentans, Clostridium straminosolvens, Thermoanaerobacterium thermosaccarolyticum, Thermoanaerobacterium saccharolyticum, Thermobacteroides acetoethylicus, Thermoanaerobium brockii, Methanobacterium thermoautotrophicum, Anaerocellum thermophilium, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus flavas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, variants thereof, and/or progeny thereof.

In particular embodiments, the present invention relates to thermophilic bacteria selected from the group consisting of Clostridium cellulolyticum, Clostridium thermocellum, and Thermoanaerobacterium saccharolyticum.

In certain embodiments, the present invention relates to thermophilic bacteria selected from the group consisting of Fervidobacterium gondwanense, Clostridium thermolacticum, Moorella sp., and Rhodothermus marinus.

In certain embodiments, the present invention relates to thermophilic bacteria of the genera Thermoanaerobacterium or Thermoanaerobacter, including, but not limited to, species selected from the group consisting of Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the present invention relates to microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

In certain embodiments, the present invention relates to mesophilic bacteria selected from the group consisting of Saccharophagus degradans; Flavobacterium johnsoniae; Fibrobacter succinogenes; Clostridium hungatei; Clostridium phytofermentans; Clostridium cellulolyticum; Clostridium aldrichii; Clostridium termitididis; Acetivibrio cellulolyticus; Acetivibrio ethanolgignens; Acetivibrio multivorans; Bacteroides cellulosolvens; and Alkalibacter saccharofomentans, variants thereof and progeny thereof.

### Methods of the Invention

During glycolysis, cells convert simple sugars, such as glucose, into pyruvic acid, with a net production of ATP and NADH. In the absence of a functioning electron transport system for oxidative phosphorylation, at least 95% of the pyruvic acid is consumed in short pathways which regenerate NAD⁺, an obligate requirement for continued glycolysis and ATP production. The waste products of these NAD⁺ regeneration systems are commonly referred to as fermentation products.

Microorganisms produce a diverse array of fermentation products, including organic acids, such as lactate (the salt form of lactic acid), acetate (the salt form of acetic acid), succinate, and butyrate, and neutral products, such as ethanol, butanol, acetone, and butanediol. End products of fermentation share to varying degrees several fundamental features, including: they are relatively nontoxic under the conditions in which they are initially produced, but become more toxic upon accumulation; and they are more reduced than pyruvate because their immediate precursors have served as terminal electron acceptors during glycolysis. Aspects of the present invention relate to the use of gene knockout technology to provide novel microorganisms useful in the production of lactate or acetate from lignocellulosic biomass substrates. The transformed organisms are prepared by deleting or inactivating one or more genes that encode competing pathways, such as the non-limiting pathways to organic acids described herein, optionally followed by a growth-based selection for mutants with improved performance for producing lactate or acetate as a fermentation product.

In certain embodiments, a thermophilic or mesophilic microorganism, which in a native state contains at least one gene that confers upon the microorganism an ability to produce lactic acid as a fermentation product, is transformed to decrease or eliminate expression of said at least one gene. The gene that confers upon said microorganism an ability to produce lactic acid as a fermentation product may code for expression of lactate dehydrogenase. The deletion or suppression of the gene(s) or particular polynucleotide sequence(s) that encode for expression of LDH diminishes or eliminates the reaction scheme in the overall glycolytic pathway whereby pyruvate is converted to lactic acid; the resulting relative abundance of pyruvate from these first stages of glycolysis should allow for the increased production of acetate. Similarly, the deletion or suppression of the gene(s) or particular polynucleotide sequence(s) that encode for expression of ADH diminishes or eliminates the reaction scheme in the overall glycolytic pathway whereby Acetyl CoA is converted to ethanol, the result being diversion of Acetyl CoA for the increased production of acetate.

In certain embodiments, a thermophilic or mesophilic microorganism, which in a native state contains at least one gene that confers upon the microorganism an ability to produce acetic acid as a fermentation product, is transformed to eliminate expression of said at least one gene. The gene that confers upon the microorganism an ability to produce acetic acid as a fermentation product may code for expression of acetate kinase phosphotransacetylase, pyruvate formate lyase, and/or aldehyde or alcohol dehydrogenase. The deletion or suppression of the gene(s) or particular polynucleotide sequence(s) that encode for expression of ACK, PTA, PFL, and/or ADH diminishes or eliminates the reaction scheme in the overall glycolytic pathway whereby pyruvate is converted to acetyl CoA and acetyl CoA is converted to acetic acid or ethanol; the resulting diversion of pyruvate from these later stages of glycolysis to be converted upstream by lactate dehydrogenase should allow for the increased production of lactate.

In certain embodiments, the above-detailed gene knockout schemes can be applied individually or in concert. Eliminating the mechanism for the production of lactate (i.e., knocking out the genes or particular polynucleotide sequences that encode for expression of LDH) generates more acetyl CoA; it follows that if the mechanism for the production of ethanol is also eliminated (i.e., knocking out the genes or particular polynucleotide sequences that encode for expression of ACK, PTA, PFL, and/or ADH), the abundance of acetyl CoA will be further enhanced, which should result in increased production of acetate. Likewise, eliminating the mechanism for the production of acetate or ethanol (i.e., knocking out the genes or particular polynucleotide sequences that encode for expression of PFL, PTA, ACK, and/or ADH) pyruvate can be converted more efficiently by lactate dehydrogenase, which should result in increased production of lactate.

In certain embodiments, it is not required that the thermophilic or mesophilic microorganisms have native or endogenous LDH, ACK, PTA, PFL, PDC or ADH. In certain embodiments, the genes encoding for LDH, ACK, PTA, PFL, PDC and/or ADH can be expressed recombinantly in the genetically modified microorganisms of the present invention. In certain embodiments, the gene knockout technology of the present invention can be applied to recombinant microorganisms, which may comprise a heterologous gene that codes for LDH, ACK, PTA, PFL, PDC and/or ADH, wherein said heterologous gene is expressed at sufficient levels to increase the ability of said recombinant microorganism (which may be thermophilic) to produce lactate or acetate as a fermentation product or to confer upon said recombinant microorganism (which may be thermophilic) the ability to produce lactate or acetate as a fermentation product.

In certain embodiments, aspects of the present invention relate to fermentation of lignocellulosic substrates to produce lactate or acetate in a concentration that is at least 70% of a theoretical yield based on cellulose content or hemicellulose content or both.

In certain embodiments, aspects of the present invention relate to fermentation of lignocellulosic substrates to produce lactate or acetate in a concentration that is at least 80% of a theoretical yield based on cellulose content or hemicellulose content or both.

In certain embodiments, aspects of the present invention relate to fermentation of lignocellulosic substrates to produce lactate or acetate in a concentration that is at least 90% of a theoretical yield based on cellulose content or hemicellulose content or both.

In certain embodiments, substantial or complete elimination of organic acid production from microorganisms in a native state may be achieved using one or more site-directed DNA homologous recombination events.

Operating either a simultaneous saccharification and co-fermentation (SSCF) or

CBP process at thermophilic temperatures offers several important benefits over conventional mesophilic fermentation temperatures of 30-37° C. In particular, costs for a process step dedicated to cellulase production are substantially reduced (e.g., 2-fold or more) for thermophilic SSCF and are eliminated for CBP. Costs associated with fermentor cooling and also heat exchange before and after fermentation are also expected to be reduced for both thermophilic SSCF and CBP. Finally, processes featuring thermophilic biocatalysts may be less susceptible to microbial contamination as compared to processes featuring conventional mesophilic biocatalysts.

The ability to redirect electron flow by virtue of modifications to carbon flow has broad implications. For example, this approach could be used to produce high lactate or acetate yields in strains other than T. saccharolyticum and/or to produce solvents other than ethanol, for example, higher alcohols (i.e., butanol).

Metabolic Engineering through Antisense Oligonucleotide (asRNA) Strategies

Fermentative microorganisms such as yeast and anaerobic bacteria ferment sugars to ethanol and other reduced organic end products, such as lactate or acetate. Theoretically, carbon flow can be directed to lactate or acetate production if the formation of competing end-products, such as acetate, lactate, and/or ethanol can be suppressed. The present invention provides several genetic engineering approaches designed to remove such competing pathways in the CBP organisms of the invention. The bulk of these approaches utilize knock-out constructs (for single crossover recombination) or allele-exchange constructs (for double crossover recombination) and target the genetic loci for ack, ldh, pfl, pta or adh. Although these tools employ “tried and true” strain development techniques, there are several potential issues that could stall progress: (i) they are dependent on the host recombination efficiency which in all cases is unknown for the CBP organisms; (ii) they can be used to knock out only one pathway at a time, so successive genetic alterations are incumbent upon having several selectable markers or a recyclable marker; (iii) deletion of target genes may be toxic or have polar effects on downstream gene expression.

The present invention provides additional approaches towards genetic engineering that do not rely on host recombination efficiency. One of these alternative tools is called antisense RNA (asRNA). Although antisense oligonucleotides have been used for over twenty-five years to inhibit gene expression levels both in vitro and in vivo, recent advances in mRNA structure prediction has facilitated smarter design of asRNA molecules. These advances have prompted a number of groups to demonstrate the usefulness of asRNA in metabolic engineering of bacteria.

The benefits of using asRNA over knock-out and allele-exchange technology are numerous: (i) alleviates the need for multiple selectable markers because multiple pathways can be targeted by a single asRNA construct; (ii) attenuation level of target mRNA can be adjusted by increasing or decreasing the association rate between asRNA; (iii) pathway inactivation can be conditional if asRNA transcripts are driven by conditional promoters. Recently, this technology has been used to increase solventogenesis in the Gram positive mesophile, Clostridium acetobutylicum (Tummala et al. (2003)). Although the exact molecular mechanism of how asRNA attenuates gene expression is unclear, the likely mechanism is triggered upon hybridization of the asRNA to the target mRNA. Mechanisms may include one or more of the following: (i) inhibition of translation of mRNA into protein by blocking the ribosome binding site from properly interacting with the ribosome, (ii) decreasing the half-life of mRNA through dsRNA-dependent RNases, such as RNase H, that rapidly degrade duplex RNA, and (iii) inhibition of transcription due to early transcription termination of mRNA.

### Design of Antisense Sequences

asRNAs are typically 18-25 nucleotides in length. There are several computation tools available for rational design of RNA-targeting nucleic acids (Sfold, Integrated DNA Technologies, STZ Nucleic Acid Design) which may be used to select asRNA sequences. For instance, the gene sequence for Clostridium thermocellum ack (acetate kinase) can be submitted to a rational design server and several asRNA sequences can be culled. In brief, the design parameters select for mRNA target sequences that do not contain predicted secondary structure.

### Design of Delivery Vector

A replicative plasmid will be used to deliver the asRNA coding sequence to the target organism. Vectors such as, but not limited to, pNW33N, pJIR418, pJIR751, and pCTC1, will form the backbone of the asRNA constructs for delivery of the asRNA coding sequences to inside the host cell. In addition to extra-chromosomal (plasmid based) expression, asRNAs may be stably inserted at a heterologous locus into the genome of the microorganism to get stable expression of asRNAs. In certain embodiments, strains of thermophilic or mesophilic microorganisms of interest may be engineered by site directed homologous recombination to knockout the production of organic acids and other genes of interest may be partially, substantially, or completely deleted, silenced, inactivated, or down-regulated by asRNA.

### Promoter Choice

To ensure expression of asRNA transcripts, compatible promoters for the given host will be fused to the asRNA coding sequence. The promoter-asRNA cassettes are constructed in a single PCR step. Sense and antisense primers designed to amplify a promoter region will be modified such that the asRNA sequence (culled from the rational design approach) is attached to the 5′ end of the antisense primer. Additionally, restriction sites, such as EcoRI or BamHI, will be added to the terminal ends of each primer so that the final PCR amplicon can be digested directly with restriction enzymes and inserted into the vector backbone through traditional cloning techniques.

With respect to microorganisms that do not have the ability to metabolize pentose sugars, but are able to metabolize hexose sugars as described herein, it will be appreciated that the ack and ldh genes of Clostridium thermocellum and Clostridium straminisolvens, for example, may be targeted for inactivation using antisense RNA according to the methods described herein.

With respect to microorganisms that confer the ability to metabolize pentose and hexose sugars as described herein, it will be appreciated that the ack and ldh genes of Clostridium cellulolyticum, Clostridium phytofermentans and Caldicellulosiruptor kristjanssonii, for example, may be targeted for inactivation using antisense according to the methods described herein.

In addition to antibiotic selection for strains expressing the asRNA delivery vectors, such strains may be selected on conditional media that contains any of the several toxic metabolite analogues such as sodium fluoroacetate (SFA), bromoacetic acid (BAA), chloroacetic acid (CAA), 5-fluoroorotic acid (5-FOA) and chlorolactic acid. Use of chemical mutagens including, but not exclusively, ethane methyl sulfonate (EMS) may be used in combination with the expression of antisense oligonucleotide (asRNA) to generate strains that have one or more genes partially, substantially, or completely deleted, silenced, inactivated, or down-regulated.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Generation of Custom Transposons For Mesophilic and Thermophilic Cellulolytic, Xylanolytic Organisms

The present invention provides methods for generating custom transposons for cellulolytic and/or xylanolytic and/or thermophilic organisms. To do this, a native promoter from the host organism will be fused to a selectable marker which has been determined to work in this organism. This fragment will be cloned into the EZ-Tn5™ transposon that is carried on the vector pMOD™-2<MCS> (Epicenter® Biotechnologies). For example, the C. thermocellum the gapDH promoter will be fused to the mLs drug marker, as well as the cat gene and then subcloned into vector pMOD™-2<MCS>.

Commercial transposons are lacking in thermostable drug markers and native promoters of cellulolytic and/or xylanolytic and/or thermophilic organisms. The mLs and cat markers have functioned in thermophilic bacteria and the gapDH promoter regulates a key glycolytic enzyme and should be constantly expressed. The combination of the above drug markers and the gapDH promoter will greatly enhance the probability of generating a functional transposon. This approach may be applied to other cellulolytic and/or xylanolytic and/or thermophilic organisms.

### Experimental Design

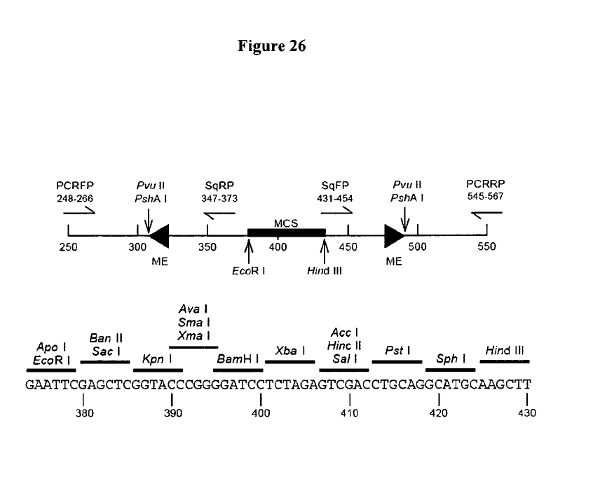

FIG. 26 is a diagram taken from the Epicenter® Biotechnologies user manual, which is incorporated herein by reference, representing by 250-550 of pMOD™-2<MCS>. In the top portion, the black arrowheads labeled ME denote 19 bp mosaic ends that define the transposon. The EcoRI and HindIII sites define the multi-cloning site, which is represented by the black box labeled MCS. In the bottom portion, the DNA sequence and the restriction enzymes associated with the MCS are shown.

The following primers will be used to amplify promoter fusion fragments from pMQ87-gapDH-cat and pMQ87-gapDH-mls: GGCGgaattc CTT GGT CTG ACA ATC GAT GC (SEQ ID NO:19); GGCGgaattc TATCAGTTATTACCCACTTTTCG (SEQ ID NO:20). The lower case letters denote engineered EcoRI restriction sites. The size of the amplicon generated will be ˜1.9 kb. Standard molecular procedures will allow the amplicon to be digested with EcoRI and cloned into the unique EcoRI site of pMOD™-2<MCS>. The transposon and subsequent transpososome will be generated and introduced into host organisms as described by the manufacturer.

### Example 2

### Constructs for Engineering Cellulolytic and Xylanolytic Strains

The present invention provides compositions and methods for genetically engineering an organism of interest to CBP by mutating genes encoding key enzymes of metabolic pathways which divert carbon flow away from ethanol and either lactate or acetate towards either lactate or acetate. Single crossover knockout constructs are designed so as to insert large fragments of foreign DNA into the gene of interest to partially, substantially, or completely delete, silence, inactivate, or down-regulate it. Double crossover knockout constructs are designed so as to partially, substantially, or completely delete, silence, inactivate, or down-regulate the gene of interest from the chromosome or replace the gene of interest on the chromosome with a mutated copy of the gene, such as a form of the gene interrupted by an antibiotic resistance cassette.

The design of single crossover knockout vectors requires the cloning of an internal fragment of the gene of interest into a plasmid based system. Ideally, this vector will carry a selectable marker that is expressed in the host strain but will not replicate in the host strain. Thus, upon introduction into the host strain the plasmid will not replicate. If the cells are placed in a conditional medium that selects for the marker carried on the plasmid, only those cells that have found a way to maintain the plasmid will grow. Because the plasmid is unable to replicate as an autonomous DNA element, the most likely way that the plasmid will be maintained is through recombination onto the host chromosome. The most likely place for the recombination to occur is at a region of homology between the plasmid and the host chromosome.

Alternatively, replicating plasmids can be used to create single crossover interruptions. Cells that have taken up the knockout vector can be selected on a conditional medium, then passaged in the absence of selection. Without the positive selection provided by the conditional medium, many organisms will lose the plasmid. In the event that the plasmid is inserted onto the host chromosome, it will not be lost in the absence of selection. The cells can then be returned to a conditional medium and only those that have retained the marker, through chromosomal integration, will grow. A PCR based method will be devised to screen for organisms that contain the marker located on the chromosome.

The design of double crossover knockout vectors requires at least cloning the DNA flanking (˜1 kb) the gene of interest into a plasmid and in some cases may include cloning the gene of interest. A selectable marker may be placed between the flanking DNA or if the gene of interest is cloned the marker is placed internally with respect to the gene. Ideally the plasmid used is not capable of replicating in the host strain. Upon the introduction of the plasmid into the host and selection on a medium conditional to the marker, only cells that have recombined the homologous DNA onto the chromosome will grow. Two recombination events are needed to replace the gene of interest with the selectable marker.

Alternatively, replicating plasmids can be used to create double crossover gene replacements. Cells that have taken up the knockout vector can be selected on a conditional medium, then passaged in the absence of selection. Without the positive selection provided by the conditional medium, many organisms will lose the plasmid. In the event that the drug marker is inserted onto the host chromosome, it will not be lost in the absence of selection. The cells can then be returned to a conditional medium and only those that have retained the marker, through chromosomal integration, will grow. A PCR based method may be devised to screen for organisms that contain the marker located on the chromosome.

In addition to antibiotic selection schemes, several toxic metabolite analogues such as sodium fluoroacetate (SFA), bromoacetic acid (BAA), chloroacetic acid (CAA), 5-fluoroorotic acid (5-FOA) and chlorolactic acid may be used to select mutants arising from either homologous recombinations, or transposon-based strategies. Use of chemical mutagens including, but not exclusively, ethane methyl sulfonate (EMS) may be used in combination with the directed mutagenesis schemes that employ homologous recombinations, or transposon-based strategies.

C. cellulolyticum Knockout Constructs
Acetate Kinase (Gene 131 from C. cellulolyticum Published Genome):

Single Crossover

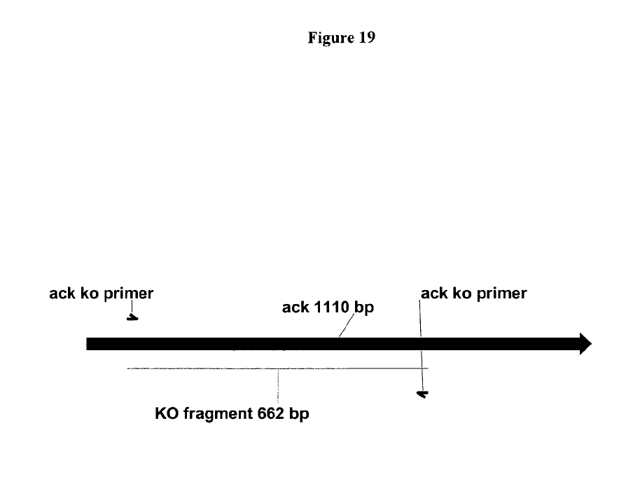

The acetate kinase gene of C. cellulolyticum is 1,110 bp in length. A 662 bp internal fragment (SEQ ID NO:21) spanning nucleotides 91-752 was amplified by PCR and cloned into suicide vectors and replicating vectors that have different selectable markers. Selectable markers may include those that provide erythromycin and chloramphenicol resistance. These plasmids will be used to disrupt the ack gene, for example, by retargeting the L1.Ltrb intron to insert into the C. cellulolyticum ack gene. A map of the ack gene and the region amplified by PCR for gene disruption are shown in FIG. 19. The underlined portions of SEQ ID NO:21 set forth below correspond to the sites that are EcoRI sites that flank the knockout fragment.

gaattctgcgacagaatagggattgacaattcctttataaagcaatcaag
gggttcagaagaggctgttattttgaataaagagctaaagaatcacaaag
atgcaatagaggctgttatttctgcactgactgacgataatatgggcgtt
ataaaaaacatgtccgaaatatcagcagtgggacacagaatagtacacgg
cggtgaaaaattcaacagttctgtagttatagatgaaaacgttatgaatg
cagtaagagagtgtatagacgttgcaccgcttcataatccgccgaatatt
ataggtatagaggcttgccagcagattatgcccaatatacctatggtagc
tgtatttgataccactttccacagctccatgcctgattatgcataccttt
acgcattgccatatgaactttatgaaaagtacggtataagaaaatatggt
ttccacggaacatcacacaaatatgttgcagaaagagcttctgcaatgct
tgataagtctttgaacgaattaaagataattacatgccatcttgggaacg
gttcaagtatttgtgctgttaacaagggtaaatcaattgatacttccatg
ggctttacacctttgcagggacttgcaatgggtacaagaagcggtacaat
agaccctgaagttgttacgaattc

These sites were engineered during the design of the “ack KO primers” and will allow subsequent cloning of the fragment into numerous vectors.

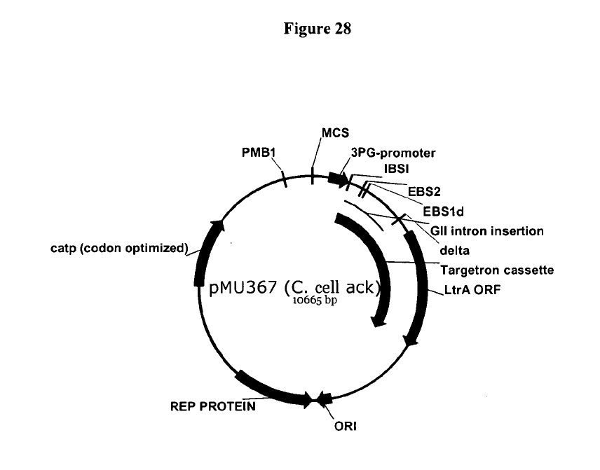

An example of a vector for retargeting the L1.Ltrb intron to insert in C. cellulolyticum ack gene (SEQ ID NO: 21) is depicted in FIG. 28. The sequence of the vector in FIG. 28, pMU367 is SEQ ID NO:30.

### Double Crossover

To construct a double crossover vector for the ack gene of C. cellulolyticum ˜1 kb of DNA flanking each side of the ack gene will be cloned. A selectable marker will be inserted between the flanking DNA. Selectable markers may include those that provide erythromycin and chloramphenicol resistance. The 3′ flanking region of the ack gene is not available in the available draft genome. To acquire this DNA, a kit such as GenomeWalker from Clontech will be used.

Phosphotransacetylase (pta):

As described above for the C. cellulolyticum ack gene, single and double crossovers are generated to disrupt the C. cellulolyticum pta gene. An example of a vector for retargeting the L1.Ltrb intron to insert in C. cellulolyticum pta gene (SEQ ID NO:22) is depicted in FIG. 12. The vector sequence this construct is SEQ ID NO:23 Lactate dehydrogenase (genes 2262 and 2744 of C. cellulolyticum published genome):

Single Crossover

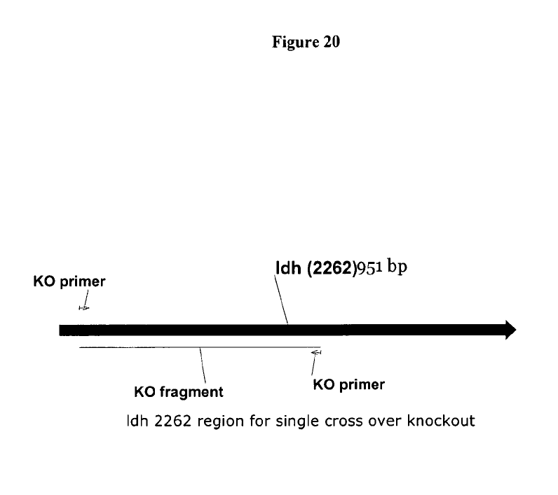

The ldh genes of C. cellulolyticum are 951 bp (for gene 2262) (SEQ ID NO:22) and 932 bp (for gene 2744) (SEQ ID NO:23) in length. A ˜500 bp internal fragment near the 5′ end of each gene will be amplified by PCR and cloned into suicide vectors and replicating vectors that have different selectable markers. Selectable markers may include those that provide drug resistance, such as erythromycin and chloramphenicol. These plasmids will be used to disrupt the ldh 2262 and ldh 2744 genes, for example, by retargeting the L1.Ltrb intron to insert into the C. cellulolyticum ldh gene. As an example, a map of the ldh 2262 gene and the region amplified by PCR for gene disruption are shown in FIG. 20.

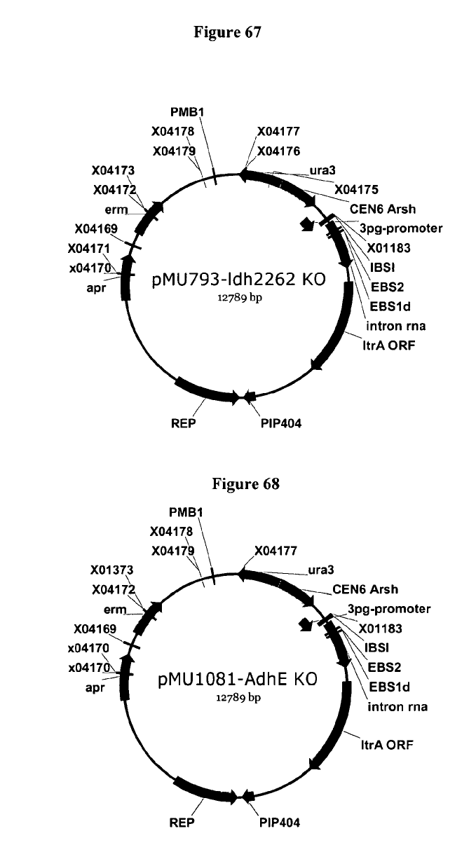

An example of a vector for retargeting the L1.Ltrb intron to insert in C. cellulolyticum ldh 2262 gene (SEQ ID NO:100) is depicted in FIG. 67. The vector sequence for this construct is SEQ ID NO: 101.

Double Crossover

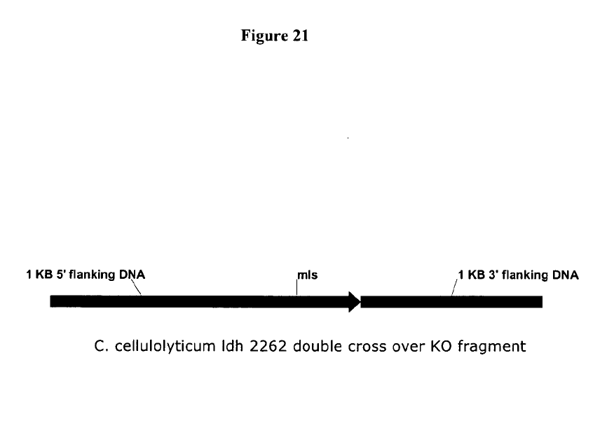

To construct a double crossover knockout vector for the ldh gene(s) of C. cellulolyticum ˜1 kb of DNA flanking each side of the ldh gene(s) will be cloned. A selectable marker will be inserted between the flanking DNA. Selectable markers may include those that provide drug resistance, such as erythromycin and chloramphenicol. FIG. 21 provides an example of C. cellulolyticum ldh (2262) double crossover knock out fragment.

In the sequence set forth below (SEQ ID NO:24) the mLs gene (selectable marker) is underlined and the flanking DNA is the remaining sequence. During primer design, restriction sites will be engineered and the 5′ and 3′ ends of the above fragment so that it can be cloned into a number of replicative and non-replicative vectors. The same strategy will be used to create a vector to delete ldh 2744.

gacgcatacaggttgtaacacccatttcccttagcttttcgggagatgaataaaacaaactttccgggtcctttacc
acaccgcccacataaagagctatgccgcatgaaagaaacgatatgttatcatttttttcgtaaactgttatttccga
acccggataaagctttaccatattattaactgctgccgtccctgcatgtgtacaccctataaccactattttcatatac
atcctcctttgtttgcttgtaaatatatcccatatataccacctaaatatattttataaacaaattcggtatatcattcttttg
gtaaataaaaagtacatccgatattagaatgtacctaaaaaaaattattattttattgtatatgctttatctgttttcattat
atggtttgctatccattctacggtaaaatcaagtaattccattaagtactgatcctgatccttgtctatcctgctataatc
cgtattactgattttctcaataaaatcatggtgttcaactttgtgggagagaagcttgcgatatcctatgctatgcatgt
attcttcttcataggtaaaatgaaagacagtgtaatcttttagttccgtaattagccgtacaatttcatcatatttgtctgt
aataagctgatttttcgtggcctcataaatttccgaagcaatctggaatagtttcttatgctgttcgtcgattttctcaatt
ccaagaataaattcgtctctccattctatcatatggaccctcctaaattgtaatgtataccaagattatacatacttcct
agaatataaacaatacaaggataaaattttaatatcgtatacctacataaatgactaacttaaagctctctaaaac
ttcttttttattatttctatactactaaaatcaaaaatattctctaaagtatttctacaaatgttgtttttgcaacaaagtagt
atacttttgcacccagaatgttttgttataacttacaaattaggggtatatttatagtaaatactaaatggaagagtag
gatattgattatgaacgagaaaaatataaaacacagtcaaaactttattacttcaaaacataatatagataaaata
atgacaaatataagattaaatgaacatgataatatctttgaaatcggctcaggaaaagggcattttacccttgaatt
agtacagaggtgtaatttcgtaactgccattgaaatagaccataaattatgcaaaactacagaaaataaacttgtt
gatcacgataatttccaactttttaaacaaggatatattgcagtttaaatttcctaaaaaccaatcctataaaatatttg
gtaatataccttataacataagtacggatataatacgcaaaattgtttttgatagtatagctgatgagatttatttaatc
gtggaatacgggtttgctaaaagattattaaatacaaaacgctcattggcattatttttaatggcagaagttgatattt
ctatattaagtatggttccaagagaatattttcatcctaaacctaaagtgaatagctcacttatcagattaaatagaa
aaaaatcaagaatatcacacaaagataaacagaagtataattatttcgttatgaaatgggttaacaaagaatac
aagaaaatatttacaaaaaatcaatttaacaattccttaaaacatgcaggaattgacgatttaaacaatattagctt
tgaacaattcttatctcttttcaatagctataaattatttaataagatcccctttacttcggatgcatgccgcaggcagg
catccgaagtagtttctccattatacaagtattctcttgagtacgtcgtcgcttctcagcagctgctttgctttttccctgtt
ttccggcacatggagataagtgtatctgttaggcttaatagtgtgtgccatgtcaattgccttttcgaagtcatctgcct
tcatttttaaggtttccacaaaattgataaaacccgtatcagtcagaaattttactacccgctgatatctgtgttcttga
accctgctcataagataggttgcaatcccaacctgaattccatgaagctgaggtgtctccagcagcttatctaaag
catgagatattagatgctcactaccgctggctggagcactgctgtctgctatctgcatggcaattccgctcattgtca
gagagtctaccatttcctttaaaaagaagttttctgtaacctgtgtgtagggcatccttacaatactgtttactgacttttt
agcaatcattgcagcaaaatcgtcaacctttgccgcattgttcctttcttcaaaataccagtcatacacagccgtaa
ttttggatattatgtctccgagacctgaataaataaatttcataggtgcattttttaatacatctaaatccactaatattcc
aaatggcatcgaggcatgtacggaagtacgcctgccatttataatcaaagagcagcctgagctggaaaaacc
atcgtttgaggttgatgtaggtatactgataaaaggaagcttgtttaaaaaagctatatatttggctgcatcaagcac
ctttcctcctcctactccgaccactgcatcggttttggagggaatagtaaaagccttgagcataagattttcaagcttt
atgtcatcatagtcgtaagtttcaagtactgcaagagattttcttgactttatggaatccagaatcttttcaccaaataa
gtcacgtattccctctccaaaaagtactacaacattactaattcctgccctttcaatatgtgc

Pyruvate Formate Lyase (pfl):

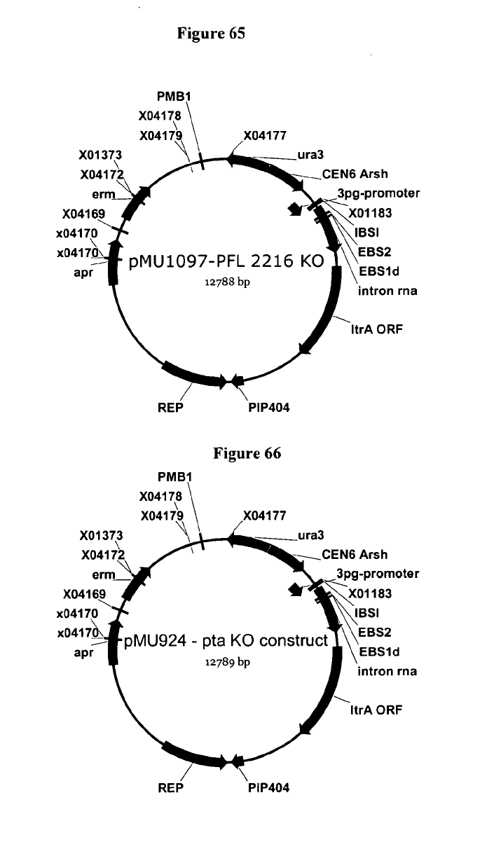

As described above for the C. cellulolyticum ack and pta genes, single and double crossovers are generated to disrupt the C. cellulolyticum pfl gene. An example of a vector for retargeting the L1.Ltrb intron to insert in C. cellulolyticum pfl gene(s) (SEQ ID NO: 94 and 95) is depicted in FIGS. 64 and 65. The vector sequence of these constructs are SEQ ID NO: 96 and 97.

Aldehyde/Alcohol Dehydrogenase (adh):

As described above for the C. cellulolyticum ack and pta genes, single and double crossovers are generated to disrupt the C. cellulolyticum adh genes. An example of a vector for retargeting the L1.Ltrb intron to insert in C. cellulolyticum adhE gene (SEQ ID NO:102) is depicted in FIG. 68. The vector sequence this construct is SEQ ID NO:103.

Sequence ID NOs: 104-106 represent additional alcohol and aldehyde dehydrogenases that are targeted which would decrease ethanol production and increase yields of lactate and acetate. TargeTron knockout constructs for these targets are in a manner that is similar to those for adhE.

C. phytofermentans Knockout Constructs
For Acetate Kinase (Gene 327 from C. phytofermentans Published Genome):

Single Crossover

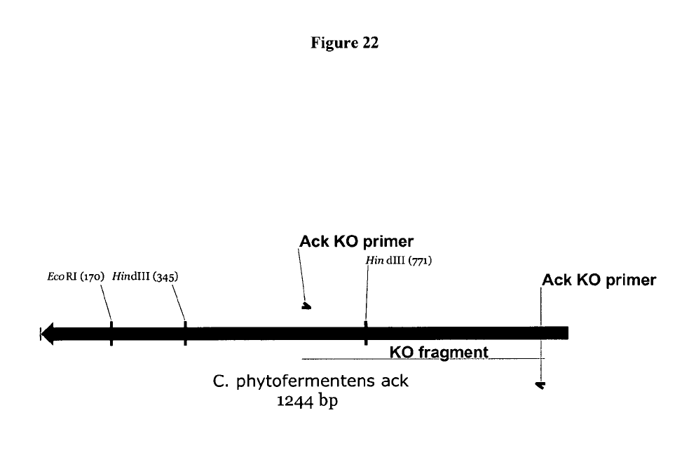

The acetate kinase gene of C. phytofermentans is 1,244 bp in length. A 572 bp internal fragment spanning nucleotides 55-626 will be amplified by PCR and cloned into suicide vectors and replicating vectors that have different selectable markers. Selectable markers to use will include those that provide drug resistance to C. phytofermentans. These plasmids will be used to disrupt the ack gene. A map of the ack gene and the region amplified by PCR for gene disruption are shown in FIG. 22. Restriction sites will be engineered during the design of the “ack KO primers” and will allow subsequent cloning of the fragment into numerous vectors. The sequence of the knockout fragment described above is set forth as SEQ ID NO:25.

### Double Crossover

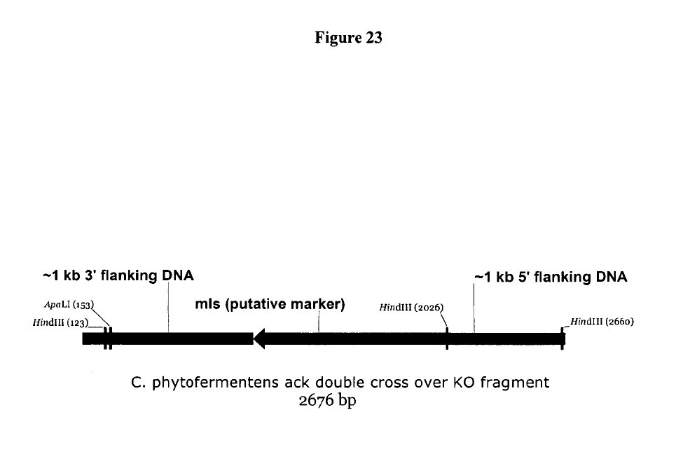

To construct a double crossover knockout vector for the ack gene of C. phytofermentans ˜1 kb of DNA flanking each side of the ack gene will be cloned. A selectable marker will be inserted between the flanking DNA. Selectable markers to use will include those that provide drug resistance to this strain. An example of a putative double crossover knockout construct with the mLs gene as a putative selectable marker is shown in FIG. 23.

The sequence that corresponds to the fragment depicted in FIG. 23 (SEQ ID NO:26) is set forth below. The mLs gene (putative selectable marker) is underlined and the remainder of the sequence corresponds to the flanking DNA. During primer design, restriction sites will be engineered and the 5′ and 3′ ends of the above fragment so that it can be cloned into a number of replicative and non-replicative vectors.

ctgagtgcaatgtaaaaaaggatgcctcaagtattcttgaaacatccttatattatactacaaaatcataaagtaa
attactcagctgtagcaatgatctcttttttgttgtaagatccacaagctttacaaactctatgaggcatcataagtgc
accacacttgctgcatttcactaagtttggagcagtcatcttccagtttgcacgacgactatctcttctagctttggaat
gtttattctttggacaaatagctcccattgattacacctccttaaacttgttaaaaatatctcggatagcagacattctt
gggtctagttctgtacggtcacacccgcactctccttcatttaggttagcaccgcagaccttgcagattcctttacagt
cttctttgcacagaaccttcattgggaaaccaatcaagacttcttcatagataagtttatctacgtctaaatcatatcc
ggaaacaaaatttgtttcatctaaatcctcggtacgctgttcctctgttttcgatacatcaatctctgtagccacgtcga
tgtcttgttggatggtttcttccttcaaacaacgatcgcaaggaacggctaacgctaatttcgtttttgcttccaccaga
atttttcggccacctagattagttaatctaagtttaaccggttctttataggtaatagaataaccgacaccatttaattc
gaatatatcaaattcaatcggtgcagtgtattctttgagaccattaggaacattcatgacttcagacatttgtatcagc
ataagtaactcctgtctaaaaaaacgcataatgtaagcgcccaaaaattcacactgttagtattataaacgcttaa
aataggtttgtcaactcctaactgttaaaaatgtcagaattgtgtaaccatattttctcttcattatcgttcttcccttatta
aataatttatagctattgaaaagagataagaattgttcaaagctaatattgtttaaatcgtcaattcctgcatgttttaa
ggaattgttaaattgattttttgtaaatattttcttgtattctttgttaacccatttcataacgaaataattatacttctgtttatc
tttgtgtgatattcttgatttttttctatttaatctgataagtgagctattcactttaggtttaggatgaaaatattctcttggaa
ccatacttaatatagaaatatcaacttctgccattaaaaataatgccaatgagcgttttgtatttaataatcttttagca
aacccgtattccacgattaaataaatctcatcagctatactatcaaaaacaattttgcgtattatatccgtacttatgtt
ataaggtatattaccaaatattttataggattggtttttaggaaatttaaactgcaatatatccttgtttaaaacttggaa
attatcgtgatcaacaagtttattttctgtagttttgcataatttatggtctatttcaatggcagttacgaaattacacctct
gtactaattcaagggtaaaatgcccttttcctgagccgatttcaaagatattatcatgttcatttaatcttatatttgtcatt
attttatctatattatgttttgaagtaataaagttttgactgtgttttatatttttctcgttcattgtatttctccttataatgttctta
aattcatttatcacggggcaacttaatatatccgaaatatagttcttctatatcgttcccccagtataatgattattatac
tatttaatcttcaacttaacaattggagtttccagttaagaaataataatttaatgccaaagcggatattcgcaatccg
cttacgctacttgctcataacctcaacaggcaatgaagctaagttaattatttactctgtgcctgaacagcagtgatt
gcaacaacaccaacgatatcatcagaagaacaacctcttgataaatcatttactggagctgcaataccctgagtt
aatggtccataagcttctgcctttgcaagacgctgtgttaacttatatccaatgttaccagcatcaaggtctgggaa
gattaatacgttagcttttccagcaatatcactaccaggagcttttgaagcacctacactaggaacgattgctgcat
ctaactggaactcgccgtcgatcttatattctgggtataattcatttgcaatcttagttgcttctacaaccttatcaacat
ctgcatgctttgcgcttccctttgttgaatgagaaagcatagctacgataggttcagagccaactaattgttcaaaa
ctcttcgctgtggaaccagcgattgctgctaactcttcagcatttggattctgatttaaaccagcatcagagaaaag
gaaagttccatttgcgcccatatcacaattaggtactaccattacgaagaaagcagaaactaacttagtatttgga
gcagtttttaaaatctgaagacatggtcttaaggtatctgctgtagagtgacaagcaccagatactaaaccatctg
catcgcccatcttaaccatcattacaccgtatgtaatgtagtctgttgttaaaagctcttttgctttttcaggggtcatgc
cttttgcctgtctaagttctacaagcttgttaatgtaagc

For Lactate Dehydrogenase (Genes 1389 and 2971 of C. phytofermentans Published Genome)

Single Crossover

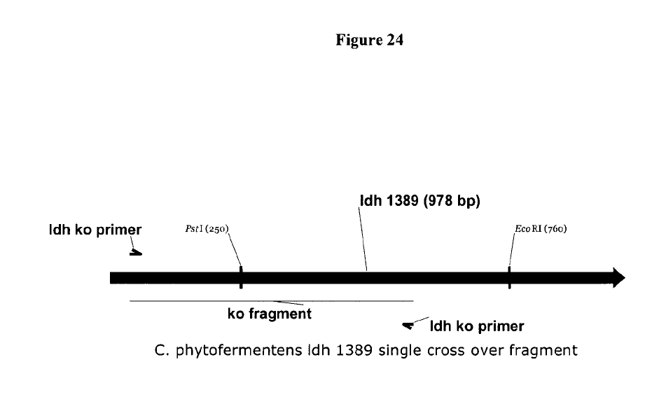

The ldh genes of C. phytofermentans are 978 bp (for gene 1389) (SEQ ID NO:27) and 960 bp (for gene 2971) (SEQ ID NO:28) in length. A ˜500 bp internal fragment near the 5′ end of each gene will be amplified by PCR and cloned into suicide vectors and replicating vectors that have different selectable markers. Selectable markers to use will include those that provide drug resistance. These plasmids will be used to disrupt the ldh 1389 and ldh 2971 genes. As an example, a map of the ldh 1389 gene and the region amplified by PCR for gene disruption are shown in FIG. 24.

Double Crossover

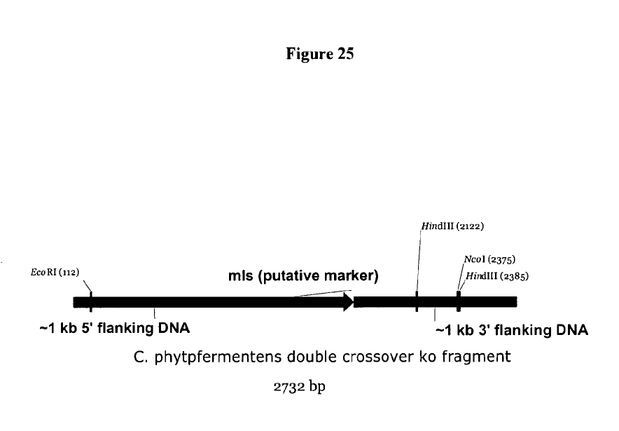

To construct a double crossover knockout vector for the ldh gene(s) of C. phytofermentans ˜4 kb of DNA flanking each side of the ldh gene(s) will be cloned. A selectable marker will be inserted between the flanking DNA. Selectable markers to use will include those that provide drug resistance to this strain. An example of a putative double crossover knockout construct with the mLs gene as a putative selectable marker is shown in FIG. 25.

The sequence that corresponds to the fragment depicted in FIG. 25 is set forth below as SEQ ID NO:29. The mLs gene (selectable marker) is underlined and the remaining portion of the sequence corresponds to the flanking DNA. During primer design, restriction sites will be engineered and the 5′ and 3′ ends of the above fragment so that it can be cloned into a number of replicative and non-replicative vectors. The same strategy will be used to create a vector to delete ldh 2971.

tggaatctcactatgcaccaatgtggtactaaattatatctttatctatggaaaattaggttttccgcgaatggagata
gagggagctgccattgctactttaatttgtagaattcttgagagtattttagttgttatttatatgtataagggtgagaag
gtacttaagatgagactttcttatatttttaagagatctaaacagtattttcgctctttggctcgttatagtgcgccagtgc
ttatgagtgaggttaactgggggcttgggattgctgttcagtctgcaatcattgggcgtatgggtgttagttttcttaca
gccgccagcttcattaatgtagtacaacagttagccggaatcattctgattggtattggtgtgggttcgagcattata
atagggaatttgattggtgagggaaaagagcatgaggcgagaatgctagccaataagttaatacgtatcagtat
gatactcggaggaattgttgcttttgcagtaatcttactacgtccaatcgctcctaactttattgaggcgtctaaggaa
acagcggatttaattcgtcagatgctatttgtttcggcttacctcttattcttccaagccttatctgtattaactatggccg
gaatattacgtggtgcaggggataccctttactgtgcaacctttgatgttttgaccttatgggtactaaaacttggagg
aggtttgcttgcaaccatagtacttcatcttccacctgtatgggtttactttatcttaagtagcgatgagtgtgttaaagc
gctatttacggtaccgcgggtcttaaagggacgttggattcatgatacaacactgcattaagatttcatatgtccag
atatttttgcacagtagcataattactagagcttattcctataatattcataggttttgatggtccattttacgttacgata
gcatatattacatcaaaaccaattctatataagatgaggttatagtatgaacgagaaaaatataaaacacagtca
aaactttattacttcaaaacataatatagataaaataatgacaaatataagattaaatgaacatgataatatctttg
aaatcggctcaggaaaagggcattttacccttgaattagttacagaggtgtaatttcgtaactgccattgaaataga
ccataaattatgcaaaactacagaaaataaacttgttgatcacgataatttccaagttttaaacaaggatatattgc
agtttaaatttcctaaaaaccaatcctataaaatatttggtaatataccttataacataagtacggatataatacgca
aaattgtttttgatagtatagctgatgagatttatttaatcgtggaatacgggtttgctaaaagattattaaatacaaaa
cgctcattggcattatttttaatggcagaagttgatatttctatattaagtatggttccaagagaatattttcatcctaaa
cctaaagtgaatagctcacttatcagattaaatagaaaaaaatcaagaatatcacacaaagataaacagaagt
ataattatttcgttatgaaatgggttaacaaagaatacaagaaaatatttacaaaaaatcaatttaacaattccttaa
aacatgcaggaattgacgatttaaacaatattagctttgaacaattcttatctcttttcaatagctataaattatttaata
agaagtaataggaaataatactcgaattattctgcaatctgttctaaaaaataaaattaagaaattactatagcaa
gccaggttaaaattactagcttgctatttttgtgcatttagtacagttttgattattaaagaataaatttaataactattttg
caataagttattgactatttcacaagttagtgttactatacaagtatgaaataaagatacataaaaaaataaataat
atgaaacataaattcatgacatgcggaatagaatgaaagaatattatgtcggttcctaatactaaatggatataac
aatctattgaaacacttatggggtgtaagtgtggagagaatttctaaagcgccaaaagactctacatatgaaattc
taaagcttcacacgggaataatctaatttatgtatcttattatcataattcaggaaggtagtgtgaaaatataaaaatt
agttttcctgtttcattcaggcagtagcatttcttaaacaaatttgctatgcattgggtgttatctgaaaaacaaaaagc
aattttctcacaacttatttctgaacaacaatggtattaaaaatttggaggaggattttactatgaaaaaaacggtaa
cattactgttggttctgaccatggtggtaagcttatttgcagcatgtggtaagaaaaatggatcaagcgaaaccgg
cacaaaagatcctgtggcaacaagcggtgcaaaagaacctgacaaacaagatccaggcaataaagagcct
gaaaaacaagaccctgttaaaatcaagatttattactctgataatgcaaccttaccatttaaagaagattggttagt
tataaaggaagctgagaagagatttaatgttgatttcgatttcgaagtaattccaattgcagattatcaaacaaaag
tttctttaacattaaatacaggaaataacgctccagatgtcatcctttatcagtcaacgcagggagagaatgcatct

Cald. kristjanssonii and C. stercorarium subs leptospartum

To the best of our knowledge, genome sequencing of the above organisms has not occurred and if it has, it has not been made available to the public. Based on our experimental results these organisms are cellulolytic and xylanolytic. The DNA sequences of genes encoding key metabolic enzymes are needed from these organisms in order to genetically engineer them and divert carbon flow to ethanol. These include such enzymes as acetate kinase and lactate dehydrogenase. In order to obtain the sequences of these genes, the genomes of these organisms will be sequenced.

With access to genome sequences, the conserved nature of the above enzymes may be used to find the encoding genes and flanking DNA. These sequences will be used to design constructs for targeted mutagenesis employing both single and double crossover strategies. These strategies will be identical to those described above. We will also determine which antibiotics can be used as selectable markers in these organisms and which protocols for transformation work best.

### Example 3

### Transformation of C. cellulolyticum

Cells were grown in 50 mL of GS media with 4 g/l cellobiose to an OD of 0.8 in anaerobic conditions, incubated at 34 degrees C. After harvesting they were washed 3 times in equal volumes with a wash buffer containing 500 mM sucrose and 5 mM MOPS with pH adjusted to 7. After the final wash, the cell pellet was resuspended in an equal volume of wash buffer 10 ul aliquots of the cell suspension were placed in a standard electroporation cuvette with a 1 mm electrode spacing. 1 ul plasmid DNA was added. The concentration of the plasmid DNA was adjusted to ensure between a 1:1 and 10:1 molar ratio of plasmid to cells. A 5 ms pulse was applied with a field strength of 7 kV/cm (measured) across the sample. A custom pulse generator was used. The sample was immediately diluted 1000:1 with the same media used in the initial culturing and allowed to recover until growth resumed, and was determined via an increase in the OD (24-48 h). The recovered sample was diluted 50:1 and placed in selective media with either 15 ug/mL erythromycin or 15 ug/mL chloramphenicol and allowed to grow for 5-6 days. Samples exhibiting growth in selective media were tested to confirm that they were in fact C. cellulolyticum and that they had the plasmid.

### Example 4

### Constructs for Engineering Cellulolytic Strains

Cellulose is one of the main components of biomass, which can be potentially used as a substrate for generation of lactate or acetate by fermentation with Clostridium thermocellum. However, in this process, much energy and carbon sources are used to form by-product acetate and lactate, and ethanol. Engineering of the metabolic pathways of cellulose utilization in Clostridium thermocellum is necessary to minimize the ethanol production and make energy and carbon flows favorable to lactate or acetate formation.

Acetate yield can be increased by partial, substantial, or complete deletion, silencing, inactivation, or down-regulation of single genes or combinations of genes in competing pathways leading to lactate, and ethanol production. Key genes to be targeted in these pathways include ldh for lactate, adh's for ethanol. In certain cases (e.g., adh) multiple homologous genes are predicted for a given step.

Lactate yield can be increased by partial, substantial, or complete deletion, silencing, inactivation, or down-regulation of single genes or combinations of genes in competing pathways leading to acetate, ethanol, and formate production. Key genes to be targeted in these pathways include pta and ack (individual and/or combined mutations) for acetate, adh's for ethanol, and pfl for formate. In certain cases (e.g., adh) multiple homologous genes are predicted for a given step.

Inactivation of the ack, pta and pta-ack Genes in C. thermocellum

SEQ ID NO: 77 and NO 78 are the pta and ack genes from Clostridium thermocellum (ATCC 27405). Pta catalyzes the conversion of acetyl-CoA+phosphate=CoA+acetyl phosphate. Ack catalyzes the conversion of ADP+acetyl phosphate=ATP+acetate. Deletion of ack and/or pta results in the elimination of acetate production which may increase the yields of ethanol, lactate, and formate. In conjunction with mutation targeting the pathways leading to formate and ethanol production, the pta, ack, pta-ack mutation(s) enhances lactate yields.

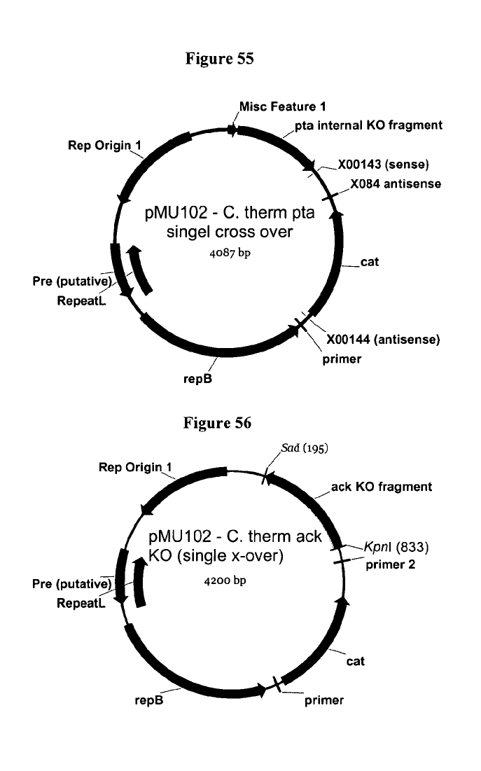

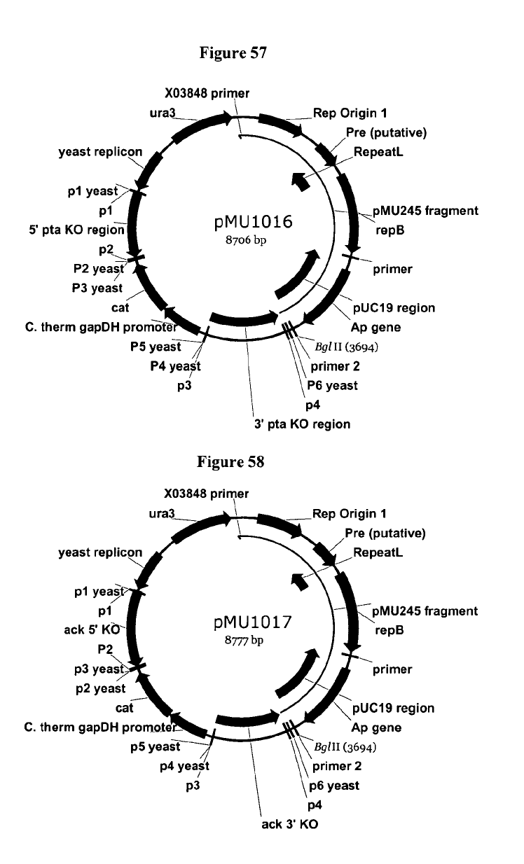

SEQ ID NOS: 79-83, depicted in FIGS. 55-59, show ack, pta, and pta-ack knockout plasmids for C. thermocellum. Single crossover and double crossover plasmids designed to partially, substantially, or completely delete, silence, inactivate, or down-regulate the Ack and/or Pta enzymes. Single crossover plasmids are designed with a single DNA sequence (400 bp to 1000 bp) homologous to an internal section of the ack or pta gene, double crossover plasmids are designed with two DNA sequences (400 to 1000 bp) homologous to regions upstream (5′) and downstream (3′) to the ack, pta, and pta-ack genes. Plasmids are designed to use antibiotic markers known to one of ordinary skill in the art as described supra for selection in C. thermocellum. One example of such a marker is thiamphenicol and derivatives thereof. Plasmids can be maintained in E. coli and constructed through a DNA synthesis contract company, such as Codon Devices or DNA 2.0.

Inactivation of the ack Gene in C. thermocellum Based on the Plasmid pIKM1

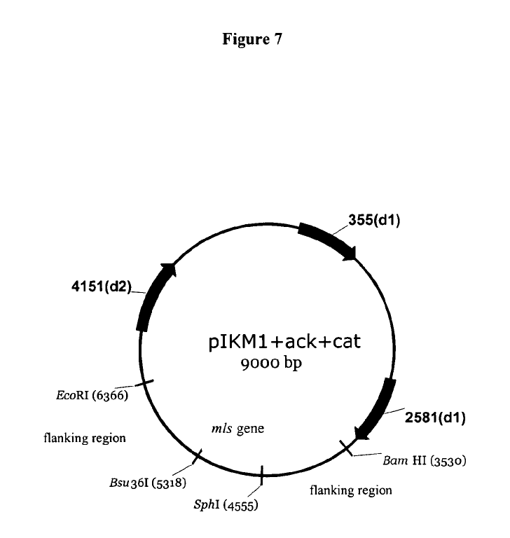

To knock out the ack gene, a vector is constructed on the multiple cloning sites (MCS) of the plasmid pIKM1, in which the cat gene, encoding chloramphenicol acetyltransferase, is inserted into a DNA fragment of 3055 bp, involving the ack and the pta genes (encoding phosphotransacetylase), leading to knockout of 476 bp of the ack gene and 399 bp of the pta gene, and forming 1025 bp and 1048 bp flanking regions on both sides of the mLs gene respectively (FIG. 7). pNW33N contains pBC1 replicon, which is isolated from Bacillus coagulans and Staphylococcus aureus, and is anticipated to be stably replicated in Gram positive strains of bacteria, including Clostridium thermocellum. The sequence of the ack knockout vector constructed on plasmid pIKM1 is set forth as SEQ ID NO:1.

Inactivation of the ack Gene in C. thermocellum Based on the Replicative Plasmid pNW33N

To knock out the ack gene, a vector is constructed on the multiple cloning sites

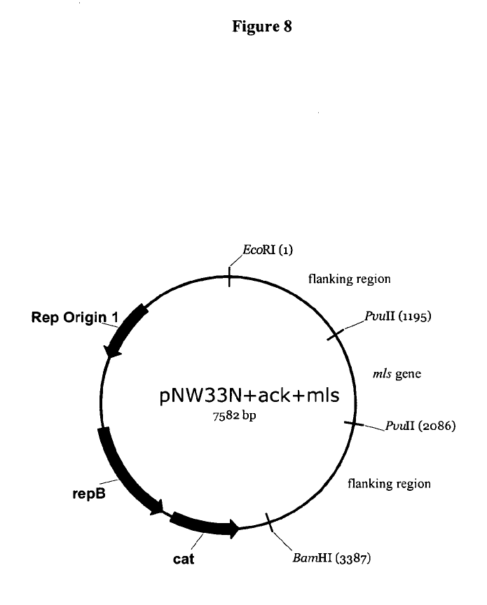

(MCS) of the replicative plasmid pNW33N, in which the macrolide, lincosamide, and streptogramin B (MLS_{B}) resistant gene mLs is inserted into a DNA fragment of 3345 bp, which includes the ack gene, the pta gene (encoding phosphotransacetylase) and an unknown upstream gene, leading to knockout of 855 bp of the ack gene and formation of flanking regions of 1195 bp and 1301 bp on either side of the mLs gene (FIG. 8). pNW33N contains pBC1 replicon, which is isolated from Bacillus coagulans and Staphylococcus aureus, and is anticipated to be stably replicated in Gram positive strains of bacteria, including Clostridium thermocellum. The sequence of the ack knockout vector constructed on plasmid pNW33N is set forth as SEQ ID NO:2.

Inactivation of ldh Gene in C. thermocellum

SEQ ID NO: 84 is the ldh gene from Clostridium thermocellum (ATCC 27405). Ldh catalyzes the NADH-dependent reduction of pyruvate to lactate. Deletion of ldh will result in the elimination of lactate production which may increase the yields of ethanol, acetate, and formate. In conjunction with mutation targeting the pathways leading to formate and ethanol production, the ldh mutation would enhance acetate yields.

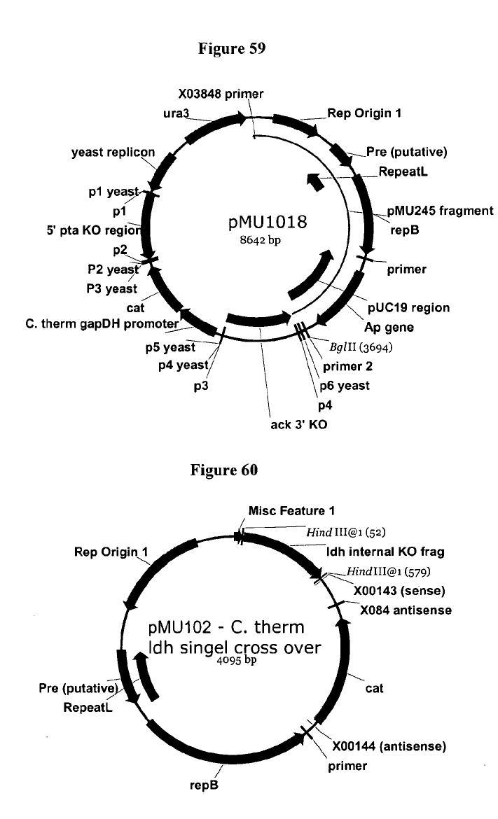

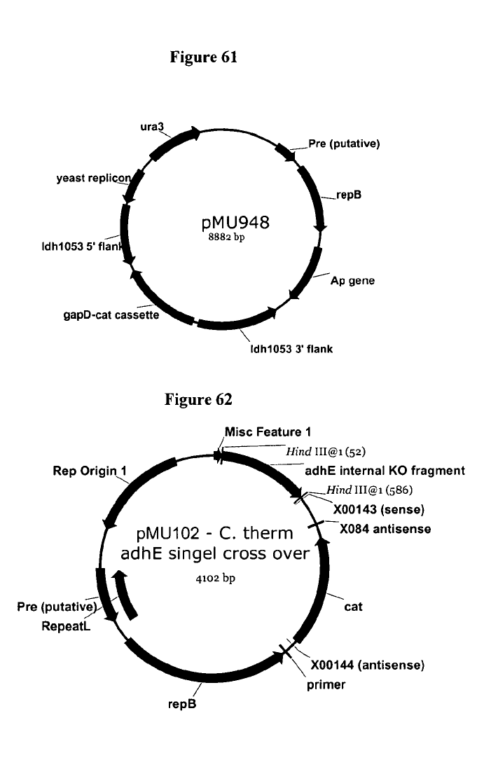

SEQ ID NOS: 85 and 86, depicted in FIGS. 60 and 61, show ldh knock out plasmids for C. thermocellum. Single crossover and double crossover plasmids designed to partially, substantially, or completely delete, silence, inactivate, or down-regulate the Ldh enzyme. Single crossover plasmids are designed with a single DNA sequence (400 bp to 1000 bp) homologous to an internal section of the ack or pta gene, double crossover plasmids are designed with two DNA sequences (400 to 1000 bp) homologous to regions upstream (5′) and downstream (3′) to the ldh gene. Plasmids are designed to use antibiotic markers known to one of ordinary skill in the art as described supra for selection in C. thermocellum. One example of such a marker is thiamphenicol and derivatives thereof. Plasmids can be maintained in E. coli and constructed through a DNA synthesis contract company, such as Codon Devices or DNA 2.0.

Inactivation of the ldh Gene in C. thermocellum Based on the Plasmid

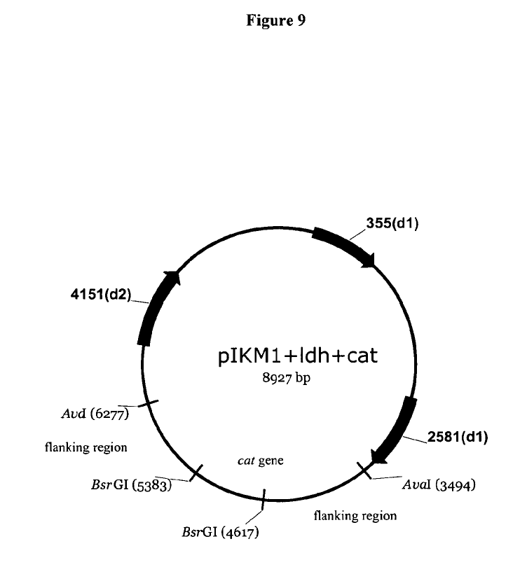

To knock out the ldh gene, a vector is constructed on the multiple cloning sites (MCS) of the plasmid pIKM1, in which the cat gene, encoding chloramphenicol acetyltransferase, is inserted into a DNA fragment of 3188 bp, involving the ldh and the mdh gene (encoding malate dehydrogenase), leading to knockout of a DNA fragment of 1171 bp, including part of the ldh and mdh genes, and forming 894 bp and 1123 bp flanking regions on both sides of the mLs gene, respectively (FIG. 9). The sequence of the ldh knockout vector constructed on plasmid pIKM1 is set forth as SEQ ID NO:3.

Inactivation of the ldh Gene in C. thermocellum Based on Plasmid pNW33N

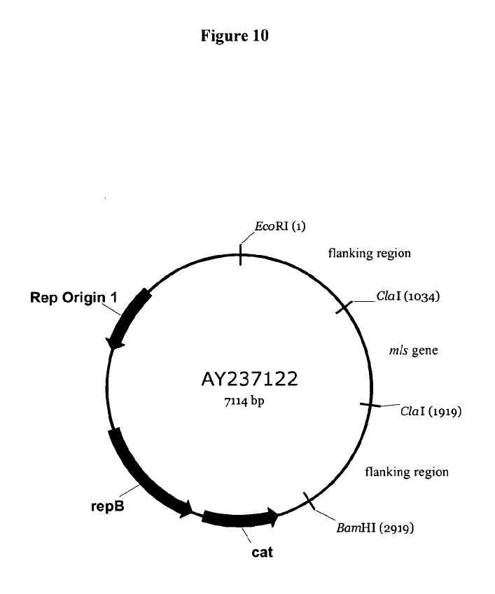

To knock out the ldh gene, a vector is constructed on the multiple cloning sites (MCS) of the replicative plasmid pNW33N, in which the macrolide, lincosamide, and streptogramin B (MLS_{B}) resistant gene mLs is inserted into a DNA fragment of 2523 bp, which includes the ldh gene and the mdh gene (encoding malate dehydrogenase), leading to knocking out of a fragment of 489 bp of the ldh gene and formation of flanking regions of 1034 bp and 1000 bp on either side of the mLs gene (FIG. 10). pNW33N contains pBC1 replicon, which is isolated from Bacillus coagulans and Staphylococcus aureus, and is anticipated to be stably replicated in other Gram positive strains of bacteria, including Clostridium thermocellum. The sequence of the ldh knockout vector constructed on plasmid pNW33N is set forth as SEQ ID NO:4.

Inactivation of the ldh Gene in Clostridium thermocellum Based on Plasmid pUC19

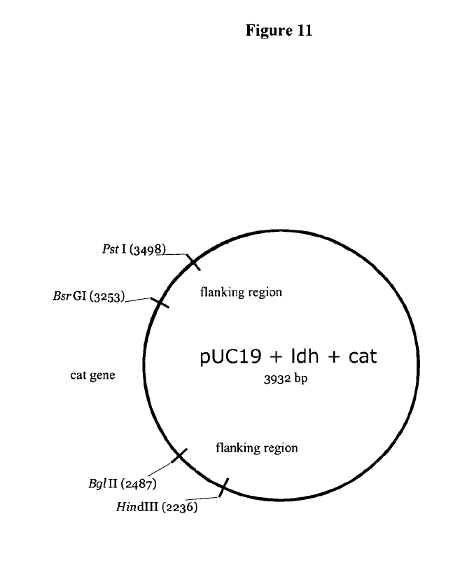

To knock out the ldh gene, a vector is constructed on the multiple cloning sites (MCS) of the pUC19 plasmid, in which a gene encoding chloramphenicol acetyltransferase (the cat gene) is inserted into a ldh gene fragment of 717 bp, leading to a flanking region of 245 bp and 255 bp on either side of the cat gene (FIG. 11). pUC19 is an E. coli plasmid vector, containing pMB1 origin, which cannot be amplified in Gram positive strains of bacteria, including Clostridium thermocellum. A similar vector may be constructed, in which the mLs gene is flanked by the ldh gene fragments. The sequence of the ldh knockout vector constructed on plasmid pUC19 is set forth as SEQ ID NO:5.

Inactivation of adh Gene in C. thermocellum

SEQ ID NO: 87 is the adhE gene from Clostridium thermocellum (ATCC 27405). AdhE is a dual function enzyme that catalyzes the NADH-dependent reduction of AcetlyCoA to acetylaldehyde and the NADH-dependent reduction of acetylaldehyde to ethanol. Deletion of adhE will result in decreased production of ethanol which may increase the yields of ethanol, acetate, and formate. In conjunction with mutations targeting the pathways leading to formate and lactate production, the adhE mutation would enhance acetate yields. Likewise, In conjunction with mutations targeting the pathways leading to formate and acetate production, the adhE mutation would enhance lactate yields.

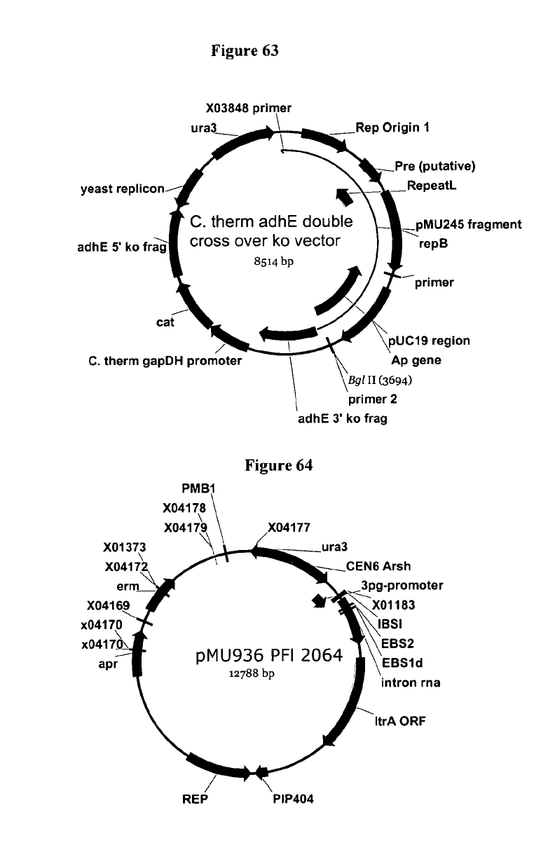

SEQ ID NOS: 88 and 89, depicted in FIGS. 62 and 63, show adhE knock out plasmids for C. thermocellum. Single crossover and double crossover plasmids designed to partially, substantially, or completely delete, silence, inactivate, or down-regulate the AdhE enzyme. Single crossover plasmids are designed with a single DNA sequence (400 bp to 1000 bp) homologous to an internal section of the adhE gene, double crossover plasmids are designed with two DNA sequences (400 to 1000 bp) homologous to regions upstream (5′) and downstream (3′) to the adhE gene. Plasmids are designed to use antibiotic markers known to one of ordinary skill in the art as described supra for selection in C. thermocellum. One example of such a marker is thiamphenicol and derivatives thereof. Plasmids can be maintained in E. coli and constructed through a DNA synthesis contract company, such as Codon Devices or DNA 2.0.

SEQ ID NOS: 90-93 represent additional alcohol and aldehyde dehydrogenases that could be targeted which may decrease ethanol production and increase yields of lactate and acetate. Single and double cross over knockout constructs for these targets would be made in a manner that is similar to those for adhE.

Inactivation of pfl Gene in C. thermocellum

SEQ ID NO: 45 is the pyruvate-formate-lyase (aka formate acetyltransferase, EC. 2.3.1.54, pfl) gene from Clostridium thermocellum (ATCC 27405). Pfl catalyzes the conversion of pyruvate to Acetyl-CoA and formate. Deletion of pfl will result in the elimination of formate production, which may increase the yields of ethanol and lactate. In conjunction with mutation targeting the pathways leading to acetate, and ethanol production, a Pfl mutation would enhance lactate production.

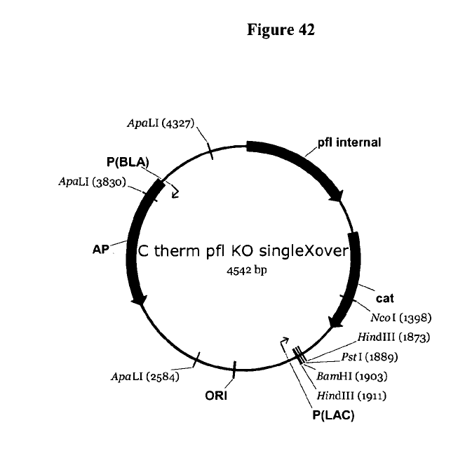

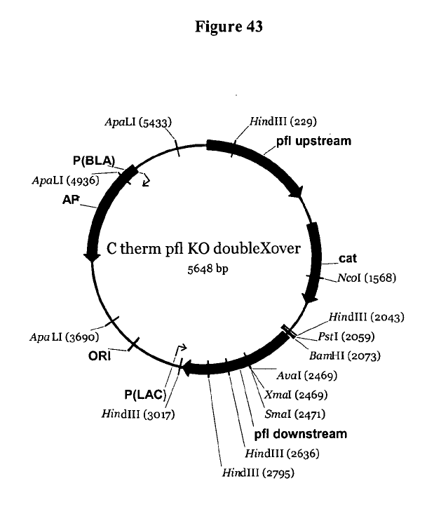

SEQ ID NOS: 49-50, depicted in FIGS. 42-43, show pfl knockout plasmids for C. thermocellum. A single crossover and double crossover plasmid designed to partially, substantially, or completely delete, silence, inactivate, or down-regulate the pfl enzyme. Single crossover plasmids are designed with a single DNA sequence (400 bp to 1000 bp) homologous to an internal section of the pfl gene, double crossover plasmids are designed with two DNA sequences (400 to 1000 bp) homologous to regions upstream (5′) and downstream (3′) to the pfl gene. Plasmids are designed to use antibiotic markers known to one of ordinary skill in the art as described supra for selection in C. thermocellum. One example of such a marker is thiamphenicol and derivatives thereof. Plasmids can be maintained in E. coli and constructed through a DNA synthesis contract company, such as Codon Devices or DNA 2.0.

Expression of Xylose Isomerase and Xylulose Kinase in C. thermocellum and C. straminisolvens (Prophetic Example)

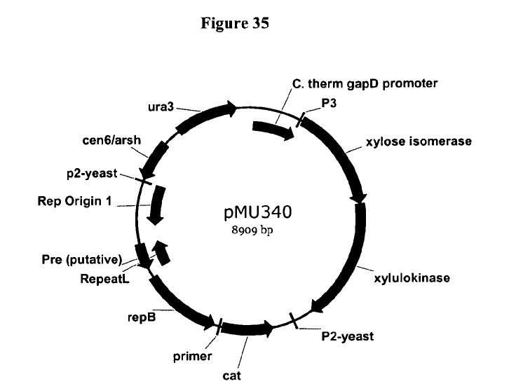

For expression of xylose isomerase and xylulose kinase in C. thermocellum, the xylose isomerase and xylulose kinase genes were cloned from T. saccharolyticum and placed under control of the C. thermocellum gapDH promoter. This cassette is harbored in a C. thermocellum replicative plasmid based on the pNW33N backbone, resulting in pMU340 (FIG. 35) SEQ ID NO:74. Upon transfer into C. thermocellum, the resulting transformation can be assayed for the ability to grow on xylose. Analogous constructs can be created using the C. kristajanssonii xylose isomerase and xylulose kinase genes. These constructs can be tested for functionality in C. straminsolvens as well.

Expression of Pyruvate Decarboxylase and Alcohol Dehydrogenase in C. thermocellum and C. straminisolvens (Prophetic Example)

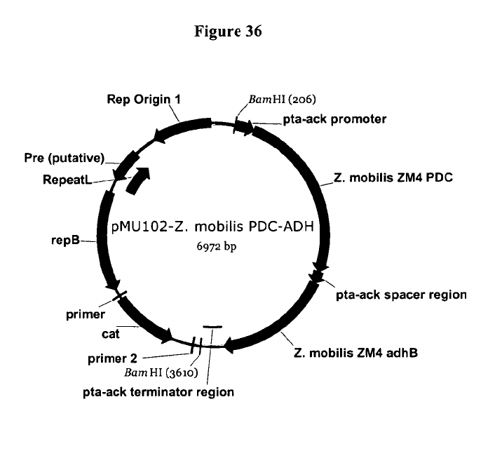

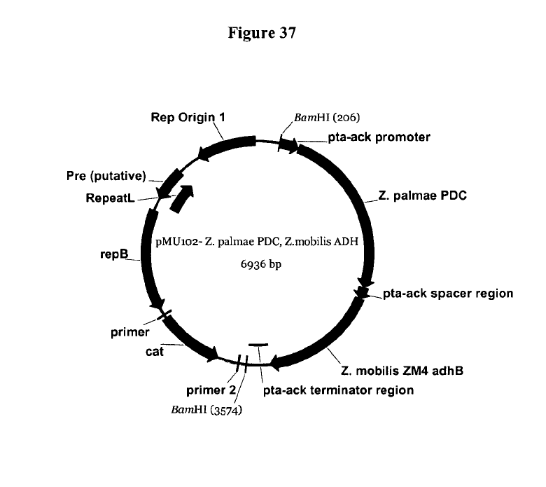

For expression of pyruvate decarboxylase and alcohol dehydrogenase in C. thermocellum, the pyruvate decarboxylase genes are cloned from sources Z. mobilis and Z. palmae and the alcohol dehydrogenase gene is cloned from source Z. mobilis. These genes (pdc and adh) will be expressed as an operon from the C. thermocellum pta-ack promoter. This cassette is harbored in a C. thermocellum replicative plasmid based on the pNW33N backbone (FIGS. 36 and 37), SEQ ID NOS:75 and 76. Upon transfer into C. thermocellum, the resulting transformation can be screened for enhanced ethanol production and/or aldehyde production to measure the functionality of the expressed enzymes. These constructs will be tested for functionality in C. straminsolvens as well.

### Example 5

### Fermentation of Avicel® Using C. straminisolvens

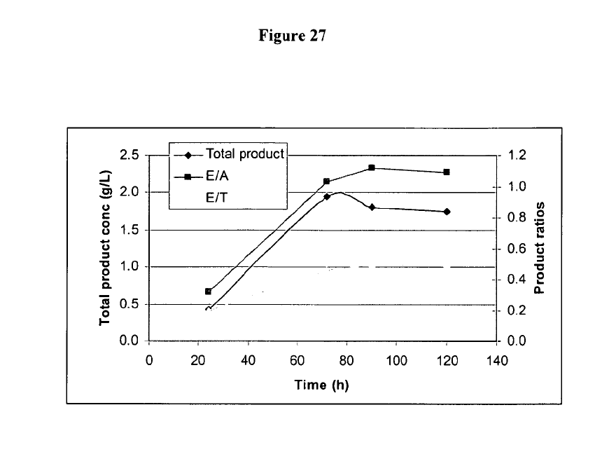

C. straminisolvens was used to ferment 1% Avicel® in serum bottles containing CTFUD medium. The product concentration profile and the ratios are shown in FIG. 27. About 2 g/L of total products was generated in 3 d with ethanol constituting about 50% of the total products. FIG. 27 shows the product concentration profiles for 1% Avicel® using C. straminisolvens. The ethanol to acetate ratio is depicted as E/A and the ratio of ethanol to total products is depicted as E/T.

### Example 6

### Design of Knockout Constructs for Mesophilic and Thermophilic Cellulolytic, and Xylanolytic Organisms: Use of TargeTron Mediated Gene Inactivation

Mobile group II introns, found in many bacterial genomes, are both catalytic

RNAs and retrotransposable elements. They use a mobility mechanism known as retrotransposition in which the excised intron RNA reverse splices directly into a DNA target site and is then reverse transcribed by an intron-encoded protein. The mobile Lactococcus lactis L1.LtrB group II intron has been developed into genetic tools known as Targetron™ vectors, which are commercially available from Sigma Aldritch (Catalog #TA0100). This product and its use are the subject of one or more of U.S. Pat. Nos. 5,698,421, 5,804,418, 5,869,634, 6,027,895, 6,001,608, and 6,306,596 and/or other pending U.S. and foreign patent applications controlled by InGex, LLC.

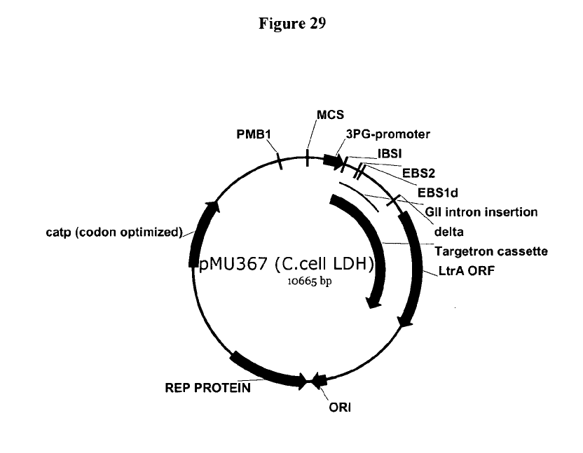

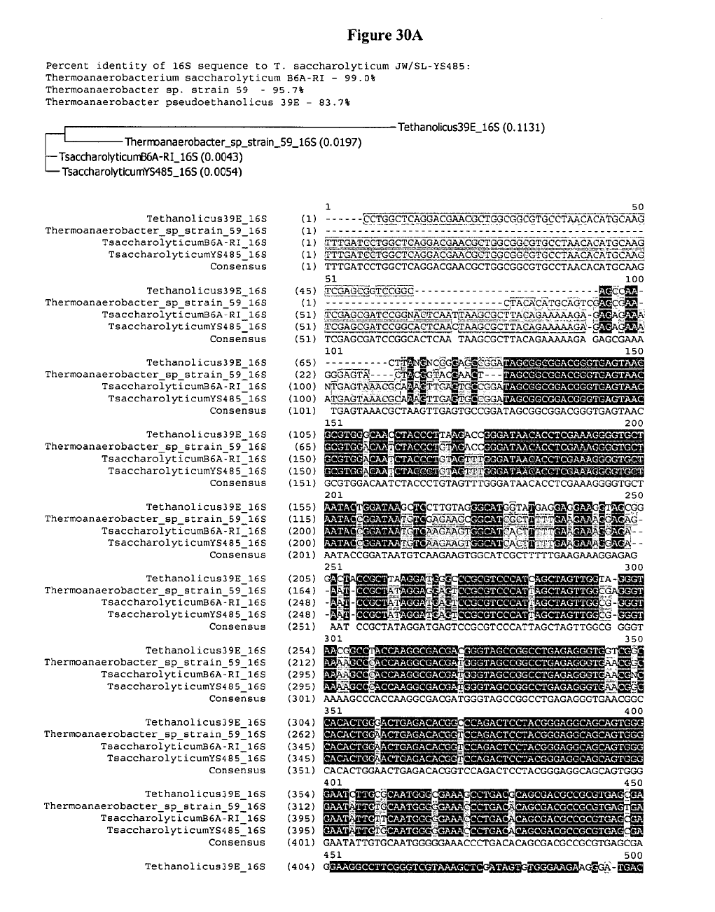

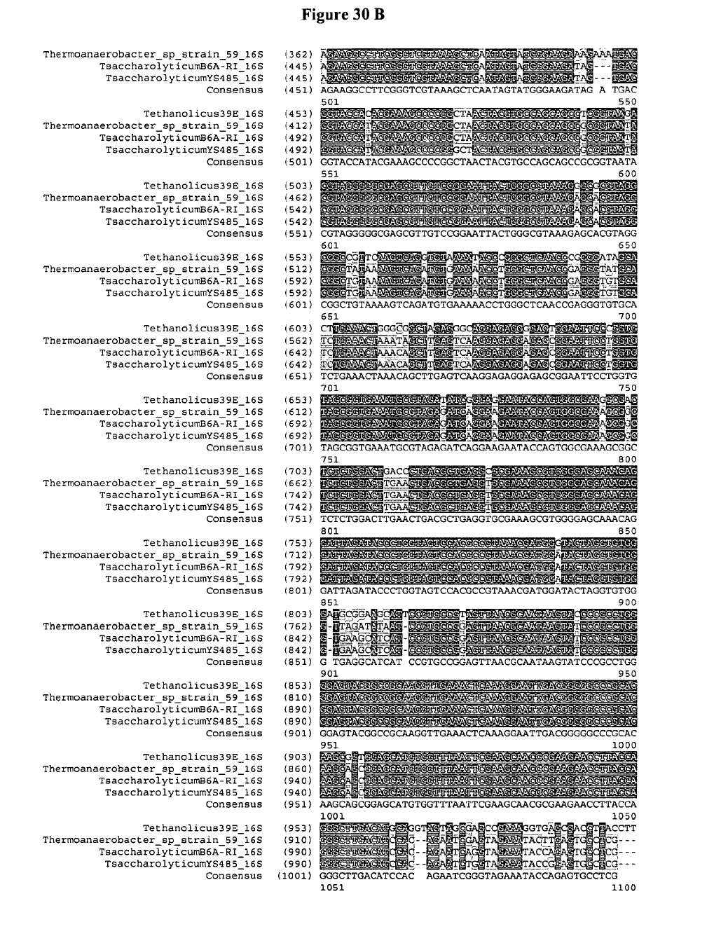

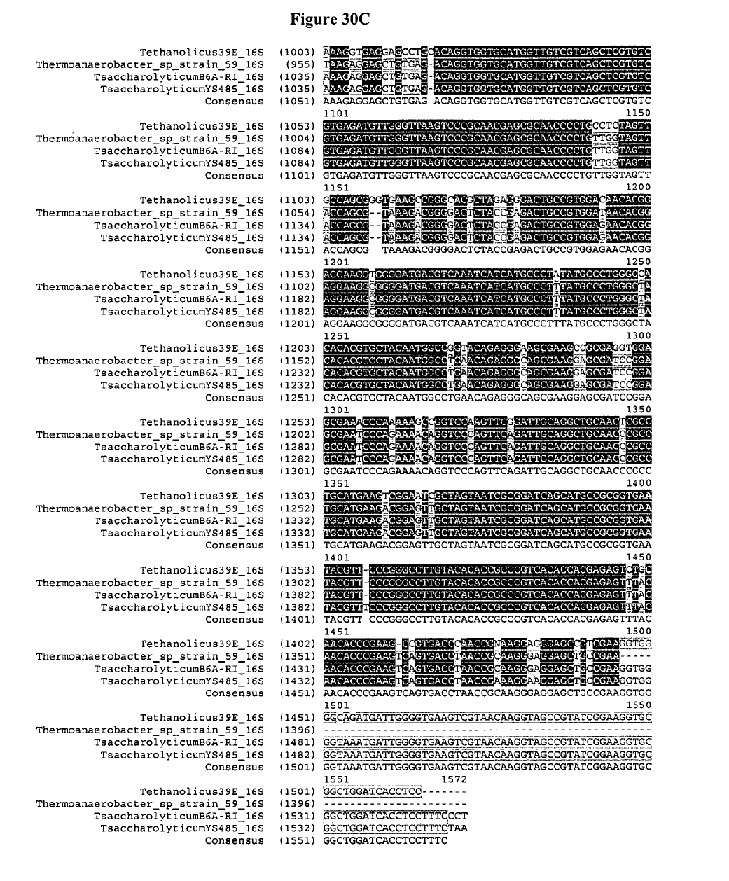

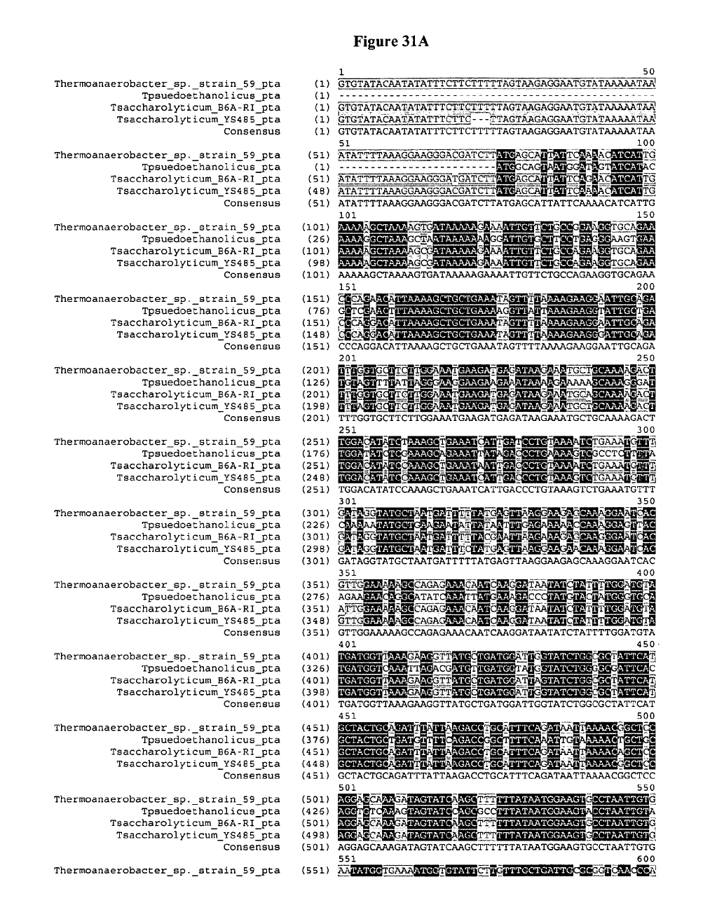

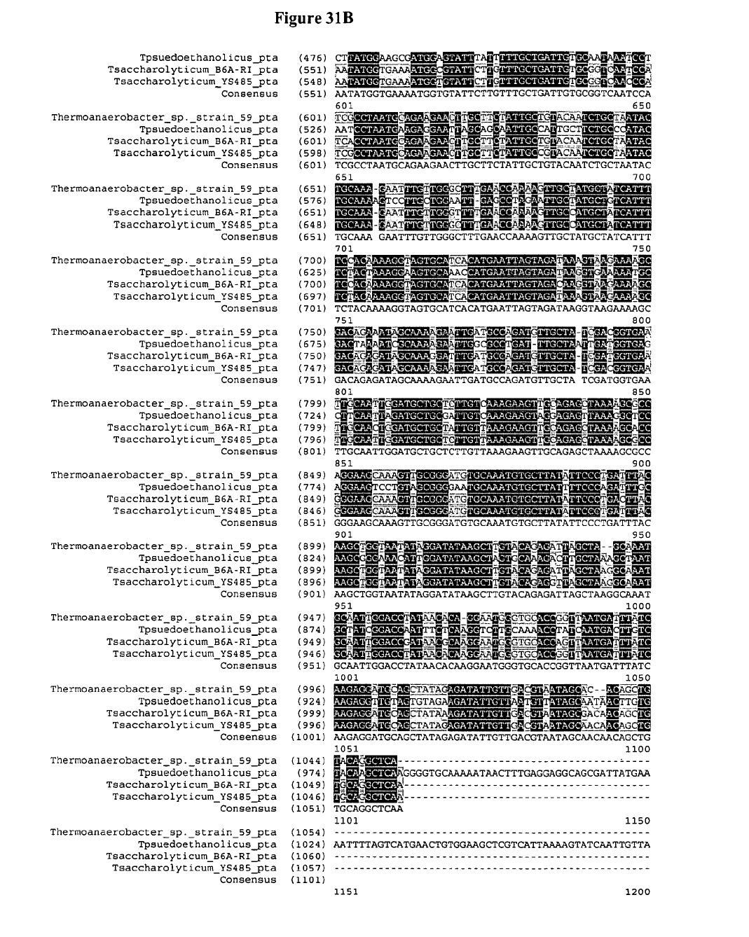

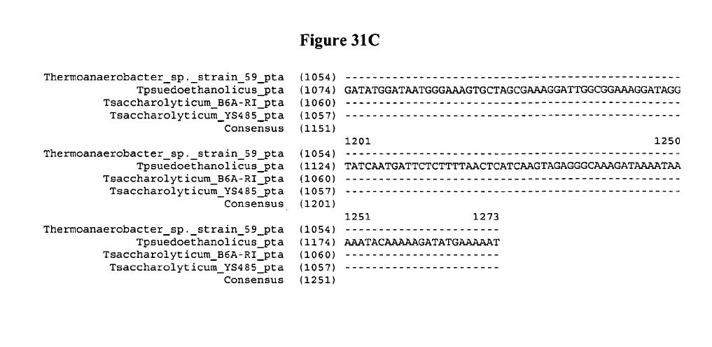

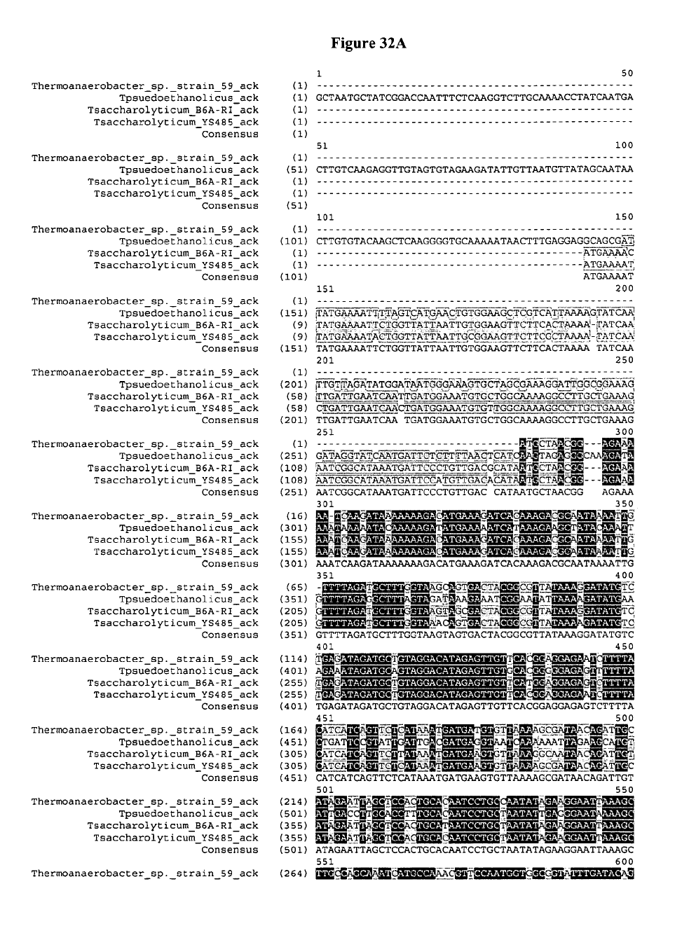

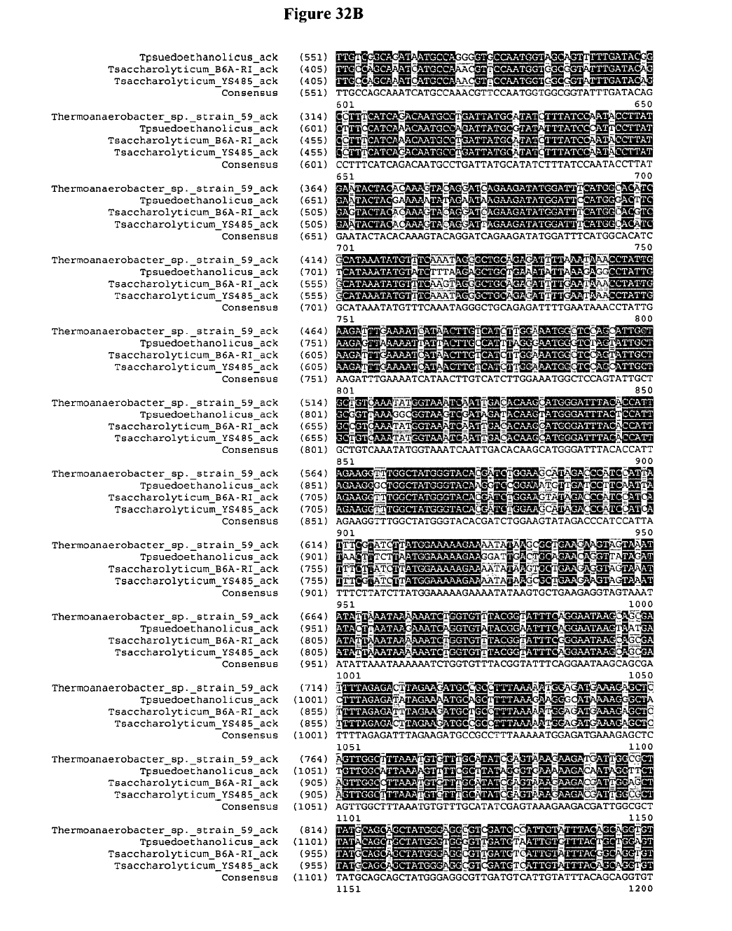

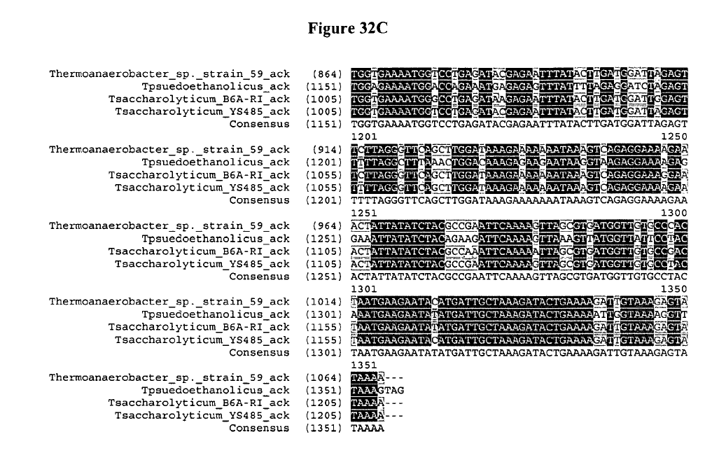

Targetrons cassettes (FIGS. 28 and 29) which contain all the necessary sequences for retro-transposition may be sub-cloned into vectors capable of replication in mesophilic or thermophilic cellulolytic organisms. The Targetron cassette may be modified by replacing the lac promoter with any host- or species-specific constitutive or inducible promoters. The cassettes may be further modified through site-directed mutagenesis of the native recognition sequences such that the Group II intron is retargeted to insert into genes of interest creating genetic knockouts. For example, the group II intron could be redesigned to knockout lactate dehydrogenase or acetate kinase in any mesophilic or thermophilic cellulolytic organism. Table 4 depicts an example of insertion location and primers to retarget Intron to C. cellulolyticum acetate kinase (SEQ ID NO:21). Table 5 depicts an example of insertion location and primers to retarget Intron to C. cellulolyticum lactate dehydrogenase (SEQ ID NO:21).

An example of a vector for retargeting the L1.Ltrb intron to insert in C. cell. ack gene (SEQ ID NO:21) is depicted in FIG. 28. The vector sequence of pMU367 (C. cell. acetate kinase KO vector) is SEQ ID NO:30.

An example of a vector for retargeting the L1.Ltrb intron to insert in C. cell. LDH2744 gene (SEQ ID NO:23) is depicted in FIG. 29. The vector sequence of pMU367 (C. cell. lactate dehydrogenase KO vector) is set for as SEQ ID NO:31.

TABLE 4

| | |
|---|---|
| Predicted Insertion location | ATTTACCTGGCTGGGAATACTGAGACATAT-intron- |
| (SEQ ID NO: 62) | GTCATTGAGGCCGTA |
| IBS1 mutagenic primer | AAAAAAGCTTATAATTATCCTTAATTTCCTACTACGTGCGCCCA |
| (SEQ ID NO: 63) | GATAGGGTG |
| EBS1d mutagenic primer | CAGATTGTACAAATGTGGTGATAACAGATAAGTCTACTACTGTA |
| (SEQ ID NO: 64) | ACTTACCTTTCTTTGT |
| EBS2 mutagenic primer | TGAACGCAAGTTTCTAATTTCGGTTGAAATCCGATAGAGGAAAG |
| (SEQ ID NO: 65) | TGTCT |

TABLE 5

| | |
|---|---|
| Predicted Insertion location | TTAAATGTTGATAAGGAAGCTCTTTTCAAT-intron- |
| (SEQ ID NO: 66) | GAAGTTAAGGTAGCA |
| IBS1 mutagenic primer (SEQ | AAAAAAGCTTATAATTATCCTTAGCTCTCTTCAATGTGCGCCCAG |
| ID NO: 67) | ATAGGGTG |
| EBS1d mutagenic primer | CAGATTGTACAAATGTGGTGATAACAGATAAGTCTTCAATGATAA |
| (SEQ ID NO 68) | CTTACCTTTCTTTGT |
| EBS2 mutagenic primer | TGAACGCAAGTTTCTAATTTCGATTAGAGCTCGATAGAGGAAAGT |
| (SEQ ID NO: 69) | GTCT |

### Example 7

### Transformation of Thermoanaerobacter and Thermoanaerobacterium Strains (Prophetic Example)

Thermoanaerobacter pseudoethanolicus 39E, Thermoanaerobacterium saccharolyticum JW/SL-YS485, Thermoanaerobacterium saccharolyticum B6A-RI, and Thermoanaerobacter sp. strain 59 will be transformed with the following protocol. Cells are grown at 55° C. in 40 mL of DSMZ M122 media (http://www.dsmz.de/microorganisms/media_list.php) with the following modifications: 5 g/L cellobiose instead of cellulose, 1.8 g/L K₂HPO₄, no glutathione, and 0.5 g/L L-cystiene-HC1 until an optical density of 0.6 to 0.8. Cells are then harvested and washed twice with 40 mL 0.2 M cellobiose at room temperature. Cells are re-suspended in 0.2 M cellobiose in aquilots of 100 uL and 0.1 to 1 ug plasmid DNA is added to the sample in a 1 mm gap-width electroportation cuvette. An exponential pulse (Bio-Rad Instruments) of 1.8 kV, 25 μF, 200Ω, ˜3-6 ms is applied to the cuvette, and cells are diluted 100-200 fold in fresh M122 and incubated for 12-16 hours at 55° C. The recovered cells are then diluted 25-100 fold in petri-plates with fresh agar-containing media containing a selective agent, such as 200 μg/mL kanamycin. Once the media has solidified, plates incubated at 55° C. for 24-72 hours for colony formation. Colonies can be tested by PCR for evidence of site-specific recombination.

### Example 8

### Fermentation Performance of Engineered Thermoanaerobacter and Thermoanaerobacterium Strains

Table 6 depicts the fermentation performance of engineered Thermoanaerobacter and Thermoanaerobacterium strains. Cultures were grown for 24 hours in M122 at 55° C. without shaking. The following abbreviations are used in Table 6: Cellobiose (CB), glucose (G), lactic acid (LA), acetic acid (AA), and ethanol (Etoh). Values are in grams per liter. YS485—Thermoanaerobacterium saccharolyticum JW/SL-YS485, B6A-RI—Thermoanaerobacterium saccharolyticum B6A-RI, 39E—Thermoanaerobacter pseudoethanolicus 39E.

TABLE 6

| Fermentation sample | CB | G | LA | AA | Etoh |
|---|---|---|---|---|---|
| YS485 wildtype | 0 | 0 | 0.77 | 1.04 | 1.40 |
| YS485 ΔL-Idh | 0 | 0 | 0 | 0.92 | 1.73 |
| YS485 Δpta/ack | 2.51 | 0 | 0.75 | 0.06 | 0.62 |
| YS485 ΔL-Idh, Δpta/ack | 0 | 0 | 0 | 0 | 2.69 |
| B6A-RI wildtype | 0 | 0 | 0 | 1.0 | 1.76 |
| B6A-RI ΔL-Idh, Δpta/ack | 0 | 0 | 0 | 0 | 2.72 |
| strain #1 | | | | | |
| B6A-RI ΔL-Idh, Δpta/ack | 0.45 | 0 | 0 | 0 | 2.49 |
| strain #2 | | | | | |
| 39E wildtype | 0.51 | 0 | 1.51 | 0.15 | 1.87 |
| Media | 5.10 | 0.25 | 0 | 0 | 0 |

### Example 9

### Construct for Engineering Cellulolytic and Xylanolytic Strains—Antisense RNA Technology Example

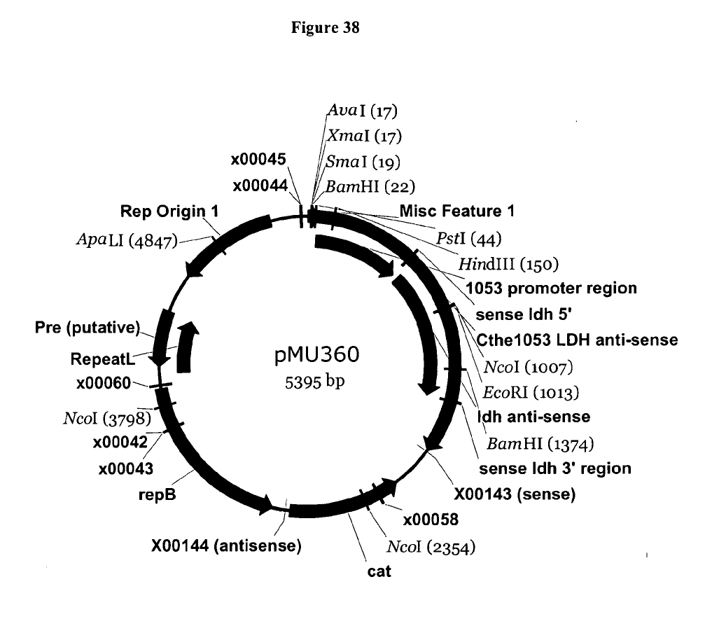

A replicative plasmid (FIG. 38) carrying an antisense RNA cassette targeting a C. thermocellum gene coding for lactate dehydrogenase (Cthe_{—}1053) was transferred to C. thermocellum 1313 by electroporation and thiamphenicol selection. The transformation efficiency observed for this plasmid was equal to that of the parent vector, pMU102. The sequence of the plasmid is shown in SEQ ID NO: 61. The asRNA cassette is depicted in FIG. 38 and is organized as follows: (i) the entire 1827 bp cassette is cloned into the multicloning site of pMU102 in the orientation shown in FIG. 38, (ii) the native promoter region is contained within the first 600 bp of the cassette, (iii) the first 877 bp of the ldh open reading frame are fused to the native promoter in the antisense orientation, (iv) approximately 300 additional by are included downstream of the asRNA ldh region.

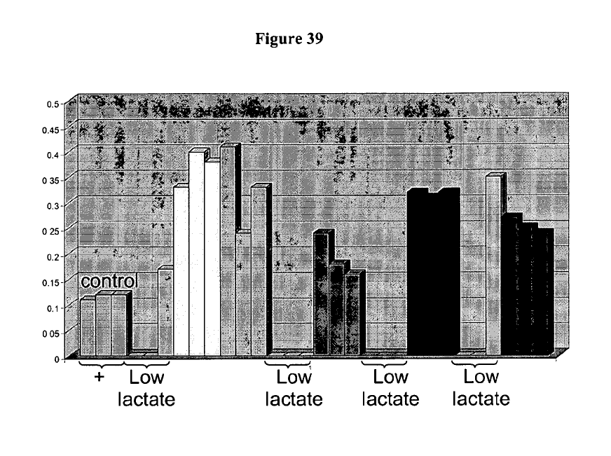

The resulting thiamphenicol resistant colonies were screened for altered end product formation by growing standing cultures on M122C media in the presence of 6 ug/mL thiamphenicol (to maintain the plasmid), as shown in FIG. 39. A preliminary screen of 9 randomly selected thiamphenicol-resistant transformants showed that 4 cultures exhibited low levels of lactate production relative to wild type. Additionally, a construct carrying antisense RNA directed to both ldh genes are to be constructed in order to partially, substantially, or completely delete, silence, inactivate, or down-regulate both genes simultaneously.

### Example 10

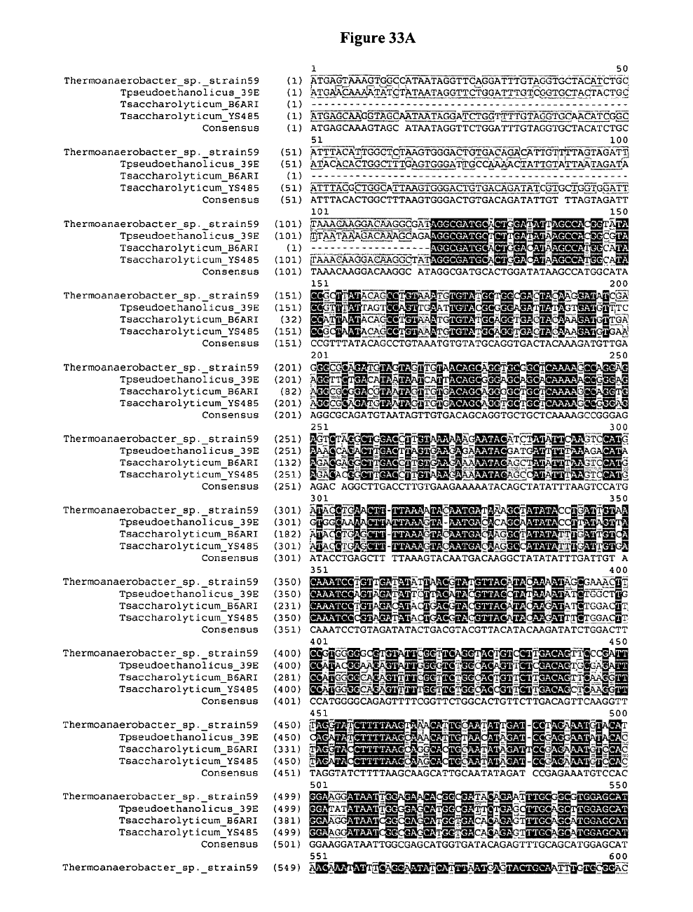

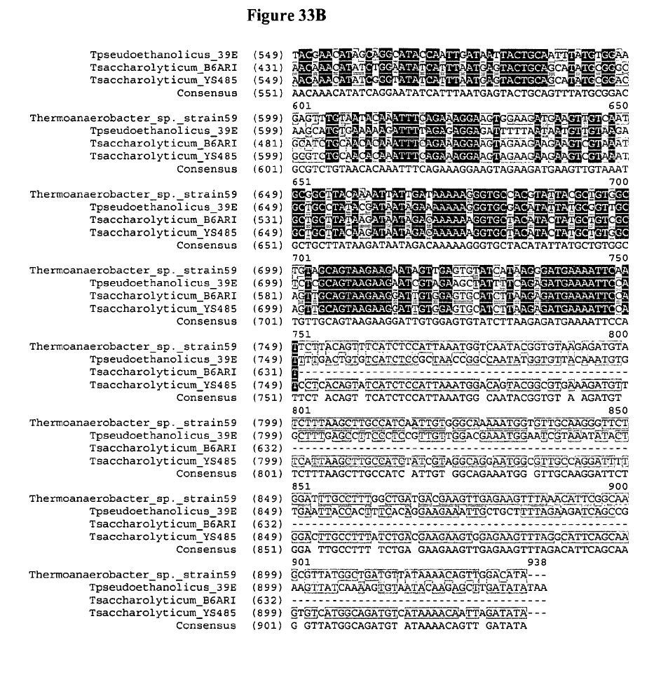

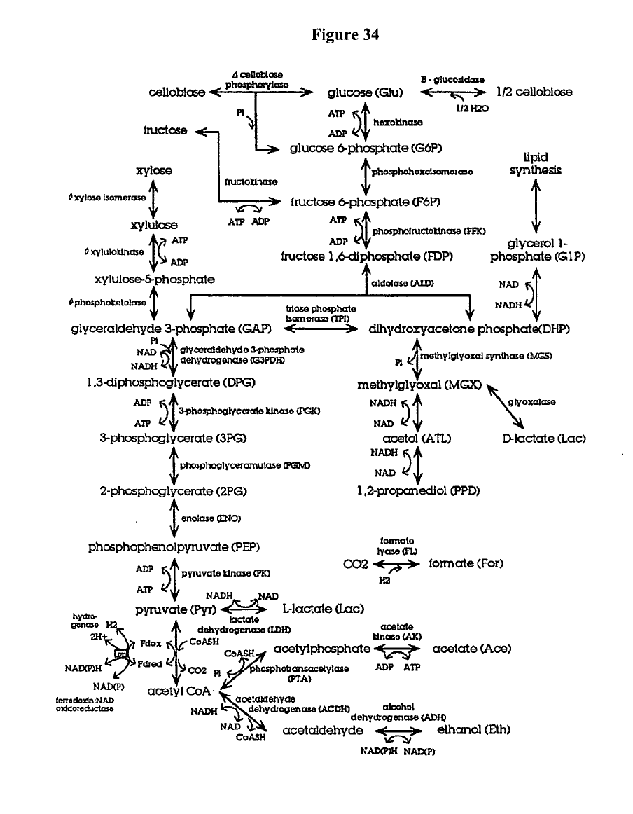

SEQ ID NOS: 44, 45, and 46 are the pyruvate-formate-lyase (aka formate acetyltransferase, EC. 2.3.1.54, pfl) genes from Thermoanaerobacterium saccharolyticum YS485, Clostridium thermocellum ATCC 27405, and Clostridium phytofermentans. Pfl catalyzes the conversion of pyruvate to Acetyl-CoA and formate (FIG. 34). Deletion of pfl will result in the elimination of formate production, and could result in a decrease in acetic acid yield in some thermophilic strains, with a resulting increase in ethanol yield.

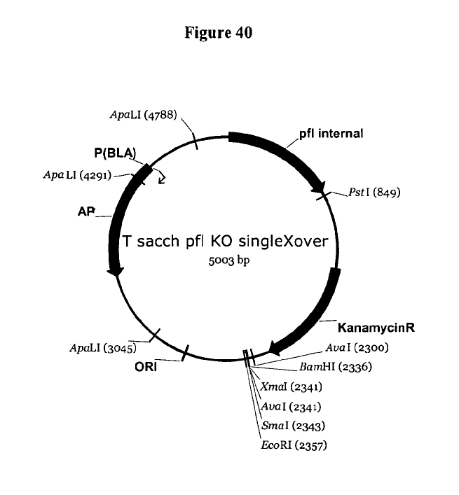

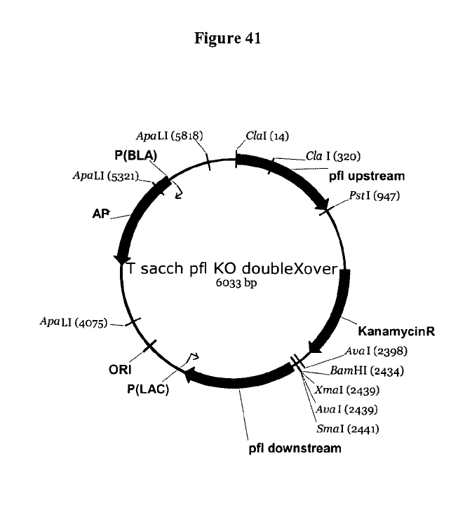

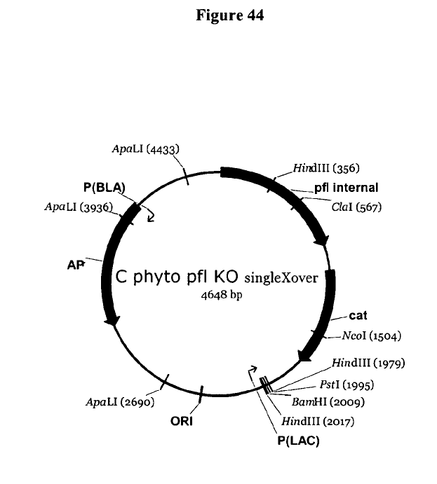

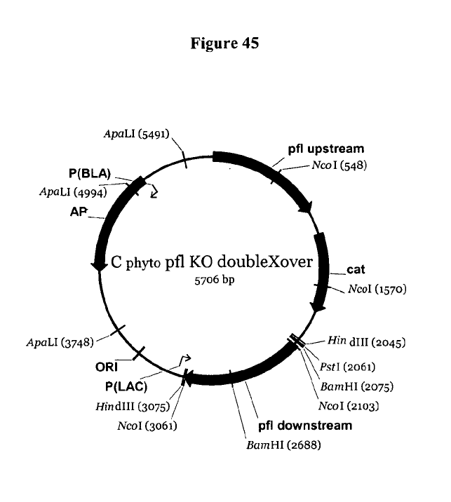

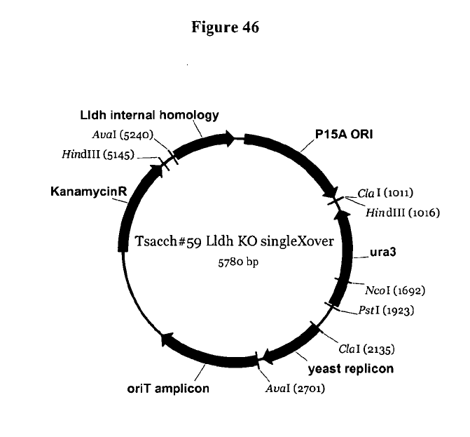

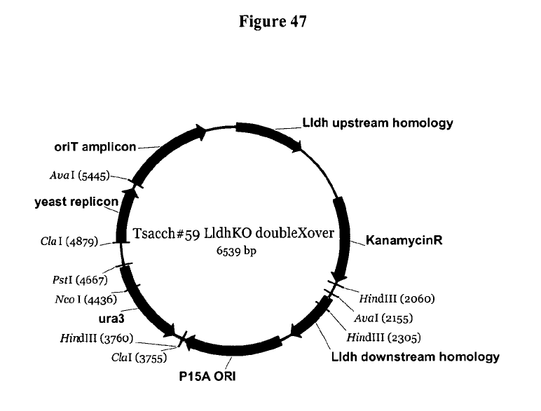

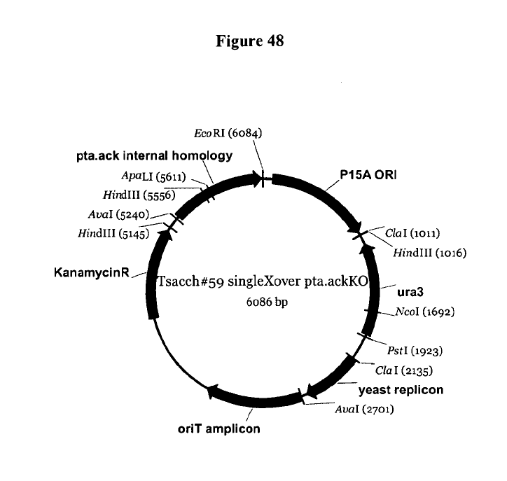

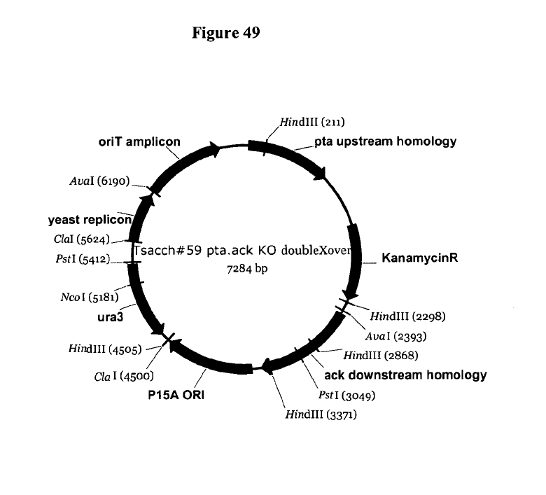

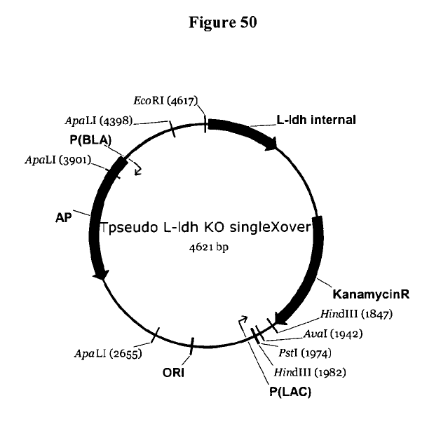

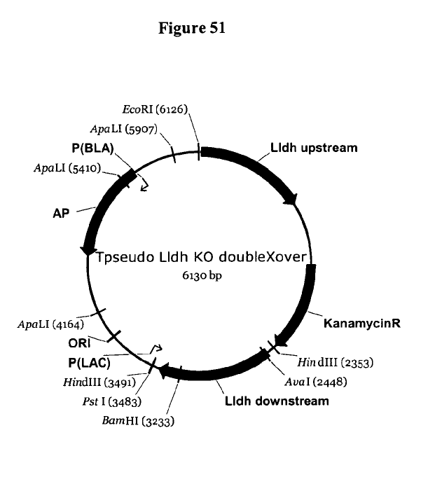

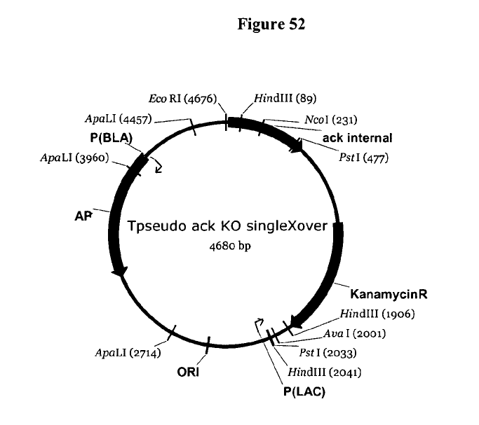

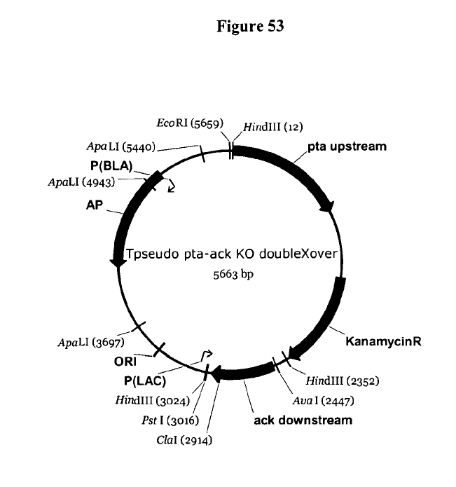

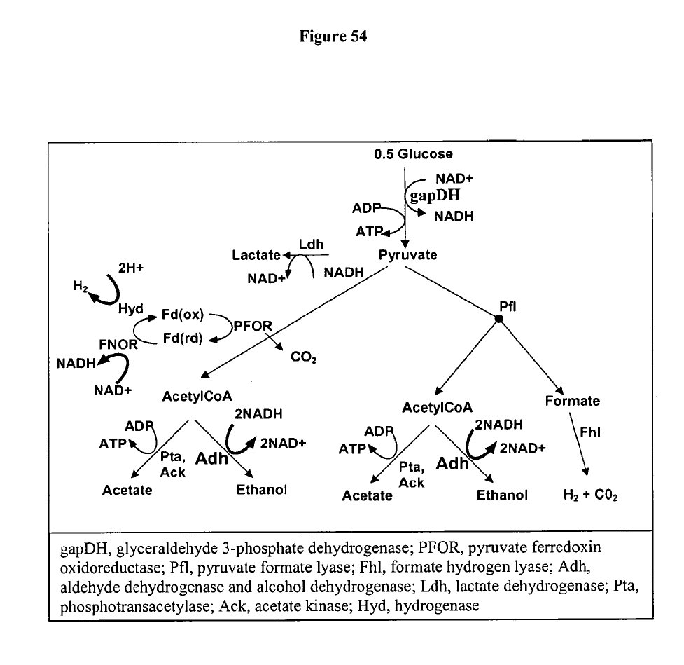

SEQ ID NOS: 47-52, depicted in FIGS. 40-45, show pfl knockout plasmids, two each for the three organisms listed above. Each organism has a single crossover and double crossover plasmid designed to partially, substantially, or completely delete, silence, inactivate, or down-regulate the pfl enzyme. Single crossover plasmids are designed with a single DNA sequence (400 bp to 1000 bp) homologous to an internal section of the pfl gene, double crossover plasmids are designed with two DNA sequences (400 to 1000 bp) homologous to regions upstream (5′) and downstream (3′) to the pfl gene. All plasmids are designed to use the best available antibiotic markers for selection in the given organism. Plasmids can be maintained in E. coli and constructed through a DNA synthesis contract company, such as Codon Devices or DNA 2.0.

Inactivation of the adh Gene in T. saccharolyticum

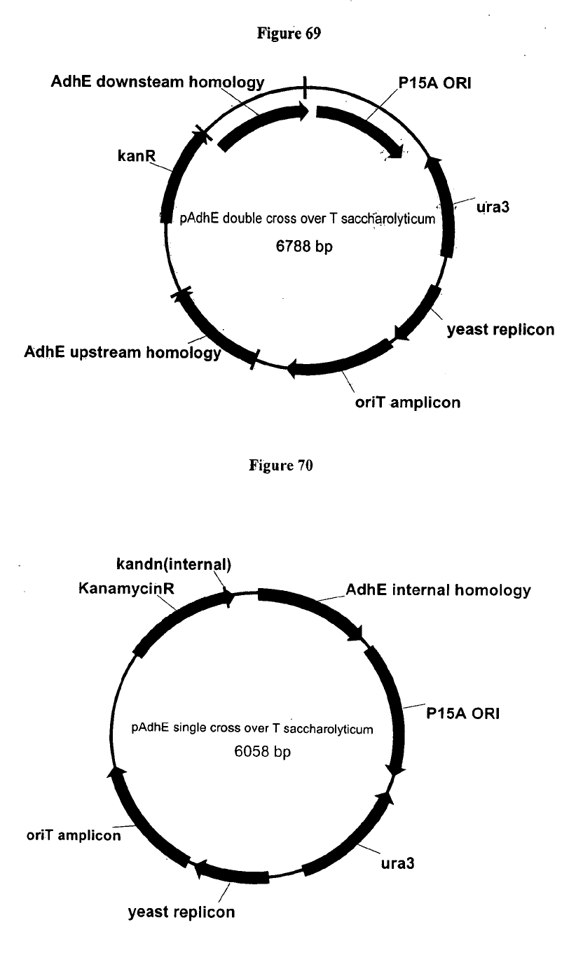

The T. saccharolyticum AdhE gene and protein sequences are depicted in SEQ ID NO 107 and 108, respectively, and deposited as GenBank accession EU313774. Examples targeting the adhE gene with double and single crossover knockout plasmids are depicted in FIGS. 69 and 70. The vector sequence for these constructs are SEQ ID NOs 109 and 110. Additional adh genes in T. saccharolyticum whose deletion may lead to reduced ethanol production and increased acetate, H2, and lactate production are included as SEQ ID NOs 111-113.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. (canceled)

2. An isolated nucleic acid molecule comprising a nucleotide sequence which shares at least 80% identity to a nucleotide sequence of any one of SEQ ID NOS:1-5, 30-31, 47-61, 79-83, 85-86, 88-89, 96-97, 99, 101, or 103, or a complement thereof.

3. (canceled)

4. A genetic construct comprising any one of SEQ ID NOS:1-5, 30-31, 47-61, 79-83, 85-86, 88-89, 96-97, 99, 101, or 103, operably linked to a promoter expressible in a thermophilic or mesophilic bacterium.

5. A recombinant thermophilic or mesophilic bacterium comprising the genetic construct of claim 4.

6. A vector comprising the nucleic acid molecule of claim 2.

7. A host cell comprising the nucleic acid molecule of claim 2.

8. The host cell of claim 7, wherein the host cell is a thermophilic or mesophilic bacterial cell.

9. A genetically modified thermophilic or mesophilic microorganism, wherein a first native gene is partially, substantially, or completely deleted, silenced, inactivated, or down-regulated, which first native gene encodes a first native enzyme involved in the metabolic production of an organic acid or a salt thereof, thereby increasing the native ability of said thermophilic or mesophilic microorganism to produce lactate or acetate as a fermentation product.

10. (canceled)

11. The genetically modified microorganism according to claim 9, wherein said microorganism is a Gram-negative bacterium or a Gram-positive bacterium.

12. The genetically modified microorganism according to claim 9, wherein said microorganism is a species of the genera Therinoanaerobacterium, Thermoanaerobacter, Clostridium, Geobacillus, Saccharococcus, Paenihacillus, Bacillus, Caldicellulosiruptor, Anaerocellum, or Anoxybacillus.

13. (canceled)

14. (canceled)

15. The genetically modified microorganism according to claim 9, wherein said microorganism is selected from the group consisting of: (a) a thermophilic or mesophilic microorganism with a native ability to metabolize a hexose sugar; (b) a thermophilic or mesophilic microorganism with a native ability to metabolize a pentose sugar; and (c) a thermophilic or mesophilic microorganism with a native ability to metabolize a hexose sugar and a pentose sugar.

16-19. (canceled)

20. The genetically modified microorganism of claim 15, wherein a first non-native gene is inserted, which first non-native gene encodes a first non-native enzyme that confers the ability to metabolize a pentose sugar, thereby allowing said thermophilic or mesophilic microorganism to produce ethanol as a fermentation product from a pentose sugar.

21. The genetically modified microorganism according to claim 9, wherein said microorganism has a native ability to metabolize a pentose sugar.

22. (canceled)

23. The genetically modified microorganism of claim 21, wherein a first non-native gene is inserted, which first non-native gene encodes a first non-native enzyme that confers the ability to metabolize a hexose sugar, thereby allowing said thermophilic or mesophilic microorganism to produce lactate as a fermentation product from a hexose sugar.

24. The genetically modified microorganism of claim 21, wherein a first non-native gene is inserted, which first non-native gene encodes a first non-native enzyme that confers the ability to metabolize a hexose sugar, thereby allowing said thermophilic or mesophilic microorganism to produce acetate as a fermentation product from a hexose sugar

25. The genetically modified microorganism according claim 9, wherein said organic acid is selected from the group consisting of lactic acid, acetic acid, and ethanol.

26-28. (canceled)

29. The genetically modified microorganism according to claim 9, wherein said first native enzyme is selected from the group consisting of lactate dehydrogenase, acetate kinase, phosphotransacetylase, pyruvate formatelyase, alcohol dehydrogenase, and aldehyde dehydrogenase.

30-35. (canceled)

36. The genetically modified microorganism according to claim 9, wherein a second native gene is partially, substantially, or completely deleted, silenced, inactivated, or down-regulated, which second native gene encodes a second native enzyme involved in the metabolic production of an organic acid or a salt thereof.

37-40. (canceled)

41. A genetically modified thermophilic or mesophilic microorganism, wherein (a) a first native gene is partially, substantially, or completely deleted, silenced, inactivated, or down-regulated, which first native gene encodes a first native enzyme involved in the metabolic production of an organic acid or a salt thereof, and (b) a first non-native gene is inserted, which first non-native gene encodes a first non-native enzyme involved in the metabolic production of lactate or acetate, thereby allowing said thermophilic or mesophilic microorganism to produce lactate or acetate as a fermentation product.

42. (canceled)

43. The genetically modified microorganism of claim 41, wherein said first non-native gene encodes a first non-native enzyme that confers the ability to metabolize a hexose sugar, thereby allowing said thermophilic or mesophilic microorganism to metabolize a hexose sugar.

44. The genetically modified microorganism of claim 41, wherein said first non-native gene encodes a first non-native enzyme that confers the ability to metabolize a pentose sugar, thereby allowing said thermophilic or mesophilic microorganism to metabolize a pentose sugar.

45. The genetically modified microorganism of claim 41, wherein said first non-native gene encodes a first non-native enzyme that confers the ability to metabolize a hexose sugar; and a second non-native gene is inserted, which second non-native gene encodes a second non-native enzyme that confers the ability to metabolize a pentose sugar, thereby allowing said thermophilic or mesophilic microorganism to metabolize a hexose sugar and a pentose sugar.

46. The genetically modified microorganism according to claim 41, wherein said organic acid is selected from the group consisting of lactic acid, acetic acid, and ethanol.

47. (canceled)

48. (canceled)

49. The genetically modified microorganism according to claim 45, wherein said second non-native enzyme is xylose isomerase.

50. The genetically modified microorganism according to claim 49, wherein said first non-native gene corresponds to SEQ ID NOS:6, 10, or 14.

51. The genetically modified microorganism according to claim 41, wherein said non-native enzyme is xylulokinase.

52. The genetically modified microorganism according to claim 51, wherein said non-native gene corresponds to SEQ ID NOS:7, 11, or 15.

53. The genetically modified microorganism according to claim 41, wherein said non-native enzyme is L-arabinose isomerase.

54. The genetically modified microorganism according to claim 53, wherein said non-native gene corresponds to SEQ ID NOS:8 or 12.

55. The genetically modified microorganism according to claim 41, wherein said non-native enzyme is L-ribulose-5-phosphate 4-epimerase.

56. The genetically modified microorganism according to claim 55, wherein said non-native gene corresponds to SEQ ID NO:9 or 13.

57. (canceled)

58. (canceled)

59. The genetically modified microorganism according to claim 9, wherein said microorganism is selected from the group consisting of: (a) a thermophilic or mesophilic microorganism with a native ability to hydrolyze cellulose; (b) a thermophilic or mesophilic microorganism with a native ability to hydrolyze xylan; and (c) a thermophilic or mesophilic microorganism with a native ability to hydrolyze cellulose and xylan.

60-63. (canceled)

64. The genetically modified microorganism of claim 59, wherein a first non-native gene is inserted, which first non-native gene encodes a first non-native enzyme that confers the ability to hydrolyze cellulose.

65. The genetically modified microorganism according to claim 59, wherein said organic acid is selected from the group consisting of lactic acid, acetic acid, and ethanol.

66-68. (canceled)

69. The genetically modified microorganism according to claim 59, wherein said first native enzyme is selected from the group consisting of lactate dehydrogenase, acetate kinase, phosphotransacetylase, pyruvate formatelyase, alcohol dehydrogenase, and aldehyde dehydrogenase.

70-72. (canceled)

73. The genetically modified microorganism according to claim 59, wherein a second native gene is partially, substantially, or completely deleted, silenced, inactivated, or down-regulated, which second native gene encodes a second native enzyme involved in the metabolic production of an organic acid or a salt thereof.

74-77. (canceled)

78. A genetically modified thermophilic or mesophilic microorganism, wherein (a) a first native gene is partially, substantially, or completely deleted, silenced, inactivated, or down-regulated, which first native gene encodes a first native enzyme involved in the metabolic production of an organic acid or a salt thereof, and (b) a first non-native gene is inserted, which first non-native gene encodes a first non-native enzyme involved in the hydrolysis of a polysaccharide, thereby allowing said thermophilic or mesophilic microorganism to produce lactate or acetate as a fermentation product.

79. (canceled)

80. The genetically modified microorganism of claim 78, wherein said first non-native gene encodes a first non-native enzyme that confers the ability to hydrolyze cellulose, thereby allowing said thermophilic or mesophilic microorganism to hydrolyze cellulose.

81. The genetically modified microorganism of claim 78, wherein said first non-native gene encodes a first non-native enzyme that confers the ability to hydrolyze xylan, thereby allowing said thermophilic or mesophilic microorganism to hydrolyze xylan.

82. The genetically modified microorganism of claim 78, wherein said first non-native gene encodes a first non-native enzyme that confers the ability to hydrolyze cellulose; and a second non-native gene is inserted, which second non-native gene encodes a second non-native enzyme that confers the ability to hydrolyze xylan, thereby allowing said thermophilic or mesophilic microorganism to hydrolyze cellulose and xylan.

83. The genetically modified microorganism according to claim 78, wherein said organic acid is selected from the group consisting of lactic acid, acetic acid, and ethanol.

84-88. (canceled)

89. A process for converting lignocellulosic biomass to lactate or acetate, comprising contacting lignocellulosic biomass with a recombinant or genetically modified thermophilic or mesophilic microorganism according to claim 9.

90. (canceled)

91. The process of claim 89, wherein said lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof.

92-98. (canceled)


SEQUENCE LISTING (129 sequences)

SEQ ID NO: 1 - DNA, 9000 bp, Artificial Sequence
  Synthetic polynucleotide
agctttggctaacacacacgccattccaaccaatagttttctcggcataaagccatgctctgacgcttaaatgcactaatgccttaaaaaaacattaaagtctaacacactagacttatttacttcgtaattaagtcgttaaaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgattatgtcttttgcgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagcttggctgcaggtcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgactctagaggatccctcagcgaagctccactatgtttcaaaatgtcagatatatcaattttcatcaaagtcacctcttaaaaccgacaaggactattatactaactaataaccctcatgtcaagaattatatgacagattggcttaaataacaaaaataattttgtttagttaaattcggaatttcttcttaatattattaacatattccacatattaatacaagaaaaaacccggcaaaaaaataaaaaaattttataagcccgtttcctaaaaaaacaggcttgtaaaattataacgcatcttttataagttttttacaagtcttaaagtctcccttgcaatctcaagctcctcatttgtcgggataaccaaagtctttactttcgcatcgggagcactgatatccgcttctttgcctttcacttcatttttatccaaatctattttaattccgaaaaagtccatatccttcaaaacttctcttcttatataagcattgttttcgccgatacctgcagtgaataccaccgcatcaacgccgttcagcactgcaatatattttccaatatatttcctaacaccatagcagaaaatatccaatgccagctgcgccctgtcatctcccttttctgcggcatcctgaacatctctgaaatcactgcttacacctgaaattccaagcacacctgatttcttgttaaggaaattgtttatatcgttaatattcattttttccttttccatcaaataagttataaccgcagggtcaacattgccgcttctggtacccatgcacaacccctgcagaggagtaaatcccattgaggtgtcaacggattttccgccttttaccgcacaaatacttgctccgtttccaagatggcaggttatcagcttcaggctctcaataggtttgcccagcatctgagccgccctgtgggccacatatttgtgggaagttccgtggaatccgtattttctcaatttatacttctcatatatctcataagggagggcataaatatatgcatgctagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataacctgaggttttgctccaaccagcatctcaaaagatttggatgcagatattgcaatttcagaaagctggtctgcatccggattttccaccaagccgcaatcggcatatacaaaggttccgttatgaccatattcacagttgggtacaaccataacaaaaaaggatgatacgagttttgtccccggggccgtctttaatatctgcaaagccggtctcaaagtatttgcagtggaattgacagcacccgccaccataccatccgcttcacctttttttaccatcataactccataataaagagggtctttgatcgtttcccttgcggcttctatagtcatacccttcgattttctaagctcatacagtgtatttgcataatcctccaatttttcggaatttaaggaatcctctatcatcactccttcaagatcaatatcccccgccagactcttaatctccttttcattgcctatcagtacaacctttgcaattccctttttcattatcatggatgcggctttaataaccctcagatccgtactttccggcaaaactatggtttttacgtctgatttcgccctttcaattatttgttccaaaaaactcataaattcttctcctttcataatcccaaaactgttatcataaaaactgtatttgtaatacttataactatatattatcaccaggtaataatacctactcactataaacagctattttactgggttccaagcaactctaattatatacaaaatgttttttgtatacaacaccctccttatctttttttcggctttagccataaataacggcaagtaactccaaaatacaggatatttcatgcttttagaaactttttattagtcttcttaattattcagattttgtggcaattaaactttgcagctcctccaaatagttgtccagctcctcttctttaagattgctgagatatgacaatctgtaatttttagcctttttggccatctctagcgcactctccgtcattcccaaatctttcaaaacacagctatagttatagtatgcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca

SEQ ID NO: 2 - DNA, 7582 bp, Artificial Sequence
  Synthetic polynucleotide
aattctgttctggctttagaccatttacgctttgggtttgccatgcatcacacctcctcgtcaccatacaacaaaccttgtgtataaataaaatcttgtacacatggaaggtactcgattttgtttttacttaaaacaaagctacactaattgtctgtaaaaaagtttttgaaaacttccagccgcggatctatatcctcatttacgcatttgcactcatcgttgttaagatccgctccacacttcgggcaatatcccttgcaggcctcgtcacaaacctgctttgccggaagattcagtatgatattgtctatcattaccttttccagctcgaggaacttaccatggtacgtataatattcctcgtcggttttgttactgccctcttctacaaagttttcctttacatcaatatgcatctttgattcaatatccttgaggcaccttgagcattttgccctgtaatccgcccagagttcaccgtcaagttttataatccctccggcatttaccaaagtgcccttaaaagttaccggtttcgcaaagtcaaaatcctcagctataaaatcattaattttaattgactcactaaagtccagtctcagcgaagctccactatgtttcaaaatgtcagatatatcaattttcatcaaagtcacctcttaaaaccgacaaggactattatactaactaataaccctcatgtcaagaattatatgacagattggcttaaataacaaaaataattttgtttagttaaattcggaatttcttcttaatattattaacatattccacatattaatacaagaaaaaacccggcaaaaaaataaaaaaattttataagcccgtttcctaaaaaaacaggcttgtaaaattataacgcatcttttataagttttttacaagtcttaaagtctcccttgcaatctcaagctcctcatttgtcgggataaccaaagtctttactttcgcatcgggagcactgatatccgcttctttgcctttcacttcatttttatccaaatctattttaattccgaaaaagtccatatccttcaaaacttctcttcttatataagcattgttttcgccgatacctgcagtgaataccaccgcatcaacgccgttcagcactgcaatatattttccaatatatttcctaacaccatagcagaaaatatccaatgccagctgctgcagtaatcgcatcagattgcagtataaatttaacgatcactcatcatgttcatatttatcagagctcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttacagctgatactttagtgatgagcttccggtattaataaccaaaatattcatttcaaaaactcactcccgtcttgttttttttaattttcctattcctaaacttcgataaacagatgtttttattaaacgctgcgcaacaccttcttcaatgtccggttttaacagaatttatgccttgacatattgagcctgaaccgcagtaattgccgcaaccccgactatatcctcggcactgcagcctcgtgacagatcatttaccggtcttgccaaaccttgtgttatcgggccgtaagcttcagcttttgccaatctctgtgtaagcttgtatgcaatatttccggcatcaagatccgggaaaataagaacattggcctttcctgcaacactgcttccctttgccttcgattttgccacttccggaacaatggcggcatccacctgaagttctccgtcaattgcaaggtggggagctttttcctttgcaagctgtgttgccttgattaccttttcggtcagctcacttttggcactgccgtaagaagaataagaaagcattgccacctgaggttttgctccaaccagcatctcaaaagatttggatgcagatattgcaatttcagaaagctggtctgcatccggattttccaccaagccgcaatcggcatatacaaaggttccgttatgaccatattcacagttgggtacaaccataacaaaaaaggatgatacgagttttgtccccggggccgtctttaatatctgcaaagccggtctcaaagtatttgcagtggaattgacagcacccgccaccataccatccgcttcacctttttttaccatcataactccataataaagagggtctttgatcgtttcccttgcggcttctatagtcatacccttcgattttctaagctcatacagtgtatttgcataatcctccaatttttcggaatttaaggaatcctctatcatcactccttcaagatcaatatcccccgccagactcttaatctccttttcattgcctatcagtacaacctttgcaattccctttttcattatcatggatgcggctttaataaccctcagatccgtactttccggcaaaactatggtttttacgtctgatttcgccctttcaattatttgttccaaaaaactcataaattcttctcctttcataatcccaaaactgttatcataaaaactgtatttgtaatacttataactatatattatcaccaggtaataatacctactcactataaacagctattttactgggttccaagcaactctaggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagcccgatatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgattcaataatttcttttactcgctcgttatagtcgatcggttcatcattcaccaaatcataattttcatgtgaccgttctttatcaatatcgggattcgttttactttcccgttctctctgattgtgaaattg

SEQ ID NO: 3 - DNA, 8927 bp, Artificial Sequence
  Synthetic polynucleotide
agctttggctaacacacacgccattccaaccaatagttttctcggcataaagccatgctctgacgcttaaatgcactaatgccttaaaaaaacattaaagtctaacacactagacttatttacttcgtaattaagtcgttaaaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgattatgtcttttgcgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagcttggctgcaggtcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagagccccatactcatgagcagtcttgttacagctatgccggcagcacctgaaccgtttacaacaacttctatatcctcgattttcttgttgacaagctttaatgcattgatcattgctgcaacagtaacaacggctgtaccgtgctggtcatcatggaatattggaatgtcacattcctctttgagtcttctttctatttcaaagcatctcggagcggatatatcttcgaggtttataccgccaaagcttccggagatgagcttgattgtctttacaatttcatctacgtcttttgatttgatacagagcggaaatgcgtccacatcaccaaacttcttgaagagtacgcatttaccttccataacaggcattccggcttcaggtcctatgtctccgagccctaaaaccgccgtaccgtcggtaataaccgctaccaggttccaacgtcttgtatattcataagaaagattaacatctttctgaattgcaagacatggttctgcaacacccggtgtataagcaagcgacaactcttccttggttgaaacaggtaccttgtgtataacctcaattttacccttccactcaccgtgaagccttagtgattcttttctgtaatccatttgattctacctccaaattatattattaaatatctgcgatattaatgcacaattataaattcttaacttcgttcaatacttttttaacctgctccgctgagaatcttaaagcttcttcttcttcaggagtcagattaaattggagaacttcctgaacaccttcggaatttacgatggatggaaggcttattgcaacatcttctattccatacatgccgtttataacggttcctacggttcttattgtattctgattcttaaggagtgtttcaactattgtgttgattgaaactgcaataccatagtatgttgcacctttgttcttgataatggttgcacccgcagttttaacatcttcagcgatttttttcttgtcttcttctgtgaaattgcatttcggatcatcgatatattcgttgatatttttaccggcgatatgtgtgcagctccacaacggaagctgtgaatcaccgtgttcgcctattatgtagccgtgtacatagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatgtacatttattggtaacattgtcttttgggtttttctttcttatatccgttcttgccgccgcggtttcggaaaaatttgaaatattgcttgtttcccttcttttgttggtacttttgataccttatattgcccattattacaaactggagaacggagttcagaggctttatgagctttataacaaaattgatgaaaaatgtgtaaggaaaaacaagaccgcctgagttctcacccagacggtcggtattggcagtttcactttcgttagtcgatgtttttcatgccggcaaagaaattattttcttgcaagaacctttttcagttttgcaaatcttggaagaccatcttcgataggaggtcttgattctccctgaattaacggaagagcatacttaataaagtcttctgtaaggcctgctccgtcaggtttaatccattccaacggaactttcttctcagtatttgcaacttcactgaggttcagaagcttgatattgcacttgtattcaggaccttccgctctttcaaaagcaaccatgtagtctgttttcccttcaacggcatattgtacggctgcctgtcctgcaagataagcttcatttacgtcggtaagagaagctacgtgagctgcgcatctttggagaaggctgaattcaatgccgcgaacctttgcgccggtcttctctttaacaatgttagccagtgttgaagcaagaccgcccaactgtgcatgtccaaaggagtcttttgttttcgcaaggtctgaaccgtattcggaaatatattttccgtttttgtctttgataccttcagatacggctacaataacctttccgttttctttgtagattcttgtcacatcttcaacaaatttgtctatgtcaaaggaaagctcgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca

SEQ ID NO: 4 - DNA, 7114 bp, Artificial Sequence
  Synthetic polynucleotide
aattcagccccatactcatgagcagtcttgttacagctatgccggcagcacctgaaccgtttacaacaacttctatatcctcgattttcttgttgacaagctttaatgcattgatcattgctgcaacagtaacaacggctgtaccgtgctggtcatcatggaatattggaatgtcacattcctctttgagtcttctttctatttcaaagcatctcggagcggatatatcttcgaggtttataccgccaaagcttccggagatgagcttgattgtctttacaatttcatctacgtcttttgatttgatacagagcggaaatgcgtccacatcaccaaacttcttgaagagtacgcatttaccttccataacaggcattccggcttcaggtcctatgtctccgagccctaaaaccgccgtaccgtcggtaataaccgctaccaggttccaacgtcttgtatattcataagaaagattaacatctttctgaattgcaagacatggttctgcaacacccggtgtataagcaagcgacaactcttccttggttgaaacaggtaccttgtgtataacctcaattttacccttccactcaccgtgaagccttagtgattcttttctgtaatccatttgattctacctccaaattatattattaaatatctgcgatattaatgcacaattataaattcttaacttcgttcaatacttttttaacctgctccgctgagaatcttaaagcttcttcttcttcaggagtcagattaaattggagaacttcctgaacaccttcggaatttacgatggatggaaggcttattgcaacatcttctattccatacatgccgtttataacggttcctacggttcttattgtattctgattcttaaggagtgtttcaactattgtgttgattgaaactgcaataccatagtatgttgcacctttgttcttgataatggttgcacccgcagttttaacatcttcagcgatttttttcttgtcttcttctgtgaaattgcatttcggatcatcgattaatcgcatcagattgcagtataaatttaacgatcactcatcatgttcatatttatcagagctcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaatcgatgagaacaagttcatttgcggtttgccgcaaagccattgtgaaggctgcagacgcacctacaaaaccagcaccaatgattgcaacttttgacctactttttaccatttccataccattcctttcaattacccagtatatttaacggttagttcgtttataaatttgagattaattctttaaattttaactgtgaacccggttcacaggtattatcattaatttcagtatatgtgtttaataaaaattagtgaaaatttgcaactgcaagcatttaaaattgtaaacgataaataaatccaggcaacaaatttcccccattttaaatagcccagttaaacacattgataacattttaacattattttatatctgcgtccataactgaaaaagggaaatccattactttatgaaatcaaattttgaagttatcaagaaattatgacgattttctccgtggcatgcaagatttcgcgatatttcattcgtttatattaattttttatgaaaactgcggtttgggctgacaattgcgatggaagtttcaattagactttttgtcaaatattatgtataataatattatctataataatgtatgaaaaaattgtcctaagatggaagacgggggtggtttcatatatggttaaatatctgaagaggcaggaagagttggtagaagaagctctctcaaaggataactgttctgactgggaaagtttgagaaattatcataagtcccaaattgaatttttgcagcatgagagacttgtacatttattggtaacattgtcttttgggtttttctttcttatatccgttcttgccgccgcggtttcggaaaaatttgaaatattgcttgtttcccttcttttgttggtacttttgataccttatattgcccattattacaaactggagaacggagttcagaggctttatgagctttataacaaaattgatgaaaaatgtgtaaggaaaaacaagaccgcctgagtggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagcccgatatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgattcaataatttcttttactcgctcgttatagtcgatcggttcatcattcaccaaatcataattttcatgtgaccgttctttatcaatatcgggattcgttttactttcccgttctctctgattgtgaaattg

SEQ ID NO: 5 - DNA, 3932 bp, Artificial Sequence
  Synthetic polynucleotide
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgtagttggtgcaggctttgtaggttccaccacagcttatacattgatgctcagcggacttatatctgaaattgtactgatagacataaatgcaaaaaaagccgacggagaagtcatggacttaaatcacggcatgccttttgtaaggcccgttgaaatttatcgtggtgactacaaagactgtgccggatccgacatagtaatcattaccgccggtgccaaccaaaaagaaggcgaaacgagaatagatcttagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatgtacatgcttatattattggcgaacacggtgacaccgaagttgcggcctggagtcttgcaaatattgcgggaattcccatggatcgctactgtgacgaatgccatcagtgcgaggagcagatttcccggaataaaatatatgaaagtgttaaaaatgcagcttatgaaatcatcaggaacaaaggtgcaacctattatgccgtagcccttgccgtaagaagaatcgttgaagccattgtactgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 6 - DNA, 1320 bp, Thermoanaerobacterium saccharolyticum
atgaataaatattttgagaacgtatctaaaataaaatatgaaggaccaaaatcaaataatccttattcctttaaattttacaatccagaggaagtaatcgatggcaagacgatggaggagcatctccgcttttctatagcttattggcacacttttactgctgatggaacagatcaatttggcaaggctactatgcaaagaccatggaaccactacacagatcctatggatatagcgaaacgaagggtagaagcagcatttgagttttttgataagataaatgcacctttcttctgcttccatgatagggatattgcccctgaaggagatactcttagagagacaaacaaaaacttagatacaatagttgctatgataaaggattacttaaagaccagcaagacaaaagttttgtggggtaccgcaaatcttttctccaatccgagatttgtacatggtgcatcaacatcctgcaatgctgacgtttttgcatattctgcagcgcaagtcaaaaaagcccttgagattactaaggagcttggccgcgaaaactacgtattttggggtggaagagaagggtacgagacgcttctcaatacagatatggagttagagcttgataactttgcaagatttttgcacatggctgttgactatgcaaaggaaatcggctttgaaggtcagttcttgattgagccgaagccaaaggagcctacaaaacatcaatacgactttgacgtggcaaatgtattggcattcttgagaaaatacgaccttgacaaatatttcaaagtaaatatcgaagcaaaccatgcgacattggcattccacgacttccaacatgagctaagatacgccagaataaacggtgtattaggatcaattgacgcaaatacaggcgacatgcttttgggatgggatacggaccagttccctacagatatacgcatgacaacgcttgctatgtatgaagtcataaagatgggtggatttgacaaaggtggccttaactttgatgcaaaagtaagacgtgcttcatttgagccagaagatcttttcttaggtcacatagcaggaatggatgcttttgcaaaaggctttaaagttgcttacaagcttgtgaaagatggcgtatttgacaagttcatcgaagaaagatacgcaagctacaaagaaggcattggcgctgatattgtaagcggtaaagctgacttcaagagccttgaaaagtatgcattagagcacagccagattgtaaacaaatcaggcagacaagagctattagaatcaatcctaaatcagtatttgtttgcagaataa

SEQ ID NO: 7 - DNA, 1566 bp, Thermoanaerobacterium saccharolyticum
atgagggcggcttcatgcttcattaaagctgccctcaacaaaaatcatggaggtaaatgtatgtattttttagggatagatttagggacatcatcagttaagataatactgatgaatgaaagcggcaatgtggtatcaagcgtttcaaaagaatatcctgtgtactatccagagccaggctgggctgagcaaaatccagaagattggtggaatggcacaagggatggaataagagagattattgcgaaaagcggcgtaaatggcgatgaaataaagggtgttggcttaagcgggcagatgcatggactggtgcttttagacaaagacaataacgttttaacgccagccatactttggtgtgaccagaggacacaggaagaatgcgactacatcacagagaaaataggaaaagaaggccttttgaagtacacagggaataaagcattgacaggttttactgcaccaaagatattatgggtaaagaagcaccttaaagacgtatatgaaagaatcgctcatatccttttgccaaaagattatataaggtttaaattgacaggtgagtacgctacagaagtttcagatgcatcaggtacacttcttttcgatgtggaaaatagaagatggtcaaaggaaatgatagacatatttgaaataccggaaaaagcccttcctaagtgctacgaatcaacagatgtcacagggtatgtcaccaaagaggcagcagatttgacagggcttcatgaagggactattgtcgtaggcggtggtggtgaccaagccagcggcgctgtaggcactggcacggtgaaaagcggcatagtgtccatcgcattaggaacttcaggcgtcgtatttgcatcacaggacaagtacgcagcagatgatgagcttaggcttcactcattctgccatgcaaacggcaaatggcatgtgatgggtgtcatgctttcggctgcatcatgtcttaaatggtgggtagatgatgtaaataattacaagaccgatgttatgacatttgatggactcttagaagaagcagagaaggtgaagccaggcagtgatggattgatattcttgccatacctgatgggtgaaaggaccccttacagcgatccttatgcgagaggcagctttgtaggtttaacaattacacacaatagaagccacatgacaagatctatattagaaggcgtcgcatttggacttagggattcgctggagcttataaaggctttaaatatacctgtaaatgaagccagggtaagtggtggtggtgctaaaagcaggctttggaggcaaatacttgccgatgtattcaatgtaaggatagacatgataaatgctacagaaggaccttcatttggtgcagcaataatggcgtctgtgggatatggcctttacaaaaatgtagatgatgcatgcaatagtttaataaaagttacagacagcgtatatccaatcaaagaaaacgtcgaaaagtacaacaaactgtatccaatctacgtgagcttgtattcaaggcttaaaggcgcctttgaagaaattgggaagttggatttgtaa

SEQ ID NO: 8 - DNA, 1407 bp, Thermoanaerobacterium saccharolyticum
atgattattgtgtacaaagatgaaaagcccaggataggttttttgggtattatgcaggagttatacgatgatatgttgcctggtattactgaaaggcaagagatgtatgcacaacaggttataggtagattaggtgatgttgctgatttttatttcccaggtgctgcaaaaaacagaaatgatatagaaaggatagttaaggaattcaacgataaagatcttgacggaataatgatcgtgatgctgacatacggaccagccacaaatcttgtgaatgctttaagaaacaataggcttccgattatgctggcgaatatacagccagaaagcactgtgacagacgattgggatatgggggacttgacctacaaccaaggtgttcatggtgcacaggatacttcaaatattattctgagaatgggcataacttgtcctgttataacagaagattggcattctgatgaatttaaagattttgtgaatgattgggcaaaaactgtaaagacagtaaaagctttgaggaatatgaagatagcacaatttggaagaatgcatggtatgtatgacataatgggtgatgatgcagcttttacaagaaaattggggccgcaaataaaccaggagtacattggccaagtttttagatatatggaagaagctacaaatgaagaaattgacaaagtgatagaggaaaacaagaagaacttttatatagatcctaaattaagtgatgagagccacagatatgctgcaaggcttcaaataggatttaagaaattgcttgaggagaaagggtactctggctttagtgctcactttgatgtgtttaaaggcgatggaagatttaagcagatacacatgatggcagcatcaaacttgatggcagaaggatatggctatgcggcagagggcgatgtagttacggcaagcctggtggcagcaggtcatgttttgataggcaatgcacactttaccgagatgtatgcgatggattttaagagagattcaattttgatgagccacatgggagagggcaattggaagatagccagaaaagatagacctataaaattagtcgatcgagagcttggcataggaaagcttgataatcctccaacagtggtgtttatggctcaaccaggcattgcgacattggcatcattagtgtctttagaaggcgaaaaatatagacttgttgtttcaaagggagaaattttagatacagaagaagcgaaaaatatagagatgccgtatttccattttagacctgaaaacggagttagggcttgtctaaatggctggcttaaaaatggtggtacacatcatcagtgcttgacattaggtgatgctactaaaagatggaagcttttatgcgaattattagatatcgagtatgttgaagtgtaa

SEQ ID NO: 9 - DNA, 642 bp, Thermoanaerobacterium saccharolyticum
atgttagagaacctaaaacaacgtgtatataaaatgaacatgatgcttcctaaaaacaatttagtcacaatgacaagcggcaatgtcagcggaagagatcctgagacaaatcttgtagtcataaagcccagcggagttttgtacgatgaaatgacgccagatgatatggtagtcgtggatttggatggcaatgtggttgagggtaagctaaaaccatctgtcgatactgctacacatctttacgtctacaggcatagaaatgatgtaaacggcattgtccatacacactcaccgtatgctacaagttttgccgcacttggccggtcaattccggtctatcttacagctattgcagacgagtttggatgcgcaattcctgtagggccttatgccaaaattggcggggaagagataggaaaagccatcgtagattatataggtgagagtcctgcaatacttatgaaaaatcacggcgtttttaccattggcaattcacctgaagcagccttaaaagctgctgttatggtagaagatacagctaagacggtgcacttatcactgcttttaggcacacctgatgtaataccagatgaagaagtaaaaagagcccatgaaagatatcttacaaaatacggtcaatga

SEQ ID NO: 10 - DNA, 1320 bp, Clostridium cellulolyticum
atgtcagaagtatttagcggtatttcaaacattaaatttgaaggaagcgggtcagataatccattagcttttaagtactatgaccctaaggcagttatcggcggaaagacaatggaagaacatctgagattcgcagttgcctactggcatacttttgcagcaccaggtgctgacatgttcggtgcaggatcatatgtaagaccttggaatacaatgtccgatcctctggaaattgcaaaatacaaagttgaagcaaactttgaattcattgaaaagctgggagcacctttcttcgctttccatgacagggatattgctcctgaaggcgacacactcgctgaaacaaataaaaaccttgatacaatagtttcagtaattaaagatagaatgaaatccagtccggtaaagttattatggggaactacaaatgctttcggaaacccaagatttatgcatggtgcatcaacttcgccaaacgctgacatatttgcgtatgcagcagctcaggttaaaaaggcaatggaaatcacaaaggaattaggcggagaaaactatgtattctggggtggtagagaaggttatgaaactctcttgaatacagacatgaagctggaacttgataatttagcaagattcttgaagatggctgttgactatgctaaggaaatcggttttgacggacaattcctaatcgaaccaaagccaaaagaaccaactaagcaccaatatgattttgatacagctacagttatcggcttcctgaagacatatggattagacccatacttcaagatgaatatcgaagctaaccatgctacattagcaggacacacattccaacatgagcttgctatgtgcagaatcaacgacatgcttggaagtattgatgctaaccaaggtgatgtaatgctcggatgggatacagaccaattcccaacgaacctatatgatgcaacactagcaatggtggaagtattaaaggccggcggattgaaaaagggaggtttgaacttcgactcaaaagttagaagaggatcattcgaaccatcagacttgttctatggacatattgcaggtatggatacttttgcaaagggtcttatcatagcaaataagatcgttgaggacggtaagtttgatgcatttgttgctgacagatactcaagctacacaaatggtatcggaaaagatattgttgaaggaaaagttggctttaaggaattggagcaatatgcacttactgcaaagattcagaacaagtctggacgtcaggaaatgctggaagctttgttaaaccagtatatcctcgaaacaaaataa

SEQ ID NO: 11 - DNA, 1608 bp, Clostridium cellulolyticum
atgaagcatgaactaaatgacgggagaaatgctattctaaatggaaagacagcaattgggattgaactcggatcaactagaataaaaacggtattgataggtgcagacaatgcacctatcgcatccggtagtcatgactgggaaaacagctatatcaataatatttggacttacagcttggaagatatctggaaaggcgtacagagcagctatcaggaaatggttaaagatgttagggacaaattcggagtaagtctaaagacaaccggagcaataggttttagcggaatgatgcacggttatatggtttttgataaggaaggtaatcttctgactcatttcagaacatggcgtaacactataactgcacaggcttccgaggaactaaccaagttgtttaattatcctattcctcaaaggtggagcattgcccatctttaccaagccatactgaacaatgaagagcatgtatccaatatcgattttatgactacattggccggatttatacactggaagttgacaggagaaaaagttcttggtgtcggagaggcatcaggtgttttcccaatagatttagatactaaggattttaattcaagtatgattaatcagtttaatgaggctaccaccaatcgaaatttttcatggaagcttcaaaatattcttccaaaagttttggtttcgggtactgaagcaggtaggctgacagaagaaggtgcaaagcttcttgatgttaccggggagcttcaggcgggtattcctttttgtccccctgagggagatgcgggaaccggtatggttgcaactaacagcgttgctgtccgtacaggcaatgtgtctgccgggacttctgtttttgctatggttgttctcgaaaaggaattatccaaagtgtattcggaaattgacctggtgactacacctgacgcaaatcttgtggctatggttcattcaaataattgtacatcggactatgacgcatggatgggtatatttgctgaggcagttaagaccttgggctttgacgtgaaaaaaccacagctatatgataccctgctgggagccgcacttcaaggtgaccctgattgcggagggttgcttgcgtacggttatatttcaggtgagcatattacccattttgaagaaggtcgcccgatggttgttcgttcatcaaacagcaaattcaacctggccaactttatcagggtcaatttgtttacatctcttggagccttgaagaccggtttggatattctttttcaaaaggaagctgttaaagtggacggtattaccggacacggcggtttctttaagacgaaggaagtaggacagaagattatggcggctgcctttaatgtccctgtatctgttatgaagactgcgggtgaaggcggtgcatggggtattgccctacttgcttcgtatatgattaatagggaaagctcacagtccttggaggattttcttaaacaaaatgtgtttggggaaagccaaggtgagactgtacagccagattcgaaggatgttgacggtttcaacgagtttatgaaaaggtacacaaagggactgggtattgaaagggctgcgataaacttcttgaactga

SEQ ID NO: 12 - DNA, 1380 bp, Clostridium cellulolyticum
atgataaccaaacaaaaaccaagaatcggatttttgggcctaatgcagggattgtatgacgaatcacagccggaactgccgaaaatgcaggaggcatttgccagagaagtggttgaacaattaaaagatgtggcagatattgattttcccggtccagcaaaagaaagagaagatatagaaagatatgtaaaatatttcaatgataaagagtacgatggaataatgatagtaaatctgttgtacagtccgggaaatcgtttaatacaggctatgaagaataataatctgccaatattgctggctaatattcaaccacttcccgatgttacatcaaactgggattggattttgtgcacaactaatcagggaattcatggaatacaggatacaagtaatgttctcatgcgttgtggtattaaaccggctattataacagatgattggaaggctgaatcctttaaagcctactttgaagattgggcattggctgccaacacgcataacagactaaaaaagacaaaggttgcgattttcggccgtatgcacaatatgggtgacatacttggtgatgatgcggcattgtgcagaaaatttggtgtagaggcaaaccatgtaacaatcggtccggtttattacaacatggaaggattgtcagataaagaagtagatgcccagattgaggaagataaaaagaattttaaaattgatcctaatcttcctgaagaaagtcatcggtatgctgcacgtatgcaattagcctttgaaaaattccttaatgataacggttatgaaggtttttcacagttcttcaacatatacaaggaagacggcaggttcaaacaaataccgatattggcaggctccagtctccttgcaaaaggttatggttattcggcggaaggtgatacaaatgtacttctcatgactgtgatcggtcacatgatgataggggatcctcattttactgagatgtactccctggactttggtaaggattcagcaatgctaagccatatgggagaaggcaactggaaggttgcaaggaaggatcgcggagtgacactgattgacaggcctcttgatattggtggtcttggtaatcctccgacaccaaagttcaacgtagaaccaggaacagctacccttgtttccctcgttgcagtagaaggagaaaaataccaactaattgtatcaaagggtactatccttgatactgaggacttgccagatgttcctatgaaccatgcttttttcagaccggattccggcatcaaaaaggctatggacgaatggttagctaatggtggtacacatcacgaagtactattcctgggtgattttagaagacgttttgaattattatgtaaattcttgacataa

SEQ ID NO: 13 - DNA, 690 bp, Clostridium cellulolyticum
atgttggaacaactaaaacaagcggtgttggaagccaatctagagctgcctgaaaaaggacttgtaacatatacatggggaaatgtaagcggtatcgacagagaaagcagacttattgcaattaaacccagtggtgttgagtataatgttatgacagctgatgatattgtattaatcgaccttacaggtaaagtggtggaaggaaaattgaagccgtcttctgatgcaccaacacatgtagctctgtataatgcatttcctgatataggaggtgtaacacacacccattccaggtgggcaactgcttttgcacaggctggtatggggattcctgcttacgggactactcatgcggattacttttatggtgaaatcccatgtactcgggaaatgacaaaggatgagattgagtccgattatgaagcaaataccggaacggtgataatagagacttttaaagatttaaatcctaactatatccctgccgtacttgtaaaaaatcatgcaccttttacatggggaaaaagtgcagcggaatcggttcataattctgttgttttagaagaagtagctatgatggctattcagtgcagacaactgaacccaaatgtaactcccatgccgcaggtgctgctagacaagcattttatgaggaagcacggcccgaaagcttattacggacaaaaataa

SEQ ID NO: 14 - DNA, 1317 bp, Clostridium phytofermentans
atgaaaaattactttccaaatgttccagaagtaaaatacgaaggcccaaattcaacgaatccatttgcttttaaatattatgacgcaaataaagttgtagcgggtaaaacaatgaaagagcactgtcgttttgcattatcttggtggcatactctttgtgcaggtggtgctgatccattcggtgtaacaactatggatagaacctacggaaatatcacagatccaatggaacttgctaaggcaaaagttgacgctggtttcgaattaatgactaaattaggaattgaattcttctgtttccatgacgcagatattgctccagaaggtgatacttttgaagagtcaaagaagaatctttttgaaatcgttgattacatcaaagagaagatggatcagactggtatcaagttattatggggtactgctaataactttagtcatccaagatttatgcatggtgcttccacatcttgcaacgcagacgtatttgcatatgctgctgctaagattaagaatgcattagatgcaacaattaaattaggcggtaaaggttatgtattctggggtggtcgtgaaggttatgaaacacttcttaatacagatttaggacttgagcttgataatatggctagacttatgaagatggctgtagagtatggccgtgcaaatggttttgatggcgacttctatattgagccaaagccaaaggaaccaaccaagcatcaatatgattttgatacagcaaccgtacttgctttccttcgcaaatatggcttagaaaaagatttcaagatgaacattgaagcaaaccatgctactcttgcaggtcatacctttgaacatgaacttgcaatggctagagttaatggtgcatttggttctgtagatgcaaaccagggtgatccaaaccttggatgggatacggatcaattcccaactgatgttcatagtgcaactcttgcaatgcttgaagtacttaaggctggtggattcactaacggcggacttaactttgatgcaaaggtaagacgtggttccttcgaatttgatgatattgcatacggttatattgcaggaatggatacttttgcacttggtttaattaaggctgctgagattatcgacgatggtagaatcgcaaaatttgtagatgatcgttatgcaagctataaaacaggaattggtaaagcaattgtggatggaactacatctcttgaagaattagagcagtatgttttaacacatagtgaaccagtaatgcagagtggtcgtcaggaagttcttgaaacaatcgtaaataatattttatttagataa

SEQ ID NO: 15 - DNA, 1599 bp, Clostridium phytofermentans
atgggcatggagcattttaaagatgcgattcttacgggtaaaacaacacttggaattgagcttggttccactagaataaaagctgttttagtaaatgaagaaaacgaaccaattgcgtcaggaagccatgattgggaaaatcaatatattgataatgtatggacttacaatctggatgatatctggaggggcgttcagaatagttatggacaaatgacaagtgatgttaagaataagtacggagtagaacttacaacaattggagccattggttttagtggaatgatgcatggctatatggcttttgacgaaagtggagagttacttgtaccatttcgtacctggagaaatacaataacaggaccagcatccgagcagttgaccaatgtatttcagtatcaaattccacaacgttggagtattgcccatctatatcaagctatcttaaatggggaatcccacgtgaaaaatattagattcctgacaacattggcaggatatattcactggaagctaacaggagaaaaagtattaggagtcggagaagcatctggaatgtttccaatcgatataaatacgaaagattttaataaatcaatgttagctcagtttaatgaactggttgcttcgaatgactattcatggaaaatagaagatattctaccgaaagtactagttgcaggagagtctgctggagtattaaccgaagaaggagtaaaacttcttgatgtttcaggtaaattaaaagcaggaattcctctttgtccgccggaaggagatgctggaactggtatggtagcaaccaacagcgtagcaaagagaactggtaatgtatctgctggtacttctgtatttgcaatggctgtattagaaaaagagctttcaaaagtttacgaagaaattgaccttgtgacgactccaagcggagatcttgtggctatggtgcactgcaataactgtacttctgatttgaatgcgtgggtttctatctttaaagaatttgcttcggcaatgggcatggaagctgatatgtcaaagatattctcaacgctatacaataaggcgttagaaggcaatgcagagtgtggaggcttactcgcatacaattatttttccggtgaacatataacacactttgaagaaggccgcccattgtttgtaagaactccagagagtaagtttaaccttgcgaatttcatgagagttcatctattcacagcacttggtgctttaaagataggtcttgatatcctattaaaacaagaatcagtacaattggatgagatttttggtcatggtggattatttaagacgaaagatgtcggacaaaaaattatggctggtgcaatcaatgttcctgtttctgtgatggagactgccggagaaggcggagcatggggaatcgcaatcttggcttcttatatgatttctaaggaagaaggtcagtccttagatgagtatctttctaaacatgtattccaaggaaagacaggtagcaagatgcagccggatccaagggatgtagaaggttttgaacagtttatcaaacgatatattgacggacttgaaattgagcgtaaagcagtggagatattataa

SEQ ID NO: 16 - DNA, 240 bp, Clostridium thermocellum
actttgccaacggtacaagggaagttgcaagagcggttgccgagtccggagcaatttcaataatcggaggcggagattctgccgcagctatagaacagcttggttttgccgataagattacccacatttcaaccggaggcggcgcgtctttggagtttcttgaaggaaaagtattgccgggaattgatgtattaatggataaataaggagagaagaggtcatgagtagaaaagttattgc

SEQ ID NO: 17 - DNA, 420 bp, Clostridium thermocellum
aattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatggcagtaaaaattggtatcaacggttttggacgtatcggtcgtcttgtgttcagggccagtctcaacaacccgaacgttgaggttgtaggtataaacgacccatttattg

SEQ ID NO: 18 - DNA, 300 bp, Clostridium thermocellum
ctgccccattaaaagctcggttccaaccgctaatatctccgcattcatattgaaagaccccttaaatttaaactttttgtaacttattatatcaattagtgttataaaataaaagggaaaaagaattaaaatcaaaggtttcaagagcagccgtatcacccgtaaaagtttcagccgattcaacctttttacacataaaactttcaaaaattgatgacttacaattatcaagtaggatataatattactaatgctaaacagttattgataaaggaggaaggaatatgaacaataacaaag

SEQ ID NO: 19 - DNA, 30 bp, Artificial Sequence
  Synthetic primer
ggcggaattccttggtctgacaatcgatgc

SEQ ID NO: 20 - DNA, 33 bp, Artificial Sequence
  Synthetic primer
ggcggaattctatcagttattacccacttttcg

SEQ ID NO: 21 - DNA, 674 bp, Artificial Sequence
  Synthetic polynucleotide
gaattctgcgacagaatagggattgacaattcctttataaagcaatcaaggggttcagaagaggctgttattttgaataaagagctaaagaatcacaaagatgcaatagaggctgttatttctgcactgactgacgataatatgggcgttataaaaaacatgtccgaaatatcagcagtgggacacagaatagtacacggcggtgaaaaattcaacagttctgtagttatagatgaaaacgttatgaatgcagtaagagagtgtatagacgttgcaccgcttcataatccgccgaatattataggtatagaggcttgccagcagattatgcccaatatacctatggtagctgtatttgataccactttccacagctccatgcctgattatgcatacctttacgcattgccatatgaactttatgaaaagtacggtataagaaaatatggtttccacggaacatcacacaaatatgttgcagaaagagcttctgcaatgcttgataagtctttgaacgaattaaagataattacatgccatcttgggaacggttcaagtatttgtgctgttaacaagggtaaatcaattgatacttccatgggctttacacctttgcagggacttgcaatgggtacaagaagcggtacaatagaccctgaagttgttacgaattc

SEQ ID NO: 22 - DNA, 951 bp, Clostridium thermocellum
atgaaaaataaatctataaataaaatagtaattgtaggtacgggttttgtcggttcaacaactgcctatactttaatggtcagcggactagtttccgagattgtacttattgaccgtaacacaagcaaagccgaaggagaggcaatggatatgaatcacggtatgccctttgtaagacctgtcagaatatacaaaggtgattatcctgattgcaaaggtgctgatattgttgtaataacaggtggagcaaaccagaagcccggtgaaaccagaattgaccttgtaaataaaaatactgaagtttttaaagacattgttggaaatatcattaaatacaatacagactgtattttacttgttgttacaaacccggttgatatcttaacctatgtaacatacaaattatccggatttcccaaaaacagagttataggctccggaacagttcttgatactgcacgtttcaaatatatgcttggtgaacacatgggagttgacccaagaaacgttcatgcttatataatcggtgaacatggagatacagaggtacctacatggagtctggcatccatagccgggataccgatggatgcttattgcaaggaatgtaaatcctgtgatgctgaaaactttaagagtgaaacttttgacaaagtaaaaaatgcagcttatgaaattattgatagaaaaaatgcaacctactacgccgttgctcttgcagtaagaagaattgtagaggctatcgttcgtaatgaaaactccatattgacggtatcaagcctattcgaaggagaatacggcctcaatgacatatgtctcagtattcccagccaggtaaattcggagggtgtttcaaggattttgaatattcctctgagcagtgaggaaacaggtttacttaataaatctgcccaggccttgaaacaggttatcagtgggctgaatttataa

SEQ ID NO: 23 - DNA, 933 bp, Clostridium thermocellum
atgggttttaaagttgcgatcataggagcaggatttgttggagcatcagctgcgtatgcgatgtctataaacaacttggtttctgaattggtattaattgatgtaaataaagagaaggcttatggtgaagcacttgatatcagccatggcttatcattctcaggaaatatgacagtttattccggcgactattctgatgttaaggattgtgatgttatagttgtaactgcaggggcagcaagaaaaccgggagaaactcgtttggaccttgctaaaaagaatactatgatcatgaagagcatagttactgatataatgaagtactacaataagggtgttattgtaagtgtatcaaatcctgttgatgtattggcatatatgacacaaaagtggtcaggattgcctgcaaataaagttataggatcaggaacagttcttgacagtgcaagactgagaactcatatcagtcaggcattggatgtagacattgctaacgttcacggttatattgttggtgaacatggtgattctcagttgccattatggagtgcaacacatatagcaggagtacaatttgacgactatgtaaaagctactggcttaaatgttgataaggaagctcttttcaatgaagttaaggtagcaggtgcaactattattaagaacaagggagcaacttactacggtatagctctttcaattaacagaatagttgaatcaatcctgaaggacttcaatactattatgcctgttggtacagttcttgacggacagtacggattaaaggatgttttattaaacgttcctacgatagttggcggaaacggagctgaaaaagttcttgaagtgaacattacagatgcagaattacaacttttgaagcattcagctgaacaggttagggcagttattaacgaagttaaagacatataa

SEQ ID NO: 24 - DNA, 2830 bp, Artificial Sequence
  Synthetic polynucleotide
gacgcatacaggttgtaacacccatttcccttagcttttcgggagatgaataaaacaaactttccgggtcctttaccacaccgcccacataaagagctatgccgcatgaaagaaacgatatgttatcatttttttcgtaaactgttatttccgaacccggataaagctttaccatattattaactgctgccgtccctgcatgtgtacaccctataaccactattttcatatacatcctcctttgtttgcttgtaaatatatcccatatataccacctaaatatattttataaacaaattcggtatatcattcttttggtaaataaaaagtacatccgatattagaatgtacctaaaaaaaattattattttattgtatatgctttatctgttttcattatatggtttgctatccattctacggtaaaatcaagtaattccattaagtactgatcctgatccttgtctatcctgctataatccgtattactgattttctcaataaaatcatggtgttcaactttgtgggagagaagcttgcgatatcctatgctatgcatgtattcttcttcataggtaaaatgaaagacagtgtaatcttttagttccgtaattagccgtacaatttcatcatatttgtctgtaataagctgatttttcgtggcctcataaatttccgaagcaatctggaatagtttcttatgctgttcgtcgattttctcaattccaagaataaattcgtctctccattctatcatatggaccctcctaaattgtaatgtataccaagattatacatacttcctagaatataaacaatacaaggataaaattttaatatcgtatacctacataaatgactaacttaaagctctctaaaacttcttttttattatttctatactactaaaatcaaaaatattctctaaagtatttctacaaatgttgtttttgcaacaaagtagtatacttttgcacccagaatgttttgttataacttacaaattaggggtatatttatagtaaatactaaatggaagagtaggatattgattatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagatcccctttacttcggatgcatgccgcaggcaggcatccgaagtagtttctccattatacaagtattctcttgagtacgtcgtcgcttctcagcagctgctttgctttttccctgttttccggcacatggagataagtgtatctgttaggcttaatagtgtgtgccatgtcaattgccttttcgaagtcatctgccttcatttttaaggtttccacaaaattgataaaacccgtatcagtcagaaattttactacccgctgatatctgtgttcttgaaccctgctcataagataggttgcaatcccaacctgaattccatgaagctgaggtgtctccagcagcttatctaaagcatgagatattagatgctcactaccgctggctggagcactgctgtctgctatctgcatggcaattccgctcattgtcagagagtctaccatttcctttaaaaagaagttttctgtaacctgtgtgtagggcatccttacaatactgtttactgactttttagcaatcattgcagcaaaatcgtcaacctttgccgcattgttcctttcttcaaaataccagtcatacacagccgtaattttggatattatgtctccgagacctgaataaataaatttcataggtgcattttttaatacatctaaatccactaatattccaaatggcatcgaggcatgtacggaagtacgcctgccatttataatcaaagagcagcctgagctggaaaaaccatcgtttgaggttgatgtaggtatactgataaaaggaagcttgtttaaaaaagctatatatttggctgcatcaagcacctttcctcctcctactccgaccactgcatcggttttggagggaatagtaaaagccttgagcataagattttcaagctttatgtcatcatagtcgtaagtttcaagtactgcaagagattttcttgactttatggaatccagaatcttttcaccaaataagtcacgtattccctctccaaaaagtactacaacattactaattcctgccctttcaatatgtgc

SEQ ID NO: 25 - DNA, 572 bp, Clostridium thermocellum
ccaaggtgacaaacgataacttttgagttatttacatctaagccagcaagcgtggttgctcttttagaaacatagctgtgacttgttccgtggaaaccatatcttcttaccttatatttatcatagtactcaaatggaataccataaaggtaagcttcttttggcattgtctgatggaatgcagtatcaaaaacagctaccattggtacatttggcataattgatttacaagcgttgataccaataaggtttgctgggttgtgtaaaggtgcaagatcattacactcttcaattgcatttaagacttcatcattgattactacggaatgagcaaatttctcaccaccatgtactactctatgtccaacagcgttgatttcatctaaggacttaatcacaccataattttcattcataagagcagcgattacatttttaatagcaacctcatggtttggaagtgcatcctcaagaactaccttctcaccgtcagctgacttgtgagtaagacggccatcaataccgattctttcacaaagacctactgctaatgcttgctctgtcacagagtc

SEQ ID NO: 26 - DNA, 2676 bp, Artificial Sequence
  Synthetic polynucleotide
ctgagtgcaatgtaaaaaaggatgcctcaagtattcttgaaacatccttatattatactacaaaatcataaagtaaattactcagctgtagcaatgatctcttttttgttgtaagatccacaagctttacaaactctatgaggcatcataagtgcaccacacttgctgcatttcactaagtttggagcagtcatcttccagtttgcacgacgactatctcttctagctttggaatgtttattctttggacaaatagctcccattgattacacctccttaaacttgttaaaaatatctcggatagcagacattcttgggtctagttctgtacggtcacacccgcactctccttcatttaggttagcaccgcagaccttgcagattcctttacagtcttctttgcacagaaccttcattgggaaaccaatcaagacttcttcatagataagtttatctacgtctaaatcatatccggaaacaaaatttgtttcatctaaatcctcggtacgctgttcctctgttttcgatacatcaatctctgtagccacgtcgatgtcttgttggatggtttcttccttcaaacaacgatcgcaaggaacggctaacgctaatttcgtttttgcttccaccagaatttttcggccacctagattagttaatctaagtttaaccggttctttataggtaatagaataaccgacaccatttaattcgaatatatcaaattcaatcggtgcagtgtattctttgagaccattaggaacattcatgacttcagacatttgtatcagcataagtaactcctgtctaaaaaaacgcataatgtaagcgcccaaaaattcacactgttagtattataaacgcttaaaataggtttgtcaactcctaactgttaaaaatgtcagaattgtgtaaccatattttctcttcattatcgttcttcccttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttctgtttatctttgtgtgatattcttgatttttttctatttaatctgataagtgagctattcactttaggtttaggatgaaaatattctcttggaaccatacttaatatagaaatatcaacttctgccattaaaaataatgccaatgagcgttttgtatttaataatcttttagcaaacccgtattccacgattaaataaatctcatcagctatactatcaaaaacaattttgcgtattatatccgtacttatgttataaggtatattaccaaatattttataggattggtttttaggaaatttaaactgcaatatatccttgtttaaaacttggaaattatcgtgatcaacaagtttattttctgtagttttgcataatttatggtctatttcaatggcagttacgaaattacacctctgtactaattcaagggtaaaatgcccttttcctgagccgatttcaaagatattatcatgttcatttaatcttatatttgtcattattttatctatattatgttttgaagtaataaagttttgactgtgttttatatttttctcgttcattgtatttctccttataatgttcttaaattcatttatcacggggcaacttaatatatccgaaatatagttcttctatatcgttcccccagtataatgattattatactatttaatcttcaacttaacaattggagtttccagttaagaaataataatttaatgccaaagcggatattcgcaatccgcttacgctacttgctcataacctcaacaggcaatgaagctaagttaattatttactctgtgcctgaacagcagtgattgcaacaacaccaacgatatcatcagaagaacaacctcttgataaatcatttactggagctgcaataccctgagttaatggtccataagcttctgcctttgcaagacgctgtgttaacttatatccaatgttaccagcatcaaggtctgggaagattaatacgttagcttttccagcaatatcactaccaggagcttttgaagcacctacactaggaacgattgctgcatctaactggaactcgccgtcgatcttatattctgggtataattcatttgcaatcttagttgcttctacaaccttatcaacatctgcatgctttgcgcttccctttgttgaatgagaaagcatagctacgataggttcagagccaactaattgttcaaaactcttcgctgtggaaccagcgattgctgctaactcttcagcatttggattctgatttaaaccagcatcagagaaaaggaaagttccatttgcgcccatatcacaattaggtactaccattacgaagaaagcagaaactaacttagtatttggagcagtttttaaaatctgaagacatggtcttaaggtatctgctgtagagtgacaagcaccagatactaaaccatctgcatcgcccatcttaaccatcattacaccgtatgtaatgtagtctgttgttaaaagctcttttgctttttcaggggtcatgccttttgcctgtctaagttctacaagcttgttaatgtaagc

SEQ ID NO: 27 - DNA, 978 bp, Clostridium thermocellum
atggcgattacaataaaccgaagtaaagttattgttgtgggtgcaggtttagttggtacttcaacggcgtttagtctaattacgcaaagtgtttgtgatgaggttatgttgatagatatcaatcgtgctaaggcgcatggggaagtaatggatttgtgtcatagtatcgagtatttaaatcgaaatgttttggtaacggaaggagattatacagactgtaaggacgctgatattgttgtaataactgcagggcctccgccaaaaccaggacagtcgcggcttgatactcttgggttatccgcagatattgtgagcacgattgtggaacctgtcatgaagagtgggttcaatggaatattcttagtcgtgacgaatccggtggattcgattgctcaatatgtttatcaattatcggggcttccaaagcaacaagttcttggaactggaacagcgattgactctgcaagattaaaacactttattggagatattttacatgtagatcctagaagcatacaggcttatacgatgggagagcatggagattctcaaatgtgtccttggtcgcttgttacggttggcggtaaaaatattatggacatcgtacgggataacaaagagtattccgatattgactttaatgaaatcttatataaggttaccagggtaggttttgatattttatcagtgaagggtactacttgttatggaatagcgtcagcagctgtggggattataaaagcaattctttatgatgagaattccatccttccggtctctaccttattggagggggaatatggtgagtttgatgtatatgcaggggtaccatgcattctaaatcgtttcggcgtgaaggatgtagtggaagtaaatatgacagaagtagagttaaatcaattccgagcctctgttcacgttgtgagggaagctattgaaaacttaaaagacagagataaaaaggcattatttttataa

SEQ ID NO: 28 - DNA, 960 bp, Clostridium thermocellum
ttatgatagcgtcaatgcatactgaaaattttctttcattgttctacaagaagcatcgaattttcccttttcttcaggtgtcaaatttagctcaatgatttcttctacaccatgaattccaagtaccgtaggaacagatgcatagacatcatgctggccatactcaccatttaagagagtagatactggtaataccttcttctcatctgagaaaatggctcgtgtaacctcagctagtgatgcaccaataccaaattccgttgagccttttccagttaggatatgccatccccctgctctagcttcatcagaaagcttagaaagatcaatctgcccatatttttcaggtttttccttgattagttccaaaattggttttccagctatagataccgttgaccatgcaaccatctggctttctccgtgttctccaagaacaaatccatagattgatttttgatcaatttcaacagcatctgcaattgctcttctaagtctggcagagtctagtaccgtacttgttgaaataattttattggatgagtactgaagtaaatgctgtaaataatgtgttattacatctgctggatttgaaatgctaacaatcataccatcaaaacctgaatttttgatatgccaagctacctctttaataattagagcagtattcgtaagggtactcattcttgtttcacccttatttttatctggattggttcctactgcaatcaccatgagatctgcatcagctgcatcactataatcacccgattttaccttaactctgtgtggtaggtatactgtagcatcgtagatatccagtgcttgtgctttcgctttttctctatcaatatcaataaagataatttcttctgcaagcccctgctctgccagtgcatatccagcatgagatcctacgtgacctgctccgataataatgacttttcttggttttgccat

SEQ ID NO: 29 - DNA, 2732 bp, Artificial Sequence
  Synthetic polynucleotide
tggaatctcactatgcaccaatgtggtactaaattatatctttatctatggaaaattaggttttccgcgaatggagatagagggagctgccattgctactttaatttgtagaattcttgagagtattttagttgttatttatatgtataagggtgagaaggtacttaagatgagactttcttatatttttaagagatctaaacagtattttcgctctttggctcgttatagtgcgccagtgcttatgagtgaggttaactgggggcttgggattgctgttcagtctgcaatcattgggcgtatgggtgttagttttcttacagccgccagcttcattaatgtagtacaacagttagccggaatcattctgattggtattggtgtgggttcgagcattataatagggaatttgattggtgagggaaaagagcatgaggcgagaatgctagccaataagttaatacgtatcagtatgatactcggaggaattgttgcttttgcagtaatcttactacgtccaatcgctcctaactttattgaggcgtctaaggaaacagcggatttaattcgtcagatgctatttgtttcggcttacctcttattcttccaagccttatctgtattaactatggccggaatattacgtggtgcaggggataccctttactgtgcaacctttgatgttttgaccttatgggtactaaaacttggaggaggtttgcttgcaaccatagtacttcatcttccacctgtatgggtttactttatcttaagtagcgatgagtgtgttaaagcgctatttacggtaccgcgggtcttaaagggacgttggattcatgatacaacactgcattaagatttcatatgtccagatatttttgcacagtagcataattactagagcttattcctataatattcataggttttgatggtccattttacgttacgatagcatatattacatcaaaaccaattctatataagatgaggttatagtatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagaagtaataggaaataatactcgaattattctgcaatctgttctaaaaaataaaattaagaaattactatagcaagccaggttaaaattactagcttgctatttttgtgcatttagtacagttttgattattaaagaataaatttaataactattttgcaataagttattgactatttcacaagttagtgttactatacaagtatgaaataaagatacataaaaaaataaataatatgaaacataaattcatgacatgcggaatagaatgaaagaatattatgtcggttcctaatactaaatggatataacaatctattgaaacacttatggggtgtaagtgtggagagaatttctaaagcgccaaaagactctacatatgaaattctaaagcttcacacgggaataatctaatttatgtatcttattatcataattcaggaaggtagtgtgaaaatataaaaattagttttcctgtttcattcaggcagtagcatttcttaaacaaatttgctatgcattgggtgttatctgaaaaacaaaaagcaattttctcacaacttatttctgaacaacaatggtattaaaaatttggaggaggattttactatgaaaaaaacggtaacattactgttggttctgaccatggtggtaagcttatttgcagcatgtggtaagaaaaatggatcaagcgaaaccggcacaaaagatcctgtggcaacaagcggtgcaaaagaacctgacaaacaagatccaggcaataaagagcctgaaaaacaagaccctgttaaaatcaagatttattactctgataatgcaaccttaccatttaaagaagattggttagttataaaggaagctgagaagagatttaatgttgatttcgatttcgaagtaattccaattgcagattatcaaacaaaagtttctttaacattaaatacaggaaataacgctccagatgtcatcctttatcagtcaacgcagggagagaatgcatct

SEQ ID NO: 30 - DNA, 10665 bp, Artificial Sequence
  Synthetic polynucleotide
gaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgcgtagaggatcgagatctcgatcccgcgaaattaatacgactcactataggggaattgtgagcggataacaattcccctctaggctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttaatttcctactacgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttgaaatccgatagaggaaagtgtctgaaacctctagtacaaagaaaggtaagttacagtagtagacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatcccgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagacaaacctgaagttgaattcatatttattaaattaagcgtatatactattgaaaatgtttttgaaatattataaaattaactttggtttaggaaaagtaaccagttcttttgtcgataagcattaatttgcttgactaattaataaaaaacttaggaggtaacactaatggtattcgagaaaattgacaagaacagttggaacagaaaagaatactttgatcactattttgctagtgtaccttgcacatacagtatgactgtaaaggttgatataacacagattaaagagaagggaatgaaattgtaccctgcaatgctttattacatagcaatgatagtaaacagacatagtgaatttaggaccgctatcaatcaggatggtgaacttggaatttatgatgaaatgattccatcatatactatattccataatgacaccgagacattctcaagtctttggactgaatgcaagtcagattttaagtcatttcttgcagattatgaatctgatactcaaagatacggtaataatcaccgtatggaaggaaaacctaatgcacctgagaatattttcaatgtttccatgataccttggtcaacatttgacggatttaatctgaatctgcaaaaaggctacgattacttaatccctatctttacaatgggcaagtattataaggaagacaataaaatcatccttccccttgcaatccaggtacatcatgcagtatgtgatggatttcatatttgtcgttttgtaaatgaactgcaagaattaataaattcctaactcgagggcagtagcgcggtggtcccacctgaccccatgccgaactcagaagtgaaacgataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttttattgtaaattccggtaacccttgtagcttagtgggaatttgtaccccttatcgatacaaattccccgtaggcgctagggacactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 31 - DNA, 10665 bp, Artificial Sequence
  Synthetic polynucleotide
gaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgcgtagaggatcgagatctcgatcccgcgaaattaatacgactcactataggggaattgtgagcggataacaattcccctctaggctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttagctctcttcaatgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcgattagagctcgatagaggaaagtgtctgaaacctctagtacaaagaaaggtaagttatcattgaagacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccatcccgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagacaaacctgaagttgaattcatatttattaaattaagcgtatatactattgaaaatgtttttgaaatattataaaattaactttggtttaggaaaagtaaccagttcttttgtcgataagcattaatttgcttgactaattaataaaaaacttaggaggtaacactaatggtattcgagaaaattgacaagaacagttggaacagaaaagaatactttgatcactattttgctagtgtaccttgcacatacagtatgactgtaaaggttgatataacacagattaaagagaagggaatgaaattgtaccctgcaatgctttattacatagcaatgatagtaaacagacatagtgaatttaggaccgctatcaatcaggatggtgaacttggaatttatgatgaaatgattccatcatatactatattccataatgacaccgagacattctcaagtctttggactgaatgcaagtcagattttaagtcatttcttgcagattatgaatctgatactcaaagatacggtaataatcaccgtatggaaggaaaacctaatgcacctgagaatattttcaatgtttccatgataccttggtcaacatttgacggatttaatctgaatctgcaaaaaggctacgattacttaatccctatctttacaatgggcaagtattataaggaagacaataaaatcatccttccccttgcaatccaggtacatcatgcagtatgtgatggatttcatatttgtcgttttgtaaatgaactgcaagaattaataaattcctaactcgagggcagtagcgcggtggtcccacctgaccccatgccgaactcagaagtgaaacgataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggccttttttattgtaaattccggtaacccttgtagcttagtgggaatttgtaccccttatcgatacaaattccccgtaggcgctagggacactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 32 - DNA, 1056 bp, Thermoanaerobacterium saccharolyticum
gtgtatacaatatatttcttcttagtaagaggaatgtataaaaataaatattttaaaggaagggacgatcttatgagcattattcaaaacatcattgaaaaagctaaaagcgataaaaagaaaattgttctgccagaaggtgcagaacccaggacattaaaagctgctgaaatagttttaaaagaagggattgcagatttagtgcttcttggaaatgaagatgagataagaaatgctgcaaaagacttggacatatccaaagctgaaatcattgaccctgtaaagtctgaaatgtttgataggtatgctaatgatttctatgagttaaggaagaacaaaggaatcacgttggaaaaagccagagaaacaatcaaggataatatctattttggatgtatgatggttaaagaaggttatgctgatggattggtatctggcgctattcatgctactgcagatttattaagacctgcatttcagataattaaaacggctccaggagcaaagatagtatcaagcttttttataatggaagtgcctaattgtgaatatggtgaaaatggtgtattcttgtttgctgattgtgcggtcaacccatcgcctaatgcagaagaacttgcttctattgccgtacaatctgctaatactgcaaagaatttgttgggctttgaaccaaaagttgccatgctatcattttctacaaaaggtagtgcatcacatgaattagtagataaagtaagaaaagcgacagagatagcaaaagaattgatgccagatgttgctatcgacggtgaattgcaattggatgctgctcttgttaaagaagttgcagagctaaaagcgccgggaagcaaagttgcgggatgtgcaaatgtgcttatattccctgatttacaagctggtaatataggatataagcttgtacagaggttagctaaggcaaatgcaattggacctataacacaaggaatgggtgcaccggttaatgatttatcaagaggatgcagctatagagatattgttgacgtaatagcaacaacagctgtgcaggctcaa

SEQ ID NO: 33 - DNA, 1209 bp, Thermoanaerobacterium saccharolyticum
atgaaaattatgaaaatactggttattaattgcggaagttcttcgctaaaatatcaactgattgaatcaactgatggaaatgtgttggcaaaaggccttgctgaaagaatcggcataaatgattccatgttgacacataatgctaacggagaaaaaatcaagataaaaaaagacatgaaagatcacaaagacgcaataaaattggttttagatgctttggtaaacagtgactacggcgttataaaagatatgtctgagatagatgctgtaggacatagagttgttcacggaggagaatcttttacatcatcagttctcataaatgatgaagtgttaaaagcgataacagattgcatagaattagctccactgcacaatcctgctaatatagaaggaattaaagcttgccagcaaatcatgccaaacgttccaatggtggcggtatttgatacagcctttcatcagacaatgcctgattatgcatatctttatccaataccttatgaatactacacaaagtacaggattagaagatatggatttcatggcacatcgcataaatatgtttcaaatagggctgcagagattttgaataaacctattgaagatttgaaaatcataacttgtcatcttggaaatggctccagcattgctgctgtcaaatatggtaaatcaattgacacaagcatgggatttacaccattagaaggtttggctatgggtacacgatctggaagcatagacccatccatcatttcgtatcttatggaaaaagaaaatataagcgctgaagaagtagtaaatatattaaataaaaaatctggtgtttacggtatttcaggaataagcagcgattttagagacttagaagatgccgcctttaaaaatggagatgaaagagctcagttggctttaaatgtgtttgcatatcgagtaaagaagacgattggcgcttatgcagcagctatgggaggcgtcgatgtcattgtatttacagcaggtgttggtgaaaatggtcctgagatacgagaatttatacttgatggattagagtttttagggttcagcttggataaagaaaaaaataaagtcagaggaaaagaaactattatatctacgccgaattcaaaagttagcgtgatggttgtgcctactaatgaagaatacatgattgctaaagatactgaaaagattgtaaagagtataaaa

SEQ ID NO: 34 - DNA, 1358 bp, Thermoanaerobacter pseudoethanolicus
gctaatgctatcggaccaatttctcaaggtcttgcaaaacctatcaatgacttgtcaagaggttgtagtgtagaagatattgttaatgttatagcaataacttgtgtacaagctcaaggggtgcaaaaataactttgaggaggcagcgattatgaaaattttagtcatgaactgtggaagctcgtcattaaaagtatcaattgttagatatggataatgggaaagtgctagcgaaaggattggcggaaaggataggtatcaatgattctcttttaactcatcaagtagagggcaaagataaaataaaaatacaaaaagatatgaaaaatcataaagaagctatacaaattgttttagaggctttagtagataaagaaatcggaatattaaaagatatgaaagaaatagatgcagtaggacatagagttgtgcacgggggagagttttttactgattccgtattgattgacgatgaggtaatcaaaaaattagaagcatgtattgaccttgcacctttgcacaatcctgctaatattgagggaataaaagcttgtcggcagataatgccaggggtgccaatggtagcagtttttgatacggctttccatcaaacaatgccagattatgcgtatatttatcccattccttatgaatactacgaaaaatatagaataagaagatatggattccatgggacttctcataaatatgtatctttaagagctgctgaaatattaaagaggcctattgaagagttaaaaattattacttgccatttagggaatgggtctagtattgctgcggttaaaggcggtaagtcgatagatacaagtatgggatttactccattagaagggctggctatgggtacaaggtccggaaatgttgatccttcaattataactttcttaatggaaaaagaaggattgactgcagaacaggttatagatatacttaataagaaatcaggtgtatacggaatttcaggaataagtaatgactttagagatatagaaaatgcagcttttaaagaagggcataaaagggctatgttggcattaaaagttttcgcttatagggtgaaaaagacaataggttcttatacagctgctatgggtggggttgatgtaattgtgtttactgctggagttggagaaaatggaccagaaatgagagagtttattttagaggatctagagtttttaggctttaaactggacaaagagaagaataaggtaagaggaaaagaggaaattatatctacagaagattcaaaagttaaagttatggttattcctacaaatgaagaatatatgattgctaaagatactgaaaaattggtaaaaggtttaaagtag

SEQ ID NO: 35 - DNA, 1196 bp, Thermoanaerobacter pseudoethanolicus
atggcagtaatggatagtatcatacaaaaggctaaagctaataaaaaaaggattgtgcttcctgagggaagtgaagctcgaactttaaaagctgctgaaaaggttattaaagaaggtattgctgatgtagttttattagggaaggaagaagaaataaaagaaaaagcaaagggattggatatctcgaaagcagaaattatagaccctgaaaagtcgcctcttttacaaaaatatgctgaagaatattataatttgagaaaaaccaaaggagttacagaagaacaggcatatcaaattatgaaagaccctatgtactatgggtgcatgatggtcaaattagacgatgttgatggtatggtatctggggcgattcacgctactgctgatgttttcagaccggcttttcaaattgtaaaaactgctgcaggtgtcaaagtagtatccagcgcctttataatggaagtacctaattgtacttatggaagcgatggagtatttatttttgctgattgtgcaataaatcctaatcctaatgaagaggaattagcagcaattgccattgcttctgcccatactgcaaaagtccttgctggaattgagcctagaattgctatgctgtcattttctactaaaggaagtgcaaaccatgaattagtagataaggtgaaaaatgcgactaaaatcgcaaaagaattggcgcctgatttgctaattgatggtgagcttcaattagatgctgcgattgtcaaagaagtaggagagttaaaggctccaggaagtcctgtagcggggaatgcaaatgtgcttattttcccagatttgcaagcgggaaacattggatataagctagtgcaaagacttgctaaagctaatgctatcggaccaatttctcaaggtcttgcaaaacctatcaatgacttgtcaagaggttgtagtgtagaagatattgttaatgttatagcaataacttgtgtacaagctcaaggggtgcaaaaataactttgaggaggcagcgattatgaaaattttagtcatgaactgtggaagctcgtcattaaaagtatcaattgttagatatggataatgggaaagtgctagcgaaaggattggcggaaaggataggtatcaatgattctcttttaactcatcaagtagagggcaaagataaaataaaaatacaaaaagatatgaaaaat

SEQ ID NO: 36 - DNA, 1053 bp, Thermoanaerobacter sp.
gtgtatacaatatatttcttctttttagtaagaggaatgtataaaaataaatattttaaaggaagggacgatcttatgagcattattcaaaacatcattgaaaaagctaaaagtgataaaaagaaaattgttctgccggaaggtgcagaacccagaacattaaaagctgctgaaatagttttaaaagaaggaattgcagatttggtgcttcttggaaatgaagatgagataagaaatgctgcaaaagacttggacatatctaaagctgaaatcattgatcctgtaaaatctgaaatgtttgataggtatgctaatgatttttatgagttaaggaagagcaaaggaatcacgttggaaaaagccagagaaacaatcaaggataatatctattttggatgtatgatggttaaagaaggttatgctgatggattggtatctggcgctattcatgctactgcagatttattaagacctgcatttcagataattaaaacggctccaggagcaaagatagtatcaagcttttttataatggaagtgcctaattgtgaatatggtgaaaatggtgtattcttgtttgctgattgcgcggtcaacccatcgcctaatgcagaagaacttgcttctattgctgtacaatctgctaatactgcaaagaatttgttgggctttgaaccaaaagttgctatgctatcattttccacaaaaggtagtgcatcacatgaattagtagataaagtaagaaaagcgacagaaatagcaaaagaattgatgccagatgttgctatcgacggtgaattgcaattggatgctgctcttgtcaaagaagttgcagagctaaaagcgccaggaagcaaagttgcgggatgtgcaaatgtgcttatattccctgatttacaagctggtaatataggatataagcttgtacagagattagctagcaaatgcaattggacctataacacaggaatgggtgcaccggttaatgatttatcaagaggatgcagctatagagatattgttgacgtaatagcacacagctgtacaggctca

SEQ ID NO: 37 - DNA, 1068 bp, Thermoanaerobacter sp.
atgctaacggagaaaaatcaagataaaaaaagacatgaaagatcacaaagacgcaataaaattgttttagatgctttggtaagcagtgactacggcgttataaaggatatgtctgagatagatgctgtaggacatagagttgttcacggaggagaatcttttacatcatcagttctcataaatgatgatgtgttaaaagcgataacagattgcatagaattagctccactgcacaatcctgccaatatagaaggaattaaagcttgccagcaaatcatgccaaacgttccaatggtggcggtatttgatacagcctttcatcagacaatgcctgattatgcatatctttatccaataccttatgaatactacacaaagtacaggatcagaagatatggatttcatggcacatcgcataaatatgtttcaaatagggctgcagagattttaaataaacctattgaagatttgaaaatcataacttgtcatcttggaaatggctccagcattgctgctgtcaaatatggtaaatcaattgacacaagcatgggatttacaccattagaaggtttggctatgggtacacgatctggaagcatagacccatccattatttcgtatcttatggaaaaagaaaatataagcgctgaagaagtagtaaatatattaaataaaaaatctggtgtttacggtatttcaggaataagcagcgattttagagacttagaagatgccgcctttaaaaatggagatgaaagagctcagttggctttaaatgtgtttgcatatcgagtaaagaagatgattggcgcttatgcagcagctatgggaggcgtcgatgccattgtatttacagcaggtgttggtgaaaatggtcctgagatacgagaatttatacttgatggattagagttcttagggttcagcttggataaagaaaaaaataaagtcagaggaaaagaaactattatatctacgccgaattcaaaagttagcgtgatggttgtgcccactaatgaagaatacatgattgctaaagatactgaaaagattgtaaagagtataaaa

SEQ ID NO: 38 - DNA, 1059 bp, Thermoanaerobacterium saccharolyticum
gtgtatacaatatatttcttctttttagtaagaggaatgtataaaaataaatattttaaaggaagggatgatcttatgagcattattcagaacatcattgaaaaagctaaaagcgataaaaagaaaattgttctgccagaaggtgcagaacccaggacattaaaagctgctgaaatagttttaaaagaaggaattgcagatttggtgcttcttggaaatgaagatgagataagaaatgcagcaaaagacttggacatatccaaagctgaaataattgaccctgtaaaatctgaaatgtttgataggtatgctaatgatttttacgaattaagaaagagcaagggaatcacattggaaaaagccagagaaacaatcaaggataatatctattttggatgtatgatggttaaagaaggttatgctgatggattagtatctggcgctattcatgctactgcagatttattaagacctgcatttcagataattaaaacagctccaggagcaaagatagtatcaagcttttttataatggaagtgcctaattgtgaatatggtgaaaatggcgtattcttgtttgctgattgtgcggtcaatccatcacctaatgcagaagaacttgcttctattgctgtacaatctgctaatactgcaaagaatttgttgggttttgaaccaaaagttgccatgctatcattttccacaaaaggtagtgcatcacatgaattagtagacaaggtaagaaaagcgacagagatagcaaaggatttgatgccagatgttgctatcgatggtgaattgcaactggatgctgctattgttaaagaagttgcagagctaaaagcaccgggaagcaaagttgcgggatgtgcaaatgtgcttatattccctgacttacaagctggtaatataggatataagcttgtacagagattagctaaggcaaatgcaattggaccgataacgcaaggaatgggtgcaccagttaatgatttatcaagaggatgcagctataaagatattgttgacgtaatagcgacaacagctgtgcaggctcaa

SEQ ID NO: 39 - DNA, 1209 bp, Thermoanaerobacterium saccharolyticum
atgaaaactatgaaaattctggttattaattgtggaagttcttcactaaaatatcaattgattgaatcaattgatggaaatgtgctggcaaaaggccttgctgaaagaatcggcataaatgattccctgttgacgcataatgctaacggagaaaaaatcaagataaaaaaagacatgaaagatcacaaagacgcaataaaattggttttagatgctttggtaagtagcgactacggcgttataaaggatatgtctgagatagatgctgtaggacatagagttgttcatggaggagagtcttttacatcatcagttcttataaatgatgaagtgttaaaggcaataacagattgtatagaattagctccactgcataatcctgctaatatagaaggaattaaagcttgccagcaaatcatgccaaacgttccaatggtggcggtatttgatacagcctttcatcaaacaatgcctgattatgcatatctttatccaataccttatgagtactacacaaagtacaggatcagaagatatggatttcatggcacgtcgcataaatatgtttcaagtagggctgcagagattttgaataaacctattgaagatttgaaaatcataacttgtcatcttggaaatggctccagtattgctgccgtcaaatatggtaaatcaattgacacaagcatgggatttacaccattagaaggtttggctatgggtacacgatctggaagtatagacccatccatcatttcttatcttatggaaaaagaaaatataagtgctgaagaggtagtaaatatattaaataaaaaatctggtgtttacggtatttcgggaataagcagcgattttagagatttagaagatgctgcctttaaaaatggagatgaaagagctcagttggccttaaatgtgtttgcatatcgagtaaagaagacgattggagcttatgcagcagctatgggaggcgttgatgtcattgtatttacggcaggtgttggtgaaaatgggcctgagataagagaatttatacttgatggattggagttcttagggttcagcttggataaagaaaaaaataaagtcagaggaaaggaaactattatatctacgccaaattcaaaaattagcgtgatggttgtgccgactaatgaagaatatatgattgctaaagatactgaaaagattgtaaagagtataaaa

SEQ ID NO: 40 - DNA, 933 bp, Thermoanaerobacterium saccharolyticum
atgagcaaggtagcaataataggatctggttttgtaggtgcaacatcggcatttacgctggcattaagtgggactgtgacagatatcgtgctggtggatttaaacaaggacaaggctataggcgatgcactggacataagccatggcataccgctaatacagcctgtaaatgtgtatgcaggtgactacaaagatgtgaaaggcgcagatgtaatagttgtgacagcaggtgctgctcaaaagccgggagagacacggcttgaccttgtaaagaaaaatacagccatatttaagtccatgatacctgagcttttaaagtacaatgacaaggccatatatttgattgtgacaaatcccgtagatatactgacgtacgttacatacaagatttctggacttccatggggcagagtttttggttctggcaccgttcttgacagctcaaggtttagataccttttaagcaagcactgcaatatagatccgagaaatgtccacggaaggataatcggcgagcatggtgacacagagtttgcagcatggagcataacaaacatatcgggtatatcatttaatgagtactgcagcatatgcggacgcgtctgcaacacaaatttcagaaaggaagtagaagaagaagtcgtaaatgctgcttacaagataatagacaaaaaaggtgctacatactatgctgtggcagttgcagtaagaaggattgtggagtgcatcttaagagatgaaaattccatcctcacagtatcatctccattaaatggacagtacggcgtgaaagatgtttcattaagcttgccatctatcgtaggcaggaatggcgttgccaggattttggacttgcctttatctgacgaagaagtggagaagtttaggcattcagcaagtgtcatggcagatgtcataaaacaattagatata

SEQ ID NO: 41 - DNA, 933 bp, Thermoanaerobacter sp.
atgagtaaagtggccataataggttcaggatttgtaggtgctacatctgcatttacattggctctaagtgggactgtgacagacattgttttagtagatttaaacaaggacaaggcgataggcgatgcactggatattagccacggtataccgcttatacagcctgtaaatgtgtatgctggcgactacaaggatatcgagggcgcagatgtagtagttgtaacagcaggtgcggctcaaaagccaggagagtctaggctggaccttgtaaaaaagaatacatctatattcaagtccatgatacctgaacttttaaaatacaatgataaagctatatacctgattgtaacaaatcctgttgatatattaacgtatgttacatacaaaatagcgaaacttccgtgggggcgtgtattcggttcaggtactgtccttgacagttcccgatttaggtatcttttaagtaaacattgcaatattgatcctagaaatgtacatggaaggataattggagaacacggcgatacagaatttgcggcgtggagcataacaaatatttcaggaatatcatttaatgagtactgcaatttgtgcggacgagtttgtaatacaaatttcagaaaggaagtggaagatgaagttgtcaatgcggcttacaaaattattgataaaaagggtgccacgtattacgctgtggctgtagcagtaagaagaatagttgagtgtatcataagggatgaaaattcaattcttacagtttcatctccattaaatggtcaatacggtgtaagagatgtatctttaagcttgccatcaattgtgggcaaaaatggtgttgcaagggttctggatttgcctttggctgatgacgaagttgagaagtttaaacattcggcaagcgttatggctgatgttataaaacagttggacata

SEQ ID NO: 42 - DNA, 936 bp, Thermoanaerobacter pseudoethanolicus
atgaacaaaatatctataataggttctggatttgtcggtgctactactgcatacacactggctttgagtgggattgccaaaactattgtattaatagatattaataaagacaaagcagaaggcgatgctcttgatataagccacggcgtaccgtttattagtccagttgaattgtacgcgggagattatagtgatgtttcaggttctgacataataatcattacagcgggagcagcacaaaaaccgggagaaaccagacttgacttagtgaagagaaatacgatgatttttaaagacatagtggcaaaacttattaaagtaaatgacacagcaatataccttatagttacaaatccagtagatattcttacatacgttacctataaaatatctggcttgccatacggaagagtattggggtctggcacagttctcgacagtgcgagattcagatatcttttaagcaaacattgtaacatagatccgaggaatatacacggatatataattggggagcatggcgattctgagcttgcagcttggagcattacgaacatagcaggcataccaattgataattactgcaatttatgtggaaaagcatgtgaaaaagattttagagaggagatttttaataatgttgtaagagctgcctatacgataatagaaaaaaagggtgcgacatattatgcggttgctctcgcagtaagaagaatcgtagaagctattttcagagatgaaaattccattttgactgtgtcatctccgctaaccggccaatatggtgttacaaatgtggctttgagccttccctccgttgttggacgaaatggaatcgtaaatatacttgaattaccactttcacaggaagaaattgctgcttttagaagatcagccgaagttatcaaaagtgtaatacaagagcttgatatataa

SEQ ID NO: 43 - DNA, 631 bp, Thermoanaerobacterium saccharolyticum
aggcgatgcactggacataagccatggcataccattaatacagcctgtaaatgtgtatgcaggtgactacaaagatgttgaaggcgcggacgtaatagttgtgacagcaggggctgctcaaaagccaggtgagacgaggcttgaccttgtgaagaaaaatacagctatatttaagtccatgatacctgagcttttaaagtacaatgacaaggctatatatttgattgtcacaaatcctgtagacatactgacgtacgttacatacaagatatctggacttccatggggcagagttttcggttctggcactgttcttgacagttcaaggtttaggtaccttttaagcaggcactgcaatatagattccgagaaatgtccacggaaggataatcggcgagcatggtgacacagagtttgcagcatggagcataacaaacatatctggaatatcatttaatgagtactgcagcatatgcgggcgcatctgcaacacaaatttcagaaaggaagtagaagaagaagtcgtaaatgctgcttataagataatagacaaaaaaggtgctacatactatgctgtcgcagttgcagtaagaaggattgtggagtgcatcttaagagatgaaaattccat

SEQ ID NO: 44 - DNA, 2229 bp, Thermoanaerobacterium saccharolyticum
atgatcaatgaatggcgcgggtttcaggagggcaaatggcaaaagactattgacgttcaagattttatccagaaaaattacacattatacgaaggcgatgatagttttttagaagggcctacagaaaagactattaagctttggaacaaagttcttgagctaatgaaggaagaactgaaaaaaggtgtgttagatattgatacaaaaactgtatcgtctataacatcccatgatgcggggtatatagacaaagatcttgaggaaatagttggattgcagacagacaaacctcttaaaagagctataatgccttacggtggcataagaatggtcaaaaaagcttgcgaagcttatggatataaagtggacccaaaagtagaagagatatttacgaagtacagaaagacccacaatgatggtgtatttgatgcatatactccagaaataagagcagcaagacatgccggcataataacaggtcttccagatgcatatggcagaggaagaatcataggtgattacagaagagttgctctttatggaattgatagactcatcgaagaaaaggaaaaagaaaaacttgagcttgattacgatgaatttgatgaagcaactattcgcttgagagaagaattgacagaacagataaaagcattaaacgaaatgaaagagatggctttaaagtacggttatgacatatcaaagcctgcaaaaaatgcaaaagaagctgtgcagtggacttactttgccttccttgctgctataaaggaacaaaatggtgccgctatgtcgctgggcagagtatctacttttttagatatatacattgaaagagatcttaaagaaggaacattgacagagaaacaagcacaagagttaatggatcactttgtcatgaagcttagaatggtgaggttcttaaggactcctgattacaatgaactatttagtggcgatcctgtttgggtgactgaatcaattggcggtgtaggcgtagacggaagacctcttgtcactaaaaattcattcaggatattaaatactttatataacttaggtcctgcacctgagccaaacttgacggttttatggtccaaaaaccttcctgaaaactttaaaagattctgtgccaaggtatcaatagatacaagttctattcaatatgaaaatgacgacttaatgaggccaatatacaatgacgactatagcatcgcctgctgtgtgtcagctatgaagacgggagaacagatgcaattttttggagcaagggcaaatctcgcgaaggcgctactgtatgctataaacggcggtatcgatgaaaggtataaaacgcaagtggcaccaaaatttaatcctataacgtctgagtatttagactacgatgaggtaatggcagcatatgacaatatgttagagtggcttgcaaaagtgtatgttaaagctatgaatataatacactacatgcacgataaatacgcttatgaaagatcccttatggctttgcatgatagagacatcgtaaggacgatggcttttggaatcgcaggtctttctgttgcggcagattcgttaagcgccataaagtatgctaaagtaaaagccataagagatgaaaatggcatagcaatagattatgaagtggaaggagatttccctaagtttggcaatgatgatgacagggttgactcaatagcagttgacattgtagaaagattcatgaataagcttaaaaagcacaagacttacagaaactctataccaacactgtctgttttgacaataacgtcaaatgtggtgtacggcaaaaagacgggtgctacacctgacggaagaaaagcgggagaaccttttgcgccaggcgcaaatccgatgcacggcagagatacaaaaggtgccatagcatcaatgaattcagtatcaaaaataccttatgacagttcattggatggtatatcatacacatttacgattgtaccaaatgcgcttggcaaggatgacgaagataaaattaataatcttgtaggactattagatggatatgcatttaatgcggggcaccacataaacatcaatgttttaaacagagatatgttgcttgatgctatggagcatcctgaaaaatatccgcagcttactataagggtttcagggtatgctgtcaatttcaataaattaacgagagagcaacagttggaggttatatcccgcacttttcacgaatctatgtag

SEQ ID NO: 45 - DNA, 2229 bp, Clostridium thermocellum
atggatgcatggcgcggatttaataaaggcaactggtgccaggaaattgacgttcgtgattttataattagaaattatactccttatgaaggcgatgaaagctttcttgtaggacctacggatagaacgcggaaactttgggagaaggtttccgaactgttaaagaaagaacgggagaacggcggggtattggatgttgatacccatacaatttcaacgattacgtctcataaacctggatatatagataaagaacttgaagttattgtcgggcttcagacggatgagcctttaaaaagagccataatgccgtttggcggtatacgtatggtgattaagggagccgaagcttatggccacagtgtggaccctcaggttgttgaaatattcacaaagtacagaaagactcataaccagggagtttatgatgtatatactcccgaaatgagaaaagccaaaaaagccgggattattacaggacttcccgacgcatacggcagaggaagaataattggcgattacagaagggttgcactttatggcgttgacaggctgattgctgaaaaagagaaagaaatggcaagtcttgaaagagattacattgactatgagactgttcgagacagagaagaaataagcgagcagattaaatctttaaaacaacttaaagaaatggctttaagttacggttttgacatatcttgtcctgcaaaggatgccagagaagcctttcaatggttgtattttgcatatcttgcagcagtcaaggaacagaacggcgcggcaatgagtattggaagaatttcgactttccttgacatatacattgaaagggatctcaaagaaggaaaactcacggaggagttggctcaggaactggttgaccagctggttataaagctgagaattgtgagatttttgagaactcctgagtatgaaaagctcttcagcggagaccccacttgggtaaccgaaagtatcggaggtatggcgctggatggaagaacgctggttacaaaatcttcgttcaggtttttgcacactcttttcaacctgggacatgcaccggagcccaaccttacagtactttggtccgtcaatcttcccgaaggctttaaaaagtactgtgcaaaggtatcaattcattcaagctccatccagtatgaaagcgacgacataatgaggaaacactggggagacgattatggaatagcatgctgtgtttctgctatgagaattggaaaacagatgcagttcttcggtgcaagatgcaatcttgcaaaagctcttctttacgctattaacggcggaaaggatgaaatgacgggagaacagattgctccgatgtttgcaccggtggaaaccgaataccttgattacgaggacgtaatgaagaggtttgacatggtgcttgactgggtggcaaggctttatatgaacaccctcaatataattcactacatgcatgacaaatatgcctatgaggcgctgcagatggcattgcatgacaaagacgtgttcaggacgatggcatgcggaatagccggtttgtctgtggtggcagactcccttagcgcgataaaatatgcaaaggttaaaccgatacgcaatgaaaacaacctcgttgttgactacgaagttgagggtgattatcctaaattcggaaataacgacgaacgtgttgatgaaattgcagtgcaagtagtaaaaatgttcatgaacaagcttagaaagcaaagggcttacagaagtgccactccgaccctttccatacttaccataacttcaaacgtggtatatggaaagaaaaccggaaacactcctgacggcagaaaagctggagaacctttggcgccgggagcaaatccgatgcatggaagggatataaacggagcattggctgtactgaacagtattgcgaagcttccctatgaatatgcccaggacggcatttcatatactttctccataattccaaaagctctgggaagagacgaggaaaccagaataaacaatcttaaatcaatgcttgacggatatttcaagcagggcggccaccacataaatgtaaatgtgtttgaaaaagagacactgttagatgccatggaacatccggaaaaatatccacaacttaccataagagtgtccgggtatgcagtgaactttataaagcttacacgggagcaacagctggatgttattaacagaacgattcacggaaagatttaa

SEQ ID NO: 46 - DNA, 2061 bp, Clostridium phytofermentans
atgatgacttcagttatgaaacaggaatgggaaggttttaaacaaggtagatggatcacttcagtaaatgttcgagacttcatacagaacaattacacaatgtatgatggtgatgaatcctttttagcaggtccaaccgaagccaccaataaactatgggcccaggttatggagctttcaaagcaggaaagtgagaaaggtggagtccttgatatggacaccaagatagtatctactattgtttctcacggtcctggttatttagataaagatattgaaacaattgttggttttcagaccgataagccatttaagagatcactacaggtctttggtggtattcgtatggcacagagtgcttgccatgaatatggatatgaggtagacgaagaggtagcacgtatttttacagactaccgcaagacacataatcaaggtgtatttgatgcatacactgacgaaatgaagctcgctagaaaatcagcaatcattactggtttgcctgatgcttatggtagaggtagaattattggcgattaccgtcgagtggcactttacggtactgatttacttattgaagacaagaaagaacaacttacaacttccttaaagagaatgactagtgataatattcgcttaagagaagaattagcagaacaaattcgtgcattaaaagaattagcgaagcttggtgaaatctatggttacgatattacgaagccagcaataaatgcaaaggaagcaattcagtggctttactttggatatcttgcagcggtaaaagagcaaaacggtgctgcaatgagcttaggccgtacttctacattccttgatatttatatccagagagatttagataatggtgttatcacagaaaaagaagcacaagagtatatcgatcattttattatgaaacttcgtctagtgaagtttgcaagaactccagaatacaatgccttattctccggtgaccctacttgggtaacagaaagtatcgctgggattggtacagatggacgccatatggtaacaaagacatccttccgttaccttcatacgttagacaaccttggaactgctccagaaccaaacatgacagttctatggtcaactagattaccaagattatttaaagagtactgtgctaagatgtcaattaagtcatcctctattcaatacgaaaatgatgatatcatgcgtccaactcatggtgatgattatgcaattgcttgttgtgtatcctctatgaaaattggtaaagagatgcagttctttggagcacgtgcaaatcttgctaagtgtcttctttacgcaatcaatggtggtgtagatgaagttcttaaaattcaggttggtccaaagtaccgtccagttgagggtgaataccttaattatgaggacgtaatgtcgaaatacaaagatatgatggagtggctagcagaactttatgtgaatactttaaatgtaatccactacatgcatgataaatatagctatgaaagaattcaaatggcacttcatgatcgtgaagtaaaacgttactttgcaactggtattgcgggtctttctgttgtagcggactctttaagtgcaattaagtatgctaaggtaaaagtaattcgtgatgagaatggcgttgtaaccgattacgaaattgaaggtgattatccaaagtacggcaacaatgatgatcgtgtagacgatatcgctgtacagttagtgcatgactttatgaacatgattcgcaagcatcatacttatcgtgatggatacccaacgatgtcaatcttaacgataacttctaatgtagtttatggaaagaagacaggtaatactccagacggacgtaagaagggtgaaccattagcaccaggtgctaacccaatgcatcgtcgtgatactcatggtgcagcagcgtccctagcatcggtagcaaagcttccattccgtgatgcgcaggatggtatttctaatacgttctctattgtaccaggagcattaggtaagaatgatgtgttatttgctggagacttagatttagacgatatgtctgagaactaa

SEQ ID NO: 47 - DNA, 5003 bp, Artificial Sequence
  Synthetic polynucleotide
agagctataatgccttacggtggcataagaatggtcaaaaaagcttgcgaagcttatggatataaagtggacccaaaagtagaagagatatttacgaagtacagaaagacccacaatgatggtgtatttgatgcatatactccagaaataagagcagcaagacatgccggcataataacaggtcttccagatgcatatggcagaggaagaatcataggtgattacagaagagttgctctttatggaattgatagactcatcgaagaaaaggaaaaagaaaaacttgagcttgattacgatgaatttgatgaagcaactattcgcttgagagaagaattgacagaacagataaaagcattaaacgaaatgaaagagatggctttaaagtacggttatgacatatcaaagcctgcaaaaaatgcaaaagaagctgtgcagtggacttactttgccttccttgctgctataaaggaacaaaatggtgccgctatgtcgctgggcagagtatctacttttttagatatatacattgaaagagatcttaaagaaggaacattgacagagaaacaagcacaagagttaatggatcactttgtcatgaagcttagaatggtgaggttcttaaggactcctgattacaatgaactatttagtggcgatcctgtttgggtgactgaatcaattggcggtgtaggcgtagacggaagacctcttgtcactaaaaattcattcaggatattaaatactttatataacttaggtcctgcacctgagccaaacttgacggttttatggtccaaaaaccttcctgaaggtcaatctatgaaatgcgattaagcttggctgcaggtcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgactctagaggatccccgggtaccgagctcgaattcactggccgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcca

SEQ ID NO: 48 - DNA, 6033 bp, Artificial Sequence
  Synthetic polynucleotide
cccattgtgcaatcgatcaattgaaatagctattcaattgatttaagcaattttatttcttcttcatttaattctcgccattccccttcttttaaattttcatctaattttaattggcctattgaaagcctttttaagtagacgacttttgagcctatcgcttcaaacattctttttatttgatgatatttaccttctctgattgagacatatactttcgatgtactgcctgaagatattatttccagttttgccggcatagtcttgtaaccatcgtctaatagtatgccatctgaaaacaaagatacgtcatcttcatcgataaaacccaaaacttctgcatagtattttttgaaaacgtgtttttttggcgataagagcttatgagatagttcaccgtcatttgtaattaaaagtaatccctctgtgtctttatcaagccttcctgctgggaaaacctttcttgcctttatatggtgtggcaaaagatctacaacggttttttctgatggatcatatgttgcacagattacacctttcggtttattcatcattatatatatgtattctttgtacgatattttttcacttctaaacgtgattatatctttatcaggttgtactgcaaaaccggggtcgtcaatcgtcacattatttattgcaacaaggccttcttttataaaatttttaatttcttttcttgtgccataacccatatttgataaaagcttatctattctcatttttgacatcttaaattcctcctaaacaatatgactgtgcttcttagtaaattatatcccaaaaatataaaatttgtagcaaaaatgtgatatatatcatattttttgcgttttcctgatgatacaattaagatgatgtttcaagataataaatttttctgaagtgtatacagtatattgactacaaagaacaaaatactgcaggtcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgactctagaggatccccgggtaccgaaaaggtgattgtcatggttatggggaagatacattcaatagagacatgtggtactgtagatgggcctggcataaggtacgtagtctttatgcaaggttgtcctttaaggtgcgcttattgccataaccctgacacatggaattataacggtggtaaagaagtatcaacagatgagatatttaacgatgcaaaaagatatataccgtacatgaaatcatcaggcggcggcgtgacgctgacaggtggagagcctacattacagcctgaattttgcgaagatctatttaaaaagcttaaagcgtctggcatacacactgcattagacacatcgggatatgtgaatatagataaagtaaaagaacttgtaaaacacactgatctttttttgcttgatataaagcacattgatgatgaaagccataaaaagcttacaggagtgtcgaatagaaagactttggagtttgcaagatacctttccgatgaaggcaagaaaatgtggataaggcatgtgatagtacctggaataacggatgatatggaagagataaggaaattggctgattttgtctcatcattgaaaaatgtagatagagttgagatacttccgtatcataaaatgggtgtgtataaatatgaggcacttgggataccatatagattgaagggaataaatcctcctgacacatcaaaaattaaagagataaaagaagagtttaggaaaagagatataaaagtggtctaaaagcctcatgattcgtatcatggggcttttcctttgaattaatttgataaagggtgtaaaattatcatgtgatgatgtgattttggaggtaatcgcatgaatttaaataagataaatagaaacacgtactacatagataatcctacgaatattggcgtttatgcctataaaaataaaaattgtctattagtagatactggtataaacgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcca

SEQ ID NO: 49 - DNA, 4542 bp, Artificial Sequence
  Synthetic polynucleotide
gtttatgatgtatatactcccgaaatgagaaaagccaaaaaagccgggattattacaggacttcccgacgcatacggcagaggaagaataattggcgattacagaagggttgcactttatggcgttgacaggctgattgctgaaaaagagaaagaaatggcaagtcttgaaagagattacattgactatgagactgttcgagacagagaagaaataagcgagcagattaaatctttaaaacaacttaaagaaatggctttaagttacggttttgacatatcttgtcctgcaaaggatgccagagaagcctttcaatggttgtattttgcatatcttgcagcagtcaaggaacagaacggcgcggcaatgagtattggaagaatttcgactttccttgacatatacattgaaagggatctcaaagaaggaaaactcacggaggagttggctcaggaactggttgaccagctggttataaagctgagaattgtgagatttttgagaactcctgagtatgaaaagctcttcagcggagaccccacttgggtaaccgaaagtatcggaggtatggcgctggatggaagaacgctggttacaaaatcttcgttcaggtttttgcacactcttttcaacctgggacatgcaccggagcccaaccttacagtactttggtccgtcaatcttcccgaaggctttaaaaagtactgtgcaaaggtatcaattcattcaagctccatccagtatgaaagcgacgacataatgaggaaacactggggagacgattatggaatagcagatggattttctattattgcaatgtggaattgggaacggaaaaattattttattaaagagtagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcca

SEQ ID NO: 50 - DNA, 5648 bp, Artificial Sequence
  Synthetic polynucleotide
cccgcaataatggaagtaaagagcatgaaagtgggaatgctggcttacaccgatatggcggaaattgtgtacaagggcaatccgaactacaagtttgcggccggagaggacaagccgggggttgcaccaagacctttgaaatttgacgattccataaaaaaagacatagaagagttacggagcaaggtggatattttaattgtttcacttcactggggagtggaggaaagctttgaagttctgcctgaacagagggaatttgcccacagtcttatagataacggagtggatgtaatattgggacaccatccccaccagttccaaggtatagaaatctacaagggcaaacctgttttctacagtctgggtaattttatttttgatcagaacgatcccgaaaaccaggagtcctttattgtgacacttgattacaaaggcagcagactgacaggaatagaggctgtacccgtgagaacaatcggaaaaatacaggtagttcctcaaaaaggagatgaagcaaaacctattttggaaagagagaaaaatttatgtaataggcttgatacaaactgcattataaaagatgacaaattatattttgaaattggaaaataatgataatataattaagttggacgtattttgacaaaataaaatcataaagtggttgcatttgtcgagatttgtgatatcattggatagtaaattatattttaggttaaaaatggaaaaatagttttttatttaaactttatttttaaactttatttaaaatatcaaaataattgcctttgtattttacttattgtacaatatatttgtacaatatattaaggaaaaaaatacttttgtagcgacttaaaagtcaattgaatggaccaataaaggaccttttcaaatttgtcaaggtattttaggacaattttttttattttggatattgttcttgtttattgggtaaataagatggattttctattattgcaatgtggaattgggaacggaaaaattattttattaaagagtagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccattaaagggcaggatacactcatttgaatcttttgggacactggacggaccgggtataagatttgtggttttcatgcagggctgtcccttgcgttgtatatattgccacaacagggatacctgggatgttaatgcggggagtgagtacactccccggcaagtaattgatgaaatgatgaaatacatagactatataaaggtctccggaggcggaataactgttaccggcggggagcctgttctccaggccgattttgtggccgaggtgttcagacttgcaaaagagcagggagtgcatacggcgctggataccaatggatttgctgacatagagaaggttgaaaggcttataaaatacaccgatcttgtattgctggatataaagcatgcccgggaggataaacataagataattaccggtgtgtccaacgaaaaaatcaagcgttttgcgctgtatctttcggaccagggagtgcctatctggataagatatgtccttgtccccggatataccgacgatgaagatgaccttaaaatggcggctgatttcataaaaaagcttaaaacggtggaaaaaatcgaagttcttccttatcacaacatgggagcatacaaatgggaaaaacttggtcagaaatacatgcttgaaggagtaaaggggccgagtgcgcaagaggtggaaaaagcaaagaggattctgtcaggcaaataataaaagcttttttcttttattatttgcttttttctattaccaatttgctttgcttaagtttaggtttggttttgatgagttttttaatgtttcttttatatttatcttttatatgaacagtgttgtaaacttccaaatccagtttgtcaaatattgatttaaaaatctttgccgtatactgggcgtcagttaatgcccggtgaagattttcgtctatttcaacgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcca

SEQ ID NO: 51 - DNA, 4648 bp, Artificial Sequence
  Synthetic polynucleotide
tttggtggtattcgtatggcacagagtgcttgccatgaatatggatatgaggtagacgaagaggtagcacgtatttttacagactaccgcaagacacataatcaaggtgtatttgatgcatacactgacgaaatgaagctcgctagaaaatcagcaatcattactggtttgcctgatgcttatggtagaggtagaattattggcgattaccgtcgagtggcactttacggtactgatttacttattgaagacaagaaagaacaacttacaacttccttaaagagaatgactagtgataatattcgcttaagagaagaattagcagaacaaattcgtgcattaaaagaattagcgaagcttggtgaaatctatggttacgatattacgaagccagcaataaatgcaaaggaagcaattcagtggctttactttggatatcttgcagcggtaaaagagcaaaacggtgctgcaatgagcttaggccgtacttctacattccttgatatttatatccagagagatttagataatggtgttatcacagaaaaagaagcacaagagtatatcgatcattttattatgaaacttcgtctagtgaagtttgcaagaactccagaatacaatgccttattctccggtgaccctacttgggtaacagaaagtatcgctgggattggtacagatggacgccatatggtaacaaagacatccttccgttaccttcatacgttagacaaccttggaactgctccagaaccaaacatgacagttctatggtcaactagattaccaagattatttaaagagtactgtgctaagatgtcaattaagtcatcctctattcaatacgaaaatgatgatatcatgcgtccaactcatggtgatgattatgcaattgctagatggattttctattattgcaatgtggaattgggaacggaaaaattattttattaaagagtagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcca

SEQ ID NO: 52 - DNA, 5706 bp, Artificial Sequence
  Synthetic polynucleotide
tcacccagcagcagccatgattataaacaccggagttccggagcttagtagcgaagcatctaaaagcggaaagccttatatttacggcggaacgggaaatggaccagtctttattgaacgtaccgctgatgtaagaaaagcggtagaggatatcattgcaagccgcacctttgattacggaatcgtgtctgcggcagaacaatatatggtagtagacagtcttattgcagctgaagtaaaagctgagatgttaagaaacggtgcctacttcatgaacgaggaagaggagaaaaagctaatagacctcctaaaccttacgagtggaaaggcagatacagaaattatgggaagaccagccgaagaacttgccaaacgagcaggatttatggtacctaataccacgactgtgctggtttccgaacagaaatatatttccgacaggaacccatttgcaaaagagcttctttgtcctgtattggcttactacatcgaaaatgactggatgcatgcttgtgagaagtgcatgagtcttttagtaaacgaaagccatggacataccctggtgattcattccagggatgaagaagtaataggccagttcgccttaaagaaaccagtaggcagagtacttgtaaatacccccgctaccctgggtagtatgggtgcaaccacaaacttgtttccggctatgaccctaggaagcattacagcaggcgccggaatcacagcggacaatgtttctcctatgaatttcatatacattcgtaaagtaggatatggagttcggggagtacaagaatttcttggttcggttgagaaaacctcaagcggatacgcgaaagctcctgaaacaatcaggaacaatgcccttgaaacaaacaaggtcaatgcctttgaaacaagcaaaggcatggaagatgctagagatcttttgaaacagattttacaagccttgtccaaagaactagatggattttctattattgcaatgtggaattgggaacggaaaaattattttattaaagagtagttcaacaaacgggattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccaacggagagtaatcaaaatggaccatggaaaatcaggagagattgaacgtaaggcgtttatatttaacgtgcagaagtacaacatgtatgacgggccgggaatcagaaccttggtattctttaaaggctgtcctcttcggtgtaaatggtgctccaatccggaaggtctggaacgaaaatttcaggtaatgtataagcaaagtttttgtacaaactgcggggcgtgcgctgatgtgtgccccgtaggaatccacgtgatgtcgaacggaacacatgaaattgttcgggaaaaggaatgcatcggctgcatgaagtgtaaaaacatctgcccaaagtcggcgcttaccattgcaggagaggtaaagaccatttcagaactgcttaagattgtggaagaggacgctgctttttatgatatgtccggaggtggcgtgacccttgggggtggtgaagtaaccgcacaaccagaagcggccttaaatcttttgatggcttgtaaacaggagggaatcaacacagcaattgaaacttgcggttattcgaatacagagaacattttaaaaattgcggaatatgtggatcttttcctgtttgatatcaaacatatggatccagtacgtcacaacgagttaacaggtgtgaacaatgaacagattcttactaaccttgaggaactgcttcaccgccgctataacgtaaaagtccgtatgccaatgttaaaaggaattaatgacagcagggaagaaattgatgcggttatcaagtttttaatgccataccgtactgataagaactttaagggaattgacttacttccataccataagctcggagttaataaatacaatcagcttgataaggtatatccgattgacggcgatcctagcttaagtgctgaggatttagaccgaattgaaggttggatgaaagaatacgattttccggttaacgtggtaaaacactaagaaaggggaaggacgccatggaagaaggcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcca

SEQ ID NO: 53 - DNA, 5780 bp, Artificial Sequence
  Synthetic polynucleotide
tctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgtacaaggatatcgagggcgcagatgtagtagttgtaacagcaggtgcggctcaaaagccaggagagtctaggctggaccttgtaaaaaagaatacatctatattcaagtccatgatacctgaacttttaaaatacaatgataaagctatatacctgattgtaacaaatcctgttgatatattaacgtatgttacatacaaaatagcgaaacttccgtgggggcgtgtattcggttcaggtactgtccttgacagttcccgatttaggtatcttttaagtaaacattgcaatattgatcctagaaatgtacatggaaggataattggagaacacggcgatacagaatttgcggcgtggagcataacaaatatttcaggaatatcatttaatgagtactgcaatttgtgcggacgagtttgtaatacaaatttcagaaaggaagtggaagatgaagttgtcaatgcggcttacaaaattattgataaaaagggtgccacgtattacgctgtggctg

SEQ ID NO: 54 - DNA, 6539 bp, Artificial Sequence
  Synthetic polynucleotide
gcacatattgatagagaacatggctggataattactataactccaagaaaacgaatagtaaaagaatggaggcgaattaatgagtaatgtcncaatacaattaatagaaatttgtcggcaatatgtaaataataacttaaacataaatgantntatcgaagatttccaagtgctttatgaacaaaagcaagatttattaacagatgaagaaatgagtttgtttgatgatatttatatggcttgtgaatattatgaacaggatgaaaatataagaaatgaatatcacttgtatattggagaaaatgaattaagacaaaaagtgcaaaaacttgtaaaaaagttagcagcataataaaccgctaaggcatgatagctaaagcggtatttttatgcaattaaaaggaaaaatgatatctgataaaccgcggaaaagtattttagaaaacaactataaagataatatttcaaagcaagaaggataaaataagattaaactattagacacttttattagaaaatgttataatattattaagagaaaatttatattatttaggaggtaattttatgagtaaagtggccataataggttcaggatttgtaggtgctacatctgcatttacattggctctaagtgggactgtgacagacattgttttagtagatttaaacaaggacaaggcgataggcgatgcactggatattagccaaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgacgtattacgctgtggctgtagcagtaagaagaatagttgagtgtatcataagggatgaaaattcaattcttacagtttcatctccattaaatggtcaatacggtgtaagagatgtatctttaagcttgccatcaattgtgggcaaaaatggtgttgcaagggttctggatttgcctttggctgatgacgaagttgagaagtttaaacattcggcaagcgttatggctgatgttataaaacagttggacatataaaataaatcattgtataaggtttataagacggcttttatcatgtatggtaaaggccgcttttttatgaatataaaaatacaaagtggaaaatctaaataaaggtgatgcaatatgcagaatatgagtcctcaagaaattatatcgagtgcctttatgaaggcaaaaaaatctgagaatattatacatgctaaggctatagattatgggaaaaatatatcagataaccagatgcaagcgatattgaagcaaatagagataacggctttaaaccatgtggacaaaatagtgacagctgagaagacgatgcatctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctgg

SEQ ID NO: 55 - DNA, 6086 bp, Artificial Sequence
  Synthetic polynucleotide
tctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcggctgtacaatctgctaatactgcaaagaatttgttgggctttgaaccaaaagttgctatgctatcattttccacaaaaggtagtgcatcacatgaattagtagataaagtaagaaaagcgacagaaatagcaaaagaattgatgccagatgttgctatcgacggtgaattgcaattggatgctgctcttgtcaaagaagttgcagagctaaaagcgccaggaagcaaagttgcgggatgtgcaaatgtgcttatattccctgatttacaagctggtaatataggatataagcttgtacagagattagctagcaaatgcaattggacctataacacaggaatgggtgcaccggttaatgatttatcaagaggatgcagctatagagatattgttgacgtaatagcacacagctgtacaggctcataaatgtaaagtatggaggatgaaattatgaaaatactggtatatgcgaagtctcactaaatatcactgatgatcatgatgaaatgtgctggcaaaggccttgctgagagaatcggcataaatgatccctgttgacacatatgctaacggagaaaaatcaagataaaaaaagacatgaaagatcacaaagacgcaataaaattgttttagatgctttggtaagcagtgactacggcgttataaaggatatgtctgagatagatgctgtaggacatagagttgttcacggaggagaatcttttacatcatcagttctcataaatgatgatgtgttaaaagcgataacagattgcatagaattagctccactgcacaatcctgccaatatagaaggaat

SEQ ID NO: 56 - DNA, 7284 bp, Artificial Sequence
  Synthetic polynucleotide
taacagggccatgaccttctgcaaagcacttttttaaaatttcaagagaatattgtggcatatatttttctaatgcagaatagtttttatcatttataacctttctaagatatgtagcacttgcaaaagattctactgtttccagagaattatatcctggaccaaatctttttaatgtcaaaggcttaatattgctgtttaacttgattaagctttttatgtattcaattccaagtatgttatttggatttccgataatattgttaatggtccttccgaaatattttgataatgctgcttcacgtgcctttgcatatgtgatgccgctttttaaataggttttcaaagagctttataatcttctggttcgtctaaaagaaattttgatatttggcataattcgtcaatagagccatgttcgcttccaaacgatatgtaatcaatgacatttaatgaatccagcagctttactgctccatatgcgaaattttctgcggtagaaactgcatatatagtgggcaattctattactaagtcaatacctgatagcaatgcagcttctgtccttgaccacttgtcaataatagacggtattcctcgctgaacaaagttaccactcataattgcaatgacaaaatctgcacctgttgtttcaattgatttttttatatggtatatgtgtccgttatggagagggttatattctacaattactcccaatatactcattattaaaaacctttctaaaaaattattaattgtacttattattttataaaaaatatgttaaaatgtaaaatgtgtatacaatatatttcttctttttagtaagaggaatgtataaaaataaatattttaaaggaagggacgatcttatgagcattattcaaaacatcattgaaaaagctaaaagtgataaaaagaaaattgttctgccggaaggtgcagaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgaaagttagcgtgatggttgtgcccactaatgaagaatacatgattgctaaagatactgaaaagattgtaaagagtataaaatagcattcttgacaaatgtttaccccattagtataattaattttggcaattatattggggtgagaaaatgaaaattgatttatcaaaattaagggacataggggccgcagcatcgaagtcaactacgtagaaaatctgagtgttcttgaggcaaatagcaatagatacgtagttataaagcctattagcgtaactggaagcataacatacgatagtgaaggaatagttttaaaacttttggcacgcggggctattaaagtaacatgcgataggtgccttgacgaatttgagtatgagttcgtaatacctattgacgaaatagtaaacgagtctgatgatgaattttcaggtgaagtggaagatgaaaagcttgatttgacgaaaattgtgattgaaaatgtggaactttctcttccgatgaagttcatttgctcgaatgattgcaagggtctatgttctacttgcggtaaaaatcttaatcatgaaaaatgcgattgccaaataaaagaaattgatccacgcctttcagttttgaataaattactgcagaagatgtaggaggtgtataatatgccagttccaaagcgtagaacatctaaggcaagaagagataaaagaaggcatagccatagtttagctgtacctgcttatgttttgtgcccacaatgtcatgaaccaaaattgccccacagagtttgtttaagctgtggttattacgacggtaaagaggtattgaaagtggaagaaaagtaatggagttttctctattacttttcttttttatttcttgacttttatgtatggcgtaatttataattatgagtaagtcataaaaacaacctatatttggagctgataatgtggccacgaagcttagtaaaagagatagattaaaaaagttaaaaattgaaatcgaaaaatatccattttacactgatgatgagttagctgatttgttttcggttagcgttcagacgataaggctggattctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctgg

SEQ ID NO: 57 - DNA, 4621 bp, Artificial Sequence
  Synthetic polynucleotide
cagcacaaaaaccgggagaaaccagacttgacttagtgaagagaaatacgatgatttttaaagacatagtggcaaaacttattaaagtaaatgacacagcaatataccttatagttacaaatccagtagatattcttacatacgttacctataaaatatctggcttgccatacggaagagtattggggtctggcacagttctcgacagtgcgagattcagatatcttttaagcaaacattgtaacatagatccgaggaatatacacggatatataattggggagcatggcgattctgagcttgcagcttggagcattacgaacatagcaggcataccaattgataattactgcaatttatgtggaaaagcatgtgaaaaagattttagagaggagatttttaataatgttgtaagagctgcctatacgataatagaaaaaaagggtgcgacatattatgcggttgctctcgcagtaagaagaatcgtagaagctattaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 58 - DNA, 6130 bp, Artificial Sequence
  Synthetic polynucleotide
aggtttatcgccgccttgtacagttttaaattgcatagaaatcatgagacccattgcaataaatacaagtatcaaagataaagtttgcaatgcctttcctttcaatttctccacatcctttctctatataaaaagacatcttcgtcttgcttttggtttcagcttatatgcacttttataaataactatgatactctataaatactataacatggaaaatgttaaaatttattaagaaattattaagtttttattacaaaaaagttacaaaacctctgacatttttcatatcagaggttgtcattttttattttattttctatagaattttttagtgacaatatttcttctaattctttattgtatttatctattttcaacatggtacttctatataggcgtatatcttcttcgtttttttgtatacattttttaagggagttttttacagtttcaaaaagcgtatcataagtaatgtaattatgcatttcaaggtcggagattggaactgcgattaattcctccccttctattttatagtgataaaaaatgttgtcgggttttaaaactattttttcatctatgcttttatcgtaaattattataccttctcctatttcaaaagtttttccgctataacttctaagttttatattttcgactcctatgtatttttttattgccaaaagtatgttttttatttctgaaatggattttacaagcacttcttgcatttttttatttgccatctctttatctttttcactttttatgagttcttccatcaaagactttatttcatgtatatctcccataaaatatcacctctttcttaatattccacagaggaatcattttaaacgttgaatattttaaattattagagaaaaaatagacttgactattttttgaaatttgatagactattattaatagaaaattaatattgaaaaggagaagatattatgaacaaaatatctataataggttctggatttgtcggaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgaagatcagccgaagttatcaaaagtgtaatacaagagcttgatatataagaggggaaaccctctttttttgtatataaaaagtcacagcgtgaaaatataataattaaaataatgattttttagggtgtgatagtcgtgcagaaaataactcagcaggagattattttaagtgcctttgttgaagcacaaaatttagaaaagatactgttggataaagtaagagaatatgggaaagaatcagtagataatcaaataaaagcattgttaaagcaaattgaaataatgataaaaaatcataaagaagacataaaaaaggcacaaaagactatgcatattaattcccttgtcaaaaaaaatatgtctcaagagcctttagacatgcttcaagatttattaaaaaatttagttaatattcaagccttttataatgaaactgttgtgaatattactaatccttacgttagacagttgtttactcaaatgagggatgatgttatgagatttatttctattcttcaaatggagattgaaagtctggaatcgaaaccttctattccaaataacacagttttaaatacaccggagatgagttaatatgaaagtggctattattggtgctggtgtttcagggctggctgcggcaattacttttcaaaggtatggcattacaccagatatttttgaaaaaaagtgcaaaataggtgaattatttaaccatgttgcggggttattaaaagtgataaataggcctataaaggatccgcttcatcatcttaaaaatgtttatggaatagaagttaaaccaattaacaccattgacaaaatagtaatgaaggggccaactgtaacagcttctgttactgggagtaatcttgggtatatgattttaagaggacaggacgcaaactctcttgaaaatcaattgtataataagttagaaataccagttaatttcaatatagaagctgattataagaagttaaaaaataattacgattatgtctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 59 - DNA, 4680 bp, Artificial Sequence
  Synthetic polynucleotide
tattgattgacgatgaggtaatcaaaaaattagaagcatgtattgaccttgcacctttgcacaatcctgctaatattgagggaataaaagcttgtcggcagataatgccaggggtgccaatggtagcagtttttgatacggctttccatcaaacaatgccagattatgcgtatatttatcccattccttatgaatactacgaaaaatatagaataagaagatatggattccatgggacttctcataaatatgtatctttaagagctgctgaaatattaaagaggcctattgaagagttaaaaattattacttgccatttagggaatgggtctagtattgctgcggttaaaggcggtaagtcgatagatacaagtatgggatttactccattagaagggctggctatgggtacaaggtccggaaatgttgatccttcaattataactttcttaatggaaaaagaaggattgactgcagaacaggttatagatatacttaataagaaatcaggtgtatacggaatttcaggaataagtaatgactttagagatatagaaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 60 - DNA, 5663 bp, Artificial Sequence
  Synthetic polynucleotide
gttttataagaagctttgtaaattttattttctaagccttcagagacatcaaatatgtgattgagaggaatgtgatttctcaacagataaattaaaatattagaaaattcctctaaagatacgggaccttgttttttttcaaaagaactttgcaaaatttcttttgtaaagtccgggatatatttttctaagccttctagtcccttttcaaaaatatgctttcttatagaagaggcactggcaaattctccttttagctctaaagaggtgtacaaagagccttttctctttatagtaaaaggtgtaatagaactacctattttctttaaagatttaaggtattctattgccaatatgttattggatgtttgtaaaatcttttctatttcattattatttataactttttgtaatgctaattcccgtgcttttgcaaaggttatgccgctttttaaatattcttttaatgcttttctataataaattggctcttctaaaagtatttcagcaatttttgtgagttcgtttaaatcgcctttttcactcccaaaggaaaaacaatctactatttttaaagagtctaatagtttcaccgctccataagcgaaattttcagctgtagaggtagcataaactactggtaactcgattaccaaatctataccggcttttaatgccatttgagttcgtttccatttgtctacaattgctggttctcctctttgcacgaagtttccactcattactgctatagtataatcgcatttggttaattcttttgaagtttgcagatggtaaaggtggccattgtgaaaaggattatattcgacaataattcctaaaattcccatacaacttcttaccctttcaaaaaattttttaagatatacttattattttacataaaatatgataaaatgtaaaagggacatcgtgtatacaatattatagtgataaaattaaaaaaggaagggagattttaaatggcagtaatggatagtaaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcgtgactttagagatatagaaaatgcagcttttaaagaagggcataaaagggctatgttggcattaaaagttttcgcttatagggtgaaaaagacaataggttcttatacagctgctatgggtggggttgatgtaattgtgtttactgctggagttggagaaaatggaccagaaatgagagagtttattttagaggatctagagtttttaggctttaaactggacaaagagaagaataaggtaagaggaaaagaggaaattatatctacagaagattcaaaagttaaagttatggttattcctacaaatgaagaatatatgattgctaaagatactgaaaaattggtaaaaggtttaaagtagataatcttgacaacgggttgtggggttagtataataggtgatgtcaattattttaaggtgtgagaagaaaaatgaaaatcgatctattaaaaatcaaaggacagcttggccgcagcataaatatagactatgtagaggacatagagaacattgaatttaaaggggaagaatacaaactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 61 - DNA, 5395 bp, Artificial Sequence
  Synthetic polynucleotide
aattcgagctcggtacccggggatcctctagagtcgacctgcaggcatgcaagggaagatatgcctgcttgacattattgtccgtcatttttcggtttatcctggtaaaaaaagttttaatcctctcaaggctttcttcgtgtaaaacaagctttttccccaaaacttccgcaacagtctcctttgtaaggtcatcctgcgtggggccgagtcctccggtcataataacaaggtcgcacctttccaaagctgcaagaagacattttttcagccgaacggaattgtcccccaccacactgtgataatacacattcacaccaatgtcattgagccttttggatatatactgggcattggtatttgctatctgccccattaaaagctcggttccaaccgctaatatctccgcattcatattgaaagaccccttaaatttaaactttttgtaacttattatatcaattagtgttataaaataaaagggaaaaagaattaaaatcaaaggtttcaagagcagccgtatcacccgtaaaagtttcagccgattcaacctttttacacataaaactttcaaaaattgatgacttacaattatcaagtaggatataatattactaatgctaaacagttattgataaaggaggaaggaatatgaacaataacaaagtaattaaaaaagtaacccggactttctgagaagctgaatttcttcatcgttgaaaggcacgttcaatatttcctcaataccgtttacacccacgattgtcggaacacttaagcatacatcgctaagtccgtactgtccttccaaaaggcttgaaacggtaaggatggagttttcatttcttacaatggcttcaacgattcttcttacggcaagggctacggcataataggttgcacctttgttcctgatgatttcataagctgcatttttaacactttcatatattttattccgggaaatctgctcctcgcactgatggcattcgtcacagtagcgatccatgggaattcccgcaatatttgcaagactccaggccgcaacttcggtgtcaccgtgttcgccaataatataagcatgtacatttcgtgcatccacttttacatgttcgcttaaaagataacggaacctggctgtgtccaaaaccgttccggaacctattactttgtttttcgggaatccggatagtttgtaagttacataggttaaaatatccaccggatttgtgactaccagaagaatacaatcgttgttgtactttacaatttcatttatgatatttttgaatacttccgtgtttcttttaacaagatctattctcgtttcgccttctttttggttggcaccggcggtaatgattactatgtcggatccggcacagtctttgtagtcaccacgataaatttcaacgggccttacaaaaggcatgccgtgatttaagtccatgacttctccgtcggctttttttgcatttatgtctatcagtacaatttcagatataagtccgctgagcatcaatgtataagctgtggtggaacctacaaagcctgcaccaactacgaaacactctaaaagaaataataaaaacactagatatatgaaagttctccttttcttttatgaaaaggagaactttcattattgataaatatataaactagtatataattttaatataaaacctattttacataatggaaattatctatcgggggaggaaatatgaacaattcagtggaaattttaaataaaatcgtgtcaaatattgaaaaagtcattgttggaaaaaagaaagctatcgagttgatattaatatcacttatttgcgatggacatgttttgattgaagatgtccccggtgtcggaaaaaccagtacttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtc

SEQ ID NO: 62 - DNA, 45 bp, Artificial Sequence
  Synthetic primer
atttacctggctgggaatactgagacatatgtcattgaggccgta

SEQ ID NO: 63 - DNA, 53 bp, Artificial Sequence
  Synthetic primer
aaaaaagcttataattatccttaatttcctactacgtgcgcccagatagggtg

SEQ ID NO: 64 - DNA, 60 bp, Artificial Sequence
  Synthetic primer
cagattgtacaaatgtggtgataacagataagtctactactgtaacttacctttctttgt

SEQ ID NO: 65 - DNA, 49 bp, Artificial Sequence
  Synthetic primer
tgaacgcaagtttctaatttcggttgaaatccgatagaggaaagtgtct

SEQ ID NO: 66 - DNA, 45 bp, Artificial Sequence
  Synthetic primer
ttaaatgttgataaggaagctcttttcaatgaagttaaggtagca

SEQ ID NO: 67 - DNA, 53 bp, Artificial Sequence
  Synthetic primer
aaaaaagcttataattatccttagctctcttcaatgtgcgcccagatagggtg

SEQ ID NO: 68 - DNA, 60 bp, Artificial Sequence
  Synthetic primer
cagattgtacaaatgtggtgataacagataagtcttcaatgataacttacctttctttgt

SEQ ID NO: 69 - DNA, 49 bp, Artificial Sequence
  Synthetic primer
tgaacgcaagtttctaatttcgattagagctcgatagaggaaagtgtct

SEQ ID NO: 70 - DNA, 1509 bp, Caldiscellulosiruptor kristjanssonii
atgtattttattggaattgacgttggaacatctggaacaaagacaatcctgattgactcaaaaggtaagattctggcttctgcaacctttgaatatcctctttatcagcctcagattggctgggctgagcaaaatcccgaagactggtgggatgcaagcgtaaaaggaattaaagctgtgcttgaaaagtcaaaagtagaccccaaggaagttaaggctgtgggacttaccgggcagatgcacgggcttgtgctgcttgacaaaaactacaacgttataagaccatcaatcatctggtgtgaccagagaacggcaaaagaatgtgatgaaataacagaaaaggttggcaaggaaaagcttgtggagattacagcaaaccctgcactgacaggttttacagcgtccaagattctgtgggtgagaaacaacgagccccaaaactatgagaaggtctacaaaattttgcttcccaaagactatataaggtttaaacttacaggcgagtttgcaacagatgtgtcggacgcctcgggtatgcagcttttggacattaaaaacaggtgctggtctgatgaggtacttgaaaagcttgagatagacaaagggcttcttggaaaagtctatgagtcgccagaggtaacgggaaaagttagcgggcaagcaagcgaacttacaggtctttgtgaaggtacgcttgttgttgcaggtggaggagaccaggcagcaggtgcagttggaaatggcatagtaaagacgggtgtgatttcatctacaattggttcgtctggcgttgtttttgcccatcttgacgagtttaagattgacccacagggaagggttcacacattttgtcatgcagtgccgggaaaatggcatgtgatgggtgtaacacaaggtgccggactttctctcaagtggtttagagacaactttgcacacatcgaaaaggctgcgtttgagtttattgacaaagacccatacattttgatggaccaggaggcagaacttgcaaacccaggcgcagacggacttgttttcctgccatatttgatgggggaaagaacgcccattttggacccatacgccaaaggaatattctttggaataacagcaaagcatacacgaagagagttcattagagctgtcatggaaggtgttgtattttcacttaaaaactgtcttgatattttgtatgagatgggcatcgaggtgaaggaggtaagagtttcaggcggtggtgcaaagagcaagctctggagacagatgcaggcagacatatttgagatggatgtatggacactgaattccaaagaaggacctgcgtttggtgcagctatcctggcagcagttggtgcaggagaatatcagaaggttgaagaagcctgtgatactatgattcaaaaggtagataactgcagcccaaatgaaaaactatttgaaatatatagaaaaacttataaactttacaacagtatatatccaagagttaaggacttattcaacatgtaa

SEQ ID NO: 71 - DNA, 1317 bp, Caldiscellulosiruptor kristjanssonii
atgaaatacttcaaagacattccagaagtaaaatatgaaggaccacagtcagacaatccatttgctttcaagtattacaatcctgacgaggttattgatggcaagcctttaaaagaccaccttcgttttgcaattgcttactggcacacgttctgtgcaaccggtagcgacccttttggacaacctacaattaatcgtccatgggacaggttctcaaacccaatggacaatgcaaaagcaagagttgaagctgcatttgaattttttgaaaagctaaatgttccatttttctgcttccacgacagagacatcgcacctgaaggagaaaatttaagagaaacaaacaagaatttggatgagatagtctctatgataaaagaatatttaaagacaagcaaaacaagagttttgtggggaacagcaaacctattttcacatccgcgatatgttcatggtgctgcaacatcctgcaatgccgatgtttttgcgtatgcagcagcgcaggtgaaaaaggcgttagaggttacaaaagagcttggcggcgaaaactatgtgttctggggcggaagagaaggttatgagacacttttgaacaccgacatggagcttgagcttgacaacttggcaagatttttgcacatggcagttgactatgcaaaagagatagggtttgacggtcagtttttgattgaaccaaagccaaaagaaccaactaagcatcagtacgattttgatgccgctcatgtttatggatttttgaaaaaatatgaccttgacaagtacttcaagctcaacatagaggtaaaccatgcgactttggcaggacatgatttccaccatgagttgagatttgcacgaataaacaacatgcttggctcaattgatgctaacatgggcgacttgcttttgggctgggatacagaccagttcccaacagatgtaagacttaccacgcttgctatgtatgaggttattaaagctggtggctttgacaaaggcgggctcaactttgacgcaaaggtaagaagaggttcttttgagcttgaagacttggtcattggtcacattgcagggatggacgcttttgcaaaaggatttaagatagcatataagcttgtcaaagacggtgtatttgataagtttattgaagaaaggtatagaagctacaaagaaggaattggagctaagattgtaagtggtcaggcagattttaagacgttagaagaatatgctttgaatctttcaaagatagaaaacaaatctggcaagcaagagcttcttgagatgattttgaacaaatatatgttcagtgaataa

SEQ ID NO: 72 - DNA, 1536 bp, Clostridium straminisolvens
ttgtcatatttactgggagtagatataggtacatcaggcacgaaaactgttttatatgatgaactgggaaataccgtagcaagcagccttgaggaatatccattgtaccagccccatattgggtgggcagagcaggaaccggaagactggtggagggcaacatgcctatctatcaaacatgttatttccaaaagaggaattgatgcttcctctattaagggaatcggactttcaggacagatgcacggggctgttcttctggacaaagacggcaaagtgctaagaaaagcaattatatggtgtgaccagagaagttttgccgagtgcgagcagattacttcaattatagggaaggaaaggctcgttgagataactgccaaccctgcactgacgggatttacagcatcaaaggttatgtgggttaaaaataatgaacctgaaatttttgagaagatttataagatacttctccctaaagactatataagatataaattaacgggagaatttgctacagaggtatctgatgccagtggaatgcagtttatggatataccggggagaaaatggagcgacgaagtcataagtaaactcggacttgataaaagcatgctgggagaactctatgagtctcaggaagttagcgggaaagtgaataagtatgctgcttcattaaccggacttaaggaaggaactcctgtcgtgggtggagcaggagaccaggcagcaggagctgtcggtaatggaattgtgagacccggggtggtttcatccactataggaacttcaggagtagtatttgcattctctgaaaaggttactattgatccaaagggtagagttcatactttttgtcatgcggtaccaaatacctggcacattatgggggttacacaaggggccgggctgtctcttaagtggttccgtgacaatttctgtatagaagaaaagagaactgcagagctaatgaaaatagacccgtacataattatggataaagaagctgaaaaagtggctccgggctgtaacggtttaatctatttaccttatctgatgggagaaagaacgccacatcttgaccctaatgccaagggtgtctttttcggattaacagcaaagcatgaaaaacaggatatcttaaggtcgattatggaaggtgttgtatatagccttagagattgccttgaaattattgaggaaatgggtgttaacgtttctgaagtaagagcttccggtggaggcggtaaaagtgaattgtggagaaaaatgcaggcggatatattcggcactgatattacaaccgtaaagtcaagtgagggaccggcacttggggtagcacttcttgccggagtaggaacgggtgtgtacaacaacattaatgaagcatgtgaagcagtaataaaagaaaatacccggcaggcttcggacccggagctatatgtaaaatacacgaagttttatgatatttataaacgtctgtataactctttgaaaaaggaatttgcagacctttcggctatgctgcaaagtttatag

SEQ ID NO: 73 - DNA, 1320 bp, Clostridium straminisolvens
atggcagagtattttaaaaatgtaccgaaaatcaaatatgaaggaaaggattcggacaatcctttagcgtttaagtactataatcccgatgaggtcattggcggtaaaacaatgaaagagcatctaaggtttgctgttgcatattggcatacatatcagggaacgggtgcagacccatttgggccgggtactgctgtaagaccgtgggatgacatatcggacccaatggatcttgcaaaggccaaagtggccgcaaatttcgagctgtgtgaaaaattgggagtaccatttttctgcttccatgacagagatattgcgcctgaagcttcaactttaagagagaccaataaaagacttgatgagattgttgcactgataaaggactatatgaaaacaagtagtgtaaaactactctggggtacaacaaatgcttttagccacccaaggtttgtccatggtgcatctacttctccgaatgcagatgtatttgcatatgcagcagctcaggttaaaaaggctatggaaattaccctggaacttggcggtcagaactatgtgttctggggtggaagagaaggctatgaaaccttacttaatactgatatgaaattggagcttgacaatatggcaaggttccttggaatggcagttgactatgcaaaagagattggttttaaagggcagctcttgattgaacctaagccaaaagaaccgacaaagcaccagtatgactttgatacagctacagttatcggtttcttaaggacttatggtcttgagaattacttcaaaatgaatattgaagcaaatcacgctacacttgcagctcatactttccagcatgaacttagggtttcaagaattaacggtgtgctaggaagtatcgatgcaaaccagggtgatcttcttttaggatgggacactgaccaattcccgacaaatatctacgatactacccttgctatgtatgaagtaattaaggcaggcggatttacaacgggaggtctgaattttgattctaaagtcagaagaggatcatttgagcctgtggacctgttctatgcacatattgcaggtatggacgcttttgcaaaaggatttaaaatagcatataaaatggtttccgacggtaagtttgacaaatttattgatgaaagatatgaaagctataagagcggtattggaaaagatattgtagatggaaaagtagggtttaaagagcttgaaaaatatgctttagagcttgatggtatcaagaatgtgtcgggaagacaggaagttctcgaagctatgttaaacaaatatattcttgaggactag

SEQ ID NO: 74 - DNA, 8909 bp, Artificial Sequence
  Synthetic polynucleotide
catgagacaataaccctgataaatgcttcaataatattgaaaaaggaagagtatccagtattctgacatgggtgtatcaataacccatgcgtttccgtattgtatcggaatggtttcggacagggcggtgggaatagacatggaaaagatttttttgcccgaggatgcattgataaagtatttcttttccgaaagagaggaaaagattctaaagagttttggaaatactgatgaatattgtgtgcagagtacaattctatggacaagaaaagaggctttgtcaaaactttttcgtctgggaatgaggatggattttaaaaagctggatactttggaggacgaggtggtttttcaggaaacaaacagggcgcgtctgttttcttttatatgcaataattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatgaataaatattttgagaacgtatctaaaataaaatatgaaggaccaaaatcaaacaatccttattcttttaaattttacaatccagaagaagtaatcgatggcaagacgatggaggagcatctacgcttttctatagcttactggcacacttttactgctgatggaacagatcaatttggcaaagctaccatgcaaagaccatggaaccactacacagatcctatggacatagcaaaggcaagggtagaagcagcatttgagttttttgataagataaatgcacctttcttctgcttccatgacagggatattgcacctgaaggagacactcttagagagacaaacaaaaacttagatacaatagttgccatgataaaggattacttgaagaccagcaagacgaaagttttgtggggcaccgcaaatcttttctccaatccgagatttgtacatggtgcatcaacatcctgcaatgctgatgttttcgcatattctgcagctcaagttaaaaaagctcttgagattactaaggagcttggcggcgaaaactacgtattctggggtggcagagaaggatatgaaacacttctcaatacagacatggagtttgagcttgacaactttgcaagatttttgcacatggctgttgactacgcgaaggaaatcggctttgaaggccagttcttgattgagccgaagccaaaggagcctacgaaacaccaatacgactttgacgtggcaaatgtattggcattcttgagaaaatacggccttgacaaatatttcaaagtgaatatcgaggcaaaccatgcgacattggcattccacgacttccaacatgagctaagatacgccagaataaacggtgtattaggatcaattgacgcaaatacaggcgatatgcttttaggatgggatacagaccagttccctacagatatacgcatgacaacgcttgctatgtatgaagtcataaagatgggtggatttgacaaaggcggccttaacttcgatgcaaaagtaagacgtgcttcatttgaaccagaagatcttttcttaggtcatatagccggaatggatgcctttgcaaaaggcttcaaagttgcttacaaacttgtgaaagatggcgtatttgacaagttcatcgaggaaagatacgcaagctacaaagacggcattggcgctgacattgtaagcgggaaagctgacttcaagagccttgaaaagtacgcattagagcacagccagattgtcaacaaatcaggcaggcaagagctgttagaatcaatcctaaatcagtatttgtttgcagaataatgaaacatgagggcggcttcatgcttcattaaagctgccctcaacaaaaatcatggaggtaaatgtatgtattttttagggatagatttagggacatcatcagttaagataatactgatgaatgaaagcggcaatgtggtatcaagcgtttcaaaagaatatcctgtgtactatccagagccaggctgggctgagcaaaatccagaagattggtggaatggcacaagggatggaataagagagattattgcgaaaagcggcgtaaatggcgatgaaataaagggtgttggcttaagcgggcagatgcatggactggtgcttttagacaaagacaataacgttttaacgccagccatactttggtgtgaccagaggacacaggaagaatgcgactacatcacagagaaaataggaaaagaaggccttttgaagtacacagggaataaagcattgacaggttttactgcaccaaagatattatgggtaaagaagcaccttaaagacgtatatgaaagaatcgctcatatccttttgccaaaagattatataaggtttaaattgacaggtgagtacgctacagaagtttcagatgcatcaggtacacttcttttcgatgtggaaaatagaagatggtcaaaggaaatgatagacatatttgaaataccggaaaaagcccttcctaagtgctacgaatcaacagatgtcacagggtatgtcaccaaagaggcagcagatttgacagggcttcatgaagggactattgtcgtaggcggtggtggtgaccaagccagcggcgctgtaggcactggcacggtgaaaagcggcatagtgtccatcgcattaggaacttcaggcgtcgtatttgcatcacaggacaagtacgcagcagatgatgagcttaggcttcactcattctgccatgcaaacggcaaatggcatgtgatgggtgtcatgctttcggctgcatcatgtcttaaatggtgggtagatgatgtaaataattacaagaccgatgttatgacatttgatggactcttagaagaagcagagaaggtgaagccaggcagtgatggattgatattcttgccatacctgatgggtgaaaggaccccttacagcgatccttatgcgagaggcagctttgtaggtttaacaattacacacaatagaagccacatgacaagatctatattagaaggcgtcgcatttggacttagggattcgctggagcttataaaggctttaaatatacctgtaaatgaagccagggtaagtggtggtggtgctaaaagcaggctttggaggcaaatacttgccgatgtattcaatgtaaggatagacatgataaatgctacagaaggaccttcatttggtgcagcaataatggcgtctgtgggatatggcctttacaaaaatgtagatgatgcatgcaatagtttaataaaagttacagacagcgtatatccaatcaaagaaaacgtcgaaaagtacaacaaactgtatccaatctacgtgagcttgtattcaaggcttaaaggcgcctttgaagaaattgggaagttggatttgtaaaataaattcatttggaaataaatttatgacagtacaagggacattgattaacaaagcttcaggttaataatagtaaagttaatatttgctatgaaatgaaagcataataatctgttccttgtactttgctttatcatgtttatttaagatactaattaataaaagtcaatttagccaataataaaatcctatatatagtaaatatttacaataaaatcactacaaaataaaaaactttatttaatctcttaaaaatatctacataagggggtgttagatgaaaaaggccgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcggggaaatgtgcgcggaacccctatttgtttatttttctaaatacattcaaatatgtatccgct

SEQ ID NO: 75 - DNA, 6972 bp, Artificial Sequence
  Synthetic polynucleotide
gacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtcaattcgagctcggtacccggggatccttaagaagactaataaaaagtttctaaaagcatgaaatatcctgtattttggagttacttgccgttatttatggctaaagccgaaaaaaagataaggagggtgttgtatacaaaaaacattttgtatataattagagttgcttggaacccagtaaaatagctgtttatagtgagtaggtattattacctgatgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgccgctgaagcgatttacaccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggcacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcattcgcttccccagcgtccatctgaaagactatctgacccgtttggctcagaaagtttccaagaaaaccggtgcattggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctagattctgttaaaaccggacattgaagaaggtgttgcgcagcgtttaataaaaacatctgtttatcgaagtttaggaataggaaaattaaaaaaaacaagacgggagtgagtttttgaaatggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgctttgaaagcagcttccatgatcgctaagaatctgaagaccgcttgcgacaacggtaaggatatgccggctcgtgaagctatggcttatgcccaattcctcgctggtatggccttcaacaacgcttcgcttggttatgtccatgctatggctcaccagttgggcggttactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaacctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaaaagatgcgttataattttacaagcctgtttttttaggaaacgggcttataaaatttttttatttttttgccgggttttttcttgtattaatatgtggaatatgttaataatattaagaagaaattccgaatttaactaaacaaaattatttttgttatttaagccaatctgtcatataattcttgacatgagggttattagttagtataatagtccttgtcggttttaagagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtct

SEQ ID NO: 76 - DNA, 6936 bp, Artificial Sequence
  Synthetic polynucleotide
gacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtcaattcgagctcggtacccggggatccttaagaagactaataaaaagtttctaaaagcatgaaatatcctgtattttggagttacttgccgttatttatggctaaagccgaaaaaaagataaggagggtgttgtatacaaaaaacattttgtatataattagagttgcttggaacccagtaaaatagctgtttatagtgagtaggtattattacctgatgtataccgttggtatgtacttggcagaacgcctagcccagatcggcctgaaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaaattctgttaaaaccggacattgaagaaggtgttgcgcagcgtttaataaaaacatctgtttatcgaagtttaggaataggaaaattaaaaaaaacaagacgggagtgagtttttgaaatggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgctttgaaagcagcttccatgatcgctaagaatctgaagaccgcttgcgacaacggtaaggatatgccggctcgtgaagctatggcttatgcccaattcctcgctggtatggccttcaacaacgcttcgcttggttatgtccatgctatggctcaccagttgggcggttactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaacctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaaaagatgcgttataattttacaagcctgtttttttaggaaacgggcttataaaatttttttatttttttgccgggttttttcttgtattaatatgtggaatatgttaataatattaagaagaaattccgaatttaactaaacaaaattatttttgttatttaagccaatctgtcatataattcttgacatgagggttattagttagtataatagtccttgtcggttttaagagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtct

SEQ ID NO: 77 - DNA, 1200 bp, Clostridium thermocellum
atgaatattttggttattaataccggaagctcatcactaaagtatcagctgattgacatgacaaacgagtctgtgcttgcaaaaggtgtgtgtgacagaattggtcttgaacattcctttttaaagcatacaaagaccggaggggaaaccgtagttatagaaaaagacctgtacaatcacaagcttgccatacaggaggtaatttcggctcttacggatgaaaaaatcggagtcataaaaagcatgtcggaaatttctgccgtcggtcatcgtattgttcacggcggagagaagtttaaggaatctgccataattgatgaagatgtaatgaaagcaatcagggattgtgttgaactggctccgctccacaatccgtcaaatataatcggaattgaagcctgtaaacagatactgcccgatgtgccgatggttgctgtgtttgacacagcttttcatcagacaatgccaaggcatgcatatatttatgccctcccttatgagatatatgagaagtataaattgagaaaatacggattccacggaacttcccacaaatatgtggcccacagggcggctcagatgctgggcaaacctattgagagcctgaagctgataacctgccatcttggaaacggagcaagtatttgtgcggtaaaaggcggaaaatccgttgacacctcaatgggatttactcctctgcaggggttgtgcatgggtaccagaagcggcaatgttgaccctgcggttataacttatttgatggaaaaggaaaaaatgaatattaacgatataaacaatttccttaacaagaaatcaggtgtgcttggaatttcaggtgtaagcagtgatttcagagatgttcaggatgccgcagaaaagggagatgacagggcgcagctggcattggatattttctgctatggtgttaggaaatatattggaaaatatattgcagtgctgaacggcgttgatgcggtggtattcactgcaggtatcggcgaaaacaatgcttatataagaagagaagttttgaaggatatggactttttcggaattaaaatagatttggataaaaatgaagtgaaaggcaaagaagcggatatcagtgctcccgatgcgaaagtaaagactttggttatcccgacaaatgaggagcttgagattgcaagggagactttaagacttgtaaaaaacttataa

SEQ ID NO: 78 - DNA, 1077 bp, Clostridium thermocellum
gtgataatatatagttataagtattacaaatacagtttttatgataacagttttgggattatgaaaggagaagaatttatgagttttttggaacaaataattgaaagggcgaaatcagacgtaaaaaccatagttttgccggaaagtacggatctgagggttattaaagccgcatccatgataatgaaaaagggaattgcaaaggttgtactgataggcaatgaaaaggagattaagagtctggcgggggatattgatcttgaaggagtgatgatagaggattccttaaattccgaaaaattggaggattatgcaaatacactgtatgagcttagaaaatcgaagggtatgactatagaagccgcaagggaaacgatcaaagaccctctttattatggagttatgatggtaaaaaaaggtgaagcggatggtatggtggcgggtgctgtcaattccactgcaaatactttgagaccggctttgcagatattaaagacggccccggggacaaaactcgtatcatccttttttgttatggttgtacccaactgtgaatatggtcataacggaacctttgtatatgccgattgcggcttggtggaaaatccggatgcagaccagctttctgaaattgcaatatctgcatccaaatcttttgagatgctggttggagcaaaacctcaggtggcaatgctttcttattcttcttacggcagtgccaaaagtgagctgaccgaaaaggtaatcaaggcaacacagcttgcaaaggaaaaagctccccaccttgcaattgacggagaacttcaggtggatgccgccattgttccggaagtggcaaaatcgaaggcaaagggaagcagtgttgcaggaaaggccaatgttcttattttcccggatcttgatgccggaaatattgcatacaagcttacacagagattggcaaaagctgaagcttacggcccgataacacaaggtttggcaagaccggtaaatgatctgtcacgaggctgcagtgccgaggatatagtcggggttgcggcaattactgcggttcaggctcaatatgtcaaggcataa

SEQ ID NO: 79 - DNA, 4087 bp, Artificial Sequence
  Synthetic polynucleotide
aattcgagctcggtacccggggatcctctagagtcgacctgcaggcatgcaagcttggtctgcatccggattttccaccaagccgcaatcggcatatacaaaggttccgttatgaccatattcacagttgggtacaaccataacaaaaaaggatgatacgagttttgtccccggggccgtctttaatatctgcaaagccggtctcaaagtatttgcagtggaattgacagcacccgccaccataccatccgcttcacctttttttaccatcataactccataataaagagggtctttgatcgtttcccttgcggcttctatagtcatacccttcgattttctaagctcatacagtgtatttgcataatcctccaatttttcggaatttaaggaatcctctatcatcactccttcaagatcaatatcccccgccagactcttaatctccttttcattgcctatcagtacaacctttgcaattccctttttcattatcatggatgcggctttaataaccctcagatccgtactttccggcaaaactatggtttttacgtctgatttcgcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtc

SEQ ID NO: 80 - DNA, 4200 bp, Artificial Sequence
  Synthetic polynucleotide
gacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtcaattcgagctcatcccattgaggtgtcaacggattttccgccttttaccgcacaaatacttgctccgtttccaagatggcaggttatcagcttcaggctctcaataggtttgcccagcatctgagccgccctgtgggccacatatttgtgggaagttccgtggaatccgtattttctcaatttatacttctcatatatctcataagggagggcataaatatatgcatgccttggcattgtctgatgaaaagctgtgtcaaacacagcaaccatcggcacatcgggcagtatctgtttacaggcttcaattccgattatatttgacggattgtggagcggagccagttcaacacaatccctgattgctttcattacatcttcatcaattatggcagattccttaaacttctctccgccgtgaacaatacgatgaccgacggcagaaatttccgacatgctttttatgactccgattttttcatccgtaagagccgaaattacctcctgtatggcaagcttgtgattgtacaggtctttttctataactacggtttcccctccggtctttgtatgctttaaaaaggaatgttcaagaccaattctgtcacacacaccttttgcaagcacagactcgtttgtcatgggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtct

SEQ ID NO: 81 - DNA, 8706 bp, Artificial Sequence
  Synthetic polynucleotide
gattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctccgaattcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaagatctgttgtgtggaattgtgagcggataacaatttcacacaggaaacagctatgaccatgattacgccaagcttgcatgccctgcaggtcgactctagagtgtcatctcccttttctgcggcatcctgaacatctctgaaatcactgcttacacctgaaattccaagcacacctgatttcttgttaaggaaattgtttatatcgttaatattcattttttccttttccatcaaataagttataaccgcagggtcaacattgccgcttctggtacccatgcacaacccctgcagaggagtaaatcccattgaggtgtcaacggattttccgccttttaccgcacaaatacttgctccgtttccaagatggcaggttatcagcttcaggctctcaataggtttgcccagcatctgagccgccctgtgggccacatatttgtgggaagttccgtggaatccgtattttctcaatttatacttctcatatatctcataagggagggcataaatatatgcatgccttggcattgtctgatgaaaagctgtgtcaaacacagcaaccatcggcacatcgggcagtatctgtttacaggcttcaattccgattatatttgacggattgtggagcggagccagttcaacacaatccctgattgctttcattacatcttcatcaattatggcagattccttaaacttctctccgccgtgaacaatacgatgaccgacggcagaaatttccgacatgctttttatgactccgattttttcatccgtaagagccgaaattacctcctgtatggcaagcttgtgattgtacaggtctttttctataactacggtttcccctccggtctttgtatgctttaaaaaggaatgttcaagaccaattctgtcacacacaccttttgcaagcacagactcgtttgtcatgtcaatcagctgatactttagtgatgagcttccggtattaataaccaaaatattcatttcaaaaactcactcccgtcttgttttttttaattttcctattcctaaacttcgataaacagatgtttttattaaacgctgcgcaacaccttcttcaatgtccggttttaacagaatttatgccttgacatattgagcctgaaccggtagttcaacaaacgggattgacttttaaaaaaggattgattctaatgaagaaagcagacaagtaagcctcctgtatttcttttccgaaagagaggaaaagattctaaagagttttggaaatactgatgaatattgtgtgcagagtacaattctatggacaagaaaagaggctttgtcaaaactttttcgtctgggaatgaggatggattttaaaaagctggatactttggaggacgaggtggtttttcaggaaacaaacagggcgcgtctgttttcttttatatgcaataattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatgatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctgcggctttaataaccctcagatccgtactttccggcaaaactatggtttttacgtctgatttcgccctttcaattatttgttccaaaaaactcataaattcttctcctttcataatcccaaaactgttatcataaaaactgtatttgtaatacttataactatatattatcaccaggtaataatacctactcactataaacagctattttactgggttccaagcaactctaattatatacaaaatgttttttgtatacaacaccctccttatctttttttcggctttagccataaataacggcaagtaactccaaaatacaggatatttcatgcttttagaaactttttattagtcttcttaattattcagattttgtggcaattaaactttgcagctcctccaaatagttgtccagctcctcttctttaagattgctgagatatgacaatctgtaatttttagcctttttggccatctctagcgcactctccgtcattcccaaatctttcaaaacacagctatagttatagtatgcctcaggaaaatttggattaagacctataagctttcgatatatttcctcggcctttttaatgttaccgattttgtaataggtatagcctaaattgtcaagtatgacaggatcagagctgttatactcatacgcctccttattgaaatttaaagctttgttcatatccccgtccagaataagaagatatccgagacttccgtatatggttgtattcttatagtttgaaaacacatcctccagcatttttatgccatctttcaactgtccctttttccaaagcaccaatgcatagttggactttgcctgcccgggtaccgcggccgcgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcggcggccgcgaattgacagatcgctgagataggtgcctcactg

SEQ ID NO: 82 - DNA, 8777 bp, Artificial Sequence
  Synthetic polynucleotide
gattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctccgaattcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaagatctgttgtgtggaattgtgagcggataacaatttcacacaggaaacagctatgaccatgattacgccaagcttgcatgccctgcaggtcgactctagagaggcaccttgagcattttgccctgtaatccgcccagagttcaccgtcaagttttataatccctccggcatttaccaaagtgcccttaaaagttaccggtttcgcaaagtcaaaatcctcagctataaaatcattaattttaattgactcactaaagtccagtctcagcgaagctccactatgtttcaaaatgtcagatatatcaattttcatcaaagtcacctcttaaaaccgacaaggactattatactaactaataaccctcatgtcaagaattatatgacagattggcttaaataacaaaaataattttgtttagttaaattcggaatttcttcttaatattattaacatattccacatattaatacaagaaaaaacccggcaaaaaaataaaaaaattttataagcccgtttcctaaaaaaacaggcttgtaaaattataacgcatcttttataagttttttacaagtcttaaagtctcccttgcaatctcaagctcctcatttgtcgggataaccaaagtctttactttcgcatcgggagcactgatatccgcttctttgcctttcacttcatttttatccaaatctattttaattccgaaaaagtccatatccttcaaaacttctcttcttatataagcattgttttcgccgatacctgcagtgaataccaccgcatcaacgccgttcagcactgcaatatattttccaatatatttcctaacaccatagcagaaaatatccaatgccagctgcgccctgtcatctcccttttctgcggcatcctgaacatctctgaaatcactgcttacacctgaaattccaagcacacctgatttcttgttaaggaaattgtttatatcgttaatattcattttttccttttccatcaaataagttataaccgcagggtcaacattgccgcttctggtacccatgcacaacccctgcagaggagtaaatcccagtagttcaacaaacgggattgacttttaaaaaaggattgattctaatgaagaaagcagacaagtaagcctcctgtatttcttttccgaaagagaggaaaagattctaaagagttttggaaatactgatgaatattgtgtgcagagtacaattctatggacaagaaaagaggctttgtcaaaactttttcgtctgggaatgaggatggattttaaaaagctggatactttggaggacgaggtggtttttcaggaaacaaacagggcgcgtctgttttcttttatatgcaataattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatgatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctctttagtgatgagcttccggtattaataaccaaaatattcatttcaaaaactcactcccgtcttgttttttttaattttcctattcctaaacttcgataaacagatgtttttattaaacgctgcgcaacaccttcttcaatgtccggttttaacagaatttatgccttgacatattgagcctgaaccgcagtaattgccgcaaccccgactatatcctcggcactgcagcctcgtgacagatcatttaccggtcttgccaaaccttgtgttatcgggccgtaagcttcagcttttgccaatctctgtgtaagcttgtatgcaatatttccggcatcaagatccgggaaaataagaacattggcctttcctgcaacactgcttccctttgccttcgattttgccacttccggaacaatggcggcatccacctgaagttctccgtcaattgcaaggtggggagctttttcctttgcaagctgtgttgccttgattaccttttcggtcagctcacttttggcactgccgtaagaagaataagaaagcattgccacctgaggttttgctccaaccagcatctcaaaagatttggatgcagatattgcaatttcagaaagctggtctgcatccggattttccaccaagccgcaatcggcatatacaaaggttccgttatgaccatattcacagttgggtacaaccataacaaaaaaggatgatacgagttttgtccccggggccgtctttaatatctgcaaagccggtctcaaagtatttgcagtggaattgacagcacccgccaccataccatccgcttcacctttttttaccatcataactccataataaagagggtctttgatcgtttcccttgcggcttctatagtcatacccttcgattttctaagctcatacagtgtatttgcataatcctccaatttttcggaatttaaggaatcctctatcatcactccttcaagcccgggtaccgcggccgcgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcggcggccgcgaattgacagatcgctgagataggtgcctcactg

SEQ ID NO: 83 - DNA, 8642 bp, Artificial Sequence
  Synthetic polynucleotide
gattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctccgaattcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaagatctgttgtgtggaattgtgagcggataacaatttcacacaggaaacagctatgaccatgattacgccaagcttgcatgccctgcaggtcgactctagagaggcaccttgagcattttgccctgtaatccgcccagagttcaccgtcaagttttataatccctccggcatttaccaaagtgcccttaaaagttaccggtttcgcaaagtcaaaatcctcagctataaaatcattaattttaattgactcactaaagtccagtctcagcgaagctccactatgtttcaaaatgtcagatatatcaattttcatcaaagtcacctcttaaaaccgacaaggactattatactaactaataaccctcatgtcaagaattatatgacagattggcttaaataacaaaaataattttgtttagttaaattcggaatttcttcttaatattattaacatattccacatattaatacaagaaaaaacccggcaaaaaaataaaaaaattttataagcccgtttcctaaaaaaacaggcttgtaaaattataacgcatcttttataagttttttacaagtcttaaagtctcccttgcaatctcaagctcctcatttgtcgggataaccaaagtctttactttcgcatcgggagcactgatatccgcttctttgcctttcacttcatttttatccaaatctattttaattccgaaaaagtccatatccttcaaaacttctcttcttatataagcattgttttcgccgatacctgcagtgaataccaccgcatcaacgccgttcagcactgcaatatattttccaatatatttcctaacaccatagcagaaaatatccaatgccagctgcgccctgtcatctcccttttctgcggcatcctgaacatctctgaaatcactgcttacacctgaaattccaagcacacctgatttcttgttaaggaaattgtttatatcgttaatattcattttttccttttccatcaaataagttataaccgcagggtcaacattgccgcttctggtacccatgcacaacccctgcagaggagtaaatcccagtagttcaacaaacgggattgacttttaaaaaaggattgattctaatgaagaaagcagacaagtaagcctcctgtatttcttttccgaaagagaggaaaagattctaaagagttttggaaatactgatgaatattgtgtgcagagtacaattctatggacaagaaaagaggctttgtcaaaactttttcgtctgggaatgaggatggattttaaaaagctggatactttggaggacgaggtggtttttcaggaaacaaacagggcgcgtctgttttcttttatatgcaataattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatgatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggctgcggctttaataaccctcagatccgtactttccggcaaaactatggtttttacgtctgatttcgccctttcaattatttgttccaaaaaactcataaattcttctcctttcataatcccaaaactgttatcataaaaactgtatttgtaatacttataactatatattatcaccaggtaataatacctactcactataaacagctattttactgggttccaagcaactctaattatatacaaaatgttttttgtatacaacaccctccttatctttttttcggctttagccataaataacggcaagtaactccaaaatacaggatatttcatgcttttagaaactttttattagtcttcttaattattcagattttgtggcaattaaactttgcagctcctccaaatagttgtccagctcctcttctttaagattgctgagatatgacaatctgtaatttttagcctttttggccatctctagcgcactctccgtcattcccaaatctttcaaaacacagctatagttatagtatgcctcaggaaaatttggattaagacctataagctttcgatatatttcctcggcctttttaatgttaccgattttgtaataggtatagcctaaattgtcaagtatgacaggatcagagctgttatactcatacgcctccttattgaaatttaaagctttgttcatatccccgtccagaataagaagatatccgagacttccgtatatggttgtattcttatagtttgaaaacacatcctccagcatttttatgccatctttcaactgtccctttttccaaagcaccaatgcatagttggactttgcctgcccgggtaccgcggccgcgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcggcggccgcgaattgacagatcgctgagataggtgcctcactg

SEQ ID NO: 84 - DNA, 954 bp, Clostridium thermocellum
atgaacaataacaaagtaattaaaaaagtaaccgtagttggtgcaggctttgtaggttccaccacagcttatacattgatgctcagcggacttatatctgaaattgtactgatagacataaatgcaaaaaaagccgacggagaagtcatggacttaaatcacggcatgccttttgtaaggcccgttgaaatttatcgtggtgactacaaagactgtgccggatccgacatagtaatcattaccgccggtgccaaccaaaaagaaggcgaaacgagaatagatcttgttaaaagaaacacggaagtattcaaaaatatcataaatgaaattgtaaagtacaacaacgattgtattcttctggtagtcacaaatccggtggatattttaacctatgtaacttacaaactatccggattcccgaaaaacaaagtaataggttccggaacggttttggacacagccaggttccgttatcttttaagcgaacatgtaaaagtggatgcacgaaatgtacatgcttatattattggcgaacacggtgacaccgaagttgcggcctggagtcttgcaaatattgcgggaattcccatggatcgctactgtgacgaatgccatcagtgcgaggagcagatttcccggaataaaatatatgaaagtgttaaaaatgcagcttatgaaatcatcaggaacaaaggtgcaacctattatgccgtagcccttgccgtaagaagaatcgttgaagccattgtaagaaatgaaaactccatccttaccgtttcaagccttttggaaggacagtacggacttagcgatgtatgcttaagtgttccgacaatcgtgggtgtaaacggtattgaggaaatattgaacgtgcctttcaacgatgaagaaattcagcttctcagaaagtccggaaacactctaaaagaaataataaaaacactagatatatga

SEQ ID NO: 85 - DNA, 4095 bp, Artificial Sequence
  Synthetic polynucleotide
aattcgagctcggtacccggggatcctctagagtcgacctgcaggcatgcaagcttcgtagttggtgcaggctttgtaggttccaccacagcttatacattgatgctcagcggacttatatctgaaattgtactgatagacataaatgcaaaaaaagccgacggagaagtcatggacttaaatcacggcatgccttttgtaaggcccgttgaaatttatcgtggtgactacaaagactgtgccggatccgacatagtaatcattaccgccggtgccaaccaaaaagaaggcgaaacgagaatagatcttgttaaaagaaacacggaagtattcaaaaatatcataaatgaaattgtaaagtacaacaacgattgtattcttctggtagtcacaaatccggtggatattttaacctatgtaacttacaaactatccggattcccgaaaaacaaagtaataggttccggaacggttttggacacagccaggttccgttatcttttaagcgaacatgtaaaagtggatgcacgaaatgtacatgcttatattattggcgaacacggtgacaccgaagttgaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtc

SEQ ID NO: 86 - DNA, 8882 bp, Artificial Sequence
  Synthetic polynucleotide
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctccgaattcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaagatctgttgtgtggaattgtgagcggataacaatttcacacaggaaacagctatgaccatgattacgccaagcttgcatgccctgcaggtcgactctagagccaccgctggaacattaacagattttattatggtatcaattatttcttcaggcgatattttcttaagccttgcttcctgctttagtataatcctgtgggaaagcacgggtattgacataagctttacatcttcgggaattacaaagtttctttcattgtataaagcccatgcctgggaggctctgtatagagaaagagaacctcttggacttgaacccagacttatttccgaatggaatcttgtccggcttactatatccactatgtagttgttgatggacctgtcaacataaacttttttaacctcgctttgaatattcagtatatcgctgctgtccgccacaggttttaaggtttccaaggggttttcgtcgtgaaatcttgacagtatttcggattcttcaaacttttcaggataacctatggaaactttcataaaaaacctgtccagctgagcttcgggaagaggataggtgccgagatactccacgggattttgagttgccagtaccataaaaggccttggaagattgtatgtcacaccgtcaacggtcacctgtttttcttccattgcttcaagaaggcttgcctgggtttttggagatgttcggtttatttcatccgcaagtattatctggctcataatgcttccgggatgatattcgaatttaccttctttctgattgtacatcgtaaatcctgtaatgtctgaaggaagaatatccggtgtaaactgtattcttttaaaagaagcatttaccgactttgcaagagatgatacaatactggtttttccgacaccggggacatcttcaatcaaaacatgtccatcgcaaataagtgatattaatatcaactcgatagctttcttttttccaacaatgactttttcaatatttgacacgattttatttaaaatttccactgaattgttcatatttcctcccccgatagataatttccattatgtaaaataggttttatattaaaattatatactagtttatatatttatcaataatgaaagttctccttttcataaaagaaaaggagaactttcatatatctagtgtttttattatttcttttagagtgtttccggactttctgagaagctgaatttcttcatcgttgaaaggcacgttcaatatttcctcaataccgtttacacccacgtagttcaacaaacgggattgacttttaaaaaaggattgattctaatgaagaaagcagacaagtaagcctcctgtatttcttttccgaaagagaggaaaagattctaaagagttttggaaatactgatgaatattgtgtgcagagtacaattctatggacaagaaaagaggctttgtcaaaactttttcgtctgggaatgaggatggattttaaaaagctggatactttggagggcgaggtggtttttcaggaaacaaacagggcgcgtctgttttcttttatatgcaataattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggcttacgggtgatacggctgctcttgaaacctttgattttaattctttttcccttttattttataacactaattgatataataagttacaaaaagtttaaatttaaggggtctttcaatatgaatgcggagatattagcggttggaaccgagcttttaatggggcagatagcaaataccaatgcccagtatatatccaaaaggctcaatgacattggtgtgaatgtgtattatcacagtgtggtgggggacaattccgttcggctgaaaaaatgtcttcttgcagctttggaaaggtgcgaccttgttattatgaccggaggactcggccccacgcaggatgaccttacaaaggagactgttgcggaagttttggggaaaaagcttgttttacacgaagaaagccttgagaggattaaaactttttttaccaggataaaccgaaaaatgacggacaataatgtcaagcaggcatatcttccggaagggtgcacagtggttgagaataacagcggcacagccccgggctgcattattgaagataagggaaagattgtggtaatgcttcccggtcctccgccggagatgatgccgatgcttgatgataccgttattccttaccttgcggaaaaatcaggatacaggatagtgtcaaaatatctgagggtttttggaataggggaatcacagcttgaagagatgattatggatttagttgacaaacaggacagggtcaccatagcaacttatgcaaaagacgggcaggtgaccgtaagacttaccacaaaagccaggacagaggaagaaggctttcgtgaaatacttcctttgcaaaatgagatagcttcaagactcaaagaggcattatacagtacggaagatgaagcccgggtaccgcggccgcgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcggcggccgcgaattgacagatcgctgagataggtgcctcactggattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 87 - DNA, 2622 bp, Clostridium thermocellum
atgacgaaaatagcgaataaatacgaagttattgataatgttgaaaagcttgaaaaggctttgaaacgtttaagagaagctcaaagtgtttatgcaacctatacacaggagcaggttgacaaaattttctttgaggcggcaatggcggccaataaaatgagaattcctcttgccaaaatggctgtggaggaaacaggcatgggagtggttgaagacaaggttatcaaaaaccactatgcttctgagtatatctataatgcgtacaaaaacactaaaacctgcggtgttattgaagaggaccctgctttcggtattaaaaaaatagcagagcctttgggggttattgcggcggttatacctactacgaatccgacatcgacagcaatctttaagactcttattgcattaaagacgagaaatgcaattattataagcccacaccccagggcaaaaaactcaacgatagaagcggcgaaaattgttttggaggcggccgttaaagccggtgctccggaaggcatcattggctggattgatgtgccgagccttgaacttaccaacctggtaatgagagaagcagatgtgattctcgcaacaggcggtcccggactggttaaagcagcttactcttcgggcaaaccggctattggtgtcggagcgggcaatactcctgcaattattgatgattcggccgacattgtcttggcagtgaactcaataatacattcaaaaactttcgacaacggtatgatttgtgcttcagagcaatcggtcattgttctggacggggtatataaagaggtaaaaaaagaatttgaaaaaagaggatgctatttcttaaatgaagatgaaactgaaaaggtaagaaaaacaattataataaacggtgcgttaaatgccaagatagtaggtcagaaagctcacacaattgcaaaccttgcaggttttgaggtacccgagactacaaaaattctgataggcgaagttaccagcgtggatatttccgaagaatttgcccacgaaaagttgtgcccggtactggcaatgtacagggcaaaggattttgacgatgcccttgataaagcagaaaggttggtagctgacggtggatttggccatacttcgtcactttatatagatacggtaacacagaaagagaaacttcagaaattctctgaaaggatgaaaacctgccgtatattggttaatacgccgtcatcccagggaggtatcggtgacctttacaacttcaagcttgctccgtctctcaccctcggctgcggttcctggggaggaaattcagtttccgacaatgtgggagtcaagcatttgttaaacattaaaacagttgccgagaggagagagaacatgctctggttcagaacacctgaaaagatttatataaaaagaggttgtctgcctgttgcattggatgagcttaaaaatgtaatgggtaaaaagaaagcatttattgtaacggataacttcctgtacaataacggctacaccaagccgattacggataagctggatgaaatgggaattgtgcacaagaccttctttgatgtgtctccagacccatcccttgcatctgccaaagccggtgcggcagaaatgctggctttccagcctgacaccataattgcggtcggcggcggatctgccatggacgcggccaaaatcatgtgggtgatgtatgaacatccggaagttgactttatggacatggcaatgagatttatggatataagaaagagagtttacaccttcccgaagatgggacagaaggcatactttatcgcaattccgacttccgcgggtacaggttcagaagtgacaccttttgcggttattactgatgaaaaaacaggaattaaataccctctggccgactatgaattgttgccggacatggctattgtagatgccgatatgatgatgaatgctccaaagggacttaccgcagcttccggtatagacgcattgacccacgctctggaagcctatgtttcaatgcttgcgaccgactatacggatagccttgcccttcgtgcaataaagatgatatttgaatatctcccgagagcctatgaaaacggtgcaagtgacccggttgcaagagagaaaatggccaatgccgcaacaatagccggaatggcttttgccaatgcctttttgggtgtatgccattcaatggcgcacaaactgggtgctttttatcacctgccccacggtgttgccaatgcacttatgataaacgaagtaatcagattcaactcatccgaggctccgaccaagatgggtactttcccgcagtatgaccatccgcgcacgctggaaaggtatgcagaaattgccgattatatcggacttaagggcaagaataacgaagaaaaagttgaaaacttgattaaagctattgatgagcttaaagaaaaggtgggcatcaggaagaccatcaaagattatgacatagatgaaaaggaatttttggacagactggacgaaatggtggaacaggcttttgacgaccagtgcacaggtacaaatccaagatacccgcttatgaatgaaatcaggcaaatgtatctgaacgcttattacggaggtgcgaagaaatga

SEQ ID NO: 88 - DNA, 4102 bp, Artificial Sequence
  Synthetic polynucleotide
aattcgagctcggtacccggggatcctctagagtcgacctgcaggcatgcaagcttggcggcaatggcggccaataaaatgagaattcctcttgccaaaatggctgtggaggaaacaggcatgggagtggttgaagacaaggttatcaaaaaccactatgcttctgagtatatctataatgcgtacaaaaacactaaaacctgcggtgttattgaagaggaccctgctttcggtattaaaaaaatagcagagcctttgggggttattgcggcggttatacctactacgaatccgacatcgacagcaatctttaagactcttattgcattaaagacgagaaatgcaattattataagcccacaccccagggcaaaaaactcaacgatagaagcggcgaaaattgttttggaggcggccgttaaagccggtgctccggaaggcatcattggctggattgatgtgccgagccttgaacttaccaacctggtaatgagagaagcagatgtgattctcgcaacaggcggtcccggactggttaaagcagcttactcttcgggcaaaccggctattggtgtcggagcgggaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttcaatactttaaaacacatacataccaacacttcaacgcacctttcagcaactaaaataaaaatgacgttatttctatatgtatcaagaatagaaagaactcgtttttcgctacgctcaaaacgcaaaaaaagcactcattcgagtgctttttcttatcgctccaaatcatgcgattttttcctctttgcttttctttgctcacgaagttctcgatcacgctgcaaaacatcttgaagcgaaaaagtattcttcttttcttccgatcgctcatgctgacgcacgaaaagccctctaggcgcataggaacaactcctaaatgcatgtgaggggttttctcgtccatgtgaacagtcgcatacgcaatattttgtttcccatactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtc

SEQ ID NO: 89 - DNA, 8514 bp, Artificial Sequence
  Synthetic polynucleotide
gattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagtatgggaaacaaaatattgcgtatgcgactgttcacatggacgagaaaacccctcacatgcatttaggagttgttcctatgcgcctagagggcttttcgtgcgtcagcatgagcgatcggaagaaaagaagaatactttttcgcttcaagatgttttgcagcgtgatcgagaacttcgtgagcaaagaaaagcaaagaggaaaaaatcgcatgatttggagcgataagaaaaagcactcgaatgagtgctttttttgcgttttgagcgtagcgaaaaacgagttctttctattcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttgattactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggattgacttttaaaaaaggattgattctaatgaagaaagcagacaagtaagcctccgaattcggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaagatctgttgtgtggaattgtgagcggataacaatttcacacaggaaacagctatgaccatgattacgccaagcttgcatgccctgcaggtcgactctagagtgaagcttgaaaaggttatagcggtaagaacggacaaaaccgtttacagagacggagacaaggcaataaaggtcttttccagcaactattcaaaggccaacgtattgaatgaagctttaaaccaggccagggtggaagaaaccgggcttaacattccgaagcttcttgaagttacaaaaattgatggcaaatgggcaattgtgacggagtttattgaaggaaagaccttagagcagcttatgaaagaaaatcccttaaaatatgatgaatatatgaacctctttgttgatattcagctcgaagtgttaagcaaaagggctcctttattaaacaagctgaaagacaaaatgcacagaaaaatctcagagagcggtcttgatgccacgacgcgctatgagcttcatacaagacttcaaagtatgcccgaccataacaaggtgtgtcatggtgatttcaatcccagcaatattattataaccaaagacggcactccttatatacttgactggtcgcacgtcacccagggaaacgccagcgccgatgccgcgagaacatacctgttgttctggctttccggcgatattgacaaggcagccaagtatttgaaccttttctgtaaaaagagcgacactgcaaagcaatatgttcagaagtggatttcaattgtagcggcatcccagcttgtaaaaggaagagaagctgaaagggagttattaatgcgctggattgatgttgtagattacgaataggaggtatttgggatgaaagtgtctatttgcatagggagctcttgccacttaaaaggagcaaagcagatagtggaacagctgcaaagcctggttgcgtagttcaacaaacgggattgacttttaaaaaaggattgattctaatgaagaaagcagacaagtaagcctcctgtatttcttttccgaaagagaggaaaagattctaaagagttttggaaatactgatgaatattgtgtgcagagtacaattctatggacaagaaaagaggctttgtcaaaactttttcgtctgggaatgaggatggattttaaaaagctggatactttggaggacgaggtggtttttcaggaaacaaacagggcgcgtctgttttcttttatatgcaataattactgtatctctctggcattgccaggttttaataaagattaaaattattgactagaaataaaaaaattgtccataatattaatggacaaaaaaacaaagaattacatcaaaggaagataaaaatactttgttaaaaaattaattattttttatctaaactattgaaaatgaaaataaaataatataaaatgaatcatagtgcaagagatacttgccagaggatgaatattttactgcattcatgctttatggcagctaatagaggcattaaattaaattttaatttacaataggaggcgatattaatgatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactagggctcgcctttgggaagtttgaagggcttgtattgactttgattgtattaaacggtaaaatgatatcgataacaaaatatcgataaaaaaataccgataaataaatatcgatatcaataatcttatagtacttgagttaaaaggggtggattggctaaaatagtttaataagtaattattaaaaaagaaaggaaccaatatatatgaataatccgaaagctatctcaaaacagactcttttaagattaccttcatatttaagttacttaaaatcgctgccaaagcaggacggtgaatatgtatcggctacaatgattgcttcggcgctgggactgaatgatgtgcaagttcgaaaggatctggcctgtgtaagcaaaaaaggccgtccaaagcttggatatgttgctgaagagttgataagggatatagagagttttcttgggtatgactctttgaacgatgcgataattgtgggcgcgggaaggcttggaggagctcttttatcctatgagggctttaaagaatatggattaaatattgttgcagcgtttgatattgatgaaagtaaaatcggcactgaaatttgcgggagaaaaattttccctttggacaaaatgaaggaactgtgccggcggatgaaaattcgtatcggtataataaccgtccctgcagattcagcccaaaaagtatgcgacatgcttgtagacagcggaatatatgcaatatggaattttgcacctgttcaccttaaagtaccggataacattttagtccagcaagaaaacatggcggcatcactggctgtactttccgcacacttggcagaagcaataaagaataatggaatcggaaaaggagatgaggaaaatgcccgggtaccgcggccgcgatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccatcgataagctagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccacttttcggcggccgcgaattgacagatcgctgagataggtgcctcactg

SEQ ID NO: 90 - DNA, 1170 bp, Clostridium thermocellum
atgaaagcatttaactattatgcacctactgaaattattttcggatgcggcagagttcaggaaattggatcaataactgcccaatatggaaagaaagctttgctggtaactgtaccggaatttccggaagtaaaggagctctatgaaaaagtaaagaagtctcttagggaaaatggtgtggaagtagtacattttgacggtgttattccaaatcccaccactgatgttgtaactgaaggagcaaatatggcaaaggctgccggggttgatgtagttattggattgggaggaggttcctcaatagatactgcaaaagccattgcagttgaagcaacccatccgggaactgcctgggattataactgccacactccgggccccacatctgctaccctccctattatcgcaataggaacaacagcaggaaccggcagccagtgtacacaatgtgcagttatcacaaaaacaagtgaaaaggataaatccgcaatttggcacaaaaatattttccctaaagttgcgatagttgatcccgaagtgacagtcacgatgcccaaaagtgtaactgcgcagaccggttttgacgcttttgcccataactttgaagcatatctttccgtgaaaaccagtccgttagttgagatgatggcaatagaagcaataaagatgattaaggaatatttgccaaaagctttggagaatccaaacgacattgaggcacggtcaaagatgtcattggctgacactttgggtggacttaccaattccaacgcaggagtaacactgccccatgggttgggaatgcaagtcggcggccatgctccccatgtatcccacggacaggctttggcgataatatatcctcaatttacaaggtacacatacgcctgggcaattgaaaagttcgccaaggttggaagaatatttaatcctgcattgaatgaactttccgatgaagaagcggcaaaagaagcgtgtgtggcaattgatgatttcctgaagaaaataggtttgtggattggttttaaagatgtgaatgttacgaaagagcaaattcgtgaaatagcagatgatggtcaggttttaggtgattacctcaacaatcctcgtgtagcaacgattgatgagatgtacgagctactgatgaattgctatgagcgcaaagaatga

SEQ ID NO: 91 - DNA, 1107 bp, Clostridium thermocellum
atgaattttaaatttaagataggtacaaaagttttcttcggaaaagaatgtgtgaaagaaaacaaagccgtatttaaagattttcgcaaaagagctttgctggttacgggaaagaactccgcaaaggcaagcggagctttttcggacgtcgttgaggtacttgaagagtatggcattgattatgagatttatgacagagtggcaaacaatccttctcttgaaaatgtcaaagagggaggggaagcggcaagaaaatttgatgccgattttatcatcggaatcggaggaggctcgcctttggacgcttcgaaggcggtagcggtgcttgcgacaaatgatattgagcctgtggacttgtacaaaaatgtgtttgaaaacaagccccttcccataatagcaattcccaccacagcggggaccggaagcgaggttaccccttattcgattcttacccgtgacgatatgaaaaccaaaaagagcttcggtaacgaggacacttttccggctgtggcattcattgatgccaggtataccgaaagtatgtcctatgaaacaacggtggatacggcactggatgcgtttacccatgcgctggagggatatttgggaagaaggagcacaccggtcagtgatattttggctgttgaagccataaggatatttggtgaatgtctggaaaaccttttaaacaacaaatttgactatgacgtgagggaaaaacttttatacatgtccatgctgggcggtatggtaatatcccatactgggacgacaataatacatggcatgggatattctctcacatattttaaagacattccccacggaagggcaaacggaatgcttgtaagagagtatcttaagtataattatgaagcggcaaaagaaaaaacggataatgtgctgaggcttctcaaagtaccgtccattgacgcctttggagaaataattgacagactgattccccaaaagcctgttttgacaaaggaggaaattgagctttatgcaagcctggcaatgaagcagaacagcactttgtccaatgcacgcacggtggttaaagaggacatggaagaaatatttaaaaatactttcgggaaaggttga

SEQ ID NO: 92 - DNA, 1170 bp, Clostridium thermocellum
atgataaactttgtttataaaaatccaacgaagataatatttggcagaggtactgaattgaaagtcggagaagaagtcagacaatacagtgggaaagtattgcttcattatggtggaggcagcataaagaagaccggtttatacgatagagtggttaattccctgaaacaggcaggagttgaagtggttgagcttggaggagtcatgcccaatccgaggcttggccttgtaaacgagggaataaaaatttgccgcgaaaaaggaattgactttattctggcggtaggcggaggaagtgccattgattcggcaaaggctatagcagtgggagtgccgtatgacggcgacgtatgggactttttctgtggcaaggccgaaccgaaagaagcccttcctgtaggtgtggttttgaccatacctgctgcgggaagtgaagcaagcccaaattctgtaataaccagggaagacggcctgtacaaacggggaatgtactctgagctgataaggccggtatttgccattatgaatcctgaattgacctatacgcttcctgcatatcagactgcgtgcggtactgctgatatcatggcgcatataatggaaaggtattttacaaacgaaacccatacagatttaacagaccgtctttgcgaggctacgctcaaaacaatgattaagaacgtgcctattgctttggaagagccggacaattataatgcaagggcggagattatgtgggcaggtacaattgcgcacaacgggcttttgggaaccggaagaattgaggactgggcatcccataatattgagcatgaaatcagcgcgatttacgatgtggcacacggagccggccttgcagtggtgttcccggcgtggatgaaatatgtttacaaaaacaatttggaccgttttgtgcagtttgccgtaagagtgtggaatgtggaaatgaattttgacgagccggaaaggacggctctggaaggtatagaacggctgaaaaagttctttaaagaaataggtcttccggtttctttgaaagaaatgaacataggcgatgacaggctggaagaaatggcgtcaaaatgcacgaacggaggaaaagctacaatcggaaattttgtaaaactcaatcgtgaagatgtgtacaatattttaaagctggcagtgtaa

SEQ ID NO: 93 - DNA, 1419 bp, Clostridium thermocellum
atggcggaaaatcatgactatgagagagaaataaacagattgtttgaattgcagaagaaaaacgttgtacggcttcgcacatcgagtatagatgagagaattgcgaaactgaaaaaattgaaagaatatatttgggaaaataaagaaaagattcaggaggcggtttataacgatttgagaaagcctccggaggaggttttattaaccgaaatatatcctgttgtctcggaaatcaggcatgtaataaagaatttgaaaaaatggacaaagcctaagaaagtcagaacacccatatctctttttggggcaaaaagctattacagatttgaggcaaaaggggtggtgctgattatttcaccgtggaactatccctttgaactctcaataggcccgttaatcactgccattgctgccgggaatgcggttgtattgaagccttcggaattgagtccccatacatccggttatataaagaaacttgtggcagacatttttgatgaaagtgaggttgctgttgttgaaggggatgcggtggtggcccaaaaactgctggagatgggttttaatcatatattttttaccggaagtacaaaggttgcgaaagctgtgctaaagaaggcctctgagacattgtcttcggtaacccttgaactaggaggaaaaagtccggtaattattgacggcaaatttgatattgaagaggctgctaaaaaaataacatggggtaaatatttaaatgcagggcagacatgcatagctccggattacgtttttgtaaaaaaagagcttttaggggattttgtaagccacttaaaacattacataaaaaaatattattattctgacggcagcggaagatgcagcaactactgcggtattatcaacgaacgtcactttaacaggctgaaaaatgtgtttgaggtgacggtaaaagagggggcaaaagtttgtgagggcggtctgtttgttgagaatgaatgctatatatcacctactgttttgacggatgtgggcagagactcatatataatggaggaggaaattttcgggccgattttgccggtgctgacttatgaaaaaatcgatgatgtcattgagtatataaactcaaagcctgctcctttggtgttgtatgttttcagcagggacaggaaattttacagacatgtgattaataacgtaatttccggggattgtctgataaatgatgtgatagcgcactttgccaatcccaggctgccttttggagggcacaacgccagtggaatcggaaagtcccatggttattacggatttagagaattttcccacctgcgttcaatcatgattcaaccaaaacgcacaatgttgcagttgctctaccctccgtacggcgagtttgtaaaaaagttgattgagtggagtacgaaatatttttag

SEQ ID NO: 94 - DNA, 2229 bp, Clostridium celluloyticum
atggatccatggagatatttcaaaaacggaaattggtccgaggaaatagatgtaagggattttattattaataattatataccatataaaggagatgattcctttttaaaaggccctactgaaaagactcaaaaactctggaaaaaagtcagtggtatccttgacgaggaaagagagaacggaggagttctcgatgttgataccaaaataatatcaactatcacttcgcatgaacccggatatattgataaggatttggaaaagattgtaggattgcagacggataaacctctaaaaaggggtataatgcctttcggtggtatcaggatggtagtaaagggcggagaggcctacggcaaaaagattgacgaaagtgttgtaaagctatttactgaatataggaagactcacaatgacggtgtttacgatgtctatactcccgaaatgatgagggcgaaaaaagcaggtattataaccgggcttcctgatgcatacggtagaggaagaataatcggtgattacagaagagttgcactgtatggggttgacaggcttatcggagataagcaaaaacaacttacaagcctagaaatggattacatggacagtgaaaccatacaggaaagggaagaaagaaaaagtcagataaaagctcttgagtacttaaagcaaatggctgaaatgtatggctttgatatttcaaggcctgctgaaaccgcacaggaagcatttcaatggttgtatttcggtttcctcggagcagtaaaggagcagaatggtgcggctatgagtcttggaagagtttcaacattccttgatatttatatagaaagggatatgaaggaggggacacttaccgaagaagaggcacaggagcttgttgaccattttgttatgaagcttagattagtaagattcctgagaactccagagtatgagaaactgttctcaggtgaccctacatggataaccgaaagcattggaggtatgggtcttgacggacgtactctggtaacgaagaattccttcagaatgctgcataccctatttaatctgggtcatgcacccgagcccaatatgacggtgctttggtcggtgcatctgcctgatggctttaagaaatactgctcatatgtttctatcaatacaagctctatccaatacgagagcgatgatataatgaggtattattggggagacgactacggcatagcatgctgtgtttcagccatgagaataggaaagcagatgcagttcttcggagcccgctgcaatatggcgaaagctttgttgtatgcaattaacggggggcgtgatgaaaaatcgggcgttcaggtaggacctataatgcaggctattgagactgaatatcttgattatgatgatgtaataaagaggttcgacgcagtactaacatgggttgcaagactttacatcaacactctgaatataatacactatatgcatgacaagtacgcttatgaaagactacagatggctttgcacgacaaggacatattaagaacaatggcctgcggaatagcaggattatctgtagttgcggattctctgagtgcaattaaatatgcaaaggtaaaggtaataagaaatgaagaggggcttgccgttgactatgacatagaaggtgattatccgaaatttggaaataacgacgaccgtgtagacaatatagccgtaatgctggtaaaaagctttatggagaagctggaaaagcagagaacctacaggcactctgttcctaccctttcaatactgactattacatcaaatgttgtatacggagctaagacaggtaatacacctgacggaagaaaagcgggagaacccttcggaccgggtgcaaacccaatgcacggaagagacttgaacggtgcacttgcagttcttaaatccatttcaaagctgccttaccagtttgcacaggacggtatatcatacactttttcaatagtaccaaaggcacttggaaaagaggaagatacaaggatcaacaaccttgtgtcactgcttgactcttactttaaagaaggcggacatcacattaatatcaatgtatttgagagagaaatgctgctggatgcaatggatcatcctgaaaagtacccacagctcacaatcagggtatccggctatgcggtaaactttataaaattgacgagggaacagcagctagatgtaattaacagaacgatacatgaaaatatatag

SEQ ID NO: 95 - DNA, 2229 bp, Clostridium celluloyticum
atgaggctaaacatttctgattttaagccgggtaaatggcaaaattctattgacgtacaggatttcatccagacaaactacactccatatgatggtgatggaagttttctatgcggagcaagttcaaaaaccagacagctctgggagacctgcagaagtcttttggttgaggaacatttaaacggtggaattcttgatattgacacagaaaccgtagcaagtataaacagtcacaaacccggctttatagacgagaagtacgaagtaataaaggggcttcagacagacacacccttaaaaagagcctttcttgcaaaaaccggttacagaatggctaaacaggcctgtcagcagtttgacgctgcaccttcacccgagtttgacaaaattttcagtaaaagcattaaaacacataatgatggtgttttcagtgcatatacgaatgaaatgcgtaaggctagaagatgtggagttataaccgggctccccgatacatatggcagaggccgtataatcggcgattatagaagagttgccctttatggtacggatgttttgataagtcaaaagcaaaaagacctggacagtcttataggtaccatgactgatggcgttataaggctcagggaagaaattcaagatcagatcgccgcactcaatgatcttgctgttcttggcggttcctatggttttgatatgaagcggcctgccctaaatgcctttgaagctgtccaatggacgtatttggcatttttaggtgctttaaaggaaactaatggtgccgcaaattctttgggcagactaaatgttttttttgatatatttatccaacgggatataaataacggtattctgacagaggaagatgctcaggagctgattgaccagtttgtaataaaactgaggtttataagacacttacgtaccaaagagtataacgaactctttgcaggtgaccctgtttggctgaccgaatccctcggcggaatgagcagtgacggaagacatatggtttccaagacctccttcagattccttcaaactttaaagaatctgggagctgcacctgaacccaaccttacaatcctttggtctgttaaactacccgaaaattttaaaaagttttgtgctgaaatatctattgtaacaagtgcaatacagtacgaaaatgatgatcttatgcagccccagtacggtgatgattacgggatttcatgttgtgtatctgctatgcgtcttggtaaggatatgcagttttttggtgcgagatgtaaccttgcaaagttacttttattatctcttaacggagggcgtgatgaaattagcggagatcaggttgcacctgaaatcaagccttacgaaggtgaataccttgaatatgagaaggttatgaaaaacttccttgagctccaggattggctgacaggtttatatgtagacacaatgaatattatacactatatgcacgacaaatatgcgtatgagcgtctcatgatggctctgcatgatacggatgtaaacaggcttatggcctttggtatatcaggactgtcagtacttgtggattccctgagtgccataaagcatacacgcgtcagggttgtaagggaccaacggggtattattacagactttgagcttgaaggaagttatccgcaatacggaaataatgacgacagagccgattgtattgcaaaagaagttgttacaagcttttataacagcctcaaggctcatcctacctaccgtaatgcaacgcacactttatcaattctaaccataacatcaaacgtcgtgtacggtaaaaaaacaggctcgactcctgacggccgcaaaaaaggagaaccttttgcccccggtgcgaaccccatgcataatagagataagtcaggaatacttgccatgatgaattcagtggcaagtattccctacaatgtgtgcagtgacggtatatcccttaccctatcggttattcctaatgctctgggcaaggcgagggagaccaaaatctcaaacctttctgctctgattgacggatatgtaatgagcagcggtcatcatataaatgtaaatgtacttgataaggaaactctggaaaatgccataaaggaaccaatgaaatacaaccagcttacggttcgtgtgtcgggctatgcagtacattttgtaaagctgaccaaggcacagcaattggaggtattatccaggacatttcatgaagctatgtaa

SEQ ID NO: 96 - DNA, 12788 bp, Artificial Sequence
  Synthetic Polynucleotide
tctagattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgagaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgattctagagcactggccgtcgttttacaacgtcgtgacgctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttatcataccacccttgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggtttatgatcgatagaggaaagtgtctgaaacctctagtacaaagaaaggtaagttataagggtggacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgtcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagttgtttttagttacagacaaacctgaagttgggttgcaagcagcagattacggttcgtgttcgtgatgggttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgactgccgggcctcttgcgggataaaaagaaaaacgaaatgatacaccaatcagtgcaaaaaaagatataatgggagataagacggttcgtgttcgtgatgcttagtgggaatttgtaccccttatcgatacaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgatta

SEQ ID NO: 97 - DNA, 12788 bp, Artificial Sequence
  Synthetic polynucleotide
tctagattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgagaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgattctagagcactggccgtcgttttacaacgtcgtgacgctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttactgtacgtcaattgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggtttacagtcgatagaggaaagtgtctgaaacctctagtacaaagaaaggtaagttactattgacgacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgtcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagttgtttttagttacagacaaacctgaagttgggttgcaagcagcagattacggttcgtgttcgtgatgggttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgactgccgggcctcttgcgggataaaaagaaaaacgaaatgatacaccaatcagtgcaaaaaaagatataatgggagataagacggttcgtgttcgtgatgcttagtgggaatttgtaccccttatcgatacaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgatta

SEQ ID NO: 98 - DNA, 996 bp, Clostridium celluloyticum
atgaactttttagagcagattgtaagcagagcaaaaaaagatatcaaaacaatagtacttcctgaaagtaacgatatcagaactttgaaggctgctgcaatgattcaggagcaaggcattgccaatgtcgtacttgtgggcaacaatgatgatattaaaaaactgggcggtgacctggatatatcaaaggcaaaaattgttgatcctacaaactttgaaagatttgacgagtatgtaaataccctgtacgaacttagaaaggcaaagggaatgacacaggaagaggcaaaaaaaatattgtctgaaaatcctttgtatttcggaattatgatggtaaaaatgggtgaggctgacggaatggttgcaggagcaattaattcaactgcaaatacacttaggccggctcttcaaatattaaagacagctcccggagcaaaacttgtatctgcattcttcgtaatggttgtacctgactgtgaatacggagaaaatggagtgttcatttatggagacagcggactagtcgaaaatccaaatgcagaagagctttcagaaatagcgatagcatcttcaaaatccttcaaaaaccttgtacaggctgaacctgttgttgcaatgctctcatattcaacatacggcagtgcaaaaagtgtattgacagaaaaggttatagaggccacaaagcttgcaaaggaaaaagctccagatcttcagttagatggagaattgcaggcagatgcagctattgttccttctgtaggagcatcaaaagcacccggaagcagtgttgcaggaaaggccaatgtattaatattccctgacttgaactgtggtaatatatcctacaagctaacacagagacttgcaaaggcagaagcctacggccctattattcagggaattgcaaaacctgttaatgacctttcaagaggctgtagtgccgaggatatagtaggtgttgttgcaataacttgtgtacaagcacaaaatatataa

SEQ ID NO: 99 - DNA, 12789 bp, Artificial Sequence
  Synthetic polynucleotide
tctagattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgagaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgattctagagcactggccgtcgttttacaacgtcgtgacgctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttaacaaactttgaagtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcgatttttgttcgatagaggaaagtgtctgaaacctctagtacaaagaaaggtaagttactttcaaagacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgtcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagttgtttttagttacagacaaacctgaagttgggttgcaagcagcagattacggttcgtgttcgtgatgggttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgactgccgggcctcttgcgggataaaaagaaaaacgaaatgatacaccaatcagtgcaaaaaaagatataatgggagataagacggttcgtgttcgtgatgcttagtgggaatttgtaccccttatcgatacaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgatta

SEQ ID NO: 100 - DNA, 951 bp, Clostridium celluloyticum; identical to SEQ ID NO: 22

SEQ ID NO: 101 - DNA, 12789 bp, Artificial Sequence
  Synthetic polynucleotide
tctagattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgagaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgattctagagcactggccgtcgttttacaacgtcgtgacgctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttaactgacacatatgtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcgatttcagttcgatagaggaaagtgtctgaaacctctagtacaaagaaaggtaagttaacatatgtgacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgtcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagttgtttttagttacagacaaacctgaagttgggttgcaagcagcagattacggttcgtgttcgtgatgggttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgactgccgggcctcttgcgggataaaaagaaaaacgaaatgatacaccaatcagtgcaaaaaaagatataatgggagataagacggttcgtgttcgtgatgcttagtgggaatttgtaccccttatcgatacaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgatta

SEQ ID NO: 102 - DNA, 2604 bp, Clostridium celluloyticum
atgactaagaaaaatatggtagtaaatagtgtagacacactgttggtaaaactggcagaaataaggaaggcacagaaagaatattcaacatttacacaggaacaggttgacaaaatatttctggctgcatcacttgctgcaaataaaaagagaattcctctttctaaaatggctcaagctgaaacaggaatgggtgttatagaagacaaggtgattaagaaccattatgcagcagagtatatttacaatgcgtataaggatactcaaacctgtggcgttattgaacgtgacgaagcttttggtatcacaaaaatagctgagccccttggtgtcgttgcagcaattgtaccaacaacaaacccaacttcaacagctatttttaaagcacttattgctttgaaaaccagaaacggaatcatattctcaccacacccaagagctaagaaatgcactattgaggctgcaaatgtaattttggaagcagctgttgcagcaggtgcaccaaagggaataatcggatggattgatgagccgtcaatagagctttcagaaagagttatgaaggaagccgatataattcttgctacaggcggaccgggtatggttaaggcggcatactcatcaggaaaaccagcaataggtgtaggaccgggtaacgtacctgcaataattgatgaaagtgccgatataaagacagctgtaagttcaattatagtttctaaaacatttgataacggtatgatctgtgcttccgagcagtcagtaattgttttagataaaatatacaatgaagttagaaaagagtttattctcagaggagcatacatgcttaataaggaagaaactcaaaaggtaagagaagtaatactggttaacggtgcattaaatgctaaaattgttggtcagacagcttctactattgcaaaattggcaggctttgaagttcctgctgaaacaaaggtattggtcggagaggttgaatcagttgaattgtccgaagcttttgcacatgaaaagctttctccggtacttgccatgtacaaggcaaaaaccttcgacgatgctgtttctaaggctgaaaggcttatagcagacggtggatttggtcatactgcttcactatatattgacactgtaaacagcaaggataaacttgacaaatttactgcggcaatgaagacctgcagaattcttataaatacaccatctgcacagggcggaataggagacctttacaactttaaacttgcaccgtcacttacccttggctgcggttcatggggtggtaactccgtttcagaaaatgtcggagtaaagcatctgatcaatattaagactgttgccgagaggagagagaatatgctgtggttcagagcacctgaaaaagtttatttcaagaaaggatgccttggtgttgcactaagagagctcaaaactgagatgaataagcaaagggcattcatagttacagactcattcctttacaacaacggatatactaaagctgttacaaacctgctggatgatatgaatatcaagcatcataccttctttgatgtagctccggacccaacccttgcctgtgcaaagcttggtgctgaggcaatgagggatttcaaacctgacgttattatagccatcggcggaggttccgcaatggacgcaggtaaaattatgtggacactgtatgaacatcctgaagttgatttccaggatctcgcaatgagatttatggatatcagaaaaagagtatacacattccctaagataggcgagaaggcatactttgtagcaatcccgacatcagcaggaacaggttcagaggtaacaccgtttgcagttattactgacgaacagtcaggtgtaaagtatcctcttgcagactacgagctcatgcctaagatggcaatagttgatgcagaccttatgatgaatatgcctaagggtcttacagcagcttcaggtatagatgcacttacgcatgctgttgaagcatatgcttcaatgcttttaacagattatacaaagggtcttgctttacaggcaactaagaacatttttgaatacctgccttcagcgtatgagaatggtgcaaaggaccctattgcaagagaaaagatggctgacgcatcaacaatggccggtatggcatttgcaaactcattcctgggtatttgccactcaatggctcacaagctgggagcattccatcacctgcctcatggtgtggcaaatgcacttctgattactgaagtaatgaagttcaatatagctgaagctcctacaaagatgggtgcgttctcgcagtacaagtatcctgaaatactcaagagatacgcagaagtagcgtcctttatcggaatcacaggttccaccgatgaagagaaatttaaaaagcttatagctaaaatagatgaattaaaagcaaaagttggattaccaaagactatcaaggaagcaggagttgatgaagagaagttcctggctactctagatgaaatggttgagcaggcattcgacgatcaatgcacaggtgctaacccaagataccctctcttgagtgaaatcaaggacatgtacctgaaagtttactacggcaagtaa

SEQ ID NO: 103 - DNA, 12789 bp, Artificial Sequence
  Synthetic polynucleotide
tctagattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgagaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgattctagagcactggccgtcgttttacaacgtcgtgacgctcataacttcacgctcctgtatatatttttatttatttaaaaatgagtcaaaatttaggaaatgattgcaatatgtataatatccaaatttccattcaaataaccaaagtaattttacctctttttatgagctatttcaatactttgttagtaaattaacatatatgagctgtatatggtttaatgaaaaaagttattttgaagggatattgtaaaaaacataatatattatatggataaattttacatttgacttatcatatgttaatatatgtaatatgaatagctaatctaagcaggctactgcctagaaaaaagcttataattatccttaatgcccgctcaagtgcgcccagatagggtgttaagtcaagtagtttaaggtactactctgtaagataacacagaaaacagccaacctaaccgaaaagcgaaagctgatacgggaacagagcacggttggaaagcgatgagttacctaaagacaatcgggtacgactgagtcgcaatgttaatcagatataaggtataagttgtgtttactgaacgcaagtttctaatttcggttggcattcgatagaggaaagtgtctgaaacctctagtacaaagaaaggtaagttaggttgagcgacttatctgttatcaccacatttgtacaatctgtaggagaacctatgggaacgaaacgaaagcgatgccgagaatctgaatttaccaagacttaacactaactggggataccctaaacaagaatgcctaatagaaaggaggaaaaaggctatagcactagagcttgaaaatcttgcaagggtacggagtactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgacgcgttgggaaatggcaatgatagcgaaacaacgtaaaactcttgttgtatgctttcattgtcatcgtcacgtgattcataaacacaagtgaatgtcgacagtgaatttttacgaacgaacaataacagagccgtatactccgagaggggtacgtacggttcccgaagagggtggtgcaaaccagtcacagtaatgtgaacaaggcggtacctccctacttcancatatcattttctgcagccccctagaaataattttgtttaactttaagaaggagatatacatatatggctagatcgtccattccgacagcatcgccagtcactatggcgtgctgctagcgctatatgcgttgatgcaatttctatgcactcgtagtagtctgagaagggtaacgccctttacatggcaaaggggtacagttattgtgtactaaaattaaaaattgattagggaggaaaacctcaaaatgaaaccaacaatggcaattttagaaagaatcagtaaaaattcacaagaaaatatagacgaagtttttacaagactttatcgttatcttttacgtccagatatttattacgtggcgtatcaaaatttatattccaataaaggagcttccacaaaaggaatattagatgatacagcggatggctttagtgaagaaaaaataaaaaagattattcaatctttaaaagacggaacttactatcctcaacctgtacgaagaatgtatattgcaaaaaagaattctaaaaagatgagacctttaggaattccaactttcacagataaattgatccaagaagctgtgagaataattcttgaatctatctatgaaccggtattcgaagatgtgtctcacggttttagacctcaacgaagctgtcacacagctttgaaaacaatcaaaagagagtttggcggcgcaagatggtttgtggagggagatataaaaggctgcttcgataatatagaccacgttacactcattggactcatcaatcttaaaatcaaagatatgaaaatgagccaattgatttataaatttctaaaagcaggttatctggaaaactggcagtatcacaaaacttacagcggaacacctcaaggtggaattctatctcctcttttggccaacatctatcttcatgaattggataagtttgttttacaactcaaaatgaagtttgaccgagaaagtccagaaagaataacacctgaatatcgggagctccacaatgagataaaaagaatttctcaccgtctcaagaagttggagggtgaagaaaaagctaaagttcttttagaatatcaagaaaaacgtaaaagattacccacactcccctgtacctcacagacaaataaagtattgaaatacgtccggtatgcggacgacttcattatctctgttaaaggaagcaaagaggactgtcaatggataaaagaacaattaaaactttttattcataacaagctaaaaatggaattgagtgaagaaaaaacactcatcacacatagcagtcaacccgctcgttttctgggatatgatatacgagtaaggagatctggaacgataaaacgatctggtaaagtcaaaaagagaacactcaatgggagtgtagaactccttattcctcttcaagacaaaattcgtcaatttatttttgacaagaaaatagctatccaaaagaaagatagctcatggtttccagttcacaggaaatatcttattcgttcaacagacttagaaatcatcacaatttataattctgaactccgcgggatttgtaattactacggtctagcaagtaattttaaccagctcaattattttgcttatcttatggaatacagctgtctaaaaacgatagcctccaaacataagggaacactttcaaaaaccatttccatgtttaaagatggaagtggttcgtgggggatcccgtatgagataaagcaaggtaagcagcgccgttattttgcaaattttagtgaatgtaaatccccttatcaatttacggatgagataagtcaagctcctgtattgtatggctatgcccggaatactcttgaaaacaggttaaaagctaaatgttgtgaattatgtgggacgtctgatgaaaatacttcctatgaaattcaccatgtcaataaggtcaaaaatcttaaaggcaaagaaaaatgggaaatggcaatgatagcgaaacaacgtaaaactcttgttgtatgctttcattgtcatcgtcacgtgattcataaacacaagtgagatatctcgagcacccgttctcggagcactgtccgaccgctttggccgccgcccagtcctgctcgcttcgctacttggagccactatcgactacgcgatcatggcgaccacacccgtcctgtggatcgccaagctcgtcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaattaaaaatgaagttttaaaacttcatttttaatttaaattaaaaatgaagttttatcaaaaaaatttccaataatcccactctaagccacaaacacgccctataaaatcccgctttaatcccactttgagacacatgtaatattactttacgccctagtatagtgataattttttacattcaatgccacgcaaaaaaataaaggggcactataataaaagttccttcggaactaactaaagtaaaaaattatctttacaacctccccaaaaaaaagaacaggtacaaagtaccctataatacaagcgtaaaaaaatgagggtaaaaataaaaaaataaaaaaataaaaaaataaaaaaataaaaaaaataaaaaaataaaaaaataaaaaaataaaaaaataaaaaaataaaaaaataaaaaaataaaaaaatataaaaataaaaaaatataaaaataaaaaaatataaaaataaaaaaatataaaaataaaaaaataaaaaaatataaaaataaaaaaataaaaaaatataaaaatattttttatttaaagtttgaaaaaaatttttttatattatataatctttgaagaaaagaatataaaaaatgagcctttataaaagcccattttttttcatatacgtaatatgacgttctaatgtttttattggtacttctaacattagagtaatttctttatttttaaagcctttttctttaagggcttttattttttttcttaatacatttaattcctctttttttgttgcttttcctttagcttttaattgctcttgataattttttttacctctaatattttctcttctcttatattcctttttagaaattattattgtcatatatttttgttcttcttctgtaatttctaataactctataagagtttcattcttatacttatattgcttatttttatctaaataacatctttcagcacttctagttgctcttataacttctctttcacttaaatgttgtctaaacatactattaagttctaaaacatcatttaatgccttctcaatgtcttctgtaaagctacaaagataatatctatataaaaataatataagctctctgtgtccttttaaatcatattctcttagttcacaaagttttattatgtcttgtattcttccataatataaacttctttctctataaatataatttattttgcttggtctaccctttttcctttcatatggttttaattcaggtaaaaatccattttgtatttctcttaagtcataaatatattcgtactcatctaatatattgactactgtttttgatttagagtttatacttcctggaactcttaatattctggttgcatctaaggcttgtctatctgctccaaagtattttaattgattatataaatattcttgaaccgctttccataatggtaatgctttactaggtactgcatttattatccatattaaatacattcctcttccactatctattacatagtttggtataggaatactttgattaaaataattcttttctaagtccattaatacctggtctttagttttgccagttttataataatccaagtctataaacagtgtatttaactcttttatattttctaatcgcctacacggcttataaaaggtatttagagttatatagatattttcatcactcatatctaaatcttttaattcagcgtatttatagtgccattggctatatccttttttatctataacgctcctggttatccaccctttacttctactatgaatattatctatatagttctttttattcagctttaatgcgtttctcacttattcacctccccttctgtaaaactaagaaaattatatcatattttcaataattattaactattcttaaactcttaataaaaaatagagtaagtccccaattgaaacttaatctattttttatgttttaatttattatttttattaaaatattttaaactaaattaaatgattctttttaattttttactatttcattccataatatattactataattatttacaaataatatttcttcatttgtaatatttagatgatttactaattttagtttttatatattaaataattaatgtataatttatataaaaaatcaaaggagcttataaattatgattatttccaaagatactaaagatttaattttttcaattttaacaatactttttgtaatattatgtttaaatttaattgtatttttttcatataataaagccgttgaagtaaaccaatccattttccttatgatgttattattaaatttaagttttataataatatctttattatatttattgtttttaaaaaaactagtgaaatttccggctttattaaacttatttttaggaattttattttcattttcatctttacaggatttgattatatctttaaatatgttttatcaaatattatctttttctaaatttatatatatttttattatatttattattatatatattttatttttaagtttctttctaacagctattaaaaagaaacttaaaaataaaaacacgtactctaaaccaataaataaaactatttttattattgctgccttgattggaatagtttttagtaaaattaatttcaatattccacaatattatattataagctagctttgcattgtacttttcaatcgcttcacgaatgcggttatctccgaaagataaagtcttttcatcttccttgatgaagataagattttctccgtctccgccggcagaattgaagcggggtactacggtatcgtctgcgtcatcttccgttgtctgatagatgatagtcataggctcattttcttccgtttcggtaaaggggataggttcgccctttgagagcagggcggcgatggaaagcattaacttgcttttcccatcgcccggatctccctgcaatagcgtaactttgccaaacggaatatacggataccacagccactttacttctttcggctcgatttcacttgccttgatgatttcaagaggtacgctgaaattcatttcgttttcatttagtttcattttttcttgttctccttttctctgaaaatataaaaaccacagattgatactaaaaccttggttgtgttgcttttcggggcttaaatcaaggaaaaatccttgttttaagcctttcaaaaagaaacacaaggtctttgtactaacctgtggttatgtataaaattgtagattttagggtaacaaaaaacaccgtatttctacgatgtttttgcttaaatacttgtttttagttacagttgtttttagttacagacaaacctgaagttgggttgcaagcagcagattacggttcgtgttcgtgatgggttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgactgccgggcctcttgcgggataaaaagaaaaacgaaatgatacaccaatcagtgcaaaaaaagatataatgggagataagacggttcgtgttcgtgatgcttagtgggaatttgtaccccttatcgatacaaattccccgtaggcgctagggacctctttagctccttggaagctgtcagtagtatacctaataatttatctacattccctttagtaacgtgtaactttccaaatttacaaaagcgactcatagaattatttcctcccgttaaataatagataactattaaaaatagacaatacttgctcataagtaacggtacttaaattgtttactttggcgtgtttcattgcttgatgaaactgatttttagtaaacagttgacgatattctcgattgacccattttgaaacaaagtacgtatatagcttccaatatttatctggaacatctgtggtatggcgggtaagttttattaagacactgtttacttttggtttaggatgaaagcattccgctggcagcttaagcaattgctgaatcgagacttgagtgtgcaagagcaaccctagtgttcggtgaatatccaaggtacgcttgtagaatccttcttcaacaatcagatagatgtcagacgcatggctttcaaaaaccacttttttaataatttgtgtgcttaaatggtaaggaatactcccaacaattttatacctctgtttgttagggaattgaaactgtagaatatcttggtgaattaaagtgacacgagtattcagttttaatttttctgacgataagttgaatagatgactgtctaattcaatagacgttacctgtttacttattttagccagtttcgtcgttaaatgccctttacctgttccaatttcgtaaacggtatcggtttcttttaaattcaattgttttattatttggttgagtactttttcactcgttaaaaagttttgagaatattttatatttttgttcatgtaatcactccttcttaattacaaatttttagcatctaatttaacttcaattcctattatacaaaattttaagatactgcactatcaacacactcttaagtttgcttctaagtcttatttccataacttcttttacgtttccgggtacaattcgtaatcatgtcatagctgtttcctgtgtgtcatagctgtttcctgtgtgaaattcttatccgctcacaattccacacaacatacgagccggaagcataaagtgtaaagcctggggtgcctaatgagtgagctaactcacattaattgcgttgcgctcactgcccgctttccagtcgggaaacctgtcgtgccagaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgatta

SEQ ID NO: 104 - DNA, 1152 bp, Clostridium celluloyticum
atgtatatgatgagaccaatgaaacttgccggaagccagctgatgtttggtgcaggttcacttgcacacctcaagaccctgacggggaagagggcagtaatagtaactggcggctcaagcatgaaaaagagcggaattctgcaaaagaccatagattatttaaaggaagctggaatagaaagcaatgtatttgaaggggttgagcctgatccactgtttagtacggtttacagaggcgcagaggtaatgaaacagttccagcctgatataattatcggactaggaggcggttctgcaatggatgctgccaaggcaatgtgggtatattatgaacaccctgggctgacaacacttaatgacattgtaccaccaaatgtaataccaaagctaaggaataaggctattctggtatgtataccttcaaccagcggtacagccagcgaagtcagccgttctgtggtaattaccgatgacgaaacccacataaagtatggggtaggaaatatggaaatgatgccggatatcgcaatatgcgatcccgaggttactgttacaatgccggcaaaaattactgctgaaacaggtatggatgcattaactcatgcactggaagcacttgcttcaaacagagcaaattatattagcaatatattcgcaaaaaacgcagcaaaggatattatagtaaatctaccaaaagcgtacagccaaggtaatgatattggaagcagggaaattatgataaatgcatccatggctgcgggaatggcgtttaccaatgtttcattgggaatagttcattctatggcacacactttgggaagcttttttggaatttcacacggacttgctgatgctgtgatactgccctatgtgatggaatttaattcacataatgctgacgcaaagaaactatacgatgaattggcaaaagagctaggtgcggcggatttagttaatgtggtatgcgagctgaataagtccatcggtattccaagccattttgcagaactcataccggacgaaaagaaatatatggatgagctggacgcaatggcagaaatggctctaaaggacggatgtactaaaacaaatcctgttattccttcaatagaacaactcaaggaactatttattaaagtatataaagcataa

SEQ ID NO: 105 - DNA, 1176 bp, Clostridium celluloyticum
atgctaaactttgatatgcacataccgactcgtataatttttggcagggatgcccaaaaagaaataggcagtttgataaagccgtatacaaaaaaggcacttttgcattacggcggaaacagtattaagaatagcggattatatgaccaggtggtcgcttctctaaaggaaaataacatagaatttgttgaacttgggggcgtagttcccaatccccgtctggccttagccgaagaaggggctgcactctgcaaaaaagaaggagtcgaccttatacttgcagtaggcggcggaagtgtaatagattcagcaaaggcaatagcattaggtgcatgtaatgatgaaatctggaagttttacgaagaaggcggtgagattaaatctgcactgcctgttgcaactatcctaactatacctgcggcaggaagtgaatccagccctcatagcgtaattaccaacgaagaaaaacagattaaaaagggttatggtagcaatctacttaaacctattctgagcatagtaaacccggaattgttttttacacttcccaagaaccaaatctctaatggtgtagcagatatgatgagccatatttttgagagatactttacaaataccaagaatacagaccttacagacggcttgtgcgagacgacacttaaaaccattatgaaaaatgcacttatcgtttcaaagaatcctcaaaattacgatgcttggtgtcaggttggatttggtggcactatggcacataacgacatactggggctcggaagagaacaggattgggcttgccacgatatggagcatgagctcagtgccatttatgatattgcacatggggcgggccttgcgatattaactcctgcatggatgcagtatgtatataagaccaacattaatatgtttgtgcagtttgcagttaacgtaatgggagtagagagcagttatcgtgaccttgatgccattgtgatggagggaatagcaagactccgtgaattttataaaaagatgggacttccgtccactctcacagaagttggcatagaagagagtaagtttgagcttatggccagaaaagcaaccggtgaggcatatggaaaagaacattcaattggaggactcaaaaaactatactggcaagatgtccttgaaatatacaagcttgcgaagtaa

SEQ ID NO: 106 - DNA, 1200 bp, Clostridium celluloyticum
atgtgggaaaacaagattgacattaatcaggtagtagaaataagggcaaaaacaacaacgttctttggtgtgggagctatcaacaagatggcggatattgcggcaaacttatctgggagaggtataacaaaagttatagtcatgacaggaaaaagttcacatattaagaccggtgcatgggacgtaacaaaaaaagctcttgaggacaatggaattaaatacttactgtacagcaaggttactccgaatcctacagttgatcaggttgatgaagcaactgcagaagctgaaacctttggtgccgaggctgttattgcaatcggagggggaagcccaatagatgcggctaagagcgttgcaatacttctggcttatcctgaaaagaatgcaactgagctttatgaactcacctttgttcctgacaaggcagttcctgttattgctataaatcttacccacggaacaggtactgaagttgacagatttgcggttgtaagtattccggctaaagaatacaagcctgcaattgcatacgactgcatttatcctttgtttgcgattgatgatccttcattgatgacaaaattacctgctgaacagaccatatatgtatcagtagatgctataaatcatgttgttgaggcttcaacaacaatagtaacaaatcctttggctatacttttggcaaaagaaacaatcagactggttgcaaaatatctgccgattgccataaaggaaccggacaatctaactgcaagatactatctattatatgcttcattgattgcaggaacttcctttgacaatggcttactgcattatacacatgcacttgaacatcctttaagtgctgtaagacctgatcttgcacacggacttggacttgctatgctccttccggctgttgtaaaggaaatttaccctgcatcaggggaggttctggcagagatttttgaaccaattcttccgggtttaaaaggtacggctgatgaggctgacaaggcgtttacaggtttgagaaactggattgagagtgttggtataaagtcacatttaagggatgaaggctttgatgacagtgttgttgataaacttgtaaatctggcattcgagactcctagtcttgatggactgttagctcttgcacctgttaaggcaacaaaggaatctgtaaaaaacatttatgttaattcgctctaa

SEQ ID NO: 107 - DNA, 2577 bp, Thermoanaerobacterium saccharolyticum
atggcaacgacaaaaacggaattagacgttcagaagcagatagatctacttgtgtcaagagcacaagaggctcagaaaaaattcatgtcttacacgcaagagcaaatcgacgcaatagttaaggcaatggctttagcaggcgttgacaaacacgtagagctggcaaagatggcgtacgaagagacaaaaatgggtgtatacgaagataagataacaaaaaatctcttcgcaacagagtacgtgtaccacgacataaaaaatgaaaagactgtaggaatcataaacgagaacatagaagaaaactacatggaagtggcagaaccgataggcgtaattgccggtgtcacacctgtcacaaacccaacatctaccacgatgtttaaatgcttaatatccataaagacgcgaaatcctataatattcagcttccatccaaaggcaataaagtgcagcatcgcagcagccaaagtgatgtatgaagctgcactaaaggcaggcgcacctgaaggatgcataggatggatagaaacgccatcaattgaggccacacagcttctcatgacacatccaggcgtatcgctgatccttgcaacgggcggtgcaggaatggtaaaagcggcatacagctcaggaaaaccggcattaggcgtaggtcctggcaatgtgccatgctacatcgaaaaatcagcaaacataaagagggctgtatcggatctcatactaagcaagacatttgacaatggagtaatatgcgcatcagagcaggccgtaataatagacgaggaaatagcagatgaagtcaaaaagcttatgaaagaatacggctgctacttcttaaacaaagatgaaataaagaagcttgagaaatttgcaattgatgagcaaagctgcgccatgagccctgcagtggtaggtcagccagcggcgaagattgctgaaatggcaggcttcaaagtccccgaaggcacaaagatattagtggcagagtacgaaggagtaggtccaaaatatcctctatcaagggagaaactaagcccgattcttgcttgctacaccgtcaaagactacaatgaaggaatcaaaaagtgcgaggaaatgactgaattcggaggtttaggccactctgctgtaatacactctgaaaatcaaaacgtcataaatgaatttgcaaggcgagtccgcacaggaagacttatcgtaaattcaccatcatcacagggagcaataggagatatatacaatacaaacacgccatcacttacattaggctgtggttctatgggaagaaactcaacgacagacaatgtaagcgtcaagaaccttttgaatattaagcgtgtcgtgataaggaatgatagaatgaaatggttcaagattccaccgaagatttactttgaaagcgggtcactccagtacctgtgcaaagtcaaaagaaaaaaagcgtttatcgtcacagatccattcatggttaagcttggcttcgtagacaaagtgacatatcaattagacaaagcaaacatcgaatacgaaatattctcagaagtagagccagatccatctgttgacacagtcatgaacggcgtaaaaataatgaattcgtacaatcctgacttaataatcgctgtaggcggtggctctgcaatagacgcagcaaagggaatgtggcttttctacgaatatcctgatacagagtttgaaacattgaggcttaaatttgcagacatcagaaaaagggcatttaagttcccagaacttggcaaaaaagcgctattcatcgcaataccgacaacaagcggcacaggctcagaagtgacagcatttgccgtaataaccgacaaaaagagaaacatcaagtatccactggcagactacgaacttacacctgacatagccataatagatcctgaccttacaaagactgtaccgccatctgtaacagcagacacaggcatggatgtgctgacacacgccatagaagcatacgtatcagtaatggcatcagactacacagatgcactggcggaaaaggctataaagatcgtatttgaatacctgccaagggcttataaaaacggcaatgatgaagaagcccgcgaaaagatgcacaatgcttcctgcatggctggtatggcattcacaaatgcattcttaggaataaaccacagcatggcacacatactgggcggaaagttccacataccacacggaagagcaaatgcaatacttctgccgtatgtaataaggtacaatgcagaaaaacctacaaagtttgtggcattcccacaatacgaatatccaaaagcagcagaaagatatgcggaaatcgccaaattcttaggactgcctgcttcaactgttgaagaaggcgtagaaagcttaatagaagctataaagaacctcatgaaagagcttaacattccgcttacacttaaagacgccggcatcaacaaagaacagtttgaaaaagaaatagaggaaatgtcagacatcgccttcaacgatcagtgcacagggacaaacccgagaatgcctctcacaaaagaaattgcagagatctacagaaaagcatacggtgca

SEQ ID NO: 108 - protein, 859 aa, Thermoanaerobacterium saccharolyticum
MATTKTELDVQKQIDLLVSRAQEAQKKFMSYTQEQIDAIVKAMALAGVDKHVELAKMAYEETKMGVYEDKITKNLFATEYVYHDIKNEKTVGIINENIEENYMEVAEPIGVIAGVTPVTNPTSTTMFKCLISIKTRNPIIFSFHPKAIKCSIAAAKVMYEAALKAGAPEGCIGWIETPSIEATQLLMTHPGVSLILATGGAGMVKAAYSSGKPALGVGPGNVPCYIEKSANIKRAVSDLILSKTFDNGVICASEQAVIIDEEIADEVKKLMKEYGCYFLNKDEIKKLEKFAIDEQSCAMSPAVVGQPAAKIAEMAGFKVPEGTKILVAEYEGVGPKYPLSREKLSPILACYTVKDYNEGIKKCEEMTEFGGLGHSAVIHSENQNVINEFARRVRTGRLIVNSPSSQGAIGDIYNTNTPSLTLGCGSMGRNSTTDNVSVKNLLNIKRVVIRNDRMKWFKIPPKIYFESGSLQYLCKVKRKKAFIVTDPFMVKLGFVDKVTYQLDKANIEYEIFSEVEPDPSVDTVMNGVKIMNSYNPDLIIAVGGGSAIDAAKGMWLFYEYPDTEFETLRLKFADIRKRAFKFPELGKKALFIAIPTTSGTGSEVTAFAVITDKKRNIKYPLADYELTPDIAIIDPDLTKTVPPSVTADTGMDVLTHAIEAYVSVMASDYTDALAEKAIKIVFEYLPRAYKNGNDEEAREKMHNASCMAGMAFTNAFLGINHSMAHILGGKFHIPHGRANAILLPYVIRYNAEKPTKFVAFPQYEYPKAAERYAEIAKFLGLPASTVEEGVESLIEAIKNLMKELNIPLTLKDAGINKEQFEKEIEEMSDIAFNDQCTGTNPRMPLTKEIAEIYRKAYGA

SEQ ID NO: 109 - DNA, 6788 bp, Artificial Sequence
  Synthetic polynucleotide
tctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggtgcagatggatacatgccttgaagatagcaataatcacatgagtaaatgcaatttaaaacattcgatgtgtggtaaaactcatcaatgccaaagttttcgcacatctgagatcctctgtatataaaatcgtaccttttctttgccagtattatatttggatatttcttctgcatgacataattctgcctttgccttaaaaatacatcgctatacctttgtatttcaataacagtgctttttttcaatgcatctaatattttttctgcaattgggtggtttaaaacatctttttcaacgtaaacatgagaaaatttcatatgccactcatcctctcaaagtgtataaagataaagataaagaaaaaagcgctcatagcgccatttaaacatcgttaagcttgtgtatatctaaaagcagttttacatccgcttctctttcaagtatgtgatcattcaaaacatcaaacttcttgtctattctcttaaaattcaattcaaacttgccaagctggcttgtatgctttgttacttcttcaaatgtctgttttaaaatgtaatcaacatcgctttgttgcctttcaaggttgtaaagtctattctctacactatcaagcctcttgtccatgccgtcaagtctattttcaacaccgtcaagccttctctcaacgttatccagtcttctctcaacgttgtccagccttttatccatattatccagtcttttgtctacatcgtccaatcttttgtctacgttgtccaatctcttttcaacactgacaagtctttcatccatacttcccagtttcccaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaacggaagtgtaggtcctaagtgaatattctttacccccaggtacagaagtgcaagtaatactattacagctttttgctcataccatgcaatgttaaatgatattggaagctcatttatatcttcaagtccaaacacttctttaagtttaagcgcaatcactgccaatgaatacgagtcattgcactgtcctgcgtcaagcactcttggtatgccgtttatatcgccaagatttaatttattgtacctgtactttgcacagcctgctgtcaatatgaccgtatcttttggaagttcttttgcaaattctgtgtagtattctcttgatttcatcctgccatcgcagcctgccattacaaagaacctctttatagcgccagtctttactgcatcaactacttgattagccaaagccaatacttggttgtgggcaaatcctcctactatttctccgctctcaatctctactggcggtttgcatctctttgcgtgctctattatttctgtaaagtcttttttcccatctgggccagcatcaatatgctttacaccttcaaatccaacgacaccagtcgtgtaaagtctgtctttgtatgaatcctttggaggcgtcaagcagttagttgtcatgagtattgggccgttaaagctttcaaattctttatcctgctgccaccatgcattgccgtagtttcctgcaaagtgtgaatacttcttaaatgctggatagtagtgggctggaagcatttcaccgtgtgtatatacgtcaacacccgttccttctgtctgctccaacagctcttccaagtcttttaagtcgtgtccgcttattaagatgccaggattatttctgacgccgatatttacctttgtaatctcaggatttccgtatgtggac

SEQ ID NO: 110 - DNA, 6058 bp, Artificial Sequence
  Synthetic polynucleotide
agcttatgaaagaatacggctgctacttcttaaacaaagatgaaataaagaagcttgagaaatttgcaattgatgagcaaagctgcgccatgagccctgcagtggtaggtcagccagcggcgaagattgctgaaatggcaggcttcaaagtccccgaaggcacaaagatattagtggcagagtacgaaggagtaggtccaaaatatcctctatcaagggagaaactaagcccgattcttgcttgctacaccgtcaaagactacaatgaaggaatcaaaaagtgcgaggaaatgactgaattcggaggtttaggccactctgctgtaatacactctgaaaatcaaaacgtcataaatgaatttgcaaggcgagtccgcacaggaagacttatcgtaaattcaccatcatcacagggagcaataggagatatatacaatacaaacacgccatcacttacattaggctgtggttctatgggaagaaactcaacgacagacaatgtaagcgtcaagaaccttttgaatattaagcgtgtcgtgataaggaatgatagaatgaaatggttcaagattccaccgaagatttactttgaaagcgggtcactccagtacctgtgcaaagtcaaaagaaaaaaagcgtttatcgtcacagatccattcatggttaagcttggcttcgtagacaaagtgacatatcaattagacaaagcaaacatcgaatacgaaatattctcagaagtagagccagatccatctgttgacacagtcatgaacggcgtaaaaataatgaatttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcg

SEQ ID NO: 111 - DNA, 1080 bp, Thermoanaerobacterium saccharolyticum
ttgctggttataggcagcggcaattctatgagaaggcatggttatttggatagagttgttaattacttaaaacaaaatggggttgattacgtcgtctatgataagatattacctaatccaattgcagagcatgttgcggaaggagcaaaagtggcaaaggacaatggatgtgattttgtcataggattaggaggcggtagtacgatagattcatcaaaagcaatagctgttatggctaaaaatccaggtgattattgggattatgtttctggcggcagcggaaaaggcatggaagttaaaaatggtgcattgcctatcgttgctatccctacgacggcaggtactggcacagagtctgatccgtgggctgttgtgacaaagacagagacaaatgagaagattgggtttggatgtaagtatacatatcctacactttcaatcgttgatcctgaattgatggtgtcaattccaccgaaatttactgcttatcaaggtatggatgcatttttccactcagtggaaggatatttggcgactgtaaatcagccaggaagtgatgtcttagcgcttcaatccataagccttattacagagaaccttccaaaagctgtggcagatggcaataatatggaagctcgtactgcgttggcgtgggcaagtactgctgcaggcatagtagaatcattgtcatcgtgcatttctcaccattcgttagaacacgctttaagtgcataccatccagagataccgcatggtgctggacttataatgctttcggtgtcttattttagcttcatggcttcaaaggctcctgaaagatttgtagatatagcaaaagcaatgggtgaggagatagttgggaatactgtagaagaacaagccatgtgctttataaatgggcttaaaaaattgattagaaatataggaatggaagatctaagcttgtcaagctttggagtaactgaagatgaagcgacaaagttagcgaagaatgctatggatacaatgggtggattgtttaatgtcgatccatacaagctatcattagatgaggtagtatccatatacaaaaattgtttc

SEQ ID NO: 112 - DNA, 1197 bp, Thermoanaerobacterium saccharolyticum
atgtgggaaacaaaagtaaatcctagcaaaatttttgaacttcgctgtaaaaatacgacttattttggtgttggcagtatacataagataaaggatatattagaaaatctaaaaattaatggcattaataatgttatttttataacaggcaaaggttcgtataagactagcggagcttgggatgttgtaaggcctgtattggaagagcttgatttaaaatattcactttacgataaagttggtccaaatcctaccgttgatatgatagacgaagcggcaaaaattggacgagaatctggagcgaaagccgtaattggaattggcggtggtagccctatagatactgcaaaaagtgtagctgtgctattaaagtatactgataaaaatgcacgagagctttataagcaaaaatttattcctgatgatgctgttcctataatagctataaatcttacacatggtacaggaacagaggtagatagatttgcagtggctactatacctgagaaaaattataaaccagctatagcttatgattgcttatatcctatgtttgctatagatgacccttcgcttatgacaaagcttgataaaaaacagactatagccgttactgttgatgctttaaatcacattacagaagctgctacgacacttgttgcatctccatatagtattcttacggctaaggaaacagtaagattgattgttcgatatcttcccgctgctgtaaatgatcctttaaatatagtggcaagatattaccttctgtatgcttctgcattagctggcatatcttttgacaatggtcttttgcatcttactcatgctttagagcatcctttaagtgctgtaaaacctgaaatagctcatggtttagggcttggtgcaatattgccagcagttataaaagctatttatccagctacagcagaagtattggctgatgtatatagtcctatagttcctggtttaaaaggactgcctgttgaggcggagtatgtagcagaaaaagttcaagaatggctttttagtgtggggtgcattcaaaagttatctgattttgggtttacgaaagatgatattcctaatcttgtaaaacttgctaaaacaactccttcccttgatgggttgttatctattgcacctgttgaagctactgaatctgtcattgaaaagatttatttaaaatctctt

SEQ ID NO: 113 - DNA, 1272 bp, Thermoanaerobacterium saccharolyticum
ttgttacatatcaagcggtatcatagcataccatcaaaatatgtttttcaaaaaaccagtggtataattatctcaaaagaaacattaaaaagagggtgtgattttatgaacaattttgattttgtttgtccgacaaagatcatctttggcagagggactgaaaatagagtaggtgaagagacaaaaaaatattcaaaaaaggtattgtttgtttacggcagtggtagcataaaaaagacaggtctttacgacaaagtcgtaaaatcgctaaaaggcaatggtgttgattacatagaattatcaggtgttaaaccaaatccaaggcttagcttggtttatgaaggaataaagatttgcaaggaaaatggcattgacttcatattggccgtaggtgggggtagcgcgatagacactgcaaaggctatcgctgttggtgcgctttatgatggtgatgtgtgggatttctttgtaggcaaagctgagattgaaagggcattaccagtaggtgtaatactgacattggctgcaacaggaagtgaagccagcgatagcgctgtcataacaaatgaagatggatggtacaagaagggcatacactctgatcttataaggcctcagtttgccatcatgaatccagagcttttgtttacacttcctcagtatcagacggctgcaggtgcagcggatataatggctcacataatggaaaggtactttacaaacgtcaggagtgtagatcttacggataggctttgcgaagcaacgcttaagaccatgataaacaatgttccaaggcttattgaaaatcctgaggattatgatgtgagagctgaagtgatgtgggcaggtactattgcacataatggacttttggatacgggaagaatcggcgattgggcatcacataggattgaacatgagttaagtgcaatctacgacatagcacacggcgcaggacttgccgtaatattcccagcgtggatgaaatacgtgtataaacacgacttggatagatttgtccagtttgccgttaaagtttgggatgtagatttgacatttaccgacagagaagcgatagcgttggaaggcataaaaagacttgaggatttctttagaaacattggccttcctgtgacactgagggaggctaatataccttacgacaggtttgaagaaatggcagacaaatgcacgtcaaatgggacagcaaccgtaggacaatttgttaaacttacgaaagaagacataataaatatatacaatcttgcaaaa

SEQ ID NO: 114 - DNA, 1509 bp, Clostridium thermocellum
tttgatcctggctcaggacgaacgctggcggcgtgcctaacacatgcaagtcgagcggggatatacggaaggtttaccggaagtatatcctagcggcggacgggtgagtaacgcgtgggtaacctacctcatacagggggataacacagggaaacctgtgctaataccgcataacggggcggcatcgtcctgttatcaaaggagaaatccggtatgagatgggcccgcgtccgattagctagttggtgaggtaacggctcaccaaggcgacgatcggtagccgaactgagaggttggtcggccacattgggactgagacacggcccagactcctacgggaggcagcagtggggaatattgcgcaatgggggaaaccctgacgcagcaacgccgcgtgaaggaagaaggccttcgggttgtaaacttctttgattggggacgaaggaagtgacggtacccaaagaacaagccacggctaactacgtgccagcagccgcggtaatacgtaggtggcgagcgttgtccggaattactgggtgtaaagggcgcgtaggcgggatgcaagtcagatgtgaaattccggggcttaaccccggggctgcatctgaaactgtatctcttgagtgctggagaggaaagcggaattcctagtgtagcggtgaaatgcgtagatattaggaggaacaccagtggcgaaggcggctttctggacagtaactgacgctgaggcgcgaaagcgtggggagcaaacaggattagataccctggtagtccacgccgtaaacgatggatactaggtgtaggaggtatcgaccccttctgtgccggagttaacacaataagtatcccacctggggagtacggccgcaaggttgaaactcaaaggaattgacgggggcccgcacaagcagtggagtatgtggtttaattcgaagcaacgcgaagaaccttaccagggcttgacatccctctgacagctctagagatagggcttccttcggggcagaggagacaggtggtgcatggttgtcgtcagctcgtgtcgtgagatgttgggttaagtcccgcaacgagcgcaacccttgtcgttagttgccagcacgttaaggtgggcactctagcgagactgccggcgacaagtcggaggaaggtggggacgacgtcaaatcatcatgccccttatgtcctgggctacacacgtactacaatggctgctacaaagggaagcgataccgcgaggtggagcaaatccccaaaagcagtcccagttcggattgcaggctgaaactcgcctgcatgaagtcggaattgctagtaatggcaggtcagcatactgccgtgaatacgttcccgggccttgtacacaccgcccgtcacaccatgagagtctgcaacacccgaagtcatagtctaaccgcaaggagggcgctgccgaaggtggggcagatgattggggtgaagtcgtaacaaggtagccgtatcggaaggtgcggctggatcacctccttt

SEQ ID NO: 115 - DNA, 1642 bp, Clostridium cellulolyticum
  misc_feature (9)..(9): n is a, c, g or t
tgatcctgngacaggncgagcgctgncggcgtgcctaacacatgcgagtcgagcggagttacctttagcnctgagtattcttgganatgatgctgncccgacagcgtcatccnnnaacaaccttaatgaaatatttagttggagttttgcatcacgcgttttatcaaagtgtcaacacataatagtagaagagaatgttcagtgctgaaggtaacttagcggcggacgggtgagtaacgcgtgggcaacctgcctgttacagggggataacacagggaaacttgtgctaataccgcataacacaacgaagaagcatttcnttgttgtcaaaggagcaatccggtgacagatgggcccgcgtccaattagctagttggtgatgtaacggatcaccaaggcgacgattggtagccgaactgagaggttgatcggccacattgggnctgagacacggcccagactcctacgggaggcagcagtggggaatattgcacaatgggggaaaccctgatgcagcaacgccgcgtgaaggatgaaggttttcggattgtaaacttctttagtcagggacgaagaaaatgacggtacctgaagaataagccacggctaactacgtgccagcagccgcggtaatacgtaggtggcaagcgttgtccggaattactgggtgtaaagggcgtgtaggcgggaatgtaagtcagatgtgaaatcccagggcttaaccctggagctgcatctgaaactatgtttcttgagtgccggagaggaaagcggaattcctagtgtagcggtgaaatgcgtagatattaggaggaacaccagtggcgaaggcggctttctggacggtaactgacgctgaggcgcgaaagcgtggggagcaaacaggattagataccctggtagtccacgctgtaaacgatggatactaggtgtaggaggtatcgaccccttctgtgccggagttaacacaataagtatcccacctggggagtacggccgcaaggttgaaactcaaaggaattgacgggggcccgcacaagcagtggagtatgtggtttaattcgaagcaacgcgaagaaccttaccaaggcttgacatatagcggaatncggcagagatgtcgtagtccttcgggactgctatacacaggtggtgcatggttgtcgtcagctcgtgtcgtgagatgttgggttaagtcccgcaacgagcgcaacccctgttgctagttgataacattaagatgatcactctagcgagactgccggtgacaaatcggaggaaggtggggacgacgtcaaatcatcatgccccttatgtcttgggctacacacgtactacaatggctataacagagggaagctaagctgcaaagtggagcaaatccccaaaaatagtcccagttcagatggtgggctgcaacccgcccacatgaagtcggaattgctagtaatggtaggtcagtatactgtcgtgaatacgttcccgggccttgtacacaccgcccgtcacaccatgagagtctgcaacacccgaagtcgatagtctaaccgcaaggaggacgtcgccgaaggtggggccgatgattggtgtgaagtcgtaacaaggtagccgtatcggaaggtgcggctggatcacctccttt

SEQ ID NO: 116 - DNA, 1552 bp, Thermoanaerobacterium saccharolyticum
  misc_feature (64)..(64): n is a, c, g or t
tttgatcctggctcaggacgaacgctggcggcgtgcctaacacatgcaagtcgagcgatccggnactcaattaagcgcttacagaaaaagagagagaaantgagtaaacgcaaagttgagtgccggatagcggcggacgggtgagtaacgcgtggacaatctaccctgtagtttgggataacacctcgaaaggggtgctaataccggataatgtcaagaagtggcatcactttttgaagaaaggagaaatccgctataggatgagtccgcgtcccattagctagttggcggggtaaaagcccaccaaggcgacgatgggtagccggcctgagagggtgaacgnccacactggaactgagacacggtccagactcctacgggaggcagcagtggggaatattgttcaatgggggaaaccctgacacagcgacgccgcgtgagcgaagaaggccttcgggtcgtaaagctcaatagtatgggaagatagtgacggtaccatacgaaagccccggctaactacgtgccagcagccgcggtaatacgtagggggcgagcgttgtccggaattactgggcgtaaagagcacgtaggcggctgtaaaagtcagatgtgaaaaacctgggctcaaccgagggtgtgcatctgaaactaaacagcttgagtcaaggagaggagagcggaattcctggtgtagcggtgaaatgcgtagagatcaggaagaataccagtggcgaaagcggctctctggacttgaactgacgctgaggtgcgaaagcgtggggagcaaacaggattagataccctggtagtccacgccgtaaacgatggatactaggtgtgggtgaagcatcatccgtgccggagttaacgcaataagtatcccgcctggggagtacggccgcaaggttgaaactcaaaggaattgacgggggcccgcacaagcagcggagcatgtggtttaattcgaagcaacgcgaagaaccttaccagggcttgacatccacagaatcaggtagaaataccagagtgcctcgaaagaggagctgtgagacaggtggtgcatggttgtcgtcagctcgtgtcgtgagatgttgggttaagtcccgcaacgagcgcaacccctgttggtagttaccagcgtaaagacggggactctaccgagactgccgtggagaacacggaggaaggcggggatgacgtcaaatcatcatgccctttatgccctgggctacacacgtgctacaatggcctgaacagagggcagcgaaggagcgatccggagcgaatcccagaaaacaggtcccagttcagattgcaggctgcaacccgcctgcatgaagacggagttgctagtaatcgcggatcagcatgccgcggtgaatacgttcccgggccttgtacacaccgcccgtcacaccacgagagtttacaacacccgaagtcagtgacctaaccgcaagggaggagctgccgaaggtggggtaaatgattggggtgaagtcgtaacaaggtagccgtatcggaaggtgcggctggatcacctcctttccct

SEQ ID NO: 117 - DNA, 1519 bp, Clostridium stercorarium
tttgatcctggctcaggacgaacgctggcggcgtgcctaacacatgcaagtcgaacgggatccgtgttacggaggtctttggaccgaagtggcatggtgagagtggcggacgggcgagtaacgcgtgagcaacctgccctatgctgggggataacaccgggaaaccggtgctaataccgcataagaccacagtgacgcatgtacagtggtaaagctgaggcggcataggatgggctcgcggtccattagctagttggtagggtaacggcctaccaaggcgacgatcggtagccggactgagaggttggccggccgcattgggactgagacacggcccagactcctacgggaggcagcagtggggaatattgcgcaatgggggaaaccctgacgcagcgacgccgcgtggaggaagaaggcctttgggttgtaaactcctttgatcggggacgaagatgacggtacccgaagaacaagccacggctaactacgtgccagcagccgcggtaatacgtaggtggcgagcgttgtccggaattactgggtgtaaagggcgtgtaggcggggtgccaagtcaggtgtgaaataccggggcttaacctcgggggtgcatctgaaactggtgctcttgagtgccggagaggaaagcggaattcccagtgtagcggtgaaatgcgtagatattgggaggaacaccagtggcgaaggcggctttctggacggtaactgacgctgaggcgcgaaagcgtggggagcaaacaggattagataccctggtagtccacgctgtaaacgatggatactaggtgtaggaggtatcgaccccttctgtgccgtagttaacacaataagtatcccacctggggagtacggccgcaaggctgaaactcaaaggaattgacgggggcccgcacaagcagtggagcatgtggtttaattcgaagcaacgcgaagaaccttaccagggcttgacatccccctgacggatgtagagatacatcttctccgcaaggagcaggggagacaggtggtgcatggtgcagctcagctcgtgtcgtgagatgttgggttaagtcccgcaacgagcgcaacccttgtcgttagttgccagcagtaagatgggcactctaacgagactgccggcgagaagtcggaggaaggtggggatgacgtcaaatcatcatgccccttatgtcctgggctacacacgtgctacaatggcgactacagagggaagcaaatccggcaggaggagcaaatcccgaaaggtcgtcccagttcggattgcaggctcgaactcgcctgcatgaagccggaattgctagtaatggcaggtcagcatactgccgtgaatacgttcccgggccttgtacacaccgcccgtcacaccatgagagctggcaacacccgaagccgtagcctaaccgagaggggggcgccgtcgaaggtggggcaggtgattggggtgaagtcgtaacaaggtagccgtatcggaaggtgcggctggatcacctccttt

SEQ ID NO: 118 - DNA, 1500 bp, Clostridium stercorarium II
cctggctcaggacgaacgctggcggcgtgcctaacacatgcaagtcgaacgggatccgtgttacggaggtcttcggaccgaagtggcatggtgagagtggcggacgggcgagtaacgcgtgagcaacctgccctatgctgggggataacaccgggaaaccggtgctaataccgcataagaccacagtgacgcatgtcacagtggtaaaagctgaggcggcataggatgggctcgcgtccgattagctagttggtagggtaacggcctaccaaggcgacgatcggtagccggactgagaggttggccggccgcattgggactgagacacggcccagactcctacgggaggcagcagtggggaatattgcgcaatgggggaaaccctgacgcagcgacgccgcgtggaggaagaaggcctttgggttgtaaactcctttgatcggggacgaagatgacggtacccgaagaacaagccacggctaactacgtgccagcagccgcggtaatacgtaggtggcgagcgttgtccggaattactgggtgtaaagggcgtgtaggcggggtgccaagtcaggtgtgaaataccggggcttaacctcgggggtgcatctgaaactggtgctcttgagtgccggagaggaaagcggaattcccagtgtagcggtgaaatgcgtagatattgggaggaacaccagtggcgaaggcggctttctggacggtaactgacgctgaggcgcgaaagcgtggggagcaaacaggattagataccctggtagtccacgctgtaaacgatggatactaggtgtagaccccttctgtgccgtagttaacacaataagtatcccacctggggagtacgaggtatcgggccgcaaggctgaaactcaaaggaattgacgggggcccgcacaagcagtggagcatgtggtttaattcgaagcaacgcgaagaaccttaccagggcttgacatccccctgacggatgtagagatacatcttctccgcaaggagcaggggagacaggtggtgcatggttgtcgtcagctcgtgtcgtgagatgttgggttaagtcccgcaacgagcgcaacccttgtcgttagttgccagcagtaagatgggcactctaacgagactgccggcgagaagtcggaggaaggtggggatgacgtcaaatcatcatgccccttatgtcctgggctacacacgtgctacaatggcgactacagagggaagcaaatccgcgaggaggagcaaatcccgaaaggtcgtcccagttcggattgcaggctgcaactcgcctgcatgaagccggaattgctagtaatggcaggtcagcatactgccgtgaatacgttcccgggccttgtacacaccgcccgtcacaccatgagagctggcaacacccgaagccggtagcctaaccgagaggggggcgccgtcgaaggtggggcacccgaagccggtagcctaaccgagaggggggcgccgtcgaaggtgggg

SEQ ID NO: 119 - DNA, 1508 bp, Caldiscellulosiruptor kristjanssonii
  misc_feature (213)..(213): n is a, c, g or t
ggctcaggacgaacgctggcggcgtgcctaacgcatgcaagtcgagcggagatggtagctgaaggtgatgagctggaagctatcatcttagcggcggacgggtgagtaacacgtgagcaacctaccctcagcacggggataacagctcgaaagggctgctaatacccgatgggaccacggcatcgcatggtgctgtggtgaaagggtagccgnagaggctatnccggctggggatgggctcgcggcccatcagctagttggtggggtaacggcctaccaaggcgacgacgggtagccggcctgagagggtgtacggccacagtgggactgagacacggcccacactcctacgggaggcagcagcggggaatcttgcgcaatgggcgaaagcctgacgcagcgacnccgcgtgagggaagaagcccttcggggtgtaaacctctttggacggggagaagtggaagatagtacccgtttaaaaagccacggctaactacgtgccagcagccgcggtaatacgtaggtggcgagcgttgtccggaattactgggcgtaaagggtgcgtaggcggcctggtaagttgagcgtgaaatttttgggctcaacccaaaaggagcgctcaagactgccgggcttgagtgcgggagaggacggcggaattcccggtgtagcggtgaaatgcgtagatatcgggaggaacaccagtggcgaaggcggccgtctggaccgtaactgacgctgaggcacgaaagcgtggggagcaaacaggattagataccctggtagtccacgctgtaaacgatggatgctaggtgtgggggagaagaactcttccgtgccgtagttaacacaataagcatcccgcctggggagtacggtcgcaaggttgaaactcaaaggaattgacgggggcccgcacaagcggtggagcatgtggtttaattcgaagcaacgcgaagaaccttaccagggcttgacatgccgggaaccctgccgaaaggcgggggtgcctgcttgttaagagcaggagcccggacacaggtggtgcatggttgtcgtcagctcgtgtcgtgagatgttgggttaagtcccgcaacgagcgcaacccctgcccttagttgccagcggttttagccgggcactctaaggggactgccgccgatgaggcggaggaaggtggggatgacgtcaaatcatcatgccccttatgccctgggctacacacgtgctacaatgggtgctacagagggcggcgaaggcgcgagccggagcgaatcccaaaaaagcacccccagttcggattgcaggctgcaactcgcctgcatgaagtcggaatcgctagtaatcgcggatcagcatgccgcggtgaatacgttcccgggccttgtacacaccgcccgtcacaccatgagagtcagcaacacctgaagacacaggatatctgtgttgaaggtggggctgatgattggggtgaagtcgtaacaaggtagccgtacgggaacgtgcgg

SEQ ID NO: 120 - DNA, 1370 bp, Clostridium phytofermentans
  misc_feature (240)..(243): n is a, c, g or t
cttagtggcggacgggtgagtaacgcgtgggtaacctgcctcatacagggggataacagtcggaaacgattgctaaaaccgcataatatagcgaaaccgcatgattttgctatcaaatatttataggtatgagatgggcccgcgtctgattagctagttggtggggtaatggcctaccaaggcgacgatcagtagccggcttgagagagtgaccggccacattgggactgagacacggcnnnnactnctncgggaggcagcagtggggaatattggacaatgggggaaacccngatccagcgacgccgcgtgagtgaagaagtatttcggtatgtaaagctctatcagcagggaagataatgacagtacctgactaagaagccccggctaactacgtgccagcagccgcggtnatacgtannnnnnnagcgttatccggatttactgggtgtaaagggagcgtaggtggtaggtcaagtcagatgtgaaagnccagggctcaaccctggnnctgcatttgaaactggctnactgagtgcaggagaggtaagtggaattcctagtgtagcggtgaaatgcgtagatantaggaggaacaccagtggcgaaggcggcnnactggactgtaactgacactgaggctcgnnngcgtggggagcaaacaggattagatnccctggtagtccncgccgtaaacgatgaatactagctgttcggggtcnnacagggcttcggtggcgcacgtaacgcaataagtattccacctggggngtacgttcgcaagaatgaaactcaaaggaattgacgggganncgcacaagcggtggagcatgtggtttaattcgaannaacgcgaagaaccttaccaagtcttgacatccctctgacaaccgagtaacgtcggnnttcttcgggncagaggngacaggtggtgcatggttgtcgtcagctcgtgtcgtgagatgttgggttaagtcccgcaacgagcgcaacccctatctttagtagccagcagttcggctgnncactctagagagactgccagggataacctggaggaaggcggggatgacgtnnaatcatcatgccccnnatgatttgggctacacacgtgctacaatggtgactacaaagagaagcaagcctgcnngggggagcaaatctcaaaaaggtcatcccagttcggattgtactctgcaactcgagtacatgaagctggaatcgctagtaatcgcgaatcagaatgtcgcggtgaatacgttcccgggtcttgtacacaccgyycgtcactccatgggagtaggtaacgcccgaagtcagtgaccyaaccgtaaggagggagctgccgaaggcgggatctataactgggg

SEQ ID NO: 121 - DNA, 57 bp, Artificial Sequence
  Synthetic oligonucleotide
gaattcgagctcggtacccggggatcctctagagtcgacctgcaggcatgcaagctt

SEQ ID NO: 122 - DNA, 1515 bp, Thermoanaerobacter pseudoethanolicus
  misc_feature (69)..(69): n is a, c, g or t
cctggctcaggacgaacgctggcggcgtgcctaacacatgcaagtcgagcggtccggcagccaacttangncgggagccggatagcggcggacgggtgagtaacgcgtgggcaacctacccttaagaccgggataacacctcgaaaggggtgctaatactggataagctccttgtagggcatggtatgaggaggaaggtagcgggactaccgcttaaggatgggcccgcgtcccatcagctagttggtagggtaacggcctaccaaggcgacgacgggtagccggcctgagagggtggtcggccacactgggactgagacacggcccagactcctacgggaggcagcagtggggaatcttgcgcaatgggcgaaagcctgacgcagcgacgccgcgtgagcgaggaaggccttcgggtcgtaaagctcgatagtgtgggaagaagggatgacggtaccacacgaaagccccggctaactacgtgccagcagcctcggtaagacgtagggggcgagcgttgtccggaattactgggcgtaaagggcgcgtaggcggccgttcaagtcaggtgtaaaatacccgggctcaacccggggatagcacttgaaactgggcggctagagggcaggagaggggagtggaattcccggtgtagcggtgaaatgcgtagatatcgggaggaataccagtggcgaaggcgactctctggactgaccctgacgctgaggcgcgaaagcgtggggagcaaacaggattagataccctggtagtccacgccgtaaacgatgggtactaggtgtgggatgcggaagcattccgtgccgtagttaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtagtagcgagccgaaaggtgagcgaccttaccttaaaggtgaggagcctgcacaggtggtgcatggttgtcgtcagctcgtgtcgtgagatgttgggttaagtcccgcaacgagcgcaacccctgcctctagttgccagcgggtgaagccgggcacgctagagggactgccgtggacaacacggaggaaggtggggatgacgtcaaatcatcatgccctatatgccctgggccacacacgtgctacaatggccggtacagagggaagcgaagccgcgaggtggagcgaaacccaaaaagccggtccaagttcggattgcaggctgcaactcgcctgcatgaagtcggaatcgctagtaatcgcggatcagcatgccgcggtgaatacgttcccgggccttgtacacaccgcccgtcacaccacgagagtctgcaacacccgaagccgtgacccaaccgnaaggagggagccgtcgaaggtggggcagatgattggggtgaagtcgtaacaaggtagccgtatcggaaggtgcggctggatcacctcc

SEQ ID NO: 123 - DNA, 1395 bp, Thermoanaerobacter sp.
ctacacatgcagtcgagcgaagggagtactacggtacgaacttagcggcggacgggtgagtaacgcgtggacaatctaccctgtagaccgggataacacctcgaaaggggtgctaataccggataatgtcgagaagcggcatcgctttttgaagaaaggagagaatccgctataggaggagtccgcgtcccattagctagttggcgagggtaaaagcccaccaaggcgacgatgggtagccggcctgagagggtgaacggccacactggaactgagacacggtccagactcctacgggaggcagcagtggggaatattgtgcaatgggggaaaccctgacacagcgacgccgcgtgagtgaagaaggccttcgggtcgtaaagctcaatagtatgggaagaaagaaatgacggtaccatacgaaagccccggctaactacgtgccagcagccgcggtaatacgtagggggcgagcgttgtccggaattactgggcgtaaagagcacgtaggcggctataaaagtcagatgtgaaaaacctgggctcaaccgagggtatgcatctgaaactaaatagcttgagtcaaggagaggagagcggaattcctggtgtagcggtgaaatgcgtagagatcaggaagaataccagtggcgaaagcggctctctggacttgaactgacgctgaggtgcgaaagcgtggggagcaaacaggattagataccctggtagtccacgccgtaaacgatggatactaggtgtgggttagatataatccgtgccggagttaacgcaataagtatcccgcctggggagtacggccgcaaggttgaaactcaaaggaattgacgggggcccgcacaagcagcggagcatgtggtttaattcgaagcaacgcgaagaaccttaccagggcttgacatccacagaatcgagtagaaatacttgagtgcctcgtaagaggagctgtgagacaggtggtgcatggttgtcgtcagctcgtgtcgtgagatgttgggttaagtcccgcaacgagcgcaacccctgttggtagttaccagcgtaaagacggggactctaccgagactgccgtggataacacggaggaaggcggggatgacgtcaaatcatcatgccctttatgccctgggctacacacgtgctacaatggcctgaacagagggcagcgaaggagcgatccggagcgaatcccagaaaacaggtcccagttcagattgcaggctgcaacccgcctgcatgaagacggagttgctagtaatcgcggatcagcatgccgcggtgaatacgttcccgggccttgtacacaccgcccgtcacaccacgagagtttacaacacccgaagtcagtgacctaaccgcaagggaggagctgccgaa

SEQ ID NO: 124 - DNA, 1552 bp, Thermoanaerobacterium saccharolyticum; identical to SEQ ID NO: 116
  misc_feature (64)..(64): n is a, c, g or t

SEQ ID NO: 125 - DNA, 1553 bp, Thermoanaerobacterium saccharolyticum
tttgatcctggctcaggacgaacgctggcggcgtgcctaacacatgcaagtcgagcgatccggcactcaactaagcgcttacagaaaaagagagagaaaatgagtaaacgcaaagttgagtgccggatagcggcggacgggtgagtaacgcgtggacaatctaccctgtagtttgggataacacctcgaaaggggtgctaataccggataatgtcaagaagtggcatcactttttgaagaaaggagaaatccgctataggatgagtccgcgtcccattagctagttggcggggtaaaagcccaccaaggcgacgatgggtagccggcctgagagggtgaacggccacactggaactgagacacggtccagactcctacgggaggcagcagtggggaatattgtgcaatgggggaaaccctgacacagcgacgccgcgtgagcgaagaaggccttcgggtcgtaaagctcaatagtatgggaagatagtgacggtaccatacgaaagccccgggctactacgtgccagcagccgcggtaatacgtagggggcgagcgttgtccggaattactgggcgtaaagagcacgtaggcggctgtaaaagtcagatgtgaaaaacctgggctcaaccgagggtgtgcatctgaaactaaacagcttgagtcaaggagaggagagcggaattcctggtgtagcggtgaaatgcgtagagatcaggaagaataccagtggcgaaagcggctctctggacttgaactgacgctgaggtgcgaaagcgtggggagcaaacaggattagataccctggtagtccacgccgtaaacgatggatactaggtgtgggtgaagcatcatccgtgccggagttaacgcaataagtatcccgcctggggagtacggccgcaaggttgaaactcaaaggaattgacgggggcccgcacaagcagcggagcatgtggtttaattcgaagcaacgcgaagaaccttaccagggcttgacatccacagaatctggtagaaataccggagtgcctcgaaagaggagctgtgagacaggtggtgcatggttgtcgtcagctcgtgtcgtgagatgttgggttaagtcccgcaacgagcgcaacccctgttggtagttaccagcgtaaagacggggactctaccgagactgccgtggagaacacggaggaaggcggggatgacgtcaaatcatcatgccctttatgccctgggctacacacgtgctacaatggcctgaacagagggcagcgaaggagcgatccggagcgaatcccagaaaacaggtcccagttcagattgcaggctgcaacccgcctgcatgaagacggagttgctagtaatcgcggatcagcatgccgcggtgaatacgtttcccgggccttgtacacaccgcccgtcacaccacgagagtttacaacacccgaagtcagtgacctaaccgaaaggaaggagctgccgaaggtggggtaaatgattggggtgaagtcgtaacaaggtagccgtatcggaaggtgcggctggatcacctcctttctaa

SEQ ID NO: 126 - DNA, 1569 bp, Unknown
  Consensus Sequence
tttgatcctggctcaggacgaacgctggcggcgtgcctaacacatgcaagtcgagcgatccggcactcaantaagcgcttacagaaaaagangagcgaaantgagtaaacgctaagttgagtgccggatagcggcggacgggtgagtaacgcgtggacaatctaccctgtagtttgggataacacctcgaaaggggtgctaataccggataatgtcaagaagtggcatcgctttttgaagaaaggagagnnaatnccgctataggatgagtccgcgtcccattagctagttggcgngggtaaaagcccaccaaggcgacgatgggtagccggcctgagagggtgaacggccacactggaactgagacacggtccagactcctacgggaggcagcagtggggaatattgtgcaatgggggaaaccctgacacagcgacgccgcgtgagcgaagaaggccttcgggtcgtaaagctcaatagtatgggaagatagnantgacggtaccatacgaaagccccggctaactacgtgccagcagccgcggtaatacgtagggggcgagcgttgtccggaattactgggcgtaaagagcacgtaggcggctgtaaaagtcagatgtgaaaaacctgggctcaaccgagggtgtgcatctgaaactaaacagcttgagtcaaggagaggagagcggaattcctggtgtagcggtgaaatgcgtagagatcaggaagaataccagtggcgaaagcggctctctggacttgaactgacgctgaggtgcgaaagcgtggggagcaaacaggattagataccctggtagtccacgccgtaaacgatggatactaggtgtgggntgaggcatcatnccgtgccggagttaacgcaataagtatcccgcctggggagtacggccgcaaggttgaaactcaaaggaattgacgggggcccgcacaagcagcggagcatgtggtttaattcgaagcaacgcgaagaaccttaccagggcttgacatccacnnagaatcgggtagaaataccagagtgcctcgnnnaaagaggagctgtgagnacaggtggtgcatggttgtcgtcagctcgtgtcgtgagatgttgggttaagtcccgcaacgagcgcaacccctgttggtagttaccagcgnntaaagacggggactctaccgagactgccgtggagaacacggaggaaggcggggatgacgtcaaatcatcatgccctttatgccctgggctacacacgtgctacaatggcctgaacagagggcagcgaaggagcgatccggagcgaatcccagaaaacaggtcccagttcagattgcaggctgcaacccgcctgcatgaagacggagttgctagtaatcgcggatcagcatgccgcggtgaatacgttncccgggccttgtacacaccgcccgtcacaccacgagagtttacaacacccgaagtcagtgacctaaccgcaagggaggagctgccgaaggtggggtaaatgattggggtgaagtcgtaacaaggtagccgtatcggaaggtgcggctggatcacctcctttc

SEQ ID NO: 127 - DNA, 1061 bp, Unknown
  Consensus Sequence
gtgtatacaatatatttcttctttttagtaagaggaatgtataaaaataaatattttaaaggaagggacgatcttatgagcattattcaaaacatcattgaaaaagctaaaagtgataaaaagaaaattgttctgccagaaggtgcagaacccaggacattaaaagctgctgaaatagttttaaaagaaggaattgcagatttggtgcttcttggaaatgaagatgagataagaaatgctgcaaaagacttggacatatccaaagctgaaatcattgaccctgtaaagtctgaaatgtttgataggtatgctaatgatttttatgagttaaggaagagcaaaggaatcacgttggaaaaagccagagaaacaatcaaggataatatctattttggatgtatgatggttaaagaaggttatgctgatggattggtatctggcgctattcatgctactgcagatttattaagacctgcatttcagataattaaaacggctccaggagcaaagatagtatcaagcttttttataatggaagtgcctaattgtgaatatggtgaaaatggtgtattcttgtttgctgattgtgcggtcaatccatcgcctaatgcagaagaacttgcttctattgctgtacaatctgctaatactgcaaangaatttgttgggctttgaaccaaaagttgctatgctatcattttctacaaaaggtagtgcatcacatgaattagtagataaggtaagaaaagcgacagagatagcaaaagaattgatgccagatgttgctantcgatggtgaattgcaattggatgctgctcttgttaaagaagttgcagagctaaaagcgccgggaagcaaagttgcgggatgtgcaaatgtgcttatattccctgatttacaagctggtaatataggatataagcttgtacagagattagctaaggcaaatgcaattggacctataacacaaggaatgggtgcaccggttaatgatttatcaagaggatgcagctatagagatattgttgacgtaatagcaacaacagctgtgcaggctcaa

SEQ ID NO: 128 - DNA, 1213 bp, Unknown
  Consensus Sequence
atgaaaattatgaaaattctggttattaattgtggaagttcttcactaaaantatcaattgattgaatcaantgatggaaatgtgctggcaaaaggccttgctgaaagaatcggcataaatgattccctgttgacncataatgctaacggnnnagaaaaaatcaagataaaaaaagacatgaaagatcacaaagacgcaataaaattggttttagatgctttggtaagtagtgactacggcgttataaaggatatgtctgagatagatgctgtaggacatagagttgttcacggaggagagtcttttacatcatcagttctcataaatgatgaagtgttaaaagcgataacagattgtatagaattagctccactgcacaatcctgctaatatagaaggaattaaagcttgccagcaaatcatgccaaacgttccaatggtggcggtatttgatacagcctttcatcagacaatgcctgattatgcatatctttatccaataccttatgaatactacacaaagtacaggatcagaagatatggatttcatggcacatcgcataaatatgtttcaaatagggctgcagagattttgaataaacctattgaagatttgaaaatcataacttgtcatcttggaaatggctccagtattgctgctgtcaaatatggtaaatcaattgacacaagcatgggatttacaccattagaaggtttggctatgggtacacgatctggaagtatagacccatccattatttcttatcttatggaaaaagaaaatataagtgctgaagaggtagtaaatatattaaataaaaaatctggtgtttacggtatttcaggaataagcagcgattttagagatttagaagatgccgcctttaaaaatggagatgaaagagctcagttggctttaaatgtgtttgcatatcgagtaaagaagacgattggcgcttatgcagcagctatgggaggcgttgatgtcattgtatttacagcaggtgttggtgaaaatggtcctgagatacgagaatttatacttgatggattagagtttttagggttcagcttggataaagaaaaaaataaagtcagaggaaaagaaactattatatctacgccgaattcaaaagttagcgtgatggttgtgcctactaatgaagaatatatgattgctaaagatactgaaaagattgtaaagagtataaaa

SEQ ID NO: 129 - DNA, 935 bp, Unknown
  Consensus Sequence
atgagcaaagtagcnataataggttctggatttgtaggtgctacatctgcatttacactggctttaagtgggactgtgacagatattgtnttagtagatttaaacaaggacaaggcnataggcgatgcactggatataagccatggcataccgtttatacagcctgtaaatgtgtatgcaggtgactacaaagatgttgaaggcgcagatgtaatagttgtgacagcaggtgctgctcaaaagccgggagagacnaggcttgaccttgtgaagaaaaatacagctatatttaagtccatgatacctgagcttnttaaagtacaatgacaaggctatatatttgattgtnacaaatcctgtagatatactgacgtacgttacatacaagatatctggacttccatggggcagagttttcggttctggcactgttcttgacagttcaaggtttaggtatcttttaagcaagcattgcaatatagatnccgagaaatgtccacggaaggataattggcgagcatggtgatacagagtttgcagcatggagcataacaaacatatcaggaatatcatttaatgagtactgcagtttatgcggacgcgtctgtaacacaaatttcagaaaggaagtagaagatgaagttgtaaatgctgcttataagataatagacaaaaagggtgctacatattatgctgtggctgttgcagtaagaaggattgtggagtgtatcttaagagatgaaaattccattctnacagtntcatctccattaaatggncaatacggtgtnanagatgtntctttaagcttgccatcnattgtnggcagaaatggngttgcaaggattctgganttgcctttntctgangaagaagttgagaagtttagacattcagcaagngttatggcagatgtnataaaacagttngatata